(19) United States
(12) Patent Application Publication
(10) Pub. No.: US 2023/0332157 A1
(43) Pub. Date: Oct. 19, 2023

(54) shRNA TARGETING UBE3A-ATS TO RESTORE PATERNAL UBE3A GENE EXPRESSION IN ANGELMAN SYNDROME

(71) Applicants: University of Connecticut, Farmington, CT (US); Ovid Therapeutics Inc., New York, NY (US)

(72) Inventors: Stormy CHAMBERLAIN, Oxford, CT (US); Noelle Germain, Watertown, CT (US); Peter Perrino, Glastonbury, CT (US)

(21) Appl. No.: 18/179,726

(22) Filed: Mar. 7, 2023

Related U.S. Application Data
(60) Provisional application No. 63/317,154, filed on Mar. 7, 2022.

(51) Int. Cl. C12N 15/113; C12N 15/86

(52) U.S. Cl. CPC C12N 15/1137; C12N 15/86; C12N 2310/122; C12N 2310/531; C12N 2740/15043

(57) ABSTRACT

Provided herein are compositions and methods for activating expression from the paternally-inherited allele of UBE3A in Angelman syndrome using viral vector delivery of short hairpin RNAs. Provided herein are compositions and methods for reducing or eliminating expression of UBE3A-ATS in Angelman syndrome using viral vector delivery of short hairpin RNAs.

### CROSS-REFERENCE TO RELATED APPLICATION

This application claims benefit and priority to U.S. Provisional Application No. 63/317,154, filed Mar. 7, 2022, which is incorporated herein by reference in its entirety.

### STATEMENT OF GOVERNMENT SUPPORT

This invention was made with government support under Contract No. 1R01HD094953 awarded by the National Institutes of Health. The government has certain rights in the invention.

### TECHNICAL FIELD

The present disclosure relates to compositions and methods for activating expression from the paternally-inherited allele of UBE3A in subjects having Angleman Syndrome using short hairpin RNAs.

### REFERENCE TO SEQUENCE LISTING

The application contains a Sequence Listing which has been submitted electronically in .XML format and is hereby incorporated by reference in its entirety. Said .XML copy, created on Apr. 7, 2023, is named “2262-97.xml” and is 717,274 bytes in size. The sequence listing contained in this .XML file is part of the specification and is hereby incorporated by reference herein in its entirety.

### BACKGROUND

Angelman syndrome (AS) is a neurodevelopmental disorder affecting ˜1/15,000 individuals. Individuals with AS have developmental delay, severe cognitive impairment, ataxic gait, frequent seizures, short attention span, absent speech, and characteristic happy demeanor. Neurons derived from induced pluripotent stem cells (iPSC) from AS patients exhibit a depolarized resting membrane potential, delayed action potential development, and reduced spontaneous synaptic activity. Fink, J. J., T. M. Robinson, N. D. Germain, C. L. Sirois, K. A. Bolduc, A. J. Ward, F. Rigo, S. J. Chamberlain and E. S. Levine (2017). “Disrupted neuronal maturation in Angelman syndrome-derived induced pluripotent stem cells.” Nat Commun 8: 15038. AS affects a relatively large patient population; a contact registry with >3,000 patients has been established and ˜250 new diagnoses of AS are made each year. Individuals with AS require life-long care.

AS is caused by loss of function from the maternal copy of UBE3A, a gene encoding an E3 ubiquitin ligase. This loss of function mutation can be caused by any type of gene mutation in the maternal allele. UBE3A is expressed exclusively from the maternal allele in neurons. All individuals with AS have a normal paternal UBE3A allele that is epigenetically silenced in neurons in cis by a long, non-coding RNA, called UBE3A antisense transcript (UBE3A-ATS) (Rougeulle et al., 1997, Nat Genet 17, 14-15; Chamberlain and Brannan, 2001, Genomics 73, 316-322). Reactivation of the paternal allele has been shown to restore UBE3A protein expression and alleviate behavioral deficits in an AS mouse model. The restoration of UBE3A expression in humans is expected to ameliorate the disease, especially if it is restored in infants.

### SUMMARY

Provided herein is a novel treatment for Angelman syndrome by inhibiting the silencing of paternal UBE3A and enabling the expression of paternal UBE3A from its native regulatory elements, thus replacing or augmenting missing maternal UBE3A. Increased expression of UBE3A in neurons is accomplished by terminating transcription of UBE3A-ATS. Since the native regulatory elements control expression, overexpression of UBE3A is prevented. This approach can improve AS symptoms through a single treatment and eliminate the need for multiple treatments.

Provided herein is a polynucleotide sequence including: 5′-GATATCACCTTACAGAAATTACTCGAGTAATTTCTGTAAGGTGATATC-3′ (SEQ ID No: 2). Expression vectors including SEQ ID NO: 2 are provided. In embodiments, the expression vector is an adeno-associated viral (AAV) vector or a lentiviral vector. Pharmaceutical compositions including the foregoing are provided.

Provided herein is a polynucleotide encoding a shRNA including a nucleotide sequence that is at least 85%, at least 90%, at least 95%, or 100% complementary to a RNA encoded by any of SEQ ID NOs: 3-489. In embodiments, the polynucleotide is SEQ ID NO: 2. In embodiments, the shRNA causes activation of, or an increase in, expression of paternal UBE3A. In embodiments, the shRNA causes a reduction of expression of paternal UBE3A-ATS. Expression vectors including the shRNA are provided. In embodiments, the expression vector is an adeno-associated viral (AAV) vector or a lentiviral vector. Pharmaceutical compositions including the foregoing are provided.

Provided herein is a method of treating Angelman syndrome including administering to a patient in need thereof the polynucleotide of SEQ ID NO: 2. In embodiments, the polynucleotide of SEQ ID NO: 2 encodes a shRNA which causes a reduction of expression of paternal UBE3A-ATS. In embodiments, the polynucleotide of SEQ ID NO: 2 encodes a shRNA which causes activation of, or an increase in, expression of paternal UBE3A gene.

Provided herein is a method of treating Angelman syndrome including administering to a patient in need thereof a polynucleotide encoding a shRNA including a nucleotide sequence that is at least 85%, at least 90%, at least 95%, or 100% complementary to a RNA encoded by any of SEQ ID NOs: 3-489. In embodiments, the polynucleotide is SEQ ID NO: 2. In embodiments, the shRNA causes activation of, or an increase in, expression of paternal UBE3A. In embodiments, the shRNA causes a reduction of expression of paternal UBE3A-ATS.

In embodiments, SEQ ID NO: 2 encodes a shRNA capable of inhibiting the silencing of paternal UBE3A. In embodiments, the SEQ ID NO: 2 is contained within an expression vector. In embodiments, the expression vector is an adeno-associated viral (AAV) vector or a lentiviral vector. In embodiments, a method is provided of inhibiting the silencing of paternal UBE3A gene by the RNA antisense transcript encoded by SEQ ID NO: 1 which includes administering to a patient in need thereof an amount of SEQ ID NO: 2 which is effective to cut the RNA antisense transcript encoded by SEQ ID NO: 1.

In embodiments, a method is provided of inhibiting the silencing of paternal UBE3A gene by the RNA antisense transcript encoded by SEQ ID NO: 1 which includes administering to a patient in need thereof, an amount of a shRNA including a nucleotide sequence that is at least 85%, at least 90%, at least 95%, or 100% complementary to a RNA encoded by any of SEQ ID NOs: 3-489, which is effective to cut the RNA antisense transcript encoded by SEQ ID NO: 1.

In embodiments, a shRNA provided herein is encoded by a portion of SEQ ID NO: 2, e.g., having the bold nucleotides, which has been shortened by one, two, three or four nucleotides at either end of the bold nucleotides. Likewise, in embodiments, the shRNA provided herein can contain a portion of SEQ ID NO: 2, e.g., having the italicized nucleotides, which has been shortened by one, two or three nucleotides at either end of the italicized nucleotides.

In embodiments, a polynucleotide sequence is provided as follows:

(SEQ ID NO: 506)
5′-GATATCACCTTACAGAAATTAnnnnnnnnTAATTTCTGTAAGGTGA
TATC-3′, wherein nnnnnnnn can be
(SEQ ID NO: 490)
CTCGAG,
(SEQ ID NO: 491)
TCAAGAG,
(SEQ ID NO: 492)
TTCG
or
(SEQ ID NO: 493)
GAAGCTTG.

In embodiments, a polynucleotide sequence is provided which includes a first portion, a second portion and a third portion, the first portion comprising any of SEQ ID NOs: 3-489, the second portion comprising any of SEQ ID Nos: 490, 491, 492, or 493, and the third portion comprising respective nucleotide sequences complementary to those in SEQ ID NOs: 3-489.

### BRIEF DESCRIPTION OF THE DRAWINGS

FIG. 1 shows chromosomal mutations in Angelman Syndrome.

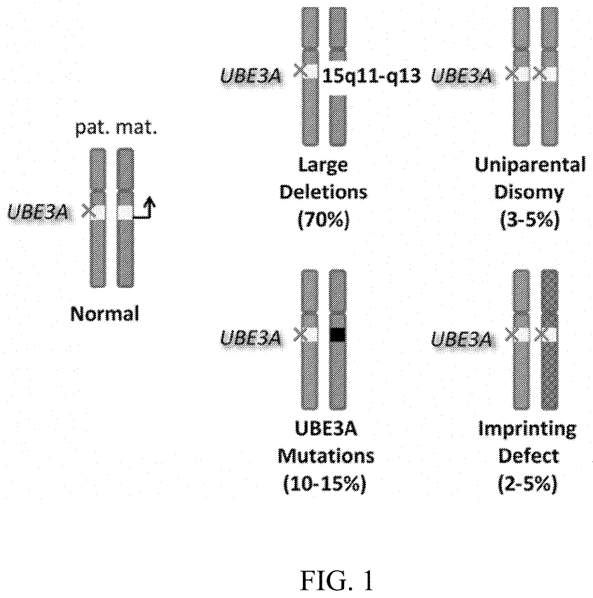

FIG. 2 shows a diagram of paternal UBE3A gene.

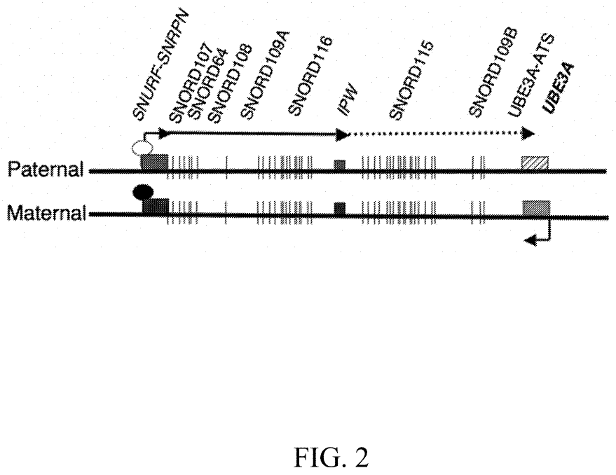

FIG. 3A and FIG. 3B show genomic locations of shRNA targets (solid callout). UCSC Genome Browser view of the 15q11-q13 region containing the imprinted SNHG14/UBE3A locus (dashed-line callout). Location of shRNA targets within the UBE3A ATS region (ATS-shRNA2) and the SNORD115 snoRNA cluster.

FIG. 4 is a bar graph showing qRT-PCR analysis of Angelman syndrome iPSC-derived neurons following treatment with either SNHG14-targeting shRNAs (551-2, ATS shRNA1-4, ATS shRNA2_3G) or non-targeting control shRNA (SCRAM). Two shRNAs, 551-2 and ATS shRNA2, knocked down UBE3A-ATS and activated paternal UBE3A.

FIG. 5 is a bar graph showing qRT-PCR analysis of Angelman syndrome iPSC-derived neurons following treatment with either SNHG14-targeting shRNAs (ATS shRNA 2), non-targeting control shRNA (SCRAM), or untreated (UTC). ATS shRNA2 knocked down UBE3A-ATS and activated paternal UBE3A.

### DETAILED DESCRIPTION

UBE3A is a gene which encodes the E3 ubiquitin ligase. The genomic coordinates for UBE3A are hg19 chr15:25,582,381-25,684,175 on the minus strand. There are three normal isoforms of UBE3A: Isoform 1 (accession number X98032); Isoform 2 (accession number X98031); and isoform 3 (Accession number X98033). In neurons, UBE3A is expressed exclusively from the maternal allele. The paternal UBE3A allele is epigenetically silenced by the long, non-coding RNA UBE3A antisense transcript (UBE3A-ATS) encoded by SEQ ID NO: 1. The genomic coordinates for UBE3A ATS are hg19 chr15:25,223,730-25,664,609 on the plus strand. The following genomic coordinates are of particular interest: hg19 chr15:25,522,751-25,591,391 on the plus strand.

UBE3A-ATS/Ube3a-ATS (human/mouse) is the antisense DNA strand that is transcribed as part of a larger transcript called SNHG14 (SNORNA HOST GENE 14) at the UBE3A locus. Human UBE3A ATS is expressed as a part of SNHG14 exclusively from the paternal allele in the central nervous system (CNS). The transcript is about 600 kbs long, starts at SNRPN and extends through most of UBE3A. SNHG14 (Small Nucleolar RNA Host Gene 14) encodes a non-coding RNA and is affiliated with the lncRNA class. SNHG14 is located within the Prader-Willi critical region and produces a long, spliced maternally-imprinted RNA that initiates at one of several promoters of the SNRPN (small nuclear ribonucleoprotein polypeptide N) gene. This transcript serves as a host RNA for two clusters of C/D box small nucleolar RNAs, SNORD116 and SNORD115. See, Runte et al., 2001, Hum Mol Genet 10, 2687-2700. This RNA extends into the ubiquitin protein ligase E3A (UBE3A) gene and is thought to regulate imprinted expression of UBE3A in the brain. The promoter of SNRPN is the Prader-Willi syndrome Imprinting Center (PWS-IC) and about 35 kbs upstream of the PWS-IC is the Angelman syndrome Imprinting Center (AS-IC). These two regions are thought to control the expression of the entire SNHG14 transcript.

SNURF/SNRPN is a bicistronic gene that encodes two protein-coding transcripts, SNURF and SNRPN. Both SNURF and SNRPN proteins localize to the cell nucleus. SNRPN is a small nuclear ribonucleoprotein, and the function of SNURF is unknown. The transcript that initiates at SNRPN/SNURF also encodes the SNHG14 transcript. Within the introns of SNHG14 are sequences for several C/D box snoRNAs. Box C/D small nucleolar RNAs (SNORDs) represent a well-defined family of small non-coding RNAs that exert their regulatory functions via antisense-based mechanisms. Most C/D box snoRNAs function in non-mRNA methylation.

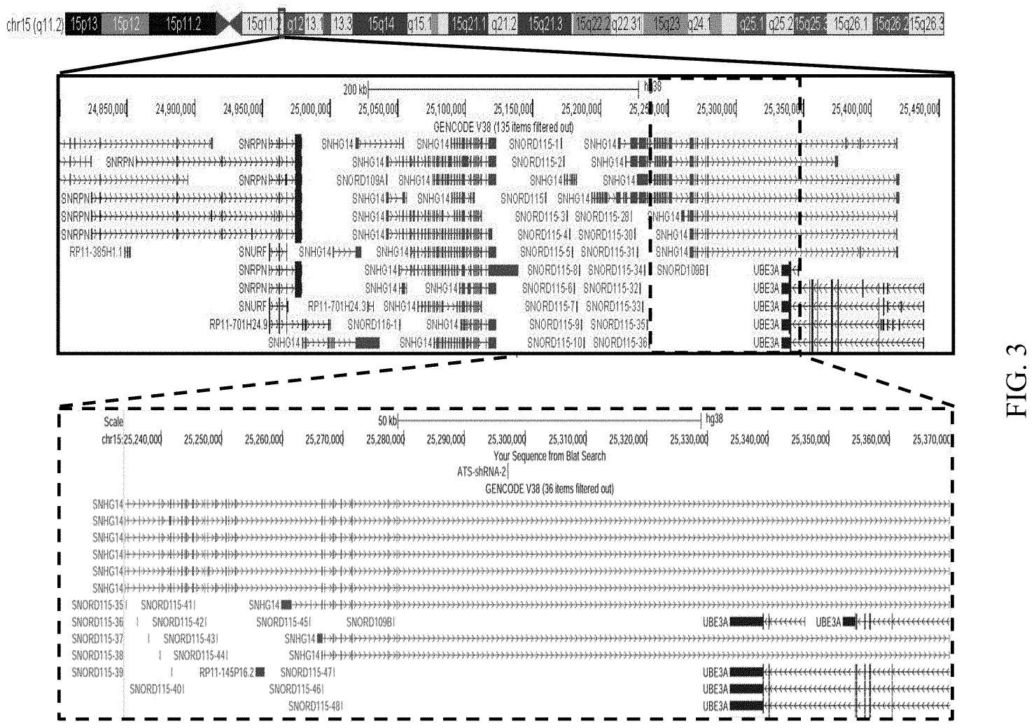

Many orphan snoRNAs are generated from two large, imprinted chromosomal domains at human 15q11q13 and 14q32. See, e.g., FIG. 3. As indicated above, the imprinted human 15q11q13 region—also known as the Prader-Willi Syndrome (PWS)/Angelman Syndrome (AS) locus or SNURF-SNRPN domain—contains several paternally expressed, protein coding genes as well as numerous paternally expressed, neuronal-specific snoRNA genes organized as two main repetitive DNA arrays: the SNORD116 and SNORD115 clusters composed of 29 and 47 related gene copies, respectively.

SNORD115 encodes a small nucleolar RNA (snoRNA) that is found clustered with dozens of other similar snoRNAs on chromosome 15. These genes are found mostly within introns of the SNHG14 transcript, which is paternally imprinted and from the PWS/AS region.

The compositions and methods described herein are drawn to targeting UBE3A ATS to unsilence the paternal UBE3A allele. Effective inhibition of UBE3A-ATS by short hairpin RNAs (shRNA) described herein result in a reduction in UBE3A-ATS expression levels and a concomitant increase in the expression levels of the paternal UBE3A allele.

In embodiments, compositions and methods herein relate to the treatment or prevention of AS. A patient in need of such treatment or prevention has AS or is at risk for developing AS. As used herein, the term “patient in need” includes any mammal in need of these methods of treatment or prophylaxis, including humans. The subject may be male or female. In certain aspects, the patient in need, having AS, treated according to the methods and compositions provided herein may show an improvement in anxiety, learning, balance, motor function, and/or seizures, or the method may return the neuronal resting membrane potential to about −70 mV, ameliorate the action potential development delay, increase spontaneous synaptic activity, and may ameliorate additional alterations in the neuronal phenotype relating to rheobase, action potential characteristics (e.g. shape), membrane current, synaptic potentials, and/or ion channel conductance.

In embodiments, a polynucleotide includes a first nucleotide sequence encoding a short hairpin RNA (shRNA) that results in decreased expression of the UBE3A-ATS sequence (SEQ ID NO: 1). For example, a portion of the shRNAs described herein may be complementary to the RNA sequence encoded by SEQ ID NO: 1 or a sequence contained therein. In embodiments, the shRNAs described herein are RNA polynucleotides encoded by a first nucleotide sequence. The polynucleotide encompassing the first nucleotide sequence may be a DNA polynucleotide suitable for cloning into an appropriate vector (e.g., a plasmid) for culturing and subsequent production of viral particles. In turn, viral particles may contain the DNA polynucleotide with the nucleotide coding sequence in a form suitable for infection. Thus, the first nucleotide sequence may be a DNA sequence cloned into a plasmid for viral particle production or encapsulated in a viral particle. As retroviruses carry nucleotide coding sequences in the form of RNA polynucleotides, retroviral particles (e.g., lentivirus) contain an RNA polynucleotide that includes the first nucleotide sequence as a corresponding RNA sequence.

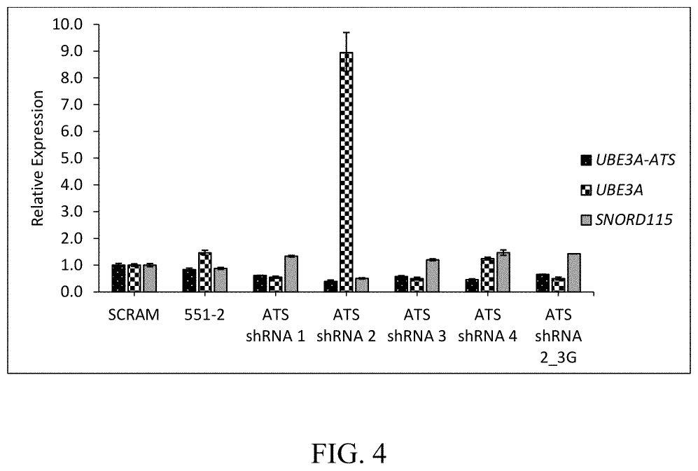

Disclosed herein are novel shRNAs that cut UBE3A ATS thereby reducing UBE3A-ATS expression and, in turn activate, the paternally inherited copy of UBE3A in neurons. This provides the UBE3A gene product in a cell type that is missing the protein in Angelman syndrome. There is a potential search space of about −60 kb in the genomic LNCAT sequence which may provide potential shRNA targets. However, not every predicted sequence actually reduces UBE3A-ATS and restores UBE3A. Accordingly, as shown by the certain examples herein, it is difficult to predict which sequences will or will not work. See, e.g., FIG. 4.

The first nucleotide sequence encodes a shRNA. For example, the first nucleotide sequence may be SEQ ID NO: 2 (5′-GATATCACCTTACAGAAATTACTCGAGTAATTTCTGTAAGGTGATATC-3′). The first nucleotide sequence may also be a modified SEQ ID NO: 2 having the bold nucleotides in SEQ ID NO: 2 replaced by any of SEQ ID NOs: 4-489 and the italicized nucleotides in SEQ ID NO: 2 replaced by nucleotides complementary to those in SEQ ID NOs: 4-489 As used herein, “targets” means an operative RNA polynucleotide capable of undergoing hybridization to a nucleotide sequence through hydrogen bonding, such as to a nucleotide sequence transcribed from a nucleotide sequence within the larger genomic sequence of UBE3A-ATS. The hybridization of an operative RNA polynucleotide to a nucleotide sequence transcribed from a nucleotide sequence with the larger genomic sequence of UBE3A-ATS may result in the reduced expression of UBE3A-ATS levels in the presence of the operative RNA polynucleotide compared to the expression levels of UBE3A-ATS in the absence of the operative RNA polynucleotide. In embodiments, the operative RNA polynucleotide encompasses the nucleotide sequence of the shRNA that is complementary to the RNA sequence encoded within the larger genomic sequence of UBE3A-ATS. For example, the shRNA contains nucleotide sequences complementary to the RNA sequences encoded by SEQ ID NO: 3 and SEQ ID NOs: 4-489. The operative RNA polynucleotide thus refers to an operative portion of the shRNA following assimilation of the shRNA into a target organism and processing into a functional state.

“Reduce expression” refers to a reduction or blockade of the expression or activity of UBE3A ATS and does not necessarily indicate a total elimination of expression or activity. Mechanisms for reduced expression of the target include hybridization of an operative RNA polynucleotide with a target sequence or sequences transcribed from a sequence or sequences within the larger genomic UBE3A-ATS sequence (SEQ ID NO: 1), wherein the outcome or effect of the hybridization is either target degradation or target occupancy with concomitant stalling of the cellular machinery involving, for example, transcription or splicing.

Without wishing to be bound to a particular theory, the shRNA herein may inhibit the silencing of paternal UBE3A by: (1) cutting the RNA transcript encoded by SEQ ID NO: 1; (2) reducing steady-state levels (i.e., baseline levels at homeostasis) of the RNA transcript encoded by SEQ ID NO: 1; and (3) terminating transcription of SEQ ID NO: 1. For example, cutting and reduction of steady-state levels of the RNA transcript encoded by SEQ ID NO: 1 may occur via a mechanism involving a RNA-induced silencing complex (RISC). shRNA may utilize RISC. Once the vector carrying the genomic material for the shRNA is integrated into the host genome, the shRNA genomic material is transcribed in the host into pri-microRNA. The pri-microRNA is processed by a ribonuclease, such as Drosha, into pre-shRNA and exported from the nucleus. The pre-shRNA is processed by an endoribonuclease such as Dicer to form small interfering RNA (siRNA). The siRNA is loaded into the RISC where the sense strand is degraded and the antisense strand acts as a guide that directs RISC to the complementary sequence in the mRNA. RISC cleaves the mRNA when the sequence has perfect complementary and represses translation of the mRNA when the sequence has imperfect complementary. Thus, the shRNA encoded by the first nucleic acid sequence increases expression of paternal UBE3A by decreasing the steady-state levels of UBE3A-ATS RNA.

As used herein, the term “nucleic acid” refers to molecules composed of monomeric nucleotides. Examples of nucleic acids include ribonucleic acids (RNA), deoxyribonucleic acids (DNA), single-stranded nucleic acids, double-stranded nucleic acids, small interfering ribonucleic acids (siRNA), and short hairpin RNAs (shRNAs). “Nucleotide” means a nucleoside having a phosphate group covalently linked to the sugar portion of the nucleoside. “Oligonucleotide” or “polynucleotide” means a polymer of linked nucleotides each of which can be modified or unmodified, independent one from another.

As used herein, a “short hairpin RNA (shRNA)” includes a conventional stem-loop shRNA, which forms a precursor microRNA (pre-miRNA). “shRNA” also includes micro-RNA embedded shRNAs (miRNA-based shRNAs), wherein the guide strand and the passenger strand of the miRNA duplex are incorporated into an existing (or natural) miRNA or into a modified or synthetic (designed) miRNA. When transcribed, a conventional shRNA (i.e., not a miR-451 shRNA mimic) forms a primary miRNA (pri-miRNA) or a structure very similar to a natural pri-miRNA. The pri-miRNA is subsequently processed by Drosha and its cofactors into pre-shRNA. Therefore, the term “shRNA” includes pri-miRNA (shRNA-mir) molecules and pre-shRNA molecules.

A “stem-loop structure” refers to a nucleic acid having a secondary structure that includes a region of nucleotides which are known or predicted to form a double strand or duplex (stem portion) that is linked on one side by a region of predominantly single-stranded nucleotides (loop portion). It is known in the art that the loop portion is at least 4 nucleotides long, 6 nucleotides long (e.g., the underlined sequence in SEQ ID NO: 2), 8 nucleotides long, or more. The terms “hairpin” and “fold-back” structures are also used herein to refer to stem-loop structures. Such structures are well known in the art and the term is used consistently with its known meaning in the art. For example, CTCGAG (SEQ ID NO: 490), TCAAGAG (SEQ ID NO: 491), TTCG (SEQ ID NO: 492), and GAAGCTTG (SEQ ID NO: 493) are suitable stem-loop structures. As is known in the art, the secondary structure does not require exact base-pairing. Thus, the stem can include one or more base mismatches or bulges. Alternatively, the base-pairing can be exact, i.e., not include any mismatches. In embodiments, a polynucleotide sequence is provided as follows: 5′-GATATCACCTTACAGAAATTAnnnnnnnnTAATTTCTGTAAGGTGATATC-3′ (SEQ ID NO: 506), wherein nnnnnnnn can be CTCGAG (SEQ ID NO: 490), TCAAGAG (SEQ ID NO: 491), TTCG (SEQ ID NO: 492) or GAAGCTTG (SEQ ID NO: 493). In embodiments, a polynucleotide sequence is provided which includes a first portion, a second portion and a third portion, the first portion comprising any of SEQ ID NOs: 3-489, the second portion comprising any of SEQ ID Nos: 490, 491, 492, or 493, and the third portion comprising respective nucleotide sequences complementary to those in SEQ ID NOs: 3-489.

In embodiments, shRNAs can include, without limitation, modified shRNAs, including shRNAs with enhanced stability in vivo. Modified shRNAs include molecules containing nucleotide analogues, including those molecules having additions, deletions, and/or substitutions in the nucleobase, sugar, or backbone; and molecules that are cross-linked or otherwise chemically modified. The modified nucleotide(s) may be within portions of the shRNA molecule, or throughout it. For instance, the shRNA molecule may be modified, or contain modified nucleic acids in regions at its 5′ end, its 3′ end, or both, and/or within the guide strand, passenger strand, or both, and/or within nucleotides that overhang the 5′ end, the 3′ end, or both. (See Crooke, U.S. Pat. Nos. 6,107,094 and 5,898,031; Elmen et al., U.S. Publication Nos. 2008/0249039 and 2007/0191294; Manoharan et al., U.S. Publication No. 2008/0213891; MacLachlan et al., U.S. Publication No. 2007/0135372; and Rana, U.S. Publication No. 2005/0020521; all of which are hereby incorporated by reference.)

shRNAs herein include a nucleotide sequence complementary to a RNA nucleotide sequence transcribed from within the full genomic UBE3A ATS sequence (SEQ ID NO: 1) and inhibit the silencing of paternal UBE3A by UBE3A-ATS. In embodiments, shRNAs include a nucleotide sequence complementary to RNA sequences encoded by SEQ ID NOs: 4-489. In embodiments, a shRNA includes a nucleotide sequence complementary to a RNA sequence encoded by SEQ ID NO: 3 (5′-GATATCACCTTACAGAAATTA-3′, UBE3A-ATS artificial/synthetic target). In embodiments, the shRNA is encoded by the nucleotide sequence of SEQ ID NO: 2. In embodiments, the nucleotide sequence included in the shRNA and complementary to the RNA nucleotide sequence transcribed from the UBE3A-ATS gene is 17-21 nucleotides in length. The complementary nucleotides may be contiguous or may be interspersed with non-complementary nucleotides. In embodiments, the complementary nucleotide sequence is 21 nucleotides in length as indicated by the bold sequence in SEQ ID NO: 2. The shRNA may include a nucleotide sequence wherein 17, 18, 19, 20, or 21 nucleotides are complementary to the nucleotides in SEQ ID NOs: 3 or 4-489. The 17, 18, 19, 20, or 21 complementary nucleotides may be contiguous or may be interspersed with non-complementary nucleotides. The overall length of the shRNA, including the loop may be 40-50 nucleotides in length, e.g., 44-48 nucleotides, e.g., 48 nucleotides.

Methods of determining whether a sequence is specifically hybridizable to a target nucleic acid are well known in the art. In embodiments, the shRNA polynucleotides provided herein include a nucleic acid sequence specifically hybridizable with a RNA sequence transcribed from the UBE3A-ATS (SEQ ID NO: 1).

The shRNA may include an RNA polynucleotide containing a region of 17-21 linked nucleotides complementary to the RNA target sequence, wherein the RNA polynucleotide region is at least 85% complementary over its entire length to an equal length region of a UBE3A-ATS RNA nucleic acid sequence. In embodiments, the RNA polynucleotide region is at least 90%, at least 95%, or 100% complementary over its entire length to an equal length region of a UBE3A-ATS RNA nucleic acid sequence.

The shRNA may include a nucleotide sequence at least 85% complementary to, and of equal length as, a RNA sequence encoded by SEQ ID NO: 3 or any of SEQ ID NOs: 4-489. The shRNA may include a nucleotide sequence at least 90% complementary to, and of equal length as, a RNA sequence encoded by SEQ ID NO: 3 or any of SEQ ID NOs: 4-489. The shRNA may include a nucleotide at least 95% complementary to, and of equal length as, a RNA sequence encoded by SEQ ID NO: 3 or any of SEQ ID NOs: 4-489. The shRNA or microRNA may encompass a nucleotide sequence 100% complementary to, and of equal length as, a RNA sequence encoded by SEQ ID NO: 3 or any of SEQ ID NOs: 4-489.

In embodiments, the shRNA is a single-stranded RNA polynucleotide. In embodiments, the RNA polynucleotide is a modified RNA polynucleotide. A percent complementarity is used herein in the conventional sense to refer to base pairing between adenine and thymine, adenine and uracil (RNA), and guanine and cytosine.

Non-complementary nucleobases between a shRNA and an UBE3A-ATS nucleotide sequence may be tolerated provided that the shRNA remains able to specifically hybridize to a UBE3A-ATS nucleotide sequence. Moreover, a shRNA may hybridize over one or more segments of a UBE3A-ATS nucleotide sequence such that intervening or adjacent segments are not involved in the hybridization event (e.g., a loop structure, mismatch or hairpin structure).

In embodiments, the shRNA provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a UBE3A-ATS RNA nucleotide sequence, a UBE3A-ATS region, UBE3A-ATS segment, or specified portion thereof. Percent complementarity of a shRNA with an UBE3A-ATS nucleotide sequence can be determined using routine methods.

For example, a shRNA in which 18 of 20 nucleobases are complementary to a UBE3A-ATS region and would therefore specifically hybridize, would represent 90 percent complementarity. In this example, the remaining non-complementary nucleobases may be clustered or interspersed with complementary nucleobases and need not be contiguous to each other or to complementary nucleobases. As such, a shRNA which is 18 nucleobases in length having four non-complementary nucleobases which are flanked by two regions of complete complementarity with the target nucleotide sequence would have 77.8% overall complementarity with the target nucleotide sequence and would thus fall within the subject matter disclosed herein. Percent complementarity of a shRNA with a region of a UBE3A-ATS nucleotide sequence can be determined routinely using BLAST programs (basic local alignment search tools) and PowerBLAST programs known in the art (Altschul et al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In embodiments, the shRNA provided herein, or specified portions thereof, are fully complementary (i.e., 100% complementary) to a UBE3A-ATS nucleotide sequence, or specified portion of the transcription product of SEQ ID NO: 1 thereof. For example, a shRNA may be fully complementary to a UBE3A-ATS nucleotide sequence, or a region, or a segment or sequence thereof. As used herein, “fully complementary” means each nucleobase of a shRNA is capable of precise base pairing with the corresponding RNA nucleobases transcribed from a UBE3A-ATS nucleotide sequence.

In embodiments, the shRNA provided herein can contain a portion of SEQ ID NO: 2, e.g., having the bold nucleotides, which has been shortened by one, two, three or four nucleotides at either end of the bold nucleotides. Likewise, in embodiments, the shRNA provided herein can contain a portion of SEQ ID NO: 2, e.g., having the italicized nucleotides, which has been shortened by one, two or three nucleotides at either end of the italicized nucleotides. For example,

(SEQ ID NO: 494)
5′-ATATCACCTTACAGAAATTACTCGAGTAATTTCTGTAAGGTGATA
T-3′
(SEQ ID NO: 495)
5′-TATCACCTTACAGAAATTACTCGAGTAATTTCTGTAAGGTGATA-3′
(SEQ ID NO: 496)
5′-ATCACCTTACAGAAATTACTCGAGTAATTTCTGTAAGGTGAT-3′
(SEQ ID NO: 497)
5′-TCACCTTACAGAAATTACTCGAGTAATTTCTGTAAGGTGA-3′
(SEQ ID NO: 498)
5′-GATATCACCTTACAGAAATTCTCGAGAATTTCTGTAAGGTGATA
TC-3′
(SEQ ID NO: 499)
5′-GATATCACCTTACAGAAATCTCGAGATTTCTGTAAGGTGATATC-3′
(SEQ ID NO: 500)
5′-GATATCACCTTACAGAACTCGAGTTCTGTAAGGTGATATC-3′
(SEQ ID NO: 501)
5′-GATATCACCTTACAGACTCGAGTCTGTAAGGTGATATC-3′.

Similarly, in embodiments, the sequences shown in any of SEQ ID NOs: 4-489 and/or their complements can be shortened by one, two, three or four nucleotides at either end and incorporated into shRNAs.

An effective concentration or dose of the shRNA may inhibit the silencing of paternal UBE3A by UBE3A ATS by at least 20%, at least 30%, at least 40%, at least 50%, at least 60%, at least 70%, at least 80%, at least 90%, or at least 100%.

An effective concentration or dose of the shRNA may terminate transcription of UBE3A-ATS by at least 20%, at least 30%, at least 40%, at least 50%, at least 60%, at least 70%, at least 80%, at least 90%, or at least 100%.

An effective concentration or dose of the shRNA may reduce steady-state levels of UBE3A-ATS by at least 20%, at least 30%, at least 40%, at least 50%, at least 60%, at least 70%, at least 80%, at least 90%, or at least 100%.

An effective concentration or dose of the shRNA cut UBE3A-ATS and reduce it by at least 20%, at least 30%, at least 40%, at least 50%, at least 60%, at least 70%, at least 80%, at least 90%, or at least 100%.

An effective concentration or dose of the shRNA may reduce expression of UBE3A-ATS by at least 20%, at least 30%, at least 40%, at least 50%, at least 60%, at least 70%, at least 80%, at least 90%, or at least 100% and induce expression of paternal UBE3A by at least 20%, at least 30%, at least 40%, at least 50%, at least 60%, at least 70%, at least 80%, at least 90%, or at least 100%.

As used herein, the terms “UBE3A-ATS” and “Ube3A-ATS” can be used interchangeably without capitalization of their spelling referring to any particular species or ortholog. “UBE3A” and “Ube3A” can be used interchangeably without capitalization of their spelling referring to any particular species or ortholog. Additionally, “UBE3A”, “UBE3A”, “Ube3A”, and “Ube3A” can be used interchangeably without italicization referring to nucleic acid or protein unless specifically indicated to the contrary.

### Viral Vector

A “vector” is a replicon, such as a plasmid, phage, or cosmid, into which a DNA segment or an RNA segment may be inserted so as to bring about the replication of the inserted segment. Generally, a vector is capable of replication when associated with the proper control elements. Suitable vector backbones include, for example, those routinely used in the art such as plasmids, plasmids that contain a viral genome, viruses, or artificial chromosomes. The term “vector” includes cloning and expression vectors, as well as viral vectors and integrating vectors.

As will be evident to one of skill in the art, the term “viral vector” is widely used to refer to a nucleic acid molecule (e.g., a transfer plasmid) that includes viral nucleic acid elements that typically facilitate transfer of the nucleic acid molecule to a cell or to a viral particle that mediates nucleic acid sequence transfer and/or integration of the nucleic acid sequence into the genome of a cell.

Viral vectors contain structural and/or functional genetic elements that are primarily derived from a virus. The viral vector is desirably non-toxic, non-immunogenic, easy to produce, and efficient in protecting and delivering DNA or RNA into the target cells. According to the compositions and methods described herein a viral vector may contain the DNA that encodes one or more of the shRNAs described herein. In embodiments, the viral vector is a lentiviral vector or an adeno-associated viral (AAV) vector.

As used herein, the term “lentivirus” refers to a group (or genus) of complex retroviruses. Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). As used herein, the term “lentivirus” includes lentivirus particles. Lentivirus will transduce dividing cells and postmitotic cells.

The term “lentiviral vector” refers to a viral vector (e.g., viral plasmid) containing structural and functional genetic elements, or portions thereof, including long terminal repeats (LTRs) that are primarily derived from a lentivirus. A lentiviral vector is a hybrid vector (e.g., in the form of a transfer plasmid) having retroviral, e.g., lentiviral, sequences for reverse transcription, replication, integration and/or packaging of nucleic acid sequences (e.g., coding sequences). The term “retroviral vector” refers to a viral vector (e.g., transfer plasmid) containing structural and functional genetic elements, or portions thereof, that are primarily derived from a retrovirus.

Adenoviral vectors are designed to be administered directly to a living subject. Unlike retroviral vectors, most of the adenoviral vector genomes do not integrate into the chromosome of the host cell. Instead, genes introduced into cells using adenoviral vectors are maintained in the nucleus as an extrachromosomal element (episome) that persists for an extended period of time. Adenoviral vectors will transduce dividing and non-dividing cells in many different tissues in vivo including airway epithelial cells, endothelial cells, hepatocytes, and various tumors (Trapnell, Advanced Drug Delivery, Reviews, 12 (1993) 185-199).

The term “adeno-associated virus” (AAV) refers to a small ssDNA virus which infects humans and some other primate species, not known to cause disease, and causes only a very mild immune response. As used herein, the term “AAV” is meant to include AAV particles. AAV can infect both dividing and non-dividing cells and may incorporate its genome into that of the host cell. These features make AAV an attractive candidate for creating viral vectors for gene therapy, although the cloning capacity of the vector is relatively limited. In embodiments, the vector used is derived from adeno-associated virus (i.e., AAV vector). More than 30 naturally occurring serotypes of AAV are available. Many natural variants in the AAV capsid exist, allowing identification and use of an AAV with properties specifically suited for specific types of target cells. AAV viruses may be engineered by conventional molecular biology techniques, making it possible to optimize these particles for cell specific delivery of shRNA DNA sequences, for minimizing immunogenicity, for tuning stability and particle lifetime, for efficient degradation, for accurate delivery to the nucleus, etc.

An “expression vector” is a vector that includes a regulatory region. Numerous vectors and expression systems are commercially available from such corporations as Novagen (Madison, Wis.), Clontech (Palo Alto, Calif), Stratagene (La Jolla, Calif.), and Invitrogen/Life Technologies (Carlsbad, Calif). An expression vector may be a viral expression vector derived from a particular virus.

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). An expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or FIag™ tag (Kodak, New Haven, Conn.) sequences typically are expressed as a fusion with the encoded polypeptide. Such tags can be inserted anywhere within the polypeptide, including at either the carboxyl or amino terminus.

Additional expression vectors also can include, for example, segments of chromosomal, non-chromosomal and synthetic DNA sequences. Suitable vectors include derivatives of pLK0.1 puro, SV40 and, plasmids such as RP4; phage DNAs, e.g., the numerous derivatives of phage 1, e.g., NM989, and other phage DNA, e.g., M13 and filamentous single stranded phage DNA, vectors useful in eukaryotic cells, such as vectors useful in insect or mammalian cells, vectors derived from combinations of plasmids and phage DNAs, such as plasmids that have been modified to employ phage DNA or other expression control sequences.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, nuclear localization signals, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically includes at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. Modulation of the expression of a coding sequence can be accomplished by appropriately selecting and positioning promoters and other regulatory regions relative to the coding sequence.

Vectors can also include other components or functionalities that further modulate gene delivery and/or gene expression, or that otherwise provide beneficial properties to the targeted cells. As described and illustrated in more detail below, such other components include, for example, components that influence binding or targeting to cells (including components that mediate cell-type or tissue-specific binding); components that influence uptake of the vector nucleic acid by the cell; components that influence localization of the polynucleotide within the cell after uptake (such as agents mediating nuclear localization); and components that influence expression of the polynucleotide. Such components also might include markers, such as detectable and/or selectable markers that can be used to detect or select for cells that have taken up and are expressing the nucleic acid delivered by the vector. Such components can be provided as a natural feature of the vector (such as the use of certain viral vectors which have components or functionalities mediating binding and uptake), or vectors can be modified to provide such functionalities. Other vectors include those described by Chen et al; BioTechniques, 34: 167-171 (2003). A large variety of such vectors are known in the art and are generally available.

A “recombinant viral vector” refers to a viral vector including one or more heterologous gene products or sequences. Since many viral vectors exhibit size-constraints associated with packaging, the heterologous gene products or sequences are typically introduced by replacing one or more portions of the viral genome. Such viruses may become replication-defective, requiring the deleted function(s) to be provided in trans during viral replication and encapsidation (by using, e.g., a helper virus or a packaging cell line carrying gene products necessary for replication and/or encapsidation).

In embodiments, the viral vector used herein will be used, e.g., at a concentration of at least 10⁵ viral genomes per cell.

The selection of appropriate promoters can readily be accomplished. Examples of suitable promoters include RNA polymerase II or III promoters. For example, candidate shRNA sequences may be expressed under control of RNA polymerase III promoters U6 or H1, or neuron-specific RNA polymerase II promoters including neuron-specific enolase (NSE), synapsin I (Syn), or the Ca2+/CaM-activated protein kinase II alpha (CaMKIIalpha).

Other suitable promoters which may be used for gene expression include, but are not limited to, the 763-base-pair cytomegalovirus (CMV) promoter, the Rous sarcoma virus (RSV) (Davis, et al., Hum Gene Ther 4:151 (1993)), the SV40 early promoter region, the herpes thymidine kinase promoter, the regulatory sequences of the metallothionein (MMT) gene, PGK (phosphoglycerol kinase) promoter, alkaline phosphatase promoter; and the animal transcriptional control regions, which exhibit tissue specificity and have been utilized in transgenic animals: myelin basic protein gene control region which is active in oligodendrocyte cells in the brain, and gonadotropic releasing hormone gene control region which is active in the hypothalamus. Certain proteins can be expressed using their native promoter. Other elements that can enhance expression can also be included such as an enhancer or a system that results in high levels of expression such as a tat gene and tar element. The assembly or cassette can then be inserted into a vector, e.g., a plasmid vector such as, pLK0.1, pUC19, pUC118, pBR322, or other known plasmid vectors. See, Sambrook, et al., Molecular Cloning: A Laboratory Manual, Cold Spring Harbor Laboratory press, (1989). The plasmid vector may also include a selectable marker such as the β-lactamase gene for ampicillin resistance, provided that the marker polypeptide does not adversely affect the metabolism of the organism being treated. The cassette can also be bound to a nucleic acid binding moiety in a synthetic delivery system, such as the system disclosed in WO 95/22618.

Coding sequences for shRNA can be cloned into viral vectors using any suitable genetic engineering technique well known in the art, including, without limitation, the standard techniques of PCR, polynucleotide synthesis, restriction endonuclease digestion, ligation, transformation, plasmid purification, and DNA sequencing, as described in Sambrook et al. (Molecular Cloning: A Laboratory Manual. Cold Spring Harbor Laboratory Press, N.Y. (1989)), Coffin et al. (Retroviruses. Cold Spring Harbor Laboratory Press, N.Y. (1997)) and “RNA Viruses: A Practical Approach” (Alan J. Cann, Ed., Oxford University Press, (2000)). In embodiments, the shRNA DNA sequences contain flanking sequences on the 5′ and 3′ ends that are complementary with sequences on the plasmid and/or vector that is cut by a restriction endonuclease. As is well known in the art, the flanking sequences depend on the restriction endonucleases used during the restriction digest of the plasmid and/or vector. Thus, one of skill in the art can select the flanking sequences on the 5′ and 3′ ends of the shRNA DNA sequences accordingly. In embodiments, the target sites can be cloned into vectors by nucleic acid fusion and exchange technologies currently known in the art, including, Gateway, PCR in fusion, Cre-lox P, and Creator.

In embodiments, an expression vector includes a promoter and a polynucleotide including a first nucleotide sequence encoding a shRNA described herein. In embodiments, the promoter and the polynucleotide including the first nucleotide sequence are operably linked. In embodiments, the promoter is a U6 promoter. In embodiments, the first nucleotide sequence included in the expression vector may be SEQ ID NO: 2. In embodiments, the first nucleotide sequence included in the expression vector may also be a modified SEQ ID NO: 2 having the bold nucleotides in SEQ ID NO: 2 replaced by any of SEQ ID NOs: 4-489 and the italicized nucleotides in SEQ ID NO: 2 replaced by nucleotides complementary to those in SEQ ID NOs: 4-489. In embodiments, the first nucleotide sequence included in the expression vector may be any of SEQ ID Nos: 490-497. In embodiments, the polynucleotide including the first nucleotide sequence in the expression vector is a DNA polynucleotide. In embodiments, the first nucleotide sequence of the expression vector is a DNA nucleotide sequence. The shRNA encoded by the first nucleotide sequence of the expression vector may be as described in any of the variations disclosed herein.

As discussed below, recombinant viral vectors are transfected into packaging cells or cell lines, along with elements required for the packaging of recombinant viral particles. Recombinant viral particles collected from transfected cell supernatant are used to infect target cells or organisms for the expression of shRNAs. The transduced cells or organisms are used for transient expression or selected for stable expression.

### Virus/Viral Particle

Viral particles are used to deliver coding nucleotide sequences for the shRNAs which target UBE3A-ATS RNA. The terms virus and viral particles are used interchangeably herein. Viral particles will typically include various viral components and sometimes also host cell components in addition to nucleic acid(s). Nucleic acid sequences may be packaged into a viral particle that is capable of delivering the shRNA nucleic acid sequences into the target cells in the patient in need.

The viral particles may be produced by (a) introducing a viral expression vector into a suitable cell line; (b) culturing the cell line under suitable conditions so as to allow the production of the viral particle; (c) recovering the produced viral particle; and (d) optionally purifying the recovered infectious viral particle.

An expression vector containing the nucleotide sequence encoding one or more of the shRNAs herein may be introduced into an appropriate cell line for propagation or expression using well-known techniques readily available to the person of ordinary skill in the art. These include, but are not limited to, microinjection of minute amounts of DNA into the nucleus of a cell (Capechi et al., 1980, Cell 22, 479-488), CaPO₄-mediated transfection (Chen and Okayama, 1987, Mol. Cell Biol. 7, 2745-2752), DEAE-dextran-mediated transfection, electroporation (Chu et al., 1987, Nucleic Acid Res. 15, 1311-1326), lipofection/liposome fusion (Feigner et al., 1987, Proc. Natl. Acad. Sci. USA 84, 7413-7417), particle bombardment (Yang et al., 1990, Proc. Natl. Acad. Sci. USA 87, 9568-9572), gene guns, transduction, infection (e.g. with an infective viral particle), and other techniques such as those found in Sambrook, et al. (Molecular Cloning: A Laboratory Manual, Cold Spring Harbor Laboratory, Cold Spring Harbor Laboratory Press, Cold Spring Harbor, N.Y., 2001).

In embodiments, where an expression vector is defective, infectious particles can be produced in a complementation cell line or via the use of a helper virus, which supplies in trans the non-functional viral genes. For example, suitable cell lines for complementing adenoviral vectors include the 293 cells (Graham et al., 1997, J. Gen. Virol. 36, 59-72) as well as the PER-C6 cells (Fallaux et al., 1998, Human Gene Ther. 9, 1909-1917) commonly used to complement the E1 function. Other cell lines have been engineered to complement doubly defective adenoviral vectors (Yeh et al., 1996, J. Virol. 70, 559-565; Krougliak and Graham, 1995, Human Gene Ther. 6, 1575-1586; Wang et al., 1995, Gene Ther. 2, 775-783; Lusky et al., 1998, J. Virol. 72, 2022-2033; WO94/28152 and WO97/04119). The infectious viral particles may be recovered from the culture supernatant but also from the cells after lysis and optionally are further purified according to standard techniques (chromatography, ultracentrifugation in a cesium chloride gradient as described for example in WO 96/27677, WO 98/00524, WO 98/22588, WO 98/26048, WO 00/40702, EP 1016700 and WO 00/50573).

In embodiments, provided herein are host cells which include the nucleic acid molecules, vectors, or infectious viral particles described herein. The term “host cell” should be understood broadly without any limitation concerning particular organization in tissue, organ, or isolated cells. Such cells may be of a unique type of cells or a group of different types of cells and encompass cultured cell lines, primary cells, and proliferative cells.

Host cells therefore include prokaryotic cells, lower eukaryotic cells such as yeast, and other eukaryotic cells such as insect cells, plant and higher eukaryotic cells, such as vertebrate cells and, with a special preference, mammalian (e.g., human or non-human) cells. Suitable mammalian cells include but are not limited to hematopoietic cells (totipotent, stem cells, leukocytes, lymphocytes, monocytes, macrophages, APC, dendritic cells, non-human cells and the like), pulmonary cells, tracheal cells, hepatic cells, epithelial cells, endothelial cells, muscle cells (e.g., skeletal muscle, cardiac muscle or smooth muscle) or fibroblasts. For example, host cells can include Escherichia coli, Bacillus, Listeria, Saccharomyces, BHK (baby hamster kidney) cells, MDCK cells (Madin-Darby canine kidney cell line), CRFK cells (Crandell feline kidney cell line), CV-1 cells (African monkey kidney cell line), COS (e.g., COS-7) cells, chinese hamster ovary (CHO) cells, mouse NIH/3T3 cells, HeLa cells and Vero cells. Host cells also encompass complementing cells capable of complementing at least one defective function of a replication-defective vector utilizable herein (e.g., a defective adenoviral vector) such as those cited above.

In embodiments, the host cell may be encapsulated. Cell encapsulation technology has been previously described (Tresco et al., 1992, ASAJO J. 38, 17-23; Aebischer et al., 1996, Human Gene Ther. 7, 851-860). For example, transfected or infected eukaryotic host cells can be encapsulated with compounds which form a microporous membrane and said encapsulated cells may further be implanted in vivo. Capsules containing the cells of interest may be prepared employing hollow microporous membranes (e.g. Akzo Nobel Faser AG, Wuppertal, Germany; Deglon et al. 1996, Human Gene Ther. 7, 2135-2146) having a molecular weight cutoff appropriate to permit the free passage of proteins and nutrients between the capsule interior and exterior, while preventing the contact of transplanted cells with host cells

Viral particles suitable for use herein include AAV particles and lentiviral particles. AAV particles carry the coding sequences for shRNAs herein in the form of genomic DNA. Lentiviral particles, on the other hand, belong to the class of retroviruses and carry the coding sequences for shRNAs herein in the form of RNA.

Recombinantly engineered viral particles such as AAV particles, artificial AAV particles, self-complementary AAV particles, and lentiviral particles that contain the DNA (or RNA in the case of lentiviral particles) encoding the shRNAs targeting UBE3A ATS RNA may be delivered to target cells to inhibit the silencing of UBE3A by UBE3A-ATS. The use of AAVs is a common mode of delivery of DNA as it is relatively non-toxic, provides efficient gene transfer, and can be easily optimized for specific purposes. In embodiments, the selected AAV serotype has native neurotropisms. In embodiments, the AAV serotype is AAV9 or AAV10.

A suitable recombinant AAV can be generated by culturing a host cell which contains a nucleotide sequence encoding an AAV serotype capsid protein, or fragment thereof, as defined herein; a functional rep gene; a minigene composed of, at a minimum, AAV inverted terminal repeats (ITRs) and a coding nucleotide sequence; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Unless otherwise specified, the AAV inverted terminal repeats (ITRs), and other selected AAV components described herein, may be readily selected from among any AAV serotype, including, without limitation, AAV1, AAV2, AAV3, AAV5, AAV6, AAV7, AAV8, AAV9, AAV10, AAVRec3 or other known and unknown AAV serotypes. These ITRs or other AAV components may be readily isolated using techniques available to those of skill in the art from an AAV serotype. Such AAV may be isolated or obtained from academic, commercial, or public sources (e.g., the American Type Culture Collection, Manassas, Va.). Alternatively, the AAV sequences may be obtained through synthetic or other suitable means by reference to published sequences such as are available in the literature or in databases such as, e.g., GenBank, PubMed, or the like.

The minigene, rep sequences, cap sequences, and helper functions required for producing a rAAV herein may be delivered to the packaging host cell in the form of any genetic element which transfers the sequences carried thereon. The selected genetic element may be delivered by any suitable method. The methods used to construct embodiments herein are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation. See, e.g., K. Fisher et al, 1993 J. Viral., 70:520-532 and U.S. Pat. No. 5,478,745, among others. All citations herein are incorporated by reference herein.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989). Of course, not all vectors and expression control sequences will function equally well to express all of the transgenes herein. However, one of skill in the art may make a selection among these, and other, expression control sequences.

### Pharmaceutical Compositions and Therapeutic Treatment

The virus including the desired coding sequences for the shRNA, can be formulated for administration to a patient or human in need by any means suitable for administration. Such formulation involves the use of a pharmaceutically and/or physiologically acceptable vehicle or carrier, particularly one suitable for administration to the brain, e.g., by subcranial or spinal injection. Further, more than one shRNA herein may be administered in a combination treatment. In a combination treatment, the different shRNAs may be administered simultaneously, separately, sequentially, and in any order.

Pharmaceutical compositions herein include a carrier and/or diluent appropriate for its delivering by injection to a human or animal organism. Such carrier and/or diluent should be generally non-toxic at the dosage and concentration employed. It can be selected from those usually employed to formulate compositions for parental administration in either unit dosage or multi-dose form or for direct infusion by continuous or periodic infusion. In embodiments, it is isotonic, hypotonic or weakly hypertonic and has a relatively low ionic strength, such as provided by sugars, polyalcohols and isotonic saline solutions. Representative examples include sterile water, physiological saline (e.g., sodium chloride), bacteriostatic water, Ringer's solution, glucose or saccharose solutions, Hank's solution, and other aqueous physiologically balanced salt solutions (see for example the most current edition of Remington: The Science and Practice of Pharmacy, A. Gennaro, Lippincott, Williams & Wilkins). The pH of the composition is suitably adjusted and buffered in order to be appropriate for use in humans or animals, e.g., at a physiological or slightly basic pH (between about pH 8 to about pH 9, with a special preference for pH 8.5). Suitable buffers include phosphate buffer (e.g., PBS), bicarbonate buffer and/or Tris buffer. In embodiments, e.g., a composition is formulated in 1M saccharose, 150 mM NaCl, 1 mM MgCl₂, 54 mg/l Tween 80, 10 mM Tris pH 8.5. In embodiments, e.g., a composition is formulated in 10 mg/ml mannitol, 1 mg/ml HSA, 20 mM Tris, pH 7.2, and 150 mM NaCl. These compositions are stable at −70° C. for at least six months.

Pharmaceutical compositions herein may be in various forms, e.g., in solid (e.g. powder, lyophilized form), or liquid (e.g. aqueous). In the case of solid compositions, methods of preparation are, e.g., vacuum drying and freeze-drying which yields a powder of the active agent plus any additional desired ingredient from a previously sterile-filtered solution thereof. Such solutions can, if desired, be stored in a sterile ampoule ready for reconstitution by the addition of sterile water for ready injection.

Nebulized or aerosolized formulations are also suitable. Methods of intranasal administration are well known in the art, including the administration of a droplet, spray, or dry powdered form of the composition into the nasopharynx of the individual to be treated from a pressured container or dispenser which contains a suitable propellant, e.g., a gas such as carbon dioxide, or a nebulizer (see for example WO 95/11664). Enteric formulations such as gastroresistant capsules and granules for oral administration, suppositories for rectal or vaginal administration may also be suitable. For non-parental administration, the compositions can also include absorption enhancers which increase the pore size of the mucosal membrane. Such absorption enhancers include sodium deoxycholate, sodium glycocholate, dimethyl-beta-cyclodextrin, lauroyl-1-lysophosphatidylcholine and other substances having structural similarities to the phospholipid domains of the mucosal membrane.

The composition can also contain other pharmaceutically acceptable excipients for providing desirable pharmaceutical or pharmacodynamic properties, including for example modifying or maintaining the pH, osmolarity, viscosity, clarity, color, sterility, stability, rate of dissolution of the formulation, modifying or maintaining release or absorption into an the human or animal organism. For example, polymers such as polyethylene glycol may be used to obtain desirable properties of solubility, stability, half-life and other pharmaceutically advantageous properties (Davis et al., 1978, Enzyme Eng. 4, 169-173; Burnham et al., 1994, Am. J. Hosp. Pharm. 51, 210-218). Representative examples of stabilizing components include polysorbate 80, L-arginine, polyvinylpyrrolidone, trehalose, and combinations thereof. Other stabilizing components especially suitable in plasmid-based compositions include hyaluronidase (which is thought to destabilize the extra cellular matrix of the host cells as described in WO 98/53853), chloroquine, protic compounds such as propylene glycol, polyethylene glycol, glycerol, ethanol, 1-methyl L-2-pyrrolidone or derivatives thereof, aprotic compounds such as dimethylsulfoxide (DMSO), diethylsulfoxide, di-n-propylsulfoxide, dimethylsulfone, sulfolane, dimethyl-formamide, dimethylacetamide, tetramethylurea, acetonitrile (see EP 890 362), nuclease inhibitors such as actin G (WO 99/56784) and cationic salts such as magnesium (Mg²⁺) (EP 998 945) and lithium (Lit) (WO 01/47563) and any of their derivatives. The amount of cationic salt in the composition herein preferably ranges from about 0.1 mM to about 100 mM, and still more preferably from about 0.1 mM to about 10 mM. Viscosity enhancing agents include sodium carboxymethylcellulose, sorbitol, and dextran. The composition can also contain substances known in the art to promote penetration or transport across the blood barrier or membrane of a particular organ (e.g., antibody to transferrin receptor; Friden et al., 1993, Science 259, 373-377). A gel complex of poly-lysine and lactose (Midoux et al., 1993, Nucleic Acid Res. 21, 871-878) or poloxamer 407 (Pastore, 1994, Circulation 90, 1-517) may be used to facilitate administration in arterial cells.

The viral particles and pharmaceutical compositions may be administered to patients in therapeutically effective amounts. As used herein, the term “therapeutically effective amount” refers to an amount sufficient to realize a desired biological effect. For example, a therapeutically effective amount for treating Angelman's syndrome is an amount sufficient to ameliorate one or more symptoms of Angelman's syndrome, as described herein (e.g., developmental delay, severe cognitive impairment, ataxic gait, frequent seizures, short attention span, absent speech, and characteristic happy demeanor). Further, AS iPSC-derived neurons exhibit a depolarized resting membrane potential, delayed action potential development, and reduced spontaneous synaptic activity. Thus, a therapeutically effective amount for treating AS may return the neuronal resting membrane potential to about −70 mV, ameliorate the action potential development delay, increase spontaneous synaptic activity, or ameliorate additional alterations in the neuronal phenotype relating to rheobase, action potential characteristics (e.g., shape), membrane current, synaptic potentials, ion channel conductance, etc.

The appropriate dosage may vary depending upon known factors such as the pharmacodynamic characteristics of the particular active agent, age, health, and weight of the host organism; the condition(s) to be treated, nature and extent of symptoms, kind of concurrent treatment, frequency of treatment, the need for prevention or therapy and/or the effect desired. The dosage will also be calculated dependent upon the particular route of administration selected. Further refinement of the calculations necessary to determine the appropriate dosage for treatment can be made by a practitioner, in the light of the relevant circumstances. For general guidance, a composition based on viral particles may be formulated in the form of doses of, e.g., at least 10⁵ viral genomes per cell. The titer may be determined by conventional techniques. A composition based on vector plasmids may be formulated in the form of doses of between 1 μg to 100 mg, e.g., between 10 μg and 10 mg, e.g., between 100 μg and 1 mg. The administration may take place in a single dose or a dose repeated one or several times after a certain time interval.

Pharmaceutical compositions herein can be enclosed in ampoules, disposable syringes or multiple dose vials made of glass or plastic. In all cases, the composition should be sterile and should be fluid to the extent that easy syringability exists. It should be stable under the conditions of manufacture and storage and preserved against the contaminating action of microorganisms such as bacteria and fungi. Sterile injectable solutions can be prepared by incorporating the active agent (e.g., infectious particles) in the required amount with one or a combination of ingredients enumerated above, followed by filtered sterilization.

The viral particles and pharmaceutical compositions herein may be administered by a parenteral route including intravenous, intraarterial, subcutaneous, intraperitoneal or intramuscular injection or infusion, intrathecal or intracranial, e.g., intracerebral or intraventricular, administration. In embodiments, viral particles or pharmaceutical compositions are administered intracerebrally or intracerebroventricularly. In embodiments, the viral particles or pharmaceutical compositions herein are administered intrathecally.

In embodiments, the viral particles and a pharmaceutical composition described above are administered to the subject by subcranial injection into the brain or into the spinal cord of the patient or human in need. In embodiments, the use of subcranial administration into the brain results in the administration of the encoding nucleotide sequences described herein directly to brain cells, including glia and neurons. As used herein, the term “neuron” refers to any cell in, or associated with, the function of the brain. The term may refer to any one the types of neurons, including unipolar, bipolar, multipolar and pseudo-unipolar.

### EXAMPLES

### shRNA Vector Generation and Lentiviral Preparation

Oligonucleotides encoding shRNAs were cloned into the pLKO.1-puro vector, which drives expression of the small RNA by the U6 promoter (Addgene plasmid #8453). Specifically, the polynucleotides to generate shRNAs encompassed the specific 21-nucleotide sequence to be targeted and its reverse complement, separated by a loop sequence of CTCGAG, and with a 5′ flank sequence of CCGG and a 3′ flank sequence of TTTTTG added for cloning into the plasmid vector. The following oligonucleotides encoding shRNAs as well as a scrambled shRNA control were utilized:

551 shRNA 2 (“551-2”) (SEQ ID NO: 502):
(5′-TGCTCTTCTTTCTACTTTATTCTCGAGAATAAAGTAGAAAGAAGA
GCA-3′);
ATS-shRNA1 (SEQ ID NO: 503):
(5′-CTCAATCCAATAACCTAATTTCTCGAGAAATTAGGTTATTGGATT
GAG-3′);
ATS-shRNA2 (SEQ ID NO: 2):
(5′-GATATCACCTTACAGAAATTACTCGAGTAATTTCTGTAAGGTGAT
ATC-3′);
ATS-shRNA3 (SEQ ID NO: 504):
(5′-TTAGTCACATCCCACAAATTTCTCGAGAAATTTGTGGGATGTGAC
TAA-3′);
ATS-shRNA4 (SEQ ID NO: 505):
(5′-TCCTAGGTCATAATGATAATTCTCGAGAATTATCATTATGACCTA
GGA-3′).

Cloning was verified by Sanger sequencing. Lentiviral particles were produced from cloned shRNAs in HEK293T cells using second generation lentiviral packaging plasmids (psPAX2, Addgene plasmid #12260; pMD2.G, Addgene plasmid #12259) and concentrated using the Lenti-X Concentrator Kit (Takara). Lentiviral titer was estimated using a qPCR kit detecting the 5′LTR (Applied Biological Materials).

### Stem Cell Culture and Neuronal Differentiation

Angelman syndrome (AS) induced pluripotent stem cells (iPSCs) and human embryonic stem cells (hESCs) were maintained under feeder-free conditions on Matrigel-coated substrates (Corning) in mTeSR-plus medium (Stem Cell Technologies). iPSCs/hESCs were cultured in at 37° C. in a humid incubator at 5% CO2. Cells were fed daily and passaged using 0.5 mM EDTA every four-five days. Glutamatergic neurons were generated from iPSCs/hESCs by doxycycline inducible expression of the human neurogenin2 (NGN2) transgene (Fernandopulle et al., 2018, Curr Protoc Cell Biol. 79(1): e51). Briefly, the doxycycline-inducible NGN2 construct was stably integrated into the safe-harbor AAVS1 locus of AS iPSCs/hESCs using a pair of AAVS1 targeting TALENS and clonal cell lines were subsequently derived. Neuronal induction was then carried out by culturing these iPSCs/hESCs in Neural Induction Media consisting of DMEM/F12, N2 Supplement, Non-essential amino acids (NEAA), L-glutamine (all Gibco products), and 2 ug/mL doxycycline for three days. Neurons were then plated for terminal maturation in Cortical Neuron Medium consisting of DMEM/F12, Neurobasal Medium, B27 Supplement, Penicillin/Streptomycin (all Gibco products), BDNF (long/mL), GDNF (long/mL), NT-3 (long/mL), and Laminin (1 ug/mL). Human iPSC/ESC-derived NGN2-induced neurons (7-10 days post-induction) were transduced with lentiviral particles at an MOI of 10.

### Quantitative RT-PCR (qRT-PCR) Analysis

Neurons were collected for RNA isolation and qRT-PCR 7 days after viral transduction. Total RNA was isolated from iPSC-derived neurons using RNA-STAT60 (AMS Biotechnology) according to the manufacturer's protocol. cDNA was produced using the High Capacity cDNA Reverse Transcription Kit (Life Technologies). Gene expression analysis was performed at least in triplicate. All qPCR assays used were TaqMan Gene Expression Assays (Life Technologies). Ct values for each gene were normalized to the house keeping gene GAPDH. Relative expression was quantified as 2\hat{ }^{-ΔΔCt} relative to the calibrator sample.

### Data Summary and Results

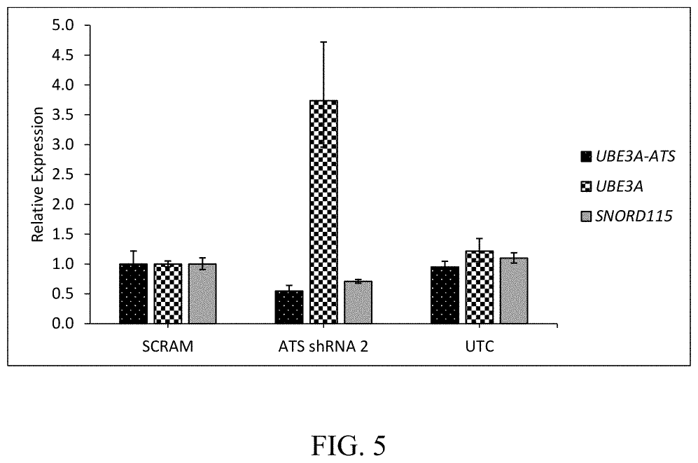

AS iPSC-derived neurons were transduced with lentiviral particles to express the selected shRNA sequences targeting the SNHG14 long non-coding RNA. qRT-PCR was used to determine the expression of UBE3A-ATS, the SNORD115 host gene, and UBE3A in SNHG14-shRNA-treated neurons relative to neurons treated with a non-targeting control shRNA (SCRAM). FIGS. 4 and 5 reflect qRT-PCR analysis of AS iPSC-derived neurons following treatment with either SNHG14-targeting shRNAs (551-2, ATS shRNA1-4, ATS shRNA2_3G) or non-targeting control shRNA (SCRAM). Expression of UBE3A-ATS, UBE3A, and SNORD115 were normalized to the housekeeping gene GAPDH, and expression is presented relative to SCRAM-shRNA treated neurons. Error bars represent standard error of the mean, n=3 biological replicates. As shown in FIGS. 4 and 5, select SNHG14 shRNAs effectively reduced RNA levels of UBE3A ATS (55%-60% reduction) and SNORD115 (45%-50% reduction) compared to SCRAM controls. This reduction in SNHG14 transcript levels was associated with a robust increase in UBE3A RNA (5- to 9-fold increase over SCRAM controls). Although ATS shRNA-1, ATS shRNA-3 and ATS shRNA-4 were predicted to reduce RNA levels of UBE3A ATS and SNORD115 and increase UBE3A expression, they did not have that effect.

It is understood that the foregoing detailed description and accompanying examples are merely illustrative and are not to be taken as limitations upon the scope of the subject matter described herein, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use, may be made without departing from the spirit and scope thereof.

Sequences

```
SEQ ID NO: 1
Human UBE3A-ATS genomic sequence.
TGAGATGACCTAAACAACTGTGGAGAATCATTGATATATTTCCTTTTTTCACTGTTCATGTTGG
GTGAAAATAATCTTGTAGTGAAATTCACATGTTCTAAATATTGTTTTTTTACATCTTTATCTGG
CACATTCATAACATAGATGTTTCTATACATATTAGTACTGTAATCATACCATATATTATTCTGT
TACCCCACTTACTCCTTAAACTTTTAGTTAATTAAAGAGTTTTTATAAAGTCCCCCAATAGATT
TTTTTTTTTTGAGACATAGTCTCACTCTGTTGCCCAGGCTGGAGTGCAGTGGCGTGATTTTGGC
TCACTGCAACCTCCCCATCCTGGGTTCAAGCAATTCTCCTGCCTCAGCCTGCCAAGTAGCTGGG
ATTACAGGTGCCTGCCACCACGCCCGACTAATTTTTGTATTTTCAGTAGTGACAGGGTTTCACC
ATCTTGGCCAGGCTGGTCTTGAACTCCTGACCTCGTGATCCACCCGCCTTGGCCTCTCAGAGTG
CTAGGATTACAGGCTTGAGCCACTGCACCCGGCCAGATTTTAATCTAATTTTATTAGAACAATT
CAGTCATATGTTTTTTCATGCTATGTATATGAGAGTTCCATTATTCAGATACTAAACAAATGTC
TACTGTACATTTACTGTTCTCACTGATGATGCATTAGATAACCATGCACAAAATAAGCCTGGCT
GTGGAAACGCTTATTTGTTGGGAGGGTGCTTGTTTGGATCGATGATGAGAATAATTGTCTGAGG
ATGCTGAGGGACTCATTCCAGATGTCAATCTGAGGTCCAGATGTGCGGCCCTCCAATAGGACAA
ATAAGACTCTCAGAGCCTGGCTCTATTTGGGGATCCCTCAGTGACAACATAGTACCCCTGTGAG
CGTGCCTTTTCTATCTCTTCGAAGAGGGCAGTGGCATCCTGTCTTATGAGTCAGTGTGCACTTT
AGTGTGCCTAGTGACCCAAGACTTGCTTTAATTGTAGATAGATACTTACATATAGGAAATATTT
CTTAAGTAACAAATGAAAAACTTTAGAAGATTGAATTAAGGGTCAAGCAACTGTGATATGTCTG
AAAATCTCATTAGTGTTGTGCTGAAAGAAGGAAATATGGCATGCCTCTATTAAATAATGACAGT
GGAACCAAGTTTATTGCTTTGTTATTTTTACTGTGGAGTATTTTCTAAGATTATTTTTGCTTTT
TTTTTCTTTCATGTTTTGCTGAGATAGAAGGCCTGGAATCTGATCCTCCACTTCAGAGAACAGG
GGTGAGTAGCTAAGCCATTATCTTTTGAAATTCATATGTCATGTGCTCTTTGCTAGGTCTTTAG
GTCGTTTTGTACATCTTTTCAGAAGCTTATTGGAGGACATTTTCATGATATGTCCTTTTCCTCA
TTGAGACCCTCACCATGTCACCTACACTATTGAATCCTTATCATTTCTCTTTTAATTTTAACTC
TCTTTTGCTTTTATGGAAAAATGTAGAATTTAAGAGAATTTTTGGCAATTTCATATTGGATCAA
AATGTATTGTAGTGAAATCCAGGTGTGCCAAAATATTAACAGATTTTCCCCATCTGTTTAATTA
TTGGGGTTTCAGAATAGAGACTCCATGGTTCATAATATCTTTGTGGTCATACTACATTATATTT
CTGCTTCTAATTTAATTATTAAATATTGACTTGAATTAGTCTTTTCCTCATTGTTGCAACAAGG
TAAGTTATATAGGAAATTTTCTTCTCTTGATGGCATGTCTGAGATAATCATAGATATAAGACAC
CTGGCTGGTTTCTAGAATGCATGTAATTTTTATTTCTTGATCTGTGTGTTGAGTACTTGCTGTG
ATTGCATCATGCAAATACATTGAGCTTTACAATTTTAGTGTATGCACTTTTCTACATGTATATT
ATTCTTCGATAGAAAGTAAAAAAAACTTATCGAACTAGTCAAAATATTGGTTAATACATAAAAA
AAGTCTCAAGTAGATTGTGTATTACATGGTGCTTGTTGATTGATGCCCTCATAATAGATCAAGT
GGGTTCTCTCTTTAGCACAGGGCTTTTTAGCAAATCATGTCATGAGTAGTTACTCAAGTATTTT
TATTTTAACACATTTATATTTTTTCTATGTATATTCTTAAATTCTCTTATACTTTTTTCTCTGT
TATAAAAACATGCTGAACAATCTCAAGTCTTAAGGATTGCAGTATTGTCCCCACATATTCATGT
ATTTTGGTACTCAATTCTTTATACTTTCTTTGACAGATCACTTGAACTGGCACATGTCTCTTGT
TTTGCAGAGAGGGAATTAATGTGATACCTTCATGCTTTTCTATTCTATGTGCTACATAATTGAA
TATACAAGCAAATATAGTTGTTAAGATTTAGTGTGATTATTTCTACACCACATGCAAAGAAGTT
TCTCATAGATCTTAATAGAGGCCCACATGCATTGTACAGTTTAGAATTTGGGGAAATATTGATG
AAGTTGGGTAAAGTATAAAGCCAAAAGTCAGAACAGTGAACTCCTTGCTTAAGGATTTCCTTGG
AGATTACTTAGTCAATACACAACTGATAAATTTAAGTGCTTTTCACCTTTTGAGTTCTCGACAT
ACTAAAGCTAAAATGTGTTTCAACTTTTAATCCTGCTTCCCTGATTTTCCCTTTTTTAGTCTGA
GATCAAAGAGTTTCAGCCATAAATTACTGCCAAGAGTAATCACTTCATTTTAAGAAAGCTTAAC
AATATAGAAGAATATAAAATTATTTATGACAGATGTATTTTTAACCTTTTCCCCATGCTTTCCA
GAGGAAATATGTTTAATCATCTGCCCTATATTAGGGAAAAACTTTCTATGCTAATACAAGTATC
TATCAATCCATTTATCTTTCTATATAAGATGTATTGATCATAACCAATTAACTTTACTGTAAAT
GAGCTTTAGATTTGACATTTTGGTAGTAATATATGTTGTACAATCTCCTGAGGTCCTATAGGTC
TTGAGGTCTCTATGTCAAAAACTATAGATGTGCCAGTGTCCTCAGTGAATGTGAAGGACACCAC
ATTTTCCTTAGCCATTTCTTGTTTTCAGAATAATGGTTATCAACATTTTGCTACTGCAAGATAC
CATACATTTATAATCGGAATATGCCAGTTTTTATGCACTCATGCCTCTGTTTCTGTAGAGCATT
CCCAGAATGAGTAATGCTTGAAAATTAGGTCCATGTGATTTCTTTAATGAGTTATAGTCAAATC
ATGAAATATTCAGGTTACCATTATTTCAGTAATGTATTAGAATGTCAAGGTAAAGTTATCTACA
TTGTATATATACACACAACATAGATATAATTTATACAACATCTATATTTATACAACAGATATAT
ATTTATGTATATATTTATACAACATAAAATATATTTTATTATTTAAACATAAAATATATTTTAT
TATTTAATATAGATTCTTAAGTGATAAATATGTTTAATATTATTAAAATAGGTTAAAATAGGTT
ATAGTTAGTACAGTGAAAATTGGCAGCCCTTTTACAAAACATATGCCATAACTATAGCATTTAT
CACTGACAGTCATACCAGGATAGTCTTTTATTTCCAATCACTTAAATATTCCTAATTGCAAAAG
AAATTTGAAGACTAAAATTCAGAAGTTTTGAAAGAGCCATTGCCTGGGTAAACTATACAGGTTT
CAGTTTTATTTATAATAATTATGAGGCCAGGCGCAGTGGCTCACACCTGTAATCCCAACACTTT
GGGAGGCCGAGGCGGGTGGATCACGAGGTCAGGAGATCGAGACCATCCTGGCTAACACGGTGAA
ACCCCATCTCTACTAAAAATACAAAAAATTAGCCGGGCGTGGTGGCGGGCGCCTGTAGTCCCAG
CTACTCGGGAGGCTGAGGCAGGAGAATGGCGTGAACCTGGTAGGCGGAGCTTGCAGTGAGCCGA
GATCGCGCCACTGCCCTCCAGCCTGGGAGACAGTGCGAGACTCCGTCTCAAAAAAAAAAATTAT
GTATATATTTATAAATTAATACTTAATAAATTAATAACTTGTGATAGGCAATGCAAAGATGACA
GTAAAAGGACAAAATTGATTAGATTGATAAAGTCCTGTTAACATGAAGAAATTGACCAGGATAC
CATCCACACTATAAAGTTAGAGAAATTTATCAGGACAATTCCCTAAAATACTCTTCTCAATTTT
AACATTGTAACAGGAATTTTTAAAATTTTGGTATTATGTGTGTTTCCTTCCAGATAATTTGAAC
AGATTCATATTTGGTATTTTTAAAAGCCATATCTTTGTCCTTAGTGCTGGCAATGTATTCTTGA
GAATGAACAAATAAGAGATACGTAAAAGCATAAGAGAAGGTATCAGGTTGAAGTAGTCAATCAG
TTATACAGAACACAAAGAATTTTATCTTGTATAATGITTATATAGCTTTATAGAAGTGTGCTGA
AAGGGCTATAAAACATGGACATTATTATCTCATTGAAAGGTCCAATACGTACTGAAATACATGC
TTTTATTTTGAACCAACCACCCTATAAACGTTGTATGGCTTATTTAGATGAGAGCCCAGGTTGT
GTGTGTCTGTGTGTGTGTGTGTGTGTGTGTGTGTGTGTGTGTGTGTGTGTGTACCTGACAGGGA
AGCAAGAACATCGAGTTGCCAATGCACTCTGTCTATGGTTAGAATCATGCTGAAAACATGGCTC
CCCCAGTTCTGGAATGAGCCCACAGATCAAGCATTCCCCAAAGACATAGCAGGCTCAAATCCCT
GTGTACACAATATTTTATGATTATCTTATGTCAGTACTTTCAAAGTATACAGTTTGTGTGAAGA
CAAATCCAATGTCATTTTTCTTGGCTAGCCTATATGTGTGGTAAATCCATTATTTACTTACTTG
CTTCCTGAAAATTACAATTAGATTAACAAACTGCAGCAAAGTGGGCATGATGAGATAGAGATTG
AAGTGTAAGCTTATGTTAATGATGCCCTTGGTTTGGATAAACACATCTAAGAGAAAAATGGAAA
AACACACATGGCAGGGAAGCCTTGATAGAGCCAAAATATAGGATTGTATGTAGTAATGCAATCC
ATAGATGAGCATTTGGCAGTAATATTATTTTTCAGATATGGATAAAAATTGCTTAGGAGAGTAA
AGAGAGACAAAGTTGAAAGCAGGTTTATAGTAGGTGTTGTTTTAGTGTTGATCCCTTTTTGCTC
CAATAATCAAAGTGATAAATATTGAAAATTGATTCATGCAGCATTACTTACTCCATTCTAATTT
TTATATATGTCAAAAGTGCCATCTCCCAAACTGTGCTATCCCCTTCAGGAGAAGAGACTCTGCT
GAAGTTTATAAGGTTGACATATTGCCAGCTTCAATAATGTAAAGATGAAGTGTATACTGAATTC
TTAATGCAAATAACAACTCTATTGGAAAGTAACCCAGTTATAGAAGTGCTAATTTGTCAGGAGC
TGCCTTACCAAGATCATGATGAGTACAGTTATCTCAGGATTCTGAAAGATTGTTTTCCGATTTC
AACTAGTCTAGCTGAATGTTCCTTGATAGAAAGAGAGGACTTTTAGAATTGGTTCAATATGATG
ACCTCCTGAATTATCTCACATAGCCCGTTTGTACATGCCTTTCTTTTCTCTCAGAAAATGGCAC
TATCATAATAGCTTTCTTACACAGACTTCACCTTAGGGTTTTACATTAAGGGAGGGGTCTGGTG
TTTCATTTATTTTGAAGTATTTGTTGTTGATTGTGTACAGTGCTTGAGTAAAAAATTGAATATA
GAAACATCTAGAATATTTTTTTAAAGGATCAGTGTTTATAAAGTGAATTATTAGTGTCAATAAT
GTTGGGAAAGTTTTAAGAGAATATAGGAAACTTGAACATTACACAACTACAATGGGACCAAATT
GTGGGGTCTCATTATAGTTAATATTTATGTATTTTTTTCCAATTGATTTGTGTGCTTTTTTTCT
GCATGTTTTTGGCAGATAGAATGGCTATAACAAGTAACAGCATGTCAGGTAATAAAAATAAGCA
GAGCCCTATTCCTTTAAAAATCTTCACTGATGGGAGGGCCATAAAATAAGTCTTAATACATTTA
AAGAATTAAATTCATGTAAACCATGTTAATTTAATTCCACAATGATATTGAATTAGAAATAAGA
GGAATATCTCTTGAACATCTCCTAAATGTTTGGAAATTTAAATTAGCATTTCTGACCTATTTAT
TGGTTAAAAAAGATACAAAGAAAGGAAAATTGAAAAGTCTTTTGAACTGAATAAAAATAAAAAT
ATAGAATCTAAAACTTTATGGGATACTGACAAAACAGGATATAGGGAATAATTTATAGCACTGA
AATGCCTATATTAGAAAAGAAAAAAGGTTTTAAATCAGTAAATTTGTATTTTACCTTAAGAAAC
TTAGAAAAGAACAAATTAACCCAGACTTAAGTAAAATAAAGGCACTAATAAAGATAAGAGCAGA
AATCAATGAAATATAAAACAACAAAACACAGAGAAAAATTGAGAAAATTTAAAAATAGCCTAGT
GAGAAGATATTGATAAACTTGTAACCAGACCAATTTAAGAAAAAAAGTCAAAACACAAATACCA
ATATTTGAAAATGTAGGAGGGCAAATCATTACAGATTCTATGAATACTAAAATGATAATAAGGA
AAAATTATTTAAAAGGGGCATGTCAGCCAGGCATGGTGGCTTACCCCTGTAATCCCAGCACTTT
GGCAGGCCGAGGTGGGAGGATTGCTAGAGCTCAGGCATTCGAGACCAGCCTGGGCAACATGTTG
AAACCTTGTCTACACAAAAAGTACAAAAATTAGCTGGGTGTGGTGTTGCACACTTGTAGTCCCA
GTCACTTGGGAGGCTGAGGCGAGAGGATCACTTGAGCCCAGGAGGTTGAGGCTGCAGTGAGCCA
TGTTTGTACCACTGCCCTCCAGCCTGGGTGACAAAGTAAGACCCTATGTAAAAAAAAAAAAATG
TATGCCAACATTTTTCAATAACTTAAATGAAATGGAAAAATTCCTTGAAAGACACGAACTACAA
AAACTCAGTGAACAAGTAAATAACCTGAATAGCCCTGTATCAAGTAAATTGAATTTGTAGTTAA
AAGCCTTCCAACAGAGAAAACTTCAGGTACCTATAGCTTCATATGAAATGAAAAAAAAAATACC
AATCCTCTACAAGATTCCAGAACATTTAAAAGAAGGGAATATTTCCCAACTTATTCCATTTGGA
CAGCAATACCCAGGTAAGAAAAAGAGACACAGAAATTTAAAAAGAAGAATATACATTATTCCTT
AGGAACATAAATGCAAAGAAATCTAATCAAAATTTTGGCAAATGAAATGTAGAAATACTTTATG
ACCAAGTGAGAGTTACCCAAAGAATTTAAGGTTGGTTTTATATGTAAAGATCAACCAATATAGG
AAAATCACTTCTGGAAAGTCAGAGTAAGAAACTCCAAAAATCTACTCCTCCATAAAACCAATAA
CAGCCTTGATAGAAATAGTTGAAATTAATTTTCCAAAACTTTGGAAATTAACCAAAGGCTTACA
AAATTCCAGAGAACATTAATTCAAGAAAAATGGCTGAATCAGTAAGAACAGCCAGCTTTGTGGC
ATTTTAATATGACCCCTTCCCATGCTTTTCTCCCTAGTGCTGAGATAGTCTTAAAAATTAGCAG
GATAGCAACCACTGGAGAAGAAAGGTTTGGAAATTTCCCAAAAAGTTCCATCCCCATAGAATTA
TCACTATTTGACCTCTAAAGCCCAATCTATAGGATTTATATTCATTTGGACTGACTCAGAGCTC
ACTCAGTAAGGAAATCTCAATTTCAAGGTATTGGTCAAAAAGAATCCATGGCAATTGTTGACTA
TCACAACTGCCTGAAGTCTTGGTAACAGTTGGGATAAACAAGAAGCTGATCAAAAACTGAAAAC
TAAAATCTTGGGAATGAGATATCTACAGGATGCTTCAAAAAGCTTTGATACATTCCTGTTTATC
TAGAAAGCTACATGCAGGCTGGGTGCAGTGGCTCACACCTGTAATCCCAGCACTTTGGGAGGCC
GAGGCGTGCGGATCATGAGGTCAGGAGTTTGAGGCCAGCCTGACCAACATGGTCTCTACTAAAA
ACACAAAAATTAGCCAGGCGTGGTGGCGTGCATCTGTAATCCTAGCTACTCAGGAGGCTGAGAC
AGGAGAATCGCTTGAACCCGGGAGGCGGAGGTTGCAGTGAGCCAAGATTGTGCCACTGCACTCC
AGCCTGGGCGACAGAGCAAGGCTCTGTCTCAAAAAAAAAAAAAAAAAAAGCCACATGCATGTAA
TTGTTTACCTCTGGCTTTCCTTTTATGCTCTGGGCAAGCTAAGGAAGAGTTGTGAACTACCTAA
GTGCTGAATGGGAACCATAACACACACACACACACACACACACACACACACAGCACCTTAGTAA
AGGGTGAGAGGCATGTTAGTTAGAAGCATTAAAGGAAATCTCTTTCTAGTCATTATCTGTGCAC
TAACCTAACTGAGCAGAGACTTCAGTATCCACATACTACAGGGCATATAAACTTTACAGAATTA
GTCCAGGAAAATCATATCTAAAAAAAAAAAAAAGCAGTAACAAAAATAAACTCTGGGAAAGGGG
AGAATATGATTTAAAGAGTTGCCACATTATACATAATATGTCTAGTGTTCAACAAAAAATTACG
AGACATGCAAAGAAATAGAAAAATATGGCACAAAGAGGATAAGATGAAGTCAGTGAAACTATCC
TCGAGGAAGACCAGATGTTGGTCTTACTAGACACAGACATTGAACCAGCTATTAAAAATACGTA
CACAGAACTAAGAAAAACATGTCAAAAGGGTTAAAGAAAGGTATAAAAATAGTGTCTTACCAAA
TATAGACTACCAATAAAGAGATAGAAATTATAAGAAAAGACAACATGAAAAAATATAAAGCAAA
AAAATTAGACAATTGAAACAAGAGGGCCTCTATTCGCAGATTTGAGCAGGCAGAAGAAAGAATC
AGTGAACTTGAAGATATGTCAACTGAGATTATCCAGTCTGAGCAACAAAGGTGGGAAAAAATGA
AGAAAACTGAGCAACAAAGAACTGTAGAACAGCATCTCTCATACCAATGGATACATAAACTGGA
GCCCTAGAAGGATGAAAAAAGGAGAAGGAAAGAAAACTTCCCAAATTTTAAGAAAAACATTAAT
TTATATACCCGAGATGACCAATAAAATCCAATTAAGATAATCTCAAAGAGACCAACACCTATAC
ACATCATAGTCAGCGTGTCAAAAGACAAACATAAGGAGAGAATTCTTGAAGATAGTAAGAAAAA
AATTATTCATAACATACACACCATCCTCAATAAGTCTGACAATTGACTTCTCACTGTAAACCAT
GCAGGCTAAAAAGGCAATGTACATAACCAAAGTGATGAAATAAAAACCTTCAACCAATCATTCT
AGATCCAACAAAACTATTGTTCAAAAAGAAGAAATGAAGACATTCCTAAACAAAATCTCAGAGA
AATGTTCTCTATAAGACTTGTCCTAATAGAAATGCTAAAGGAACTCCTTCGGTCTGAAATAGAA
AGGCACTGGAGAGTAAATCAAATCCACGAGAAGAAATAAAGAGAACCAGTATAAGTAACTACAT
GTGTAAGTTTAAAACAAAGTATAAATTTATTTTGTTTGTAACATTTGTCTTTTCCTATTTGATT
TAAAATATAATCTCAATTATAAACTTGTGTTGATGGTATTATATAAAGATGTAATTTTGGGTTA
TAGCACCAAAATGGCAGAATAGGAATTTTCTGTAGGTGTTTCCCACATAAGTATCAATTTTGAC
AACCATCCATGGGCAAGAGTACCTTGTGGGAGTTCAGGAGTTGACAGTAAAACTTCAGCACACC
AGAGGAGTAAAGAAATCTAAGAATAGATTCATTGGAAAGGGTATAAACAGTTTCACTTTACCTG
CATCACCAACCCCCAAAAGTGGCACAGCTCAGTAACAAGAGCCCATTATTTCTTCCACAGAGGA
AAAGGAGAGTATAATAAGTAAGTGTCCAGTTTCTCAAGACATACAGGCCCCTGCCCAAGAGATC
CACTTTATTTTCATCTCACCCAGAATATTGAGGTGATCAGCAAGGTGGAGTGGTTGGGAGAGGG
TAAAAGCAGGAAAGAGAGATGGGGACTCAAACAGCAGCCCATACTTGGAACTGCCATAGATCCT
ACCAGTTACTTCATGGACTCCATCAGGAACCCACCTATAAGCCACAGGGGATGCATCCCTCCCA
TCTTGCCAACAAAACCCCGAATGCTCCAAATGCCTCACCCACTCTTTGGCTGGCTCCCAAGTGC
GCTCCTGTGAAAAGCGAGTGAGTATCTCTGCAGATGGCTTGCAAGCACATGTTGACAGCTGGCT
CCACTCTGTAGAACTGGAAAAAAGCTCACATACATGAGAATTTCAGGACACTACCCTAAAAAAA
AAAATGAGACTTCAGCACCTGGCCTGGCTTTATGCAACCTAGAGAAGGTGATATGATTTGGCTC
TGTGTCCCCACGTAAATCTCATGTCAAATTGTAATCCCCACGTGTTGGACAAGGGACTTGGTGG
GAGGTGATTGAATCATGCGGGTGGACTTCCCTCTTGCTGTTCTTGTGATAGAGTTGTTATGAGA
TCCAGTTATTTGAAAGTATGTAGCATGTCCCCCTTCACTCTCTTGCACTCCTGCTCCACCTTGG
TAAGACTTGCTTGCTACCCCTTTGCCTTCTGCCATGATTGTAAGTTTCCTGAGGCCTCCCAGCT
ATGCTTCCTGTATGGCCTGCAGAACTGAACTGTGAGCCAATTAAACCTCTTTTCTTCATAAATT
ATCCAGTCTTAGGTAGTTCTTTATAGCAGTGTGAGAATGGACTAATGCAGAAGGCATACAACCT
TTAGAATTTGCCCCCTTGAGGGAACAAGATGTGTGAAGCAGGTTCATCCATAGAAAATGTCTGA
GAGAACCTCAAAATCCCTAACCTGACTAACTGATGAAAGTGTTTCTCTCCTAAGGCCAGTCAGT
AAAGACCAGAGGGGGTGACTGTTTCTTTAAATGCAAAGGCAGCAGCACAATAATTCAAGAAACA
TGAAAAATCAAGAAAACATGACACCACCAGAAGAACACAATCATTTTCCAATAACCAACTCCCC
AAAAATGGAGATTTACAAATTGGTTTATAATGAATTCAGAACAATTATGTTAAGGAAGCTCAGC
AAACTAAAAGGAACACCAATAGACTACTCTGTGAAGTCAGGCAAACAATTCATGAACAAAACTA
GAAATTCAAAAAAGAGAAAAATTATCTTAAAAGAAAACCCAGAAGTTATGGAGCTAAAGAATAC
AATGCATGAAATGAAGGAGCGTATCAACAGCAAAGTTGATCAAGCATAAGAAAAAAAAAATCTG
TGAAACTGAAGACTGGCTATTTGAAATTATTCATCAGAGGATTAAAAAAAAAAGAATGAAAAGA
AATAAAGAAAGCCTACAGGATGTATAAAACACCATCAAGAGAACTAATATAAGGATTATTGGAG
TCATAAAGGAGAAGAGAGAAAAGGGTAGAAAACTTATTTAAGAAATAATGGCTGAAAACTCTCC
AAATCTAGGAAAAGATATGAGCATCCAGGTATATGAAGCTCAAAGATCCCCGTACAGGATACAT
TCCAAAAAGACTTCACCAAAACACATGATAATCAAACTGTCAAAAGCAAAATCAAGACGATGAA
TAAACCACCAATCACTAAGAGGGAGACAGGATTCTTATGTTGTCATTATTATTTACATATAATT
TCAGTAAATGTTATTGGAAAATTTATAATGTTTTAAAAAAAGAAATTTGAAAGCACCGAAAGAA
AAGAGACTCATCACATACAGGGAACCCTTTTAAGGCATTCAAGAGATTTCTCAGTAGAAACCTT
ACAAAATAGGAGAGAGTGGGATGAACTATACAAGTGCTGCAAGGAAAAAAATGCCAACCAACGC
TTTACCTGGCAAATCTGTTCCTCAGAAATGAAGGAGAGAGAAGAACTTTCCTAGACAAACAAAA
GCTGAGGCAGTTCATCACCACTAGACCTGCCTTACAAGACATACTAAGGGGAGTTCTTCAAGCT
GAAATGATATGGCAATAGTTAGTAATATGAAATGATAAACCTCACTGGTAAAGGAAAGTACATA
GTCAAATTTAGAACACTTTGATACTATAATGATGGTGTATAAATAATTTTACTGTGCTATGAAG
GTTAAAAGACAAAAGTATTAAAAAAAACCCATAGCTGCAATAGCTTGTCAATGCATACTACAGT
ATAAAAAGATGTAAATTAGAACATTAAAAACATAGCATGCAAGGGTAGGGAAGTAAAAGTGTAG
TTTTCATATGTAATCAAATTTAATTTGTTATCAGCTTAAAATAAATTGTTATACCTATGTTTTA
TGTAAGTGTCATGGTAACTATAAAGGAAAAACCTCTAGTAGATACACAAAAGAAAAAGAGAAAG
GAATCAAAACATAACACTACAGAAAATTATCAAATTACAAAGGAAGACAGCAAGGGAGGAACAA
AGTAAAAAGAGCAAGAAAAAATTTAACATAATGAAAACAGTAAGTCCTTACGTGTCAATAATTA
CTTTAAATGTAAATGGATTAAATTATCCAAACAAAAAAACAGACTGGACAAATGGATTTTAGAA
ACAACAACAACAACAAACACCGCACACACACACACACACACACAAACCACCCAGCCCCAACTAT
GTGCTGCCTACAAGAGATTTACTTCCACTTTAAGGACACATACAGGCTGAAATTAAAAGAACAG
AAAAAGATATTGCATGCAGATAGAAACCAGAAGAGAGGAGAGGCATCTATACTTACAGCATACA
GAAAAGATTTTAAGTTAAAAACTATATCAAAAGGCTAAGAAGGTCAAAATGGTGAAGCAGTTAA
TTGTTCAAGAAGACATAAAAATTGTAAATATTTATACACCCAATATTGAAGCACCTAAATATAT
AAGGCAAATATTAATACATATAAAAGGAGAAATATACAGCAATACAGTAATAGTAGTGAACTTC
AGTGCCTCCCTTTCAAAAATGGATAATCCAGACATAAAATCAATAAGGAAACATTTAACTTAAA
CTTCACTTTAGACCAAATGGATCTAACAGACATTATACTGAACATTTCATCCAACAGTGGTAGA
ATTCACATTCTTCTCAAGCACACATGGAACATTCTCCAGGATAGATTATATGTTAGCTCACAAA
ATAATATTACAAAATTTAATAAAGCTGAAATATCAATTATTTTGCACCACAATAGTATAACACT
AGAAATCAATAACAAGATGGAAACTGGAAATTTACAAATATATAGCATTAACATATTCCTGAAC
AACCAATGGGTCAAAGAAAAAAATCAAAATAATTTTGTGACAGCAAAGTAGAAACACAACATAC
CAAAACATACAGGACACAGCAAAAGCAGTTCTATGAGGTAAGCTTATATTGATAAACACATTTA
AAAAAAGATTTTAAATAAACAACATTACACCTCAAGGAACTACAAGGAAGAAAAAAAAACAAGC
CCCATGTTATCAAAGGGAAGGAACTAACAAAGATCAGACAGAAATAAATGAAACATAGACTAGA
AAAACAATAGAGACTATTAATAAAACTTAGAGTTAGTTTTTTAAAAAATAAAATCAACAAACCT
TTAGCTAGACTAAAAAAAGAGAAGACTCAAATAAAATAAAAAATGAAAGAGGAGACATTACAAC
TGATACCACAGACATACAAATTAAGAGAAAACTATATGCCAACATATTAGTTAACTTGCAATGG
GTAAATCCCTAGAAACATACAACCTACAAAAACTGAATCATGAAGAAATGGAAGATCTGAACAG
ATCAATAATGAATAAGGGAATTGAATCAATATTCAAAAATCTCACAAAAAGAAAAGCTCAGGAT
CAGATGGCTTCACTGGTGAAGACTGCCAACCATTTAAAAAAATTAATACCACTCTTTATTAAGC
TCTTCCAAAAAAATTGAAGAGGAGAAAACACTTTCAAATTCATTATAAGAGGCCAGTTTTACCT
TGATATCAAAGATTTAAAAAGAACACTTTGAGAAAGGAAAATTACAGGCCAAAACCCTTGATAA
ATATAGATGCAAAAATGCTCAGCAAAATACTAGCAAACCTAATTCAGCAACACATTATAATGGC
ATACATCATGACCAAGTGAGATTCATGCCTCGGATGCAGGATAGTTCAATATAATCAAATCAAC
AAATGTTACACTACTTTAACAGAATGAAGGATAAAAATCATATGATCATCTCGATGGTTGAACT
AGTTTACAGTCCCACCAACAGTGTAAAAATGTTCCTATTTCTCCACATCCTCTGCAGCACCTGT
TGTTTCCTAACTTTTTACAGATCACCATTCTAACTGGTGTGAGATGGTATCTTATTGTGGTTTT
GATTTGCATTTCTCTGATGGCCAGTGATGGTGAGCATTTTTCAAGTGTCTGTTGGCTGCATAAA
TGTCTTCTTTTGAGACGTGTCTGTTCATATCCTTCACCTACTTTTTGATGGGGTTGTTTGTTTT
TTTCTTGTAAATTTGTTTGAGTTCTTTGTAGATTCTGGATATTAGCCCTTTGTCAGATGAGTAG
ATTGCAAAAATTTTCTCCCATTCTGTAGGTTGTCTGTTCACTCTGATGGTAGTTTCTTTTGCTG
TGCAGAAGCTCTTTAGTTTAATTAGACCCCATTTGTCAATTTTGTCTTTTGTTGCCATTGCTTT
TGGTGTTTTAGACATGAAGACAGTGTGGTGATTCCTCAAGGATCTAGAACTAGAAATACCATTT
GACCCAGCCATCCTGTTACTGGGTATATACCCAGAGGATTATAAATCACGCTGCTATAAGCCAT
AAAAAATGATGAGTTCATGTCCTTTGTAGGGACATGGATGAAGCTGGAAACCATCATTCTCAGC
AAACTATCACAAGGACAAAAAACCAAACACCGCATGTTCTCACTCATAGGTGGGAATTGAACAA
TGAGAACACATGGACCCAGGAAGGGGAACATCACACACTGGGGATGGTTGTGGGGTGGGGGGAG
GGGAGAGGGATAGCATTAGGAGATATACCTAATGCTAAATGACGAGTTAATGGGTGCAGCACAC
CAACACGGCACATGTGCACATATGTAACAAACCTGCACGTTGTGCACATGTACCCTAAAACTTA
AAGTATAATTAAAAAAAATAATGCTGCTATAAAGACACGTGCACACGTATGTTCATTGCGGCAC
TATTCACAATAGCAAAGACTTGGAACCAACCCAAATGTCCATCAATGATAGACTGGATTAAGAA
AATGTGGCACATATACACCATGGAATACTATGCAGCCATAAAAAAGATGAGTTCATGTCCTTTG
TAGGGACATGGATGAAGCTGGAAACCATCATTCTCAGCAAACTATCACAAGGACAAAAAACCAA
ACACTGCATGTTCTCACTCATAGGTGGGAATTGAACAATGAGAACACTTGGACACAGGAAGGGG
AACATCACACACTGGGGCCTGTCGTGGGGTCAGGGTAGGGGGAGGGATAGCATTAGGAGATATA
CCTAATGTAAATGACGAGTTAATGGGTGCAGCACACCAACACAGCACATGTGTGCACATGTACC
CTAGAACTTAAAGTATAATAAAAAATAAATCATATCATCATCTCAGTAGATTTAGAAAAGCATT
TAACAATATTCAACATCCTTTCAGAACTAAAAACTCTCAATAAATCAGGTATAGAAAGAATGTG
CCTCAACACTATAAAAGCCACATATGACAAACCTGGAGGTAATATACTCAATGGTGAAAAGTAA
AAAGCTTTGACTCTAAGATCAGAACCAAAACAAGGATGTCCATTCTCACCACTTATATTTAACA
TAGTAGTTGAAATTCTAGCTAGAGCAATTAGGCAAGAAAAAAGGCACCCAAGTTGGAAAGAATG
AAGTTAAATTGTCTCTGTAGATGACATGATCTTATATATAGAAAACACTAAAGACTCCACCAAA
ATGCTGTTTTAATTAGAGCTTAAAAAAATAATTCACTAAAGTTGCAGGATACAAAATCAGTATA
CAAAAATCAGTTGCATTTCTAAACACCAAAAACAAGTTATCCAAAAAATTAAGAAAACAATCCT
ATTTGTGATATCATCAAAAAATAAAATACTAAAGAATACCAAAGAAACTGAAAATAAATGGAAA
TAAATGGAAAGATAGCCCATGTTCATGGATTAGATGAATTAATACTGTTAAAATGTTCATACTA
CCCAAAGCAACCTACAGATTAAGTGCAATTCCTACTAAAATTCCAATGACATTTTTCACAGAAA
TAGAAAACACACTCCTAAAGTTTGCATGGAACCGCAAAAGACTCAAATAGATGAAGCAATTTTG
AGCAAGAACAGTAAAGCTGGAGACATCACACTACCTAACTTCAAAATTTTATTAATCAAAACAG
CATGACATAAAAACAGACAGAAAAGACCAATGGAACAGAATAGAGAGCCCAGAAATAAACTCAC
GTTTATAGAGTCAACTAATATTCAACGAAGGTGCCAAGAGTACACAATGGGGAAAGTATAGTCT
CTACAATAAACAGCACTGGGAAGACAATATCCAAATGCAAAAGAATGAAATTACACCCTTATCT
TATACCATACACACAAATCAAATCAAAATTGATAAAGACTTAAATATAAGACCTGAAACCATAC
AATCTTTAGGCAAAAACTCATTGACATTGGTCTTGGCAATGATTTTTTTGATATGACACCAGAA
GCACAGGCAACAAAAGCAAACCTAAACAAGTGGGACCCTAACAAACTAAAAAGTTTCTGCACAG
CAAAGGAAACAATCAATCATCAGAATTAAAAGGCAATTTATGAAATGGGAGAAAATGTTTGCAA
ACCACATATCTGATAAAGGGTTAATATCCAAAAATATATAAGGAATGCATAAAATTCAATAGAA
ATAAACAAACAAATAATCCAATTTTAAAATGGGTGAAGAACCTGAATAGACATTTTTTCAAAGA
AGACTTACAGATAGGCAACAGGCATATGAAAAAATGCTTGACATCACTAATAATCAGGGAAATG
CAAATCAAAGCTCCAGTGAAATACCACCTCCAACTATTAGGATGGCTATTATCAAAAACTCAAA
AGAAAACAAGCTGGGGGAATGTAGAGAAAAGGGAAAAGAAGATCCTTATACACTGTTGGTGTGA
ATTTAAACTGGAATAGCCCTTATGGAAAACAGCATGGAGGTTCCTCAAAAAATTAAAAATAGAA
CTACTATATGATCCAGCAATTTCACTATTGAGTATATATCCAAATGAATTAAAATCACTGTCTT
GAAGAGGTATTTGCACACTCATATTTATTTCAGCATTATTCACAATAGCCAAGACATGGAATCA
ACCTAAGTGTTCATCAGTAGATGATTAGATAAAGAGAACGTGGTATATAGACACAGTGGAATCT
ATTTAGTGTTCAAAAAGAAGGAAATCCAACTTTTAAAATCCTTTAAAAAGTTAAACTCATAAAA
ACAGAGAGGAGAATGGCGGTTTCCAGGAACTGGAGGGTGGGAGAATGGGGAGATGTTGGTCAAA
AGGTACAAACTTTCAGTTATAAAATGAATAAGTTCTAGAGATCTAGTGTACAACAGCATTACTA
TAGTTAATAATAATATTTTTTATACTTGAAATTTGCTAAGAGTAAATATCAATATTCTCAATAC
ACACAAAACAGAACTATCTGAAGGCACTGATATGTTAATTATCTTCATTATAGTAATCATTTCA
CAATGTATAATGAATATCAAAACAATAGTGTACATCTTAAATATATACAGTTTTGATTTGTTAA
TCATACATCAATGAAGCTAGAAAAAATGTTGTAATTTTTAAAACAATAGTAATATAAATTAGGG
GTGAAGGAATTGACCTATGTTGGAGAAAAGTTTTTGTAAACTATTTAAATTAATTGGTATCCAT
TCAAGCTAGATTATTTTTAATTGTTAATTTAATTGTAATACTAAGGCAACCACTAAAAAAGCCT
TTAAAAAAATATAGCACTTGAGGCTGGGCATGGTGGCCCTCAATTATATATATAATATATATAA
AATATATATTATATATATAGTAGATGAACAACAATGGGATTTAAATGGTACACTAGAAAATATC
TGTTTAACAAAAAGAAAGCAATAGTGGAGAAATATAAGAACAAAACCATGTAAGATTTATAGAA
AATGAATAGCAAATTGGTTGACCTAAACCCTATCTTATAATTATATTAAAGATAAATGAAATAA
ATACTACATCAAAAGGCAGAGATTATCAGAATAGAGAAGAAAAATCCAAACCATAATTCAACCT
TATGTTATCTGTATTTAGAATATTTAGAGTCAAGACACAAATAGATAGAGTTCCATTTGGGCGT
GAAAATATTTACCATGCAAAATGTAATTAAAATAGAGCTAGAGCAGCAATACTAATATCTGACA
AAATATACTTTAACAAAAATTGTTGCTAAAGACAAAAAAGAACATTTTATAATAAGACACAACA
ATTATAAACATATACACCAAACAATAGAGCCCAAAATATACAAAGCAAAAACTGCTAGAATTGA
AGAAAGATAGAAAAATCAATGATTACAGTTGGAGGTGTCAATACCAAACTTTCAGTAATACACA
GAACAACAAGTCAAAAGCAAAAAGGAAATAAAAAACTACACATTATCATACAACACCTTAATAC
GATATCCAACAATAGCAGAATACACATTATTCTGAAGTGCACATGGAATATTCTTCAGGATGAC
ACATATTAGGCTGTAAAACATATCTTAATAATTTAAAAGAATTGAAATAATACAGCACATATTC
TCTGACTGCAAAATATTAAACCAATTACAGAAGAAAATGTGGGAAATTCACAATGATATGGAGA
TTTAAAAATACTTCAAAGTAACCAATGAATCAAAGAACAAATCACAAGAGAAATTAGAAAATAT
TTTAGATGAATAAAACTGAAGAAACAACATACCAAAATTTTGTGGTTGTAGCTAAAGCAGTGCT
TCAAGGGAAATGTATAACAGTATATCCCTAAATTTTAAAAAAATCTCAATCCAATAACCTAATT
TTTCACCTTAAGAAATGAGAGTGAAAGAGCAGACTTAACCCAAAGTAAACAGAAGGAAGAAATA
ATAAAGATTAGCATGGAGATAAGTAGTGAAAATAAAAACAATAGAGAAAATTAATAAACTTGAA
AGTTGGTTCTTCTGATATATCAGTAAAATTGACGTATTATTAGTTTGACCGAGAAAGAACAAAA
GAGAGAAGATTCAAGTTACTAGACATAAATGAAAGTATAGATATCACCTTACAGAAATTAAAAG
AATTCTAACAGAATATTGTGAAAAAGTGAATGTCAAAAAATTAAATTATATGAGATACACAAAT
CCCTCTAAAGGCACAAACTACCACAGCTGGTGTAAAAACATGAATACACCATTTACAGTTAAAA
AGACTGAATAGATAAAAATTTTAAGAAGAAATTGAGTAAGTAATTTAATTTTCAAACTATAATC
CCAAGACCAGATGTTTTCATTGGTGAATTCTACCAAACTTTAAAAGGATTAATATCTATTTTTC
ATACACTCTTTCCAGAAAATAGAAAAGGAGGGAACACTCTATAACTCACTGTATGAGGTCCGTA
TTACCCTTATATCAAGACCAAACATCATAAGAAAAGAAGACTAAAGACTTATGGTTTCTGCTCT
GATATGTTTGGAAGTCATCACTACTATTGTCACAAGGAAAAATCTGAACAAACTAAAGTCAACG
ATTTCTTAAACTAACCATAGAATTGAGGTAACGGGCGAAAACTGGAGATGTAGGCAAATACAAA
AAAAATCACAGTTTATCAGGAGCAGAAACTGCTGAAACCAGCAACTCGTATGAACATGTTAAGT
GGTAATTGACAAATTTCTGGAGATTGAATGTGGACTGGATTGAGAGTTAGGAACTCCTAAGTGC
CCAGTTTTTGATGACCCCACACACTTTTGTAAACTAGACTTCCAAGAGCCCCAGCAAGTTTCTT
ACAGTGAAGACTGCAGAAAAATCCCCTGATGCTTCAGATAGGAGGAAGGGAAAAGCAACTATTT
TGAAATAAGCCCAGGGGACAAGTAGTTATTTCTAAACACTCTCAGAGCATTTTCTTTCACACGG
CAGGGGGCTCCCTGCAAGGGAAGCTACTTTGCCTGAGCCTTGTCTGATGTAGGAGAAAAGGAAT
TGGATGGCTCAAGCTCCATCTAGCCTTTCTGATTTATATAAGGGAAGCAAAAAATAGGTTAAGA
AACTCTTCTGAAAGTCACAACTCAGATTTATTTTATATTTATTTATTTATTTATATTTTTTGAG
ACGGAGTCTCGCTCTGTTGCCCAGGCTGGAGTGCAGTGGCGCGATCTCGGCTCACTGCAAGCTC
CACCTCCCGGGTTCACGCCATTCTTCTGCCTCAGCCTCCTGAGTAGCTGGGACTACAGGCGCCC
AGCTAATTTTTTGTATTTTTAGTAGAGACGGGGTTTCACCACGTTAGCCAGGATGGTCTCGATC
TCCTGACCTCGTGATCCACCTGCCTCTGCCTCCCAAAGTGCTGGGATTACAGGCGTGAGCCACC
GCACCTGGCCTCACAACTCAGATTTAACCATAAGATTATAGAACACTTCTCCTCCCAAAACAGA
GATATAGATCAATGGAACAGAACAGAGCCCTCAGAAATAACGCCGCATATCTACAACTATCTGA
TCTTTGACAAACCCGAGAAAAACAAGCAATGGGGAAAGGATTCCCTATTTAATAAATGGTGCTG
GGAAAACTGGCTAGCCATATGTAAAAAGCTGAAACTGGATCCCTTCCTTACACCTTACACAAAA
ATTAATTCAAGATGGATTAAAGACTTAAACGTTAGGCCTAAAACCATAAAAACCCTAGAAGAAA
ACCTAGGCATTACCATTCAGGACATAGGCATGGGCAAGGACTTCATGTCTAAAACACCAAAAGC
AATGGCAACAAAAGACAAAATTGACAAACGGGATCTCATTAAACTAAAGAGCTTCTGCACAGCA
AAAGAAACTACCATCAGAGTGAACAGGCAACCTACAAAATTTTCACAACCTACTCATCTGACAA
AGGGCTAATATCCAGAATCTACAATGAACTCAGACAAATTTACAAGAAAAAAACAAACAACCTC
ATCAAAAAGTGGGCAAAGGATATAAGCAGACACTTCTCAAAAGAAGACATTTATGCAGCCAACA
GACACATGAAAAAATGCTCATCATCACTGGCCGTCAGAGAAATGCAAATCAAAACCACAATGAG
ATACCATCTCACACCAGTTAGAATGGCGATCATTAAAAAGTCAGGAAACAACAGGTGCTGGAGA
GGATGTGGAGAAATAGGAACACTTTTACACTGTTGGTGGGACTGTAAACTAGTTCAACCATTGT
GGAAGTCAGTGTGGCGATTCCTCAGGGATCTAGAACTAGAAATACCATTTGACCCAGCCATCCC
ATTGCTATATATATATATATATACCCAAAGGACTATAAATCATGCTGCTATAAAGACATATGCA
CAGGTATGTTTATTGCAGCACTATTCACAATAGCAAAGACTTGGAACCAACCCAAATGTCCAAC
AATGATAGACTGGATTAAGAAAATGTGGCACATATACACCATGGAATACTATGCAGCCATAAAA
AAGATGAGTTCATGTCTTTTGTAGGGACATGGATGAAATTGGAAATCATCATTCTCAGTAAACT
ATCGCAAGGACAAAAAACCAAACACTGCATGTTCTCACTCATAGATGGGAATTGAACAATGAGA
ACACATGGACACAGGAAGGGGAACATCACACTCTGGGGACTGTTGTGGGGTCGGGGGAGGGGGG
AGGGATAGCATTAGGAGATATACCTAATGGTAAATGACGAGTTAATGGGTGTAGCACACCAGCA
TGGCACATGTATACATATGTAACAAACCTGCACATTGTGCACATGTACCCTAAAACTTAAAGTA
TAATAATAATAAAAAAAGGCTACCTAAAAAAAAAAAAAAGAACACTTCTCCTCCCAACACCATA
TCACCACATCAACCAGGACTCCAGTGTAATAGCAGTGAATTCTAACTGAAAGAGGTGAAAGACA
CTGATTGTATTTAAGAAAGATCTTCTAAGGAAATCCAAAAATAGTAGGGGAGATCAAAACAAAG
ATACTAGAGGAAATTGAATATGTGACACCTATAGCTACAAAAAAATTAAACATAACATAGCCCT
AACCATATAAACATAAAACCTCACACAAAGACCTATTATCTGAGATTCTGTTGCCTGATACATT
GCGTTTTATTTCAATAAAAAAATTAGAGGGTATGTTAAAAAGCAGGAAAAGTTAGTCTAAAGAG
ACAAATTGAGCCTCAGAAGTAGGCTCAGATATGGCAGAGATTTGGCAATTATACCTAGAGTTTA
ATATAAATGATTAATATAATAAGTGTTCTAACAGAAAAAGGCAACATGCAAGAACGGATGGGTA
ATGTGATCAGCAAGAAGGAAACTCTAAGAAAGAAGTCAAAAGGAAATGCTAGGAATAAAAACCT
ACAAGAAATAAAGAATGCCTGTGATGGGTTCCTCAGTAGACTGGACAAGGTCAAAGAATCAGTG
GATTTGAAAATATGTCAACAAAAACTGCCCCACTGAAATACAAAAGAAAAATAGAATTTTAAAA
ACGTAACACAATCTCCAAAACAGTGGGACAATTACAAAAGATGTAATGTGCCTAATGCAAATGA
CAGTAGGAGTATAAAGGGAGAAAGGAATAGAAAATCTGAAGTAATAATGGCAGAGAGTTTTCCA
AAATTAATGCTAAACCACAGATACAGCAAGCCCAGAGAACAACAAGGAGGAAATTTAGTAAAGC
GTCTGCAACCAAGTATGTCATATTCAGACTGACAAAACCAAAGGTGAAGAGAAAATATTGAAAG
AAGACAAAGAGGAAAATAAATATCAAGAAAATACATACGAAATACATCATACATACATAAGAAA
TACATCAGACCATACAAGCAAGAAGAGAATGGAGTGAAATGTTTAAAATGTTGAAAAAAAAACT
ATCAATTTGCAATTCTGTATCCAGTGAGATTATCTTTCAAAAGTGAAGAGGGAAATGGCAGAGA
AGTCATCTCCAAGACCTATGGTTTCCCTTCACAGAAACACTGAAAAATATGAACAAAAGTGGTC
AGAATTAACTTTCTAAGAATTCTATAAAATGGTAAAATGTTTACACCAGTAAAGCAAATGCTGA
ATTGAGAAGGCAACTTAAAAAGGTGAAGAAAACTTCGTATTATTTTTATGTGTCCTTGCCCCAC
GTCCTTCCCTACCTTAGTCTTGAAGATGGCAGCCCACATTTCTACTGTGGGGCTCTGGTTTCTG
TTTCCTGGTTCAAGAGGGAGAATAACAGACCTTACTTTTAGTCATTATTATTTCCTTCTTTCTG
ATTTCCTTGGGTTTATTTTGCTCTTCTTTCTACTTTATTGAAATGAGAACTAAGATTATGATTT
GAGACATTTTTCTAATGTAAGCATTTAGTGCTATAAATTTCCATCTCAACACTGCTTTAGTCAC
ATCCCACAAATTTTTATATGTTGTAATTTCACTTTCATTTAGTTCTATTTTTAAATTTTTTCTT
TTTATACTTCCTCTGACTCACAGATTACTTAGAATTGTGTTGTTCAGTTTTCAAGGATATTGTA
GATTTTCCTGTTTCTCTGTTGTCTAATAGTTCTGTTCCATTTTGTACAGATAGCTCACGCTGTA
TGATTTCAATTTTTTAAAAAATTGTGCTTTGTTTTATGGCCCAGATATGGTCTGTGCTGTGAAT
ATTCCATGTTATTATAAAGTATGCCTGTTATATTATTATATATATATAATATATATAATTATAA
AGCATGCCCGTTTTGTATTGTTAGCAGAGTATTCTAGAAATGTCAATGAGATCTTGTTGGTTGA
TGGTGCTTTTCAGTTCTATATCTTTGCTAATTTTTTTTTTTTTTGCTTAGTAGCTATATGAGAT
TCTGAGAGAGGAAATTGAAGTCTCCAACCATAATTGTGGATTTGTCTATTTCTCCTATCAGTTC
TATCAGTTTGTGCATCACATATTTGAGGCTCTGTTGTTTGGTGCATACACAAGTGGAATCATTG
TGCCCTCTTGGTGGCTTATTTTATGATTATATAGTGCCTATCTTTGTGGTATTTTTATTTGCTC
TTAAATCTACTTTGTGTTATATTCATATACCCATTCTTTTTTAAAAAAAATTGTTTGCGTGATA
CATCTTTTCCATTCTTTTAATCTCAGCCTATCTGTGCCATTGAATTTGAAGTGAGTTTTCATAT
AGAGAACATATTATTGAATCATCATTTTAAAAATTCCTTTTGCCAATCTTTTTTATACTGAGGT
AAAATTGACATAAAATTTATCATTTTAAAGTGTACAATACAGTGGCATTTGGTAATACACATGT
TATGCAACGTTAACTCTACCTGGCTCCTAAATGTTTTCATCATCCCCAAAAGGAAACTTCATAC
TCATTAAGCAGTTAATTCCCATTCCTTCTCTCGGCCACTGGCATCCGCAAACCTACTTTTCTGT
CTCTATGAATTTACCTATTATGGATATTTTGTATAAATTGAATTATACAATAAGTGACCTTTAT
GTTTGGCTTCGTTCGCTTCGCATACTATTTTTCGATATTCAACCATGTTGTAGTATGTATCAGT
TTTATTTGAATAACTCAATTCTTTTTGTTGTATAGCTAAAAGTTGATTCCTAGGTCATAATGAT
AATTCTATGTTTAGTTTATTGAATAGCTGCCAAAGTTTTTCCACAGTGGCTCTGTCATTTTAAA
ATCCCACTAGCAATGGATGAGAGTTCCAATATCTCCACATCCTTACCAATATTGTTATTTTATA
TTTTTATAATTATAATTTTCCTAGTGAATACGCAATGGTATCTCATTGTGTTTTTGGTTTGCCT
TTCCCTAATGACTAATGATGTTGAGCATCTTACAATGTACTTGTTAACTATTTGTGTTCTTTAG
AGAAATGTCTATTCAAGTGCCTTGTCCATTTTAAAAATCGAGTIGTCTTGTTGACTTATGAGTT
CTTTAATACAGTAAACGCTTATCAGATATGATTTATAAGTATTTTAACCCATTCTGAAGGTCAC
CTTTTCACTTTTGTGGTAGACCATTATGCACAAAGGTTTTAATTTTGATAAATCCAATTTATCC
GCTTTTGTTGTTGTTTTTGTTGTTCGTGCTTTTGCAAAACCTAGTGTCATGAGGTTTTCTCATT
ATCTTTGGAGAATTTTATAGTTTGGGTCTATACATGTAGATTATTGATCTAATTTCCATTAATT
TGTGTGTATGCTACGAGGTAGGGGTCCAAATTCAATTTTTGCATTGAATTGAAAATTCATATTT
TCAGTTTCAAATTTCAACTGCATATTCAGTTGTTGCAGCACAATTTGTTGAAGAGATCATTCTT
TACCACAGGGAATGATCTGGGACCCTTGTCAAAAATCAATTGATCATAGATGTATGGGATTATT
TCAGACTTTAGATCTTGTTCCATGAATATGCCTATTTTTATGCCAGCACTGCAGTATTTTCATT
ACTGTAGCTTTATCATAAATTTTGAAATCAGGAAGTATGTATCCTCCAAGTGTGATTTTCATTT
GCTAGAGTGTTTTGACTATTTGGGGTCTTTGCAATTCCATATGAATTTCAGAATTGGCTTTTCA
TTTAAAAAAATGGTAGTTGAGATTTTCATAGGAATTATATTGAATCTACAGATCACTTTGGGTA
GTATTGCCATCTTAACAATATTGTATTCCAATCCATAAACACGGATGTATTGCCATTCATATCT
TTTTTTCTTTTCTTTCGGCAACATTTAGTATATGACACTTGTAACTCCTTGGTTAAATTTATAC
CTAAGCATTTTATCCTTTCTGATGCTGTTTAAATGGAATTATTTTATTAATTTCTCTTTTAGAT
GGCTTGTTGCAGGTGTATAGAAATAGAACTGATTATTGTGCTTTTATTGAATTATCTGAAACTT
TGCTGCATTTATTAACTCTAGTAGGTTTTTTTTTTCTTTAAAGTTGTCTATATATCTTGCTCTG
TGAATAGATAATTTTACTTCTTGATCTCCAATATGGATGCCTTTCTTTCTTTTTCTTACCTAAT
TGCTGTAGCTAGAATTTTCAGTATAATGTATGATAGAAGTTGTTCACAACTATCCTTGTCTTCT
TTCTGATCCTAGGGGTATAGCTTTCAGTCTTTCACCATTAAGCACAATGTTAGCTGTGGGGTTT
TCTTAGATGCCATTTAATATATTAAGGAAGTGCTCTGCTATTTCTAATTGGTTGAGTATTTTTA
TTATAAAATGGTGTTAGATTTTATGACTTTATGTACTGCATAATTGAGATTATCATGTGGTCTT
TTCATTCGATTAATGTGGTATATTTTATGATTTTCATATGTTGATCCACCTTTATATTCTTGGG
GCAAATCCCACTGTGTACACGGGTTTTTTAGGGTCTCCTAATTTTCTTCATTCTTTTTCTTTTC
TCTCCCTGAGACTGAATAACGTTAACTGACCTATCTTCAAGTTTACTATTTTTTCTTCTGCTGT
TCAAATCTGCTTGAACCTATAGAGTGAATTTTTCATTTTACTTATTGAACTTTTCAGCTCCAAA
ATTTCTCTTTGGCTACTTTGTATAATATCTATCTCTCTATAGATATTCTCTATTTGGAAAGACA
TTTTTCTCCTGGTTTTCTTTACTTATTTGTATTTTTTAAAGTCTTTAAGCATATTTGAGACAGT
TGATTTAAGTATTTGTGTACTAAGTCCATTGCCTAAGCTTTTGCATAGAGATTTTATATTAATT
TCTTTTTTTTCTGTGAACAGGTCTTTTTTTTTTTTTTTTTTTTTTTTTTTTTTTTAGAAAACTG
GACATTTTGAGTAGTATAAACGTGGCTATTTTAGAAATAATTTTCCTCCCTCCTTAGGTTTTGT
TTCCACTTGTTGCATGTTGCTGTTGTTTGCTCATTAGTGACTTTTCTAAAGTATTTTGAAAAGT
CTGTGTTCTTGATCATGTGGGTCCATTGAATTCTGTATTCTGTTAATTTCATAGTCAGCTAGTG
TTCTGAAAGTTCCCTTAAGTGCATAGAGCCAATAAAAGAAAAAGAAAAGAAACACAGAAAAAGA
AAAGAGGAAAAAAGGAGGGAAAGAGATTTAAAAAAATAATGTCGGTTGGGCATGGTGGCTCATG
CCTGTAATCCCAGCACTTTTGGGAGGCCGAGGCAGGCAAATCACTTAAGGTTGACCATCCTGGC
CAACACGGAGAAACCTTGTCTCTACTGAAAATATAAAATTAGCCGGGCATGGTGGCACATGCCT
GTAATCCCAGCTACTCGGGAGGCTGAGAGGTAGGAGAATCACTTGAACTCGGGAGGTGGAGGTT
GCAGTGAACTGAGATCACACCACTGCACTCCAGCCTGGGCAACAAGAATGAAACTCCATCTCAA
AAAAAAATAATAATAATAATGCCTTTGCCGATTTGCTCTGTGTTTGGGCCCTCATTCAATGCCT
AGTCAGGCCATTTACAACTCTCTCTTAACTTTCACTACCTGCTTGTGTACTGACTGAAGGCCAC
CTATAGGTGAAAGCTTAACATCATCTCAGATCTTTTTGGACCATGAATCCTACCCTGGGTATGC
ACATGATCTTCTTAATTTCCCAGTAGATGCAAGAGTTTTAGTGTTTTAAAAGTCCTTATTCCCT
CATCTATCTTCTTTTCTGACCTTTTTCAGTCTGCTTATTGTTCATCTGAACTGACATCCTTTGC
CCCAAGCGGCTGTGGCAAAAACTTTTACCTTTAAATGCTTTCACCACAAGCCACTTGGGAAGCT
GCCCCAGACCTGGGACTGCTCTGACCCTGATGAAACAAAGACAAGACCTTGTACAGCCAGGCAG
CCATAAGACAAGTCCATACCCAAACCACAGTTCTTTCAGAATAAGGTCTATATTGGATTCTCTG
GCCCTAGTAACCAGCATGAGTCTGGGCTTGCCATCTTCATGGCCACTTGTCTTAGTTTGCTAGG
GCTGCCATAACAAAATATGAGAGACTGAGTGGCTTAAACAAGAGAAATTTATTTTTTCACAATT
CTGAAGACCAGAAGTCCATTACTCTGAAATCTCTCTCCTTGGCTTGCAGATACTGCCTTCTTGC
CGTGTCCTCACACAGCCTTTTCTCTGTGTACACATCCCTGGTGTTTTTTAAGCCTGTCCAAATT
TTCTGTTCTTCTGAGGACACCAATCAGATTGGATTAGCGACCACCCATATGATCATTTTACCTT
AAGTACCTCTTCAAAGGTATCAGAGTAGTATATTTGCTACAGTTGATGAACCTGCATACAACAT
CATCACTTGAGGTCTGCAGTTTACATTAGAGTTCATCGTTAGTGTTATATATTCTAGGGTTTGT
TTTGTTTTGAGACAGAACAGAGTCTTGCTGTGTTACCCAGGCTGGAGCGCAGTGGTACAATCTC
ATTGCTACCTCTGCTTCCCAGATTCAAGCAATTCTCATACCTCAGCCTCCTAAGTAGCTGGAAC
TACAGGCGCACACCACCATGCCCAGCTAATTTTCGTATTTTTAGTGGAGATGGGGTTTCACCAT
GTTGGCCAGGCAGGTCTTGAACTCCTTGCCTCAAGTGATCTGCCCGCCTCAGCCTCCCCCAGTG
TTGGGATAACAGGCATGAGTCACCATGCCTGGCCTTATTCTATGGGTTTTGAAATGTATAATGA
CATGTATCCATCGTTGTAGTATTAGATAGAATAGCTTCATTACCCTAAAAGTCTTCTTTGCACT
GGGTTGAGTTTTAAAAGCTCTTTGATTATTTTATGACAGTTCCTTATTAGATATATCTTTTGCA
AGTATTTTTATCAGTCTGTGGTTATCTTGTCTTTTACAGAGCAGTAATTTTTAATTTTAATAAA
ATCCAATTTGTCAATTACTTATCTCATATGACTCTGGTGTTACATCTAAAATGTTACCACCATA
CTCAAGGTCACCTAGGTTTTCTCTAGGAATTTTATGGTTTTGCATTTTACATTTGGTGTATGAC
CCATTTGAAGTTAATTTTTGTGAGGTTGTAAGGTCTGTGCCTAGATTCATTTTTTTTTTTTTTG
GCATGTTACTTCAGTATTCATAAGAAACATTGTTCTGTAATCTTCTTTCTTATAGTATCTTAGT
CTTGCTTTAGTTTTTGGGCAATGCTGGCCTCACTGAATAAATTCAAAGTGTTCCCTCCTCTTCA
ATTATTTGGAAAAGTTTGAGAAAGACTATTGTTAACTGTTTTCTAAATTTTTGGTGGAATTTAC
CAGTGAACCAACTGGTCCTAGGCTTTTCTCCAGTAGGTGGTTTTGATTATGCTTTCAATCTCTT
TACAAGTTACACATCTATACAGACTTTTATAATTCAGTCTTGGTAGGTTGTGCGTATTTAGGAA
TCTGACCACTTCATCTAAGTTATCCAATTAGTTGGCATGCAATTATTCGTAGTTCTCTGAATAA
TCATTTTTATTTCCACAAAATTGGTAATATCCCAGTTTCCATTTTTTATTTCATTGAATCTTCT
TTTTTCTTAGCTAATCTAGCTAAATGTTTGCCAATTTTGTTGATCTTTTGGAAGAACCAACTTT
TGATTTATTAATTTTCTCTACTCTTTTTCTGTTCTTTATATTATTTATTTCCACACTAATCTTT
ACTATTTTCTTCCTTCTGTTGGCCTTTAATTTTTTTTTTTTAATTTTTAAGGTGTAAATTTAGG
TTGAGAAATTTTTTAAATGAAAGCATTTAGAGCTATAAATTTTCCTTCTGGTGTTCCTTTCACT
ACCTGCCATAAATTTTGATATGTTGAGTTTTTGTTTGTCTTGGAGTATTTTCTAATTTGTCTTC
TAATTTCTTCCTTGACCTATTGGTTATTTAAATGTATTTAATTTTTGCATATTGTGGATTTCCC
AGTTTTCCTTCTGTTATTGATTTCTAGTTTTATTCCATTGTGATCACAGAAGATATTTTGTATA
ATCGCAGTCTATTAACATTTATTAAGTCTTGTGGCCTAACAGAGGATCTATGTTGGAGAATGTT
CCAAGTGCAATTGAGAATACTATTCTGGTGCTATTAGGTGAAGTATTCTCTATATGTCTGTTAA
GTCCAATTCATCTATAGTGTTGACGTTTCCTGTTCCTTACTGATTTTCTGACTTATTATTCTAT
CCATTATTAAAAGTGGAGTGGTAAAGTCTCTATTATTGTAGAACTCTCTGTTTTTCAAGTCTAT
CAATATCTGTTTCATATATTTTGGAGCTCTGTTTGCTGCATATGTGTTTACAATTGTTATATCT
TCTTGGCAAATTGACCAGTTTCATCAACATAAAATATAATTCTTATTGTCTTCTAACAGTTTAT
TTTTCTTTTTACATAAAGCCTATTTTATCTGATCTTAGATTCCCTCACACTCCACCCCAGCACG
CTTTTGGTTACATTTACATAATATATCTTTTCCATCCTTTCATTTTCAACCTGTTTGTGTCTTT
AGATCTAAAGTGAATGTCTTACAGACAGCATAAGCTATGTCATTAAAAAAATCCATTCTGCTTA
TCTCTGCCTTTTGACTGGGGAGTTTAATCCATTTGCATTTAAAGTAATCACTGATCATTAAATA
CTTTCAGTATTTTGTTGTTTTATGTATGTCTTATAACTCTTTTGCTCTTCATTTCCTTCAATAT
TGCCTTTGTGTTTAGCTTATCTTTTTGTGTCACACTTTGATCCCCTTCTCATTTCTTTTTTATA
TTTTCTTTGTGGTTACCAGGAGGACAATGTATCAACTTTTAAAGTTATTACAATTTTATTTTTT
TAAATCTCTCCCATTCGGGGATTTTAGGAAGGTTAAATAATAATGTAAATGAGATACCTAGAAC
AATATAAGCATTCAGGAATTATTAACTCAATTCCAATCCTTCCTCCACCTCCACCTCTTTCTCT
GTGAGATTATAGAAAAGATGACAAAAAGGATGTTTTCTGAGCCCTTTAATTGTTGAGAATGATC
TTTGAGAAAAAGAAAAAAAATGAAAGCACTAGGAATGTACAACAGCCTGGAAGTATAATTAAGT
GTAAATTAAATAGATAAAAGTTATAAGCAGAGGAAAGTATAGTAGAACTCAGTATTTAAAAGAG
AATCAATGTGAAAATTATATAAATTTATGTAAAATAAAACTACCAGACAAATCTGATATCCTTA
GGATTTTTCTTTCTTTCATGTGATTTCTAATTGCTACATATGACACTAAACCATTGATCTGAGC
TGTAAGAGAAACTGGAAATTGTTCTGTTATCTTTTGTAAGATTTCTAGAACATTTTGCCCTCAG
ACTTAAATGCCAACGTATTTCTCACTTATTGTTTACTGCTTTTGGATTTACATATGATTTGATT
CTTTCTTATCTCTTATCCTTACAATGTAATTCAAACTGATGCCAATTTAAGTTCAATTGCGTAC
AAAAACTCTACTCCTATGCAGCTCCGCCCCATCTAATTTACATTATTGATGTCGCAAATTCCAT
CTTTGTACATAGTTTACATATTAACATGGATTTATACATTTTTATGTATTTGGTTTTTAAATCC
TGTAGAAAATAAAAAGTCAACACACCAATATTAAAATAATACTGGTTTTTATATTTGTCCATGT
GCTTACCTTTATCAGTGTTCTTTACATCTTTATACGGGTTTGAGTTACTGTCTTGTGTCCTTTA
GTTCCAACCTAAAGAACTCCCTTTAGCATTTTTATAGGGCAGGTCTAGTGGTAATGAACTCTCT
GAGTTTTTATTTAGGGATGTCTTAATTTCTAGCTCCTGTTTGAAGTAAATTTTTCTGGATATAC
AATTCTCTGTTGATTGATTTTTGTTCATTTTCCCTTCAGCTCTTTTAAATACATTATCCCACTT
TCTTCTGCCTTCCAAGGTTTCTATTAACAAAATTCAGCTTATAATCTTATTAAAGATCTCATGT
ACATGAGTGGCTTCTCTCTTGCTGTTTTCAAGATTCTGTGACTTTGGTTTCTGATAGTTTAAAT
ATAATGTGTCTTATTGTGGGTCTCTTTGGATTTATCCTAGAGTTTCTTGGTCTTCTTACGTTGG
TATATCCATGTATTTCAAGACATTTGAGTAGTTTTCAGCCATTATTTCTTCAAACAATCTCTCC
TCTTTGGGGACTTCCATTTTACCTATATTGGTTCTTTTGATGGTGTGGCACCAGTCCCCTAGAC
TTTGTTCACTTTTTTCCAGTCTTTTATTTCTGCTTCTCAGACTCAACAGCTTCAAGTGTTCTGT
ATTCAAGTCTGCTGACTCTTTCTTCTTCCAGCTCAAATCTGCTGTTGGATCCCCCCTTGTAAAA
TTTTTAATTCCATTTTAGTGTTTTTCAAGTTAAGTATTTTTATTAGGTTCCTTTTTATAATTTC
TTTTTGTTGATATTCTCATTTTATTACACATAATTTGTCTGATTTCCATTAGTTTTTTTCTTTG
TTTTCCTTTAGCTCTTTGAAAATATTTAAGACATTTTAAAAGTCTTTATCCAAGTTCAATTTCT
ATGGTTCTGTAGAGATATTTTCTGCCAGTTTATGTTCTTCTTTTCCATGGGCCATGTTTTCCTG
TTTCTTTGTATACTTTCTAATTTTTGGTTGAAAACTGAGCATTTGAAAATAGAGCCAACTTTCC
CAGTTTCTGCAGAGAGTCTTTATGCCACAGTATTCGTTCACTGATTAACTGGGTATATCTAAGC
TTAGGGAGCAGCTGAGTCAAAAGTTTAAGGTCTTCTCAGGTCTTTTCTGAGTATACATGTTTCC
TATGCCTGTGTGAAATGTTCTCAATTTCCCTATATAAACAGCTACTTCTTCTTTTTTTTTTTTT
TTTGAGATGGAGTCTCGCTCTGTCACCCAGGCTGGAGTGCAGTCATCTCAGCTCACTGCATCCT
CCACCTCCCAGGTTCAAACAATTCTCCTATACAGGTGTGTGCCACCACGTCTGGCCAATTTTTG
TATTTTTAGTAGAGACAGGGTTTCGCCGTGTTGGCCAGGCTGGTCTCAATCTCCTGACCTCAGG
AGGATTACAGGCTTGAGCCACAGTGTCCAGCCTAAACAGCTACTTTTGAATGCTTTAATTTCCT
GAATAGTCTCAACCCAGTTTTTCCTTGAGGTCTTAGGTGGTCCATTGTATGTCTCCACCCATAG
TTGCTTGCCCCAGGCATCTGTGGGTCTGTGGTACCACTGCAGCTTTCACCACCTGTAGCTGCCA
CCTTTCCCTATCTGAGATCCAGGTTAGGTGAGAGAGATCATTCCTTCACGCAGTCCCATGACAG
GTTGGAACATTTCAAATAAGGTCTGTTCTGCTCCTCTGGTTGAAGGGAGAAAATTGGGAACCGG
TTTCCCACCTTCTACAAACCAAGATCTCATGTTGCCACGGGAGTGGCAGGGCAAGTGCAAGTGA
AAATGCCATACAATTTTCTACCATTTTGAACGCGGGTTTTTCTTCAATGGTCATTTGCTTGGTT
GCTGTAGGCCTTTCACTGTTTTCCAGAGCTCCCATAAGATTACTTTAGCCAGTTTTTTGTTCTT
TCCTGATGCTTCCCTGGCAGAGTAAGGGTTGGAACTTCCACCATTTTGCTGATTCATAACTCTG
TAGTCAGTTTTAAATATATTGATACTTGAGTTTGTTTTATGGCCCAGAATATGGTCTTGGTAAA
TGTTTCACATATACTCAGAAAGAATGTGTATTCTGATGTTGTTACATGGGCTGTTCTATAAATG
TTACTTAAGGTGGTTAATAATGTTGCTCAAGTCTTCTATATTCTTGCTGATTTTCTTTTATTTA
TTTATTTTATTTTTTTTATTATACTTTTAAGTTCTAGGGTACATGTGCACAACATGCAGGTTTG
TTATATATGTATACATGTGCCATGTTGGTGTGCTGCATCCATTAACTCATCATTTACATTAGGT
ATATCTCCTAATGCTGTCCCTCCCTGCTCCCCCCACCCCATGACAGGCCCCAGTGTGTGATGTT
CCCCTTTCCTGTGTCCAAGCGTTCTCATTGTTCAATTCCCACCTATGAGTGAGAACTTGCGGTG
TTTGTTTTTTTGTCCTTGTGATAGTTTGCTGAGAATGATGGTTTCCAGCTTCATCCATGTCCCT
ACAAAGGACGTGAACTCATCCTTTTTTATGACTGCATAGTATTCCATGGTGTATATGTGCTACA
TTTTCTTAATCCAGTCTATCATTGATGGACATTTGGGTTGGTTCCAAGTCTTTGCTATTGTGAA
CAGTGCTGCAATGAACATACGTGTGCATGTGTCTTTATAGCAGCATGATTTATAATCCTTTGAG
TATATACTCAGTAATGGGATGGGTGGGTCAAATGGTATTTCTAGTTCTAGATCTTGAGGAATCA
CCACACTGTCTTCCACAATGGTTGAACTAGTTTACAGTCCCACCAACAGTGTAAAAGTGTTCCT
ATTTCTTCACATCCTCTCCAGCACCTGTTGTTTCCTGACTTTTTAATGATCGCCATTCTAACTG
GTGTGAGATGGGATCTCATTGTGGTTTTGATCTGCATTTTTCTGATGGCCAGTGATGATGAGCA
TTTTTTCATGTGTCTTTGGCTGCATAAATGTCTTCTTTTGAGAAGTGTCTGTTCATATCCTTTG
CCCACTTTTTGATGGGGTTGTTTTGTTCTTGTATATTTGTTTGAGTTCTTTGTAGATTCTGGAT
ATTAGCCCTTTGTCAGATGAGGAGATTGCAAAAATTTTCTCCCATTCTGTAGGTTGGCTGTTCA
CTCTGATGGTAGTTTCTTTTGCTGTGCAGAAGCTCTTTAGTTTAATGAGACCCCATTTGTCAAT
TTTGGCTTTTGTTGCCATTGCTTTTGGTGTTTTAGACATGAAGTCCTCGCCCATGCCTATGTCC
TGAATGGTATTGCCTAGGTTTTCTTCTAGGGTTTTTTATGGTTTTAGGTCTAACATTTAAGTCT
TTAATCCATCTTGAATTAATTTTTGTATAAGGTGTAAGGAAGGGATCCAGTTTCAGCTTTTTAC
ATATGGCTAGCCAGTTTTCCCAGCACCATTTATTAAATAGGGAATCCTTTCCCCATTTCTTGTT
TTTGTCAGGTTTGTCAAAGATCAGATGGTTGTAGATGTGTGGTATTATTTCTGAGGGCTCTGTT
CTGTTCCATTGGTTTATATCTGTTTTGGTACCAGTACCATGCTGTTTTGGTTACTGTAGCCTCG
TAGTATAGTTTGAAGTCAGGTAGTATGATGCCTCCAGATTTGTCCTTTTGGCTTAGGATTGTCT
TGGCAATACAGGCTCTTTTTTGGTTCCATATGAATTTTAAAGTAGTTTTTTCCAATTCTGTGAA
GGAAGTCATTGGTAACTTAATGGGGATGGCATTGAATCTATAAATTACCTTGGGCAGTATGGCC
ATTTTCACGATACTGATTCTTCCTATCCATGAGCACGGAATGTTCTTCCATTTGTTTGTGTCCT
CTTCTATTTCGTTGAGCAGTGGTTTGTATTTCTGCTTGAAGAGGTCCTTCACGTCCCTTGTAAG
TTGGATTCCTAGGTATTTTGTTCTCTTTGACGCAACTGTGAATGGGAGTTCACTCATGATTTGG
CTCTCTGTTAGTCTGTTACTGGTGTATAAGAATGCTTGTGATTTTTGCACATTGATTTTGTATC
CTGAGACTTTGCTGAAGTTGCTTATCAGCTTAAGGAGATTTTGGGCTGAGACGATGGGGTTTTC
TAAATATACAATCATGTCATCTGCAAACAGGGACAATTTGACTTCCTCTTTTCCTAATTGAATA
CCCTTTATTTCTTTCTCCTGCCTGATTGCCCTGGCCAGAACTTCCAACACTATGTTGAATAGGA
GCGGTGAGAGAGGGCATTCCTGTCTTGTGCCAGTTTTCAAAGGGAATGCTTCCAGTTTTTGCCC
ATTCAGTATGACATTGGCTGTGGGTTTGTCATAAATAGCTCTTATTATTTTGAGATATGTCCCA
TCAATACCTAATTTATTGAGAGTTTTTAGCATGAAGGGCTGCTGAATTTTGTCGAAGGCCTTTT
CTGCATCTATTGAGATAAACATATGGTTTTTGTCTTTGGTTCTGTTTATATGATGGATTATGTT
TATTGATTTGTGTATGTTGAACCAGCCTTGCAACCCAGGGATGAAGCCCACTTGATCATGGTGG
ATAAGCTTTTTGATGTGCTGCTGGATTCAGTTTGCCAGTATTTTACTGAGGATTTTTGCATCAA
TGTTCATCAGGGAAATTGGTCTAAAATTCTCTTTTTTTGTTGTGTCTCTGCCAGGCTTTGGTAT
CAGGATGATGCTGGCCTCATAAAATGAGTTAGGGAGGATTCTCTCTTTTTCCTATTTACTGTAC
ATTTATTCCACCAGTGACTAAAACAGGTGTATCAATAAAATCTGTTCCCTCAGGTTTTGCTTCA
GGTATTTTGAGGGTCTGTTATCAGGTGCATAAACAAGATTGTTATGTCCTATTCTTAAATTAAT
CTCCTTATAATTATGAAGTTAATTTTTTTTTTCTTGAGATGCAGTTTTGCTCTGTCGCCCAGGC
TGGAGTGCAGTGGCACAATCTCGGCTCAGTGCAACCTCTGCCTCCTGGGTTCAAGCATTTCTTT
GCCTCAGCCTCCCGAGTAGCTGGGGTTACAGGTACCTGCCACCACGCCCGGCTAATTTTTTTGT
ATTTTTAGTAGAGATGGGGTTTCACCATCTTGGCCAAGCTGGTCTTGAACTCCTGAACTCTTGA
TCCACCCACCTTGGCCTCCCAAGGTGCTGGGATTACAGGTGTGAGCCACTGCGCCTGGACCTGG
CCCGAAGTAAACTTCTTTACCCTTGCTAATGATCTTTGCTCTGAAGCATGCTTTGCTGGTATTA
ATATAGTCATTCCTTCTTTCTTTGATTCATGTTTGCAGGGTATATCTGTTTCCATTCTTTTACT
TTTAACCTATTGTCTTTATATTTAAAGTGCATTTCTTGTAAGTATAATTGGTTTCTTAAAATCC
AATTATCTGCCTTTTAAATGTCATTTTTATATGATTTGCATAAATATGATTATTATTACAGCTA
AATTGAAATCTGTCATCTTGCTATTTGGTTTCTATTTATCCCATTTTTTTCCCCTCTTTTTTTG
CTTTCCTTGAGATTGAACATTGTATTAGTTTTCTAGGGTTGCTGTAAGAAAGTGACATAAAGTG
GATGGCTTAAAACAACAGAAATTTATTGTTTCAGTTTGGAGGCTAGTCATCTGAAACCAAGGTG
TCATCAGGGCCGTATTCTCTCTGAAACCTGTAGGGAAGAATTCTTTCCTGCCTGTTCTAGCTTC
TAGCATTTTCCAGCAATTCTTGGCATTCCTTTGCTTGTAGATGTATCCCTCCAATCTCTGCCTC
TATCATAACATAGCCATCTTCTCCCTTTATCTGTCTATTCTTCTCATCTTATAAGAACATTAAT
TACTGAATTGGGGCCCAGCATAGATTAGGCCTAATCTCATCTTGAATAGGTTACATCTGCCAAA
GATTCTTCTTCCAAATAAGATCACTTTTACAGCTTTTACAGGTACTGAGAGTTAGAACCTCAAT
ATATCCTTTGGTGGGGACCGACCTCTTACCCATAAAAAGTATTTTATATGATTCCATTTTATCT
CCTTTTTAGGTTATTAACTACAATTTTTTTTTCTTTTTTTTGAGATGGAGTTTTGCTCTTGTAG
CCCAGGCTGGAGCGCGATTTTGGCTCACTGAAACCTCTGCCTCCCGGGTTCAAGTGATTGTCCT
GCCTCAGCCTCCTGAGTAGCTGGGATTACAGGCATGTGCCACCGTGCCTGGCCAATTTTTGTAT
TTTTAGTAGAAACAGGGTTTCACCATGTTGGCTAGGGTGGGTCTCAAATTCCTGACCTTAGGTG
ATCCACCCACCTCGGCCTCCCAAAGTGCTGGGATTACAGGCGAGAGCCACCACGCCTGGCTTTA
TAATTTTTTTTTAATTCAGTGGATGTTTTAGGGTTTATAGTATACATCTTTATCACAGTCTAGC
TCCAAGTGATATATCCCTTTATGTATAGTACATGACCCTTACAGTAGTGCATTTCCATTTTTCC
TCTCTGGCATTTAGGCTATTGCACACACATACACTCAATTCATCCCTTTGTGTAGATCCGTATT
TCCAGCTGCTATCTTTTGCTTCTGTCTGAAATATATGTATGATTTCTTTTATGACTATTTTGAG
AAATTTGGTTATGTGCATTTGTCTATTTTTCTTATGTACTTTTAGCTCATCAATCTTAAGTCTG
TGAGTTTATAGTTTTTAAAAACAAATTTGAAATTATTTGGCTATTATTTCCTCAAATATTTTTT
TCTGCCGCCCCTGCTTTCCCTTTCCTTAGGGATCTCTGATTTCCACCTATATTACTCTGATTGA
AGTTGTTCCACGTCTCTTTTGAAAATCTCTGAAAAATCTTTTATCATTTGGATAATCTGTATTT
GTACATCTTCAAGTACATTAATATTTTCTTTTGCAATGTTTAATCTGCTGTTAATCCCATGTAG
TGGATTCTTCATCTCAGGTATTGCTGTTTTAATCTCTAGAAATTCCATTGAAGTCTTACTTGAT
GAAAAAGGCAGATAAAAACAAATGTTGGCAAAGATATGGAGAAATCAGAATCTGCACACACTGA
TGATGGGAATGTAAAATGGTCAAGGCAATTTGGAAAGCAGTCTGGCAGTTTCTCAAAAGGCTAA
ACGTAGTTCCCATATGACAGCAATCATTCATCTAGGTATATACTCCAGAGAAATAAAAACATAT
CCACACCAGAACTTGAACATTAATTTTCATAGCAACATTATTCCTAGGGGTTTAAAATTTTTTA
CATTATTTTTATTTAAAATAGAGACAGGGTCTCACTACGTTGCATAGGCTGGTCTGGAACTCCT
GGAATTAAGCATTCCTCCTGCCTTTGTCCTGTTTTCTCCACTGGAAAAGAATAAAACTTTGTAC
ACTTGGACTAACAACTCCCGATTCCCTCCTTTACCAACATGCCCCACAGCCTCTAGTAACTTAC
TCTCTACTTTCATGAATTCAACTTTTTTAAGATTCCACATATAAGTGAGATCATACAATATTTG
TCTTTCTGTGCCTGGCTTATTTCTCTTAGCATAATGTCCTCCAGATTCATACATGTTGTCTTAA
ATGACAGGATTTACCTTCCTTTAAAGGCTGACTAGTACTTCATTGTGTATATGTATCACATTTT
CTTTATCCACTTATCTGTTGATGGGCACTTAAATTGTTTCAATGTCTTGGCTACTGTAAGTAAT
GCTTCAATAAACATGGGAATGAAGATATCCCTTCAACATATTGATTTCTGTTCTTTTGGATAAT
ACTCAGAAGTGAGATTACTGGATCATATGGTTGTTCTATTTTTTTCAGAAACCTCCATACTGTT
TTTCATAGCGGCTGTACTAATTTACATTCCCACTAACAATGCATGAGTTCACTTTTCTGGACAT
CCTCCCCAACACTTGTTATCTTTCATCTTTTTCATAAAAGCCATTATATAATAGGIGTGAAGTG
ACATCTCACTGTGGTTTTGATTTGCATTACTCTAATAATTAGTGTGAGCATTTTTTTTTTTTCA
TGTACCGAATGTCTTTTGAGAAAGGTCTCTTCATTCCTTTGCCCATTTTAAAATCAGGTGGTTT
TCTTGCTCTTGAGTTGTTTGAGTTCCTTATGTATTTTAGATATTTACCCATTTCCAGATATATC
ATTTATATTTTTTCCTATTCTTTGAGTTCCCTCTTCACTGTGTTGTTTCCATTGCTGTGCAGGT
CTTTTATTTTGATGCCACCCCATTTGTCTATTTTTGCCATGCTTTTGCAGTCATATCCAAAAAA
ATCATTCCCAAGACCAATGCTGTGGAGATTTCCCCCTATGTTTTCTTCAGTAGGTGTACAGTTT
TAGGTCTTATATGTTAAGTTTTAAATCTATTTTTTTATATGGTGTAAATAAGGGTCTAATTTAA
TTCTTTTGCATGTGGATATCCAGTTTTCCCAACACCATTTATTGAAGACCCTGTCCTTTTATAC
TTTTCAGTATGCAGATCTTTTACCTCCTTAAATTTACACCTCAGTATTTAATATTTGTTGCTAT
TATGAGATTTTCATAATTTCCTTTTCAGATAGCTCATTAATAGTAGATGGAAACACTACTGATT
TCTGTAAGATGATTTTGTATTACGGAACTTTACTGAGTTTGTGTATCAGTTCTACCAGGTTTTA
GTTTTGTTCTGGTGGAGACATTACAGTTTTTTGTATATGGTTATGTCATCAGTAATTACAGATG
ATTTAACCTATTCCTTTCCTATTAGGATGCCTTTTTTTTCTTTCTCTTGTCCAACTGCTCTGGT
TAGGACTTCTAGTACTATGTCAAAAAGTGATGAGGGTCGTACATGGCCTCCATACCTAATCTGT
TGAGAGTTTTTACCATGAAACCAGGTTGAATTTTGTCAAATGCTTTTTCTGCATCCATTGAGAT
GATCATATGATTTGATTTACACCCTCCATTTTGTTATGTGGTATATCACACTTTTTGATGTGCA
TATGTTGAACCACCCTTGCATCCTAAGGATAAATCCCACTTCATCATGGTGAATCATTCTTTGT
ATTCGTGAATCCAGTTTGCTAATATATTGTTGAGGATTTTTGCATCCATGTTCATCAGGGATAT
TACTTTGTAAGTTTCTGTCCTTAAAGTGTCTTTCTCTGGCTTTAATAACAGTGTAACACTACCC
TTGTAAAATGAATTTAGAAGTATTCCCTCTGCTTCATTGTTTTGGAAAAGTTTGAGAATTTTTA
TTAGTTCTTTAAATGTCTGGTAAAATTCAGTAGTGAAGCTGCCTAATCCTGGGCTTTCCTTTGG
TGGGATACTTTTTATTACTGGCTCAATCTCTTTTCTTGTTATTGGCTTATTCAGATTTGTTTCT
TCATGATTCACTCTTTGTAGGTTGTATATGTCTAGGAATTTATTCATTTCTTTAGGTCATCCAA
TTTGATGGTGCATAACTTCATAGTAGTTTCTTATAATCCTTTGTATTTTGGTGATATCAGTAGT
AAATGTCTCCTCTTTCATTTCTGATCTTATTTGAGTACTCTTTTTTTCTCCTAGTCTAGGTAAG
AATTTGTTGATTTTATCTTTCAAAAAAAAAAAAAACCAACTCTTAGCAACTCTTAGTTTTGTTC
ATTTTTTTCCAGTCTTTATTTCAACTGTGATCTTTGTTACTTACTTCTTTATGCTAACTTTCGG
GCTTAGTCTGTTCTTTTCCTAGTTCCTTTAGGTGAAAAGTGAGATTGTGATCCTTCTTCTTTAT
TGGCGTAGGTTTGTATCGCTATAAATTTCCATTAGGACTGATTTTGCTGCATCACATAAGTTTT
GTTTCCATTTTCATTTGTCTCAAGGTAATTTTTTATTTACTTTTTGACTTCTTCTGTGAACTAT
TAGTTGTTTGGGAGCATATTGTTTAATTTCCACATATTGCTGTATTTTCCACCAGAATTGATTC
TTGTTCTTGATTTCTAGTTTCACGCCATTGTAATCAGAAAAGGGATTTGATATGATTTCTGTCC
ACTTAAACTTAAGATTAGTTTTGTGGACTAACATATATCCTGGAGAATGTTCCATGGGCATTTG
AGAACAAAATGTATTTTGCTGCTTCTGGATGGAATGTTTCATATATGCCTGTTAAGTCCGTTTG
GTCTAAAGTGTAATTGAAATCCATTGTTTCTTTATTGATTTTCTGTCTAGGTGATCAATCTGCC
CATGGTGAAAAGTAGAGTATTGAGGTCCCGTATTATAGTATTGCAGCCTATCTCCCTCTTCACA
TCATTTAAAAATTGCTTTATGTATTTAGGTGGGTCAATGTTGGGTGCATATACTTTTACAATTG
TTATGTCTTCTTGGTGAATTAATCCCTTTATCATTATATAACAAACTTCTTTCTTTTTATAGTA
TTGACTTAAAGTCTATTTTGTCTGATAGAAGTATAGCTACCCCTGCTCTCAATTTCCATTTATA
TAGAATATCTTTTTCCATCCCTTCACTTTCAGTCTATGTGTATGTTTAGTAGAAAAGTGAATCT
CTTGCCGGGCGCAGTGGCTCACTCCTGTAATCCCAGCACTTTGGGAGGCCAAGGCGGCGGGATC
ACCAGAGGTTGGGAGTTAGAGACCAGCCTGACCAACATGGAGAAAACCTGTCTCTATTAAAAAT
ACAAAATTAGCCAGGCGTGGTTGTGGGCCCTTGTAATCCCAGCTACTCGAGAGGCTGAGGCAGG
AGAATTGCTTAAACCCGGGAGGTGGAGGTTGCGGTGAGCTGAGATCATGCCATTGCACTCCAGC
CTTGACAATAGCAAAACTCCGTCTCAAAAAAAGAAAAAGAAAAAGAAAAAAAAGTGAGTCTCTT
ATAGGCAGTATGTGGTTGTGACTTAAAAAAAAAAAAAAATCCATTCTGTCATTCTATGTCTTTT
TGTTGGAGAATCTAATCCGTTTACATTCAAGGTAACTATTGGTAAGAAACTGCTAGTGTCATTT
TGTAATTTGTTTTCTGATTGTTTTGTAGGTCCCTTGTTTCTTTTTTCCCTATTGCTACCTTCCT
TTGTGGTTTGGTGGTTTTCTGTGGTGGTGTGCTTTGAATCCTTTCTTTTATAGTATATGCGATT
ACCATTGTATTTGCTGTAAGGCTAACTTAAAACATTTTATCCTTAGGCTACTTTAAGCGATAAA
AACTTGCCTTTAGTTGCATACAAAAACTCTACGCTTTCACAACCCCCCCGCCTTTCGTGATTTT
GATGTCAAAGTTTACACTTTTTTAAATTTGTATCTCTTATTGTAGCTACAGTTACAATTACTTC
TTGTAGCTACAACTTATTGTAGCTACAGTTGTTTTTAATAGTTTCATCTTTTAAACCTCCTAAT
AGGAATAACATTGCTTTACCTGCCACCTTTACAATACTAGAGAATTCTGACTATGAATTACTTA
TACCATTTAGTTTTTTACTTTTATGTTTCTCATTAACTAGCAGTCTTTTATTTAAGCCTAAAGA
ACTCCCTTTTAGTAATTCCAGTAGAGCAGGCCTAGTAGTGACAAACTCCCTTAGGCATTGTTTA
TCTGGAAAAGTGTTTATTTCTCCCTTTGCCAAGTAAAGTATTCTTAATTAACAGCTTTGTTTCC
TTCAGCACTTTGAATATACCATCCCACTCTCTACTGGCCTGTAAGGCTTTTGCTGATAAAGCCA
CTGAATCTGTATTGGGGCTACCTTGAATGTGATGTTTCTTATCTTTGCTGCTTTCAGTATTCTT
TGTCTTTGATTTTTGATAACTTGGTTGTGATGTGTCTTGGTGAACTCTTTAGGTTGAATCTGAT
CGGTGACCTCAGCTTCCTGTTCCTGAATTTTGTCATCTTTTCTCAGATTTGGGAATTTTTCAGC
TATTACTTCCTTAAATATGCTTTCTAGGCCTTTTTCTTTCTTTTCTCCTTAAGGACCTCCTATT
ATGCAAAAGTTAGCTAGCTTGATGTGTCCTGTAATTCTTATAGGCATTCTTTTTTGTTTTTGTT
TCTCATTGGATAATTTCAAATGTCTTACCTTTGAGCTCATTGATTCTTCTGCTTGATCAAGTCT
GCTGTTGAAGCGTTCTACTTAGATTTTCAGTTCAGTTACTGTATTCTTTATCTTTAGAATTTCT
ATTGTTTTTGTTTATATTTGTTAAACTTCTCATTCTGTTCAGATGTTGTTTTCCAAATTTCATT
TTTCTATCCATATTTTCTTGTACTTTGTTGAACTTAAGAGGATTAGTCTAAATTATTTGTCATT
TCACAGGTCTCCATTTCTTCTGAGTCTGTTACTGGAGCTTTCATATCCAATGTACACCAGAAAC
TTTGCTAATTCCACAGTAAATGTTAGAAATGGGTTTTTATTGATAAAATCAGTGGCTTGCTAAT
TAGTATGTTAGAAGCTGGCTTGCATTGGTGAAGTTGAGGGTATTCGATATACGTATCCCGGCTA
AAATACTTCTAAAGAACCTCTCCAGCTTTGCAAGAATAGAGTATAGGAATCTGAGGAGGCCTTT
AACATGGCCCTCAGAAAGGACACACAGGGCTTGACCTCTAAACATATAAACATATACAGAAACG
AGGTCAGAACATGTTTTGACTGATGCCAGTAGTAACACAGCAGAGCCTATTGCTGGTCACTGTT
ACCAATACCTTGTGTAATAAACCTTTCATTTAAATTGAGAGGTTGTAAGCCCCGAAAAAGCAAA
GGGCTTACAGTACCTCTTGCCTGAACACTCCTGTGTGTGCTTTCTAAGAAATGCTTTTAAAATA
ATGCCTGTGGCACAAATTGATTTGACACTGGCTCTCTTTATAAGCTGCTATCAAGGTTGTGGGG
AATAGCTAACTCTTACCCACTTTCCCTGACAAATAGGAACCCGTAGTTACCTACCAAAAGTATG
TAAAAGCTATATCCGACCCAAAGGCCAGTTAGGTCAAAACACCTTGAAAGGAGCTCAGTTTAAG
GAAATCAGTGGTGTCACAGTGCCCCACTAGCGTGTAAAAGTTTTCATAGTTTGACTATCGGATG
TGCTTAGTTTCACAGCTGAGTCTTACCTTGTGATACTTTTAGAGCAAAAGTCAAATCAAAGATG
ATTTAAAAAAACATTTCAAAGATTCATAAAGAATAAATGCCTCCTCAGGAAGTCTTCTGGAGTT
CTGCCCACTTCCCTTAGGTGGCTCTTTACTGTGCCCATCTCATAGCCTGTCTCCTTCCACTTGG
TGTATTGCAGAGTAAAGCTCACTGTTTACAGGGGTGGTAGAAAGTGTGGTCCTTTCCCAGACTC
CTTTTTCCCTTCCTCTTCTCATTTTTCAGAAGATGTGTTTGATAATAAACGAAACAAAATGACT
AACACATTGAGCTGAGCTACATAAGCAGATGTCAGTTTGACGTGAAGAGTTTAAAAGATCTATG
CATTATCIGGGGACTCCTCCCCCAGACCTGAAGGATCAGGTGCTGCCTTCTATGCCACCTGTGC
AGACAGCAAAAGAGGAAAACCATACCCACGTTCAGTATGAACAAAGGGGACATTTGAACTCTGT
GTGGACCCCTCATTGGAAGGGTGTTTCTTCTCCTGCTGCATCCACAAAGAGCACTCCTTAGCCT
TGCCTTTTGTCAGTTCTCTCTCCAATAAGGCTTGAGCAGAGACAACCCAGTGCAGTTCAGAGAG
ACTGAAGTCTGGTGTTCCAGGTCTGAGTCCTAGCTCTAGCTCTCTGTGTAACTTTGGGATGTCC
CAAAGTAACTTTTCACAACTTGATAGGIGTTAACTTGAATTTTGGATACAGGTGACTCTTAGCC
CATCCCCTCTCTGTGCTTCAGATATGTCATCACTTGGGCCATATGACCTCTGGACACCTTTCCT
ACTTTCCACAATTTCAGAGCAGCAGAGCAGACTGGAGCTCCTGCTGCCTCTGAGCTTCAGTGAA
TTATCACTCGTTGGAGGGAAGCTTCAAGCATTTTGTTATCTTTCAAGAGCAAACACAGTGTCTG
TCAGCAAGAATATGTAGCAGATGCTAGTGAACAGCAGTGATTAGGGTTGAATGCTGGATTTAAA
TATGGAGCTTAGGCTGTGAAGGAAGCCTGAAGAACCTAGAGCCCCATGAAGCTGCCCTCTGTGA
TATGTGAGTGCAATACAGTGAAAGCAAAGAGAATAAAATGATGGCTAACATGATGTTCCAAACT
TTAAACAGGAGAAAAACACACAATTCCATTATGTATAAGAACCCACACAGAGATCAGGAGAATA
ACCTCATTGGAGAATGAATGACCTGTGTGGGGAATTTAGGGTAGAGTTGAGATTGAAAAAATGG
GCCGAAGTCAGGTGGCCAAGGGCCTTAATACCTTGTATACAAGATGTGTAGTCAAGGAAGACCA
TGCCTTACTTATGCATCAATTCCCTTGGGCCTACAAAGAGCTGCCTAGCCTGGGACTGTTGTAG
AGAAAAGCTACAGTGTTCCAATGACACAGGGACTCCTCCATGTATGTATGAGTGCCCAGCTGGC
TCTGTAATAAATCTTATTTTTATTTATTAACTTTTCTTGCGCATTGGCTTGATGCATCAGTTGG
AAGTCAGAGGCCAAACGAAGTGAACACTGAGCCAAAGGAAGTTCTCAGCCTTTAGGGAGGCAGA
ATTCACTTTAAACACAATAAACAAATGAACTCACATTATACAGGAGAGAGTCAGAAGATCCCAG
TGGCTGGTGTCATCGGGCCATATTTGCCCGAAGTGCCTATTCCTTATAGGAACCCACTCCCAGG
GTTGATGGGCTACATCCTTAGGAGGCTTTATGCCTATGTTCTCCTGACCACTGGCTCCTCCAGG
GCTGGCCTTTTTTAGTCTCTCTGTAGAGGTTCCTGTAGCTGGTTGGATATAGGCTTTCACAGAA
GGGTCAGTGCCTTGGGTCTGGTTACAGGACTGTTAATCTTGCTTTGTTAAGAGTAATGTTATTT
CCCCATTTCCAAATTCTCCAGGGAGATGGAATGTCAAAGATAGTATGACTGTAGCACCTAAATC
CTGGGTTCCAGAAGCAGAGAAGAGAATACACTGAAGCTGTAGAAAGGCCTATTAGTCCATGTCA
GAGATTAACTGGATGCGAGGACCATTICTGGGATGGTGTATACAGAACTGGAGAACTGGATAGG
GAGTCAGAAACAAAGAGCTGAAGATGATGCCTTTGAAGTTTCCAACGTGGATAACTAGGTCAAC
AGAATATTACTCAAGAAAGTATTAACATAGGATTGAGATGCAGTCAGTAGTAATGGAATTGAAA
TTTCAAAGTATATACCTCATGGATCTCAGGGGGTGTTGAGCTGATCACCTGGGCTAACACTCCT
ATGACCCTGGGGAAAATCAAATGACCTGGTACTGTAGCCATGGTAGGGGTGTCATCACCTTAAT
CCAACTGGGACAGTGCTGTTTGATATTCATCTGGAACTTGGTGCAGACCCACATTTTGCTGGGT
TTCACCACAACCAAGGCTTTTTTGATTCTTTTCTCTTTTAACATCAGTACATCACTGCAAAGTT
AATCCTCATATAATAGGAGATGAAACTAATTGCTTATAAAAACAAGATTTTTACAACACTAAAA
TTGTTCAAGCATATGGGCATATTTATAGTTGCAGGCAGTGTTTCAGATGCAGACTGTTCTTGGC
TGCAGTGGTTGTTTACAGGCAGCATCTGTTCTGATTAAATATTTGATGATTATCCCCGAATGTT
TTAAAGCATAGTACTGGGCTCTGCTGACTGTACAACAAACTGGCATTTTTGACCTATAGGGCAC
TGGGCTAGGAGATACAGTTCTGAGGGAAGTGAAAGATAGTTAACAACTGCACAACTGACCCTTT
ATTAGTTGCAATAAAGCAATCCAACCACCCAACCCACTGTGATGGCTTTCCTACTATTTAAGGT
TGGTGGTGTCAAAGAGACACCCTCCTGTACAGTGTGCAGTGAATCAACATCATTTCCACAAAAC
CTCCTTCCTGCACAAAGGAATTATCATACTTTGTTACGAAGTAAAATTTTCCTGTATCAGTCAC
AGGAGTTCACCAGTTAAGATACTGTTAGTTGAAGACTTCTGGGGTGACTTAATGAAATAGCTCA
GCCATCTGGTTTAAAAACTGGATTCTTCTATCCCTCCACACAGCTGTCCATGCACCTGCATCAT
CTCAAGGCTGGTCTCCCTGGTGGTAGAAAGGCTGACAGTAAAAACTGGAGCCACATGATTCCTT
GCTTAGGCAGTGTTTCTTCATACTCTCACATGAGAGCAGGCATGTTCTTTCCCTAGGCTCACAG
TAAACACTCTGTCAAATCTCACAGGCCCAAAGTGCTTTTGGTTATCCCCATTCCAAGCCAATCT
GTGGCATGGAAGATAGCATTACCCTGACTGCCTTAGACTAATATACCTACTCCACTTCTGGGGC
TGGGAATAATTTTGAGGTCAACCATCCAAACTGCATGACAGCCATTCCATGGAAGAGGTATGGC
CTAAATCTTTGGGGCAACCTGAATTCATGAAAACTCTCTTAGATTTATGTAACTATTTTTAGAA
TTCACTTCTGTATCATTTAATTTTACTAATAAAAATACCACCTTTACCCTAAATGTCAGCCAAG
CGTAAGGCTCCGTTGGGACAGAAGGAACTATCAAAGCTTTGTGTTTTTATACATTAGCAGCATT
TGACAAAGAAATAACTCTGAAGGAAGGAGAATAACCAGGCAGAGTCTAGATGCATGGAAAAGAA
GTCTTTGAGAAGGCTTCGCAGGCTGAAGGAAAGGGCAGGACTACTTCAGGAATACAACGTTTAA
GTAAAAGAGGTTGGGGTCTGTTGATCTTGAGGAGAGATGAGGATGGACTGGAAAATAGGAGTGA
GATATAGTAGGAGAGGAAAGGATATGGATGATGTATATACTGTATGGGTAACCATCAGTCGACC
CTAAGAAGATAATAGTTGTTAAATGGTTAGCTATTTAATGAAAGAAACCTGAACAAGTAATAAT
CTTGAGTTGCAAAGTGGCTGGAGTCACACAACAGACTAAATTTCTGGTAGAACAAATCCAGCAG
CTTATGTGAAGGTTACCATGTCTGAAGCTGGATAGAAAAGGTCTAACTTCCAACCAAAGTCACA
GTTCTTGAGCTCGGTACACAGAGACAGACTGCTAACAGCTGATGTGTCCTCAGCGAGAGTGTCC
TCATTTATATCCTCGTTCTCTTCTGCCTCCTTTTTTTGTTTTAAACTTTTGGGAAGTCTCATCA
TTCAATACAGTTCTAATATATCACAATAACAGAGGCGGCCCAATTTCTACAAATTGACTAATTC
TATCCCTGAAAAGTTCATACAATAAAACTATACAAAGCATCATTTTCAACCATCCTATAGAAAA
ATTTCCCTATTAAATTTTAACTCTAAATCCTCTATCTCTGTTAATAACCTTACTAAAAACTTCC
CCATCACTGCCTGCCAGGGAGATCAAAAGAAACCAAATTTAAGAAACCTCCAACACCTGTACCT
GACTGAAAAGCAAACATACAGACCTTTCAGTCCTGCCCCTACTATTCAGCTCCTATCACAGAGA
CATGGTGGATGTCCCCTTGGGAAGCGGATAGCTCTTAGGTGGAAGCTAGGCCTGCAATAAGCAG
GCCAGGAAACATGTCTCCCAGGCCCCATACTCTTGGCAGAAGCACTTGGCCACCAGACAGCATG
TCCTGTCAACCTACAGAGTTCTTAAAAACAAACACTGGGACCCAGAATAGTACCCTGTGGTCAT
AGTGCCCACAGTTCACTAAGCACCCTCACAGGTCTTTGACAGAACACTGACTGCCAGGTCACCT
GGTGGGCAGAGAAATGGAAGATTCCCAGGCCCAACTAGCATCTCAGGGAAGAACCACAAGCAGA
GCAACTTTCAGAGCTGGTCGGCCAGCGTTGGCACCCAGGGAAGCAAGTGCTATTCCATATTTGG
AGAGAACATAAAACTCAAGGAAACAGAGAAGTCCTACTCAATACCGTTCTCAACTGAAAACAAG
AGAACTTGCAAAAAAGAAACCCAGTTTTCTGGAGTCCATGGAGAAATATGAAGCCAATGCTCGG
CTAACAGAGCAGAAAGCCTTTTATAAATAATGCCAGTCAAAGCTCCAAAGGTCAGAGCTGATGC
ATGCCGGATTGTTCTGACAATTTCTTAGGTTTCTAGGCAACAAGGAGCTGGTCAAACAGCTCAC
CTCCAAGGACATACTTTATAATACCACCCTGGTAGACGAGAGCGAGGCAGCAGTGAAGGCAATA
GTCAAGAACAGGAGAGGGAGGTAAAGAACAAGATGCTCTTCAGACAGTCTGGACAAAGACAGTC
CCTACCCAGCTCTCAGGAGTTGCCTCCTTAAGAAGGTGGAGGCCCAGGCAGCTCCCAGGGTGAC
CCTAAAGACAGACTCCAAGGAAAAGGGTGTGAGGGCAATGGTGAATATAAGGAGGTCCCCTTTC
AGTAGGCAGAGCAAGTCAGTTCAGGTCTTATTTTTAGAGTCTCTGAACAGTGAAGAGAAGCTTT
CTGTGGACAGCATTCCACCACCATGGGAGGGGAAAGGTGCCATGAGAGACTTCTCCAGTGGGGC
ATACAAGCATTGTGCAGTGATCCCCAAGATCCAGCCACTGGCAGGAAATCGAAAGGCAAGCTTC
TTAAATACATAGTATATGGAGACAGAAGTGTGGAGCACTCCCAAAATGAAAGGCCAAGACCCAG
GAACAACCTCCACAACCTGGAGTATATACAAATGAACCCAGCCCTGCTGACTCAGATCCACACC
ATCCTAAAGCAGGGGTTTCTCATGAATGAAAGGGCTAAGTATTTGGTGGAGAGAATGAGAAGTC
TCCCACTGCCCACACATTTTTTCTTCAGAGATTTCTTTTCCAAGAGCCCCTTGAATAAAGGAAG
GGAGGGAGCACTGAATGCCCCAGAAATCAGAATGCATGGTGCGGGAAGACGACAGGAAATAGTT
CTCAAAGAGATCAGAAAATAAATTGGAAGCAATTTCATTACCACAAAGAACAGACATTTTTTCT
AAGGCACCCCTCCCCTTTCCACCCAATTGTCATTCAGCAACTACTGAATACTTACCAATGAAAA
ACGTTACCCCTGACCTCAAGGCTGCTCCTGAGCTCTGGTAGAAAATGCTTTCCTTGTCTATAAA
ACATGGCAAGCAAGGCAGGATTTAACAGTAAGGGCACAGTAGCACTGCAAGCCTTCAAATGGAA
ACCTGGAGACAAGCAGTGAGAACAGGAAGCAAAAGCAGGGCAGGTGAAGCTAGGACCAGGGCAT
CTGGAACTTTCCACACAGGTTGGATCTCCATGCCAGACAACAGTTTTCAAGGAAAAATATCTAA
GAGGAACATGACTTTGGGAAACTTTTTGGCAGTACTGCTTACTGTATACTAGAGAGTAAAAGAA
TTTGGGGAACATTCACCAATTTGCTTCTTCAGGGGCTTGGGTAGGGAACGTGAACAGGAACCTG
GCTCTAATTTCTGAACTTTTTTATCAGTAAAAACAATCCAACAAACGAAAGCTAGTCAGTGAGA
GAACTGGGAGGGTCTGCCCTCCTTCCCTGAGTCAAGCCTTCTGGGGGGACCTCCTGACATTTAA
TTAAGCAAAGACAACGCCCACTGAAGGAAGCTGACCTGAAAGTGACACGCTACTGTGAAATGAG
CATGAAGTGGGAGCTTGTTACATATATGAAATGGCCAGCGATCCTGAGCAAAGCGCTTCAGAGC
TTGAGACCTAAGTCTTCTCATCTATATACTGAGGGCTGGACAAGATGATCTGTCAAGCCATTTT
TATCCCTAATCCACCAAAATCCAATGCTTTAGTTTATTGTCACAAAAGCAGGTATCGAATGGCT
ATCCTGCAGTGCCTCCAATCAACATTCAGACTTTTTCCCTGAGGCAATATAAGATAACAGTTAA
CATGTTTTTATCAATTAGGTGGTCATGAGATAAATATATATGGGAAGTGGTAGTTTTTCACTTA
AATGCATATAATAATGGTACAGCTCTCTTTGAATAGTATTTGTTTATTTCTTAAATATTTAAGT
TCCTAAAGACGGTAAGAATAACCCAAGGAAGTGAAATCAATGTCACAAAGCACATGGCTAAATA
ACTGCAGGTTTGCAGTGCCATGTGTGAGATCAGATGACAGAAGGGAGAACTACCTTTAGGCAGA
GGCTTCTCATGTCCCCTGGAGTGGCCATGTGCTGTTCTACATGACTACTTCCACTTCGGTTATG
TAGAAGCTATTTAAAGCACACAGATGTTTGTGATGAGAAAAAAGCCACCCTTAATTGAATAATG
GAAATTATAAGCATGATTTGAGGGTGGGGGTGGAGGTGGGAGTAGAGATGGGTAGAAAGGAGTG
CAATGGAAACAAAGGAGCCTTCATAAAATTCAAGTCACTTCTTAGGATAACGTGATTGATTTAC
TCACCACCTTCTTAGGAACATAAAGCAAACAAGTGGGTTTTCCTTTTACTGCTTTTCTGAAATG
AGCTACACTCAAGAAAGCAGCACGGGGGTTGTGCTGTCCCTGCACAGTGGCAGGAGAGTATGAG
GAGCAGGTGAATGCCACAACAGCTCCATCCAAGATCATTTTTCACATGCAGGAACCATTCTTAT
ACTACCCTTTACTGGTAATTTCTGTAGAAATCTGGAAGTCTGGTTGACACCCTCCTGTACAGTG
TGCAGTGAATCAACATCATTTCCCTTGGACTTTGCAATCACGGTGGCATTCATACATTCATTCA
ACAAGTATGTATGTACAGGACAGTGGAGTAAAGAAAACAGATAGTTCTTACTCTCACGAGGCTT
AAAATTTCAGGAGGGAACTAGGCAGTCATGAAGTAAACATAAAAACACAGATTGTAATAGGCAC
TAGAGAATAATGAAGACTTTGGGGAACACAATTTAAATTGGAAAAATATCTCAGTTCCGTTAAC
ACATGTTGAAGGGGAATAGCATTGTTTTGCTGGATTTGGGGCCTGGATGCAAGCATGTTGGGTA
TGCATTGTAGGGTAATGCATTTCCTTCCATTTGGGCCCAAGTGTATATTTACCACCCAGTIGTG
ATGAGCTGGGATCCTCCTGCTCAATCTCAGCTTGAAGCACTTGGAGGTTATCTGCCTGCTGTGG
GTGATTATTTTGGAGCAAGGTACTTCATTTGCCTCAAGAAACAGATTTGATACCACTACTGTGC
CCTTTTGGAACAGAGAAGTAGGCAAGACCCCAGTGTGAGGCAGAGTGATGGGATCTTTAGGGAC
ATAATTGATGATGTAACTGATGATGATTTTGGAGTTTATACATTTCCAAAGTTTCAAAATATTT
TCACTTGGTTGATTTATCTTTATGGTGATGACACTACGTAGATATATGCCCTTCTTAAAAGTTA
CAGTAAGAGGCTGGGAGCGGTGGCTCACGCCTATAATCCCAGCACTTTGGAAGGCCGAGGTGGG
CAGATCATGAGGTCAGGAGATTGAGACCATTCTGGCTAACACGGTGAAACTCCGTCTCTACTAA
AAATACAAAAAAATTAGCCGGGCGTGGTGGCGGGCGCCTGTAGTCCCAGCTACTCAGGAAGCTG
AGGCAGGAGAATGGCGTGAACCCGGGAGGCGGAGCTTGCAGTGAGCTGAGATTCCGCCACTGCA
CTCCAGCCAGGGCAATGAGCGAGACTCCGTCTCAAAAAAAAAAAAAAAAAAAAAAAAAAAGTTA
CAGTGAGAGTTGACATTGAGAAAAGGGAGGCCCAGCCAGGGTTATGCAAAGACACAGTAGGGAG
GAGGATGGGAAGTTTCTGAGACACCCCAGTAACTGCATGGGTCCACAAAGCACATGTACAGTCT
GCTCTATGCACTGCAGGTACAGCCACGAATAAGACAAAGTCTCTGCCCTCATGGAGCTTGGTGT
CTGTCTACTGGAGCAGACAAAAACAAACCTGCTTTTTCTCATTTGCCTCTACTGTGGGTTCCGT
GTATAACTCTCAAATGCTAGCATTTCCATGCATTCCATCCTTGTCCTTCTCTCCTCTCTGCCTT
TTCCAGGAGAATTTTCCTAGCCACAGGTTCAGCTATGGTCTATGTGCTGGAGTCATACATTTTT
ATCTTCTGTGTACGTTTTTCTCTAGACCCATATTTTTAATTGCCTTAGGACAGCTCTCCCTGGA
TATCCCTCAGACACAACTAAAACATCATTCAATTGTACTATTAATAATTTTCCCTTCAAAATCC
ACTCCTCTTCTCTATAGACCTAACAGCAACACTGTCCGTCCAGCTCTCAAAACCTGAAATGTGG
GAGTTGTCTTTGACTCTTATCTTTCCCATGCATGAGCACTGAATCAATCAGAGTCCCTAGCATG
TCTTGGACTGTGGCCTCCCTTCTATCTTCAGATTATCTGCCTTTTTAGAATTATGGCAATAACT
TAACTGCAAAGACCACATGTGCACTTGTGCTCTTTCAGTTTGTAATAAATATAAAATGAAAAGT
GATTATGTCTTTGCTTAACGTCTGCAAAATAAAAGTCCAAAGTCCTTGGTATGGTCTATAAGGC
CCTCATTATCTGACCTGCCTGCCTTCCCAATCTCATCTTAATCCCTTACAGCCTGACACTCAGA
CATACTAGACTTTCCACCTAACTCACTCACATACACCTATCCTGTATGTCAAGATTCGGCTTGA
CCTTCATCACCTACCTATCTGCAGTATTTTTGGATCTGAATTCTCTGCCCACATTGTACTTTCA
CATACTTCTTTTACCCTTAGTGGTTTGTACTTATGCCTGGTCTAACTAGATTGTCTCCCTGTAA
CAGACTTCTTGATTCAACAAAGCAGCTCTGAATCAGCCTGAAACCTATGGCACACTGCAAAATG
GCAAACATTCAATAGGTATTTGCCCAGTAAATGTTAATGAAAGGAAAAAAATTCAAACTTCAGT
TGGAATAGGATTAGAGACAAGTTAAAAAAAAGTTTCCCATAGAAATCTTCCTCATCGTAAATAT
CATCATCCTAAATGTCCCGAGTCTTTCCATGGGTCTAATTTACCAAATCATGAAGACTCTCTTT
CTCACTGTGTATGTGTAGTGGGAGAGGCAGAGACAGAACGGTTTTTTTTTTTTTTTGCAAGTCT
GCTCTCTGGATTCGTTTTTCTGGGGATGAAATCTCAGGCTATGTAGCTTTTCTGGTCCCTTTTT
GGTAATCAACAAATCATCAGTTTCTCTAGGTATTAAAAAGCCTACACTTTTGAAACACCAAGAG
GCCAAACTCTCTCTTAATTGAAAACATAATTCTGCCTCTTACTGAGGTCTTTGGGTGGGAAGCT
TAAAGACAGCAGTGTTGGGGCAATTGGTTTTTCTTTTCCTCCCTCCACCTTCTTTCCCATTCAA
GAGCTGTTGCTGCCCTTTCTGGAGGGGAGGGAATTAGAAAAAAAGATCCTGCCTTACCCAGTCA
CTGTTAATTATTACTTTGAGCCAGGGTGGGGAATGACTTTCTTTCCTGGAATACACCCTGCTGG
AAACCACAGCTGAGATTCTCTAAGCTGGCAGCTTCCAACCTCTCCTCCCTCAACAGCTTCAACA
TTATATGGCACTCAGCTGCAGGGTGCCTGGCTCCAGGCCGGCAGTCCATTTGTGCAGGGTAGTT
TTCAAGATGGCTCATAGCCCATCTCTTTCAGTGACCAGCGTGGCCTATGGGAAACATACATCTT
GACTCCATTATTGGTAGGAGTACTCTTTAGTTAACTGCCACAGGCCAGTTCAGACACGTCTTAC
CCTGAGAGCCTTTTCAGAGTCAGGTACCATCCCTGTGTCCCCCAGCATCTGAAGATCACAGGTG
AAGGTCTCCAAGTAGTTCACTTAAAAAATACCTCAGTAGGTTTAAGAACAGGGAAAGCTCCCAA
TTCATTCTGTCAGGAGTATGACTCTCATCCCCAAACTGGACAGATATATAATATGAAAACTACA
GACCAATATTCCTTATGAATATAGATGCAAACATTCTAAACAAAATACTAGCAAACCAAATCCA
GCAGCATAGAACAAGGTTTATGTAGCATGGACAACTGAGATTATCCCAGGAATGCAAAGTTAAT
TCAATATACAAAAGTCCATTAATACAACATATTAATAAAGGACAAAACAACATGAAAATCTTGA
TACAGAAGAAGCATTTAACAAAACCCACAGCCCCTCCTTTTTTTTTTTTTGATTAAAAAACACT
CAGTAAACTAGGTTTAATAAAAGAATTTCCTCAACTTGAAGCAAACCCACAGCTAGCTAACATC
ATACTCAATGGTGAAAGACTGAATGCTTTCCCCTTAAAATCAGGAACAAGAAAAAGATGTCTGC
TCTTGTCACTTCTATCCAATATTGTACTGGAGGTGCTAGCCAGCACACTAAGGCAAGAAAATGA
AATAATAGGAATCTAGACTGGAAAGGAAGAAGTAAAAGTATTTCTGTTCACAGATGCATGATCT
CATATGCAAAAAATACTAAGTGACAAAAAAAGAATTTTAGAAAAGCTACAATATACAAAATCAA
TATACAAAAATTAATTTTATTTCTAACACCAGTGATGAATACAAAAATAAAAATTAGAAAATAA
TCATATAATTAAAGTGGCATCAGTAAAATACTTAGAAAAAAATTAAAGACGTCAAGACTTGCAC
ACTGAAATCTCTAAAACATCACTGAAATAAAGACCTAAGCAAATGCAAAGACATCCCACAGTCA
TGGAACAGAAAACCTAACACCATTAAAATAGCAGTTATTTCTCAAATTTATCTACCAATTCAAC
TAAATCCCTATCAAGATCCCAGCTGTTTTGCAGGAATTGACACAATGATCATATAAAAATTCAT
ATGCAATGCAAGGGACCCAAAACAGACAAAATGATTTTGGGGAAAAAAAAAAAAAAAATGGAGG
ACTTGCACATCCCAAATCTAAACTTACTACCAAGCTACAGCCATCAAGACAGTGCGGTGCTGGT
ATAACGACAGACATATAGGGCAATGGAGTAAGACTGAGAATCCAGAAAGTCTTATATTTATGGT
CAACTGTTCTTTGACAAGGGTACCAGGACCATTCATGGGGAAATAATAGTCTTTTCAACAACTG
GTGCTTGGGCAGATGGATATACAGATACCAATGCACTTATGCAAAAAATGGATGAAATAGGAAA
CTTCACTACATTCTACTGCATGCTCGGTCATTTTCAATCATTTAGGTGGCAACACTGACAAGAT
AACAGAAAGATGGAGGTAATAACATGTGAAAGGCAAAATGGTTTGTTTTTTTTTTTAAAAATGA
CAGTCTCTATCATGAATTTACTTACACTCCAGGCAAAGGTTATTAGAAGAAAAAAAGATGTAAG
AAAATTCCTTAACTGAAATGTGGAAAGAGTATCAAGAGGAGACCCTAAGACACTCTGTAAGAAT
CCCAGTGACTCCTCACTGTTCAACTAAGAAATGTACCCCATTATGCTGTGCTACCACGGAAGCA
TTGGAGGCACTTTGGGGGTTGATGAAGTCTTCATGGATGAGGTACTTTAAATATCTGGTCATGA
AGAGTATTTGAGAAATATGACAAGTGAAGATGCTGGGTAGGAATGCAGCGAGGAAAGTGTGTAA
CACAAGGCCAAATTGGAAAGGTCAGTGAGGGGGCAATCTGTGGAGGCACTGAATGCAGAGGATA
TTAAAATTAGCAAGATAGTGTTCTAGCACAATGAAAAGGATTGGAAGAGGGAGATGAGAGTCAG
GGAGTGAAATTAGGCAGCTGCTAAGAATGTCCGGGTGAGTCAGAGGATCAGGACCTGTAAGGGC
AGTATAAAGAAGGAAGGAATGATGGGAGAGGTTTAGGGGGAAAGAAGTGACAACCTGTGACAAT
TGAGGAATGAGAGATTTTGATGATTGGGGTGAAGGTTACATTTCTTAAAAAGAAACAAAGAATG
GTCGGTCGATAGAGATGCAAGATGAAGAATTTTGTTTTTAGGACTACTGACTATGAGGTAACAA
AGAAATCCCAGAGACAGAGGTCTGAGCAAAAGATATGGGATTGGGGAAGAATCTTTGGGAGTGA
CTGAGGTTGACTAGAGGGAACTGGGCAAGAACAGGTAAGGACTTTAAGCAGAATTGGAGGAGTA
TCCATCTAAAATCTGGGTAAACTGGGATGGAAAAACAAGTAGCCAGAGAAGCAACAGCCCAAGC
TAGGGTGTTGTCAAGGTTATAGATGTTACTGATTTTGGCAATGAGGAGATTATAAGGATCCTTG
AAGACTGTACTTTCGGTGAAACTACCATGCATTAGTAGCCTTTCACAGTGATATCTACCCCACA
CCTGCATCAAAATCTTGCAGTGCCTATTAATAAATGCCAACCCACTAACGGGGAGGGGAGAAAA
GACTTGGGACTCTGTACTTGTAAAACCCTCTACCCTCCCCAGGCAATTCTTTACACACTAACAC
TGTTGAGGTAAAAGGAGACAAGTGAGGGAGCAAATGGAAGGTGTGTTTTTGGATTATACAGTGG
CTCATGGAAGGGTGGGAGGGGTACAGATGGCCCTTAGACACTGGCGGAAAGTCAGAGAAAAAAA
ATTACAGTAAGCAGGTAAAGGGATCAAGACTACACAGGGACTAGTCTTGGGACGACATTTCTTC
CTCTGACAGTTTGTATGGAATTCTTGAGAAAAATTCCTCGAAGGGGCCTGAAATCTCAGAATGG
TCATGTTTTTAATGGGAATAGGGAGTGATGCTGCCCCATTACAACCATCTGTTCTAACAGAATG
TCTGTACCGAGGAGGGATGAGTAACATCGGCAAGTTCTGTTCGAAGCCTTTTTCAAGTTTCTTT
TTGATATGTATCTATCTATCTATCATCTCCCTATACAAGCAAGCATCCCCAAAAGTAGTTGTCT
CAGGAAACCAGGGTTAGGATGACCAGCTCATTTGCTGGAGCTGCCAAGGTCCAGAAAAGTTTGG
CTGCAGGCTGTTTTCATGTTTTATGTATGTTTGAAATGTTCTATAATAATAAAAGGTTAAAAAA
GTTTACATTTATTTGGAAAACCAGTTACTTTAGTTTATGGTTCCTTTTTTTCCCTCCAGAGCTT
CCTGGAGATTGAGGTCTAATTCAAAGAAAACCAAAATATATAATAGAGTACCTGGGCAAAAAAA
GTACTTTTATAACATAACATTTGGGGTAGAGGAAGTATCCACTGTAGTCAAAATGTCTATGTTT
TGCTCTTCCTTATTGTTCAGGGACATTCCATTAAATAGTAATGAAAAGGCAGCAAAAGTAAGAG
GAGTGACAACATGCCCGGCATAATTAAGCAAGCTAGAGCAGCTATTCTGTGCAACCGACGATTT
TTTTTCTCTAAAATTTTAAGGGTAGGTTCATTCTGACTCTGTTAAAAGTCTACTTGATGTGAAC
AACTCTATATCTGATAACCTATTTCAATTACCACTTTAAAACTTGTCATATGGATACGTTATTA
CAATTGTAGAACTTTAATAAATACCATAATAATAAAACTTGAGAACTGAAGAGCACACATTTCT
TCACGAATTTATTATATAAAACGCCCTCAGAGTATTTAATTTCTCCTCACTTTAATTACACATT
AAGAAGCACAGTGGATGAGAAGCCTTTAAGATGACTACAGTTGCACGAAGGTCCCTTTCATCAA
GGTAGCGTATGTACCCTAACAGTGTTCTAAAGGCTGGCCCAGAAAAACCCCATGTTACCTTATC
ACAATATGGAAAGCATTGTCTTCTTTTTCCACTAAATTAAATTATGGTGAAAAGTGCCACAGTT
TTATTTAGCATTATGGTACATAACAAACAGTTCTGTCTCAATTATGAAAAAAATTAATTAAAAT
AATCCTGAAAGACATCCTTTTTCTCCCCCCAATGATTTGAAAGCTGCATTTTTCCTGCCAATTT
CAAACAAACAAATCATCAGGTTGATCTACAGTAATCAGTTAAAACAATCAGTCAATCAATCAAT
CAATCACCAAGGCACAAGCTCAGCACATTAGCTATAGCTTGTAGCAAAAGGATATATCAATGTC
TCACCTTAGTTAAAAATACATAATCCTTTTATTTTATAATGCAATAAAAGAAATTAACAACATC
ACATACACAGAAGACTAGGAAAGGGGAAACTACTTACTTCTGGAAATCAGTAATGTAAACCTAC
TTGTACTTTTCCATAGTACATGAAAGTAACGTTTAACATGTTTTGAATTAATTAATTAAATTTA
ATCTGTGGGGCTATACAATGTAATTCTTAGGAGTAATAGTTTCATTCATTTCCAGGTCAGCTTA
CTGTATGATTAAGTAACACAAGGCACAGTAGCCATCTTTTTCATTATGTTGCAACACTGATCAC
GTGCCTCGATAAAATGGCTGATTCAACAAGATGATGGCAACACGAAGGGGAGACTTTGGATTGT
CTATTTAAAATCTAGGTAATAAGTAAGTAATTAATAAAAACTCTATCTTAAGTGCACTTTCACA
TGCTTTTTGTTTATAATAAACAAACAACAAACTTCCTAACTTTGTTGCAATAGGCTTGACTACC
ATTTCATTTGGCCAAATGCACTTTCCCCAGTAAACTTAAAACAACAACGAGAACAACAAGAACA
AAAATCCCTGTCCTTTCATATACTAAGAAAGAGGATTGGCTACTGAAACAGTTCATTGCAAGAC
ACATGAAGACGACATACTGTGGCATGAGTTGTTTTTGTTTTTAATTTGTTGTGCTGTTACTAAA
GTTCTGAGGGCTGCAGTTAAAACATTCCAATTTCTCCCTTCCTTCCATCTTTCTTTATTGATTG
ATTCTCAAGATTTTGCACAGAAAACTCTTTGGGGGCTAGAACAGCAGTAATTGCATCACACTGT
TTTCAAGACTTCAAGTTTCAAAAGCAAATCATTAAAAAAAATACAGTTCCTGATTTGAGTTAGA
TACAGGGACAAAAAAGTAGCACATACTTGAAGGTTACGTGGTCTACAAATGGTGGCAATATTTT
CCTTGGGAGAGTAGTTCTGTTGGTATATATTTTTTAAATACTCAAAAGGCTCAACCTCAAGCAG
TAATAAACACAAGCAAAAGTGATTTAACCCTTAAAATAAATATTCAGAAAAACCTCTCTGTACA
TACAAGTGAAAGAATATGTAACACTTTCACGCAAAAAAATAATTATAATAATAATAAAGGATTT
GTTCATATATGTAGCTGAAATCTGCTGTTCCAGCCCACATGTCCCCAATAAAGAAGGGAGGCAC
AGACATAGGTGACTACTGTGGTTGACTATCTTACAGCCTTTTTGTACTGGGACACTATCACCAC
CAAAAATTTATCCCTCGTTATATTTTTAAAATTTTTTAAATTTTTTCTTTTTTTTTCCTTCCTT
TTTTTTGTTTTATTTTGTTTTGTTTTGTTTTACAGCATGCCAAATCCTTTGGCATACGTGATGG
CCTTCAACAATCTCTCTTTAAGTTTTTCTTTGCTTGAGTATTCCGGAAGTAAAAGCACATTAAA
GCAAGTATGAGATGTAGGTAACCTAAATAGAGAAAAGGGGAAAAAAACAGGAAAACTGTAAGTC
ATGGGAAATACACTTAGAATTAAATGCTCCTATTTTTAGATTGTATATAGTTGAGACGGTCTGC
AATGCAAACTATACATTAATGCAAATCATAAACTTTTTGTTGTGTAACTACCAAGTTGCCTTTA
TCCTATAAATTACTCAAAGCTAGTGACGATGATAAGATACTGTATCCATTGAGTTTTTACTACA
TAACAGATACCATTTTAGGTACTGAATTCTTACAGTTCATTTAACTAAATCTTTCCACAACAAA
ACCACAGAGAGGACATCAGTAAATGCACTTTAGAGGTTAGGTCACTGAGAAGTCAAGTAACTTC
CTCTAAGGTGGAGAAAATACTCAAACCTGTTTTACAAGACTGCAAAGTGTGTGCTCTTAAATGC
TTATTAGAAACACTGCTGGCAATATGACTAAGAAAATGATTTGATAACAGGATTCTAGCACAAT
CAAATGATAATCTTCCGAGCCTCAATGTAACCATTCTAAATAGATGATCATGTTATATGGCTTT
CAATTAACAAGCTGGGAATCAAAAAAGTAAATGAATCACACTAATTTGATTCCAAACCAATGTG
AGCCCCATAATAATTTTTAACTAGGGCAATTTCTTAAAAGTTTCCTCACACAATGACAGCAAAG
TATTTTCTCAATTGTCTAATATGATTTGGGGATTTGTATATAAAATCACGAATGTGCTCAGAAA
CTATAAAGACAGTTCATATGTATGTGACGAGGAATGCAAGGTTTTCGGTAGGTATACAGTCACA
AGTTAATAATTACCTACCTTTCTGTGTCTGGGCCATTTTTGGCTATAATCATCTTTAATTTTCC
TAGTCCTCCCACAGGTGCTCTGTCTGTGCCCGTTGTAAACTGCAAGAAGAGTCTTTTCTGTTCA
TCTGTAAATGAATGAACGATTTCCCAGAACTCCCTAATGAGAAAAAATACAATACTGGTTTCAG
TTTGGCATTCATTATGACTGGTACTAACATAAGCTTATGATTTGCATTAAAACTATATTAAGAG
ACAACTTGGAAGTTAATTATCAGGATAGTATCACTTCTGGTGATTTAAAAATTTCCAAGCAAAT
TTATCCTGAACAGCTCTGAATACGTAAAAATTTAGATTAGATTACAATATAGTAAAATATTAGT
ACTACAATAGTAAAAAATTGAGAAAACCCAAGTGTGTAGTAACAGGAAGTGACTATTTAAACTA
TGGTATAATCACATTATGCAGTCAGTAATGAGCAAAAGACAAAATCCTATGAATTGAAAAAGAC
TGAAATGAATATTTGGAAAAATTAACTCCAGGTGCCTTTGGGTATATATTTTATTTATCCTTTC
TCCTTTATTCTCCTGGTCTACTCATCCTTTAACTCAACTTTGGGAGGAAAAAGTGATACAGATT
AAGGACAAAAGAAAAAAAGACCCCCTGCCCCAACCAACTGGCCCCAAATGCAAACAACTACATA
CCTAATAGCAGTAATACAAAAGTTCAATTTTTATATGAACTAGAATACTTTAAACAAGTAATAT
GTGCTGTGTATGGAGGAGGAGAATTATTCTGACATTTCTGCCACCTGCAGTTATTTTAAGAGAA
TGATTTATCCTGTGGCATTCCTAAAATCTATGTAATAAAAGCTATGTTTTAATGACACTTATGT
TAGTTTGAGTTCTAAAAAACGAAATACAAGCTCATAAAGTGCAATCTTGAAGTTTATTTGAATA
ATCAGGCATCTATAAAACATATATACACTAGTTCATAGCTAAATAAATTTTTTTTTTTTTGAGA
CAGAGTCTTGCTCTGTCGCTCAGGCTGGAGTGCAGTGGCGCGATCTCGGCTCATTGCAAGCTCC
GCCTCCTGGGTTCGCGCCATTGTCCTGCCTCAGCCTCCCAAGTGGCTGGGACTATAGGTGCCTG
CCACCACGCCTGGCTAATTTTCTGTACGTTTAAGTAGAGGCAGGATTTCACCATGTTAGCCAGG
ATGGGTCTCGATCTCCTGACCTCGTGATCCGCCCACCTTGGCCTCCCAAAGTGCTGGGATTGCA
GGCATGAGCCACCGCGCCTGGCCTATAGCTAAATAATGTTAAGATTAAAAAATTAAAAAAAAAA
TTAAAAGTATTTTTAGTTGCTTATATAATATAAAATGCATTTTAATAGTTATTTAGAAAGTTCT
TTCCAGAGCCAGGTGTGGTGGCTCATGCCTGTAATCCCAGCACTTTGGGAGGCTGAGGCCAGTG
GATCACCTGAGGTCAGGGGTTCAAGATCAGCCTGACCAACGTGGTGAAACCCTGTCTCTACTAA
AATACAAAAATTAGCCGGGCGTGGTGGCAGATGCCTGTAATCTCAGCTACTTGGGAGGCCAAGG
CAGAAGAATTGCTTGAACATGGGAGGCGGAGGCTGCAATGAGCTGAGATCACACCATTGCACTC
TAGCCTGGGAAACATTTTGAGCCATCTCAAAAAAAAAAAAAAATTTCATCTCAAAAAAAAAAAA
AAAAAAAATTCCAGAATGTTAGCAGAGAACATCACACATCCCAAACCAGTAATTACAATTTTCT
ACCTTTTGATATTCTAATATCCCAACATACTATTTTTATTACCAAAATGCTGGCATTTTTGGTG
CTGCAACACCATTTATCTGAAATAACTTAAAAGTTTTATTAGAATTCTTGGCTTTCTCCAATCT
CTTGCCACTTCCCTTCCCTGCCCCTTTACAAATCCACCAGTCAGCAAGAGAAAACAAAAAAGAA
ACACACAACAACAGAAAACTGGACATGAAACTTGCAGGAGCATCTCTCAGGTAAGGAGGAGAGG
AATCGGACCCCAGTTCATGATACTCCTTTTGGGGAGGCTGACAAGAGAAAACAAGTCTGCTGTA
GCACAGAACCCTCCAACATAGCAGAAAAGCTGCCCTGCAGTGGGTGGTAATAAAGCCTCCTCAC
ACTGCAGGAGGGCTCTGAAGCTTAACTGAACCAGCTACACCTAAGGAGGTAGACCAAAGAGCAC
CACCGAAGAACAGAGACAGACCCTGCAGGAAGTGGCAGATCAGCAGTGGTCACAATGACCAGTC
TGGCTATATTATTACGGAGCTGTTTCTTTGGACTTTTAAACGAAACCTTTAAAAATTTTATACA
ATGAAAACAGGGAACAAAATGCATCTCTATTCCTATAAGTGTTATGTGTGTTACATTAACATTT
TGAATTAAACAAGAATGCATATATTTAGAAAGCTGAAGAAAACACGAAGAAGCTTCCAGGTTTC
CACATAAAAGTGGTGGCTGTATCGATCACATCCTACTCACATCTCTAAAAATCTACCCGGATCA
AAAAGGGGAAAATAAAAAAAAAACCTATGACTTAGGTCTAGAGTAAAACTAGGAGACAGAACAA
TAAACTCCAAATACCAAAGAAGTGGGGAAATGAGCAGGGTCCAGCAGGAGCTACATCGAAGAGT
GGCTTGTGCGGAATCATACTGATTAGGGATCACCCAAGGCCAGACCCCACCCCCTCCACCACCT
GCCCCTCAACAGAGCACTACTGAGTGTAGGTTAGAGCACTGGCAACAGGGTGGTTAAAGGAAGG
ACTACAGAAAAGTACTTGGGTCCAGCAATGGCAGGCTCAGGAAGGCACCATGCATGAAAGGAAG
GGGGGTACCTTCAAAAGCAGAAGTGTCCCTTAAGCGGCTGTAAAGGGGTGGAAGCAGCTAATGA
AAAAGAGTCTTCATTAAGCTACATGGAGCTGAAAAATCAGAAAGTGGTGATTCTGCCCTTACTA
AAGCAACAGAAGAGGGATCCCTTCAACCAAGACCCCTAAAGCCACACAATTCCTCCCTGCTCCA
CCATGAGGTCTAGTAATAACAGGTCCAGAAAAAAGTAACATCTGTTATAGAAACATAAACAAGA
AAAACAGAATTCGGAAGTCTAAATAAAGTTATTATGGGAAGAGTCTGGCGAATGAGAAGCAAAA
CTTTCCGGTAGACAAAAGTAGACCAGAAAAATTCAGTCATAAACAATGGGAAACTAGTAACACC
ACATTCCAACACAAAAGGAGAATGCAGCAGCAGACAAGACAGACCACCAGTGATGAGAAATACA
AATTCAGAAAGAAATGAGCACATCTTAAATAAAAATAAGAATACTAAAAAATACTGAAGAAATA
TGCAAGGTAACTTTGGAAATAAAGGCTAATTTTATTATAAGGTGACCAAAGAAGAAGCAGTATG
ACTAAAATTTCTACTGAGGACAGACTCTAGAAAATTAAAAAACAAAGAAAATGAATAAAATAAA
CATAACTAAAGAAATGCATATCAAAGCATAATAACGAAAAAGATTAATAGCTAACTACATAAAC
ATAAAACCTTTCATTTGGCAAAAAAATCCTATAAGCAAGGTTAAAAGACAAATGACAAACTGGG
AAAAAATATTTGCGAATTTACCAGCGTTAAAGGACTAATCTCCTTAACATATAAAGTTTCTAAA
AGTGAAGAAAACGACCAGCTGAGCAGCAAAATGAGCAAAAGACTATATAAAACTATAAACAGCT
CTACAAGAAAAAATGTCCATTATCATTCATAATTGAGGTAAGCTCAAAAATTGTAACTATAGTA
AAATACCATTTCTCAATTGGCAAATAAGTAAACATCCACATTTAACAACACATTCTGTTTGGAA
AGTTTTAAGAAAAGAGATACTGTTGTAAACTGCTGGTGGGAATGCAAAATGTTATTTTTCTGTG
AAGGAGGATTTGGCAGTATCTAGCAAAATTACACATGCATCTACCATTTGATGCAACAATCCCA
CTTCTAACAATTTATCTTAATGATACTTTACATATGTACGGAATAATGCATGAACATTAAGAGG
ATTCACTGTGACATTAGTACTAACAGCAAAAGGTTAAAAATAACCTCGATGCCCATAAATATTG
ATAAGCTATGCTGCATCCACACAAAGGAGCAGTATTCAGTCATACAAAACAAAATGGAAAACAC
CTCTGCAATAATGTGGGATGATTCTCAGGATACACTAGGAAGATTATTTTCCTAGTTCTGTCCT
CTGATAAGGCCTACAAGCAGTAACGTTCAAAAGCAAAGGCCATACTTTGCATCTAAAATCTGAC
TTCTAAATGCCATTCTCCAACAATTTATTGGAAAAATAACTTATTCCAGGCCTGAGAATGAATG
TTCGAGATTAGTTAGAAATCTCAAAATCTAATAGGGTCATTTTAAAAGCACACGATAGCTAAGC
AATTTGAATATCATTTAGAGTAATGACTGTGAAACGTATTAAATACAAAAACATTCATTAGTTC
GTAATATTCAAAAAGGCAGCAAAAACCAACCAAAAAACAAAATTAAATTGTCACTATTAAAATT
ATTACATTAACTCCTTACTCTAAAAACCTTAAATCTATTTTATCATGCCTTTTCTGTAGAACTG
TTTTTCAAGGTAATCAAATGGCACCCAATGATGAGAAAAAAGAATGCCAGGTATATATGTAGGA
CAAGCAGATGGAAATTTTTTTTCCCCAAACAGCCATTTTGCAATCCCCAATGAGATAACAGATT
AAGGTAATGATACTGATAAATACGAAAATCAGGTGAAGGGCAGGTAGCAGGTTCTGGAAAGATG
ATGATGGCAACGACATAGTTATTATTATTATTATTTTTTGTTTTATTCCCTCCCAACCTCCCCT
ACAAAAACAGCAAACTGAATGAGAAAACCAAAGACCCAGAGACATTATCTACAACAAAACCATG
AACCATTGCATATAATTGGACAGAAACAAAGCTCTGACAACTACAAGACTGGTTAGTAAGGAAG
CAGATGCAAGGTAACTGACTGGGTTTCTGACAGCCCTGAGAACACTGCCAACCCACTGGAAAGC
ACAGGCCAATCTGAAAACAGGGCTTAAAGTTTTAAAAAGTTCTACAGGATCTAATTTGCAGATG
ATTACAAAGGGATCATGATGTAGTAAGCTCTGGGCCCTTAAAACACCCGAATACCAAACCCCCA
CCAGAAACAAACCTTGTACCGAGGAAAAACTTCTGGGAATAACTTCCGAATGGACCAGGTCAGT
AATAAAAACCAAAAAAAAGTTCAAATATATGTGTGGGATAGAGGAGTAAAGAAGGCAGTTTCAG
AAAGCACAAGGGCATATTTTTGACCATTTTACAAAAACACAGACTTCTCCTCGTCCCTAAAAAG
CGAAAAAAGTTATCCTGGCCCATCTCTCCCTCGTCCAAGTATGAGAAACTCATTTCACTCACAC
ACACACACACACACACACAAATAAATAAATAACAGAACAGAGTGAAACAGTGTAATTATTAGAG
AAAAAGTATGTGCATATACATGAGAATGGTGTCCCTACAGAAAATCAAAGTACATGAAACAATA
TGCAAATAAAACATGAAAACTGTAAACAAATTTCAAACTGAGCTAAAAAGAATTTAAAAAATAA
CAAATCATTAGAAATGGGAAATTTCTGAATGAGGGTAACTGAAAAAAAGGAAGACATGACACAA
CTAAGGAGTAATTAGAAATGCAAGGAAAAAAATCCCATCAAATACAAAGAAACTAAAACTAAAC
TAGAAGAAACATAAAGGTGAATAAAACAGAACAATAATACCAGAACCTGAAGGCAGAAGAAAAA
TCTTAAAATCAAAAAGAAACAAACCCATGCTTGAGACTCTCCTTGCTGGTCCAGAGCCACAGTG
CTGCTGTGTACTGGCAGGAGGAATTCTGCTATAATTGGTCCTGGCAGTGTTCACCTTTCCTGCA
AGTGTTCCACAGCCCAGGGACACAATGTGGTCAGGAGCACTGCCAGGAACACCAGCAAGGGGGA
GCCTGCCACAACAGGCACAAGGCTCAGGAAGCACTCTCCAGCCCACGAAAATCTAGTGGGGGTC
CTCTTCCCTCACCCAAACACACTCTGCGCAGCT
SEQ ID NO: 2
shRNA artificial/synthetic sequence.
5′-GATATCACCTTACAGAAATTACTCGAGTAATTTCTGTAAGGTGATATC-3′
SEQ ID NO: 3
UBE3A-ATS artificial/synthetic target sequence.
5′-GATATCACCTTACAGAAATTA-3′
SEQ ID NOS: 4-489
UBE3A-ATS artificial/synthetic sequences.
    4. CTGAAGTGCACATGGAATATT
    5. TCTTCAGGATGACACATATTA
    6. CACAATGATATGGAGATTTAA
    7. CTCAATCCAATAACCTAATTT
    8. GTAAACAGAAGGAAGAAATAA
    9. AGGTCCGTATTACCCTTATAT
  10. CATCTAGCCTTTCTGATTTAT
  11. ATCTAGCCTTTCTGATTTATA
  12. TCTAGCCTTTCTGATTTATAT
  13. CTAGCCTTTCTGATTTATATA
  14. AGTCACAACTCAGATTTATTT
  15. GGCCTCACAACTCAGATTTAA
  16. GAACAGAGCCCTCAGAAATAA
  17. GGAAAGGATTCCCTATTTAAT
  18. GAAAGGATTCCCTATTTAATA
  19. GACAAACGGGATCTCATTAAA
  20. CATCTGACAAAGGGCTAATAT
  21. ACAATGAACTCAGACAAATTT
  22. CAATGAACTCAGACAAATTTA
  23. GTTAGAATGGCGATCATTAAA
  24. CAGCCATCCCATTGCTATATA
  25. AGCCATCCCATTGCTATATAT
  26. GCCATCCCATTGCTATATATA
  27. CCATCCCATTGCTATATATAT
  28. CATCCCATTGCTATATATATA
  29. ATATACCCAAAGGACTATAAA
  30. TATACCCAAAGGACTATAAAT
  31. ATATGCACAGGTATGTTTATT
  32. GAAAGACACTGATTGTATTTA
  33. ACATAGCCCTAACCATATAAA
  34. CCTCACACAAAGACCTATTAT
  35. TGGCAGAGATTTGGCAATTAT
  36. GGCAGAGATTTGGCAATTATA
  37. AGAACAACAAGGAGGAAATTT
  38. GAACAACAAGGAGGAAATTTA
  39. GAATGGAGTGAAATGTTTAAA
  40. TCTGATTTCCTTGGGTTTATT
  41. CTGATTTCCTTGGGTTTATTT
  42. TGCTCTTCTTTCTACTTTATT
  43. GTAAGCATTTAGTGCTATAAA
  44. TTAGTCACATCCCACAAATTT
  45. ATGGTCTGTGCTGTGAATATT
  46. TTGAAGTCTCCAACCATAATT
  47. CAGTTTGTGCATCACATATTT
  48. TGCCCTCTTGGTGGCTTATTT
  49. TCCTAGGTCATAATGATAATT
  50. CTCCACATCCTTACCAATATT
  51. CGCTTATCAGATATGATTTAT
  52. GGTCTATACATGTAGATTATT
  53. TCATAGATGTATGGGATTATT
  54. CATAGATGTATGGGATTATTT
  55. CTTGTAACTCCTTGGTTAAAT
  56. TTGTAACTCCTTGGTTAAATT
  57. TGTAACTCCTTGGTTAAATTT
  58. GTAACTCCTTGGTTAAATTTA
  59. TCCTTTCTGATGCTGTTTAAA
  60. CCTTTCTGATGCTGTTTAAAT
  61. CTGAAACTTTGCTGCATTTAT
  62. TGAAACTTTGCTGCATTTATT
  63. GAAACTTTGCTGCATTTATTA
  64. TTGCTCTGTGAATAGATAATT
  65. TGCTCTGTGAATAGATAATTT
  66. ATATGTTGATCCACCTTTATA
  67. TATGTTGATCCACCTTTATAT
  68. ATGTTGATCCACCTTTATATT
  69. TCTTTGGCTACTTTGTATAAT
  70. CTTTGGCTACTTTGTATAATA
  71. TAAGTGCATAGAGCCAATAAA
  72. ATGCACATGATCTTCTTAATT
  73. TGCACATGATCTTCTTAATTT
  74. TGGCTTAAACAAGAGAAATTT
  75. GGCTTAAACAAGAGAAATTTA
  76. AGGTATCAGAGTAGTATATTT
  77. TTAAAGAGTTGCCACATTATA
  78. GTTGCCACATTATACATAATA
  79. AGACATTGAACCAGCTATTAA
  80. GACATTGAACCAGCTATTAAA
  81. AGGGTTAAAGAAAGGTATAAA
  82. ATACCCGAGATGACCAATAAA
  83. CAAATCCACGAGAAGAAATAA
  84. ACTTGTGTTGATGGTATTATA
  85. CTTGTGTTGATGGTATTATAT
  86. ATTCATTGGAAAGGGTATAAA
  87. CAGTAACAAGAGCCCATTATT
  88. AGTAACAAGAGCCCATTATTT
  89. GCCCAAGAGATCCACTTTATT
  90. CCCAAGAGATCCACTTTATTT
  91. TTTCATCTCACCCAGAATATT
  92. TGTTATGAGATCCAGTTATTT
  93. ACTGAACTGTGAGCCAATTAA
  94. CTGAACTGTGAGCCAATTAAA
  95. AGTCTTAGGTAGTTCTTTATA
  96. CAAAGGCAGCAGCACAATAAT
  97. AGACTGGCTATTTGAAATTAT
  98. GACTGGCTATTTGAAATTATT
  99. CACCATCAAGAGAACTAATAT
100. ACCATCAAGAGAACTAATATA
101. CCATCAAGAGAACTAATATAA
102. GGAAAGTACATAGTCAAATTT
103. CAATGCATACTACAGTATAAA
104. AGTCCTTACGTGTCAATAATT
105. GTCCTTACGTGTCAATAATTA
106. GACACATACAGGCTGAAATTA
107. ACACATACAGGCTGAAATTAA
108. CACATACAGGCTGAAATTAAA
109. CAATATTGAAGCACCTAAATA
110. ATGGATCTAACAGACATTATA
111. CATTCTCCAGGATAGATTATA
112. ATTCTCCAGGATAGATTATAT
113. CAAGATGGAAACTGGAAATTT
114. TTCTATGAGGTAAGCTTATAT
115. TCTATGAGGTAAGCTTATATT
116. ACAAAGATCAGACAGAAATAA
117. CAAAGATCAGACAGAAATAAA
118. ATACCACAGACATACAAATTA
119. TGAAGACTGCCAACCATTTAA
120. GAAGACTGCCAACCATTTAAA
121. CTAATTCAGCAACACATTATA
122. GATGCAGGATAGTTCAATATA
123. ATGCAGGATAGTTCAATATAA
124. TGCAGGATAGTTCAATATAAT
125. GTGTCTGTTGGCTGCATAAAT
126. CTTTGTAGATTCTGGATATTA
127. GCAGAAGCTCTTTAGTTTAAT
128. CAGAAGCTCTTTAGTTTAATT
129. CATCCTGTTACTGGGTATATA
130. GTATATACCCAGAGGATTATA
131. TATATACCCAGAGGATTATAA
132. ATATACCCAGAGGATTATAAA
133. TATACCCAGAGGATTATAAAT
134. CCCTAGAACTTAAAGTATAAT
135. TGACAAACCTGGAGGTAATAT
136. GACAAACCTGGAGGTAATATA
137. GTCCATTCTCACCACTTATAT
138. TCCATTCTCACCACTTATATT
139. CCATTCTCACCACTTATATTT
140. CATTCTCACCACTTATATTTA
141. GTAGATGACATGATCTTATAT
142. GAATAGAGAGCCCAGAAATAA
143. ATGCTTGACATCACTAATAAT
144. TACACTGTTGGTGTGAATTTA
145. ACACTGTTGGTGTGAATTTAA
146. CACTGTTGGTGTGAATTTAAA
147. GGTATTTGCACACTCATATTT
148. GTATTTGCACACTCATATTTA
149. ATAAAGAGAACGTGGTATATA
150. AGACACAGTGGAATCTATTTA
151. GGTACAAACTTTCAGTTATAA
152. GAAGGCACTGATATGTTAATT
153. AGGCACTGATATGTTAATTAT
154. ATCCATTCAAGCTAGATTATT
155. TCCATTCAAGCTAGATTATTT
156. GGCATGGTGGCCCTCAATTAT
157. GCATGGTGGCCCTCAATTATA
158. CATGGTGGCCCTCAATTATAT
159. ATGGTGGCCCTCAATTATATA
160. TGGTGGCCCTCAATTATATAT
161. GGTGGCCCTCAATTATATATA
162. GTGGCCCTCAATTATATATAT
163. TGGCCCTCAATTATATATATA
164. GGCCCTCAATTATATATATAA
165. ATGAACAACAATGGGATTTAA
166. TGAACAACAATGGGATTTAAA
167. GAACAACAATGGGATTTAAAT
168. AGCAATAGTGGAGAAATATAA
169. GACCTAAACCCTATCTTATAA
170. ACCTAAACCCTATCTTATAAT
171. CCTAAACCCTATCTTATAATT
172. CTAGAGCAGCAATACTAATAT
173. GTGTAGCTATGACTGATATTT
174. TGTAGCTATGACTGATATTTA
175. GTAGCTATGACTGATATTTAT
176. TAGTCTTTGCTTGCCTATTTA
177. AGTCTTTGCTTGCCTATTTAT
178. GTCTTTGCTTGCCTATTTATT
179. CTGTGTTTAGTTGTCTTATTT
180. CTACCCTGGATAGGGAATATA
181. TTCTATTGCAACAGGATAAAT
182. TCTATTGCAACAGGATAAATA
183. CTATTGCAACAGGATAAATAA
184. ACTGGTGAACTCCTCAATTAT
185. TCAAGGAGGAAACAGATTATA
186. CAAGGAGGAAACAGATTATAA
187. AGGAGGAAACAGATTATAAAT
188. TGCCTGTATGATAAGAAATTA
189. GCCTGTATGATAAGAAATTAT
190. GAACCAATCTTGTGCTTTATT
191. GCATTCTCTGATCTGATTTAT
192. CATTCTCTGATCTGATTTATT
193. AGAGGCTTAAAGGAGATTATT
194. TTGTTCAAGGATTCCTTATTA
195. TGTTCAAGGATTCCTTATTAT
196. GTTCAAGGATTCCTTATTATT
197. TGTGGAGAATCATTGATATAT
198. GTGGAGAATCATTGATATATT
199. GTATATGAGAGTTCCATTATT
200. TGGCTGTGGAAACGCTTATTT
201. TGGATCGATGATGAGAATAAT
202. GGATCGATGATGAGAATAATT
203. GCCCTCCAATAGGACAAATAA
204. TGACCCAAGACTTGCTTTAAT
205. GACCCAAGACTTGCTTTAATT
206. TGTGCTGAAAGAAGGAAATAT
207. ATATGGCATGCCTCTATTAAA
208. TATGGCATGCCTCTATTAAAT
209. ATGGCATGCCTCTATTAAATA
210. TGGCATGCCTCTATTAAATAA
211. GGCATGCCTCTATTAAATAAT
212. GACAGTGGAACCAAGTTTATT
213. GAGACTCCATGGTTCATAATA
214. AGACTCCATGGTTCATAATAT
215. TTGTGGTCATACTACATTATA
216. TGTGGTCATACTACATTATAT
217. GTGGTCATACTACATTATATT
218. TTGCAACAAGGTAAGTTATAT
219. TGCAACAAGGTAAGTTATATA
220. TTGATTGATGCCCTCATAATA
221. TGAAGTTGGGTAAAGTATAAA
222. GTCAATACACAACTGATAAAT
223. CAAAGAGTTTCAGCCATAAAT
224. CATGCTTTCCAGAGGAAATAT
225. TCCAGAGGAAATATGTTTAAT
226. TTTAATCATCTGCCCTATATT
227. TTAATCATCTGCCCTATATTA
228. TGCAAGATACCATACATTTAT
229. GCAAGATACCATACATTTATA
230. GGTCCATGTGATTTCTTTAAT
231. TGCCATAACTATAGCATTTAT
232. GGATACCATCCACACTATAAA
233. TGTGTTTCCTTCCAGATAATT
234. GTGTTTCCTTCCAGATAATTT
235. GTGTGCTGAAAGGGCTATAAA
236. TTGAACCAACCACCCTATAAA
237. TAAACGTTGTATGGCTTATTT
238. ATCCCTGTGTACACAATATTT
239. ATGTGTGGTAAATCCATTATT
240. TGTGTGGTAAATCCATTATTT
241. ATGAGCATTTGGCAGTAATAT
242. TGAGCATTTGGCAGTAATATT
243. GAGCATTTGGCAGTAATATTA
244. AGCATTTGGCAGTAATATTAT
246. GCATTTGGCAGTAATATTATT
245. GAGACTCTGCTGAAGTTTATA
247. AGACTCTGCTGAAGTTTATAA
248. CAGTTATAGAAGTGCTAATTT
249. TATGATGACCTCCTGAATTAT
250. GGGTCTGGTGTTTCATTTATT
251. GGTCTGGTGTTTCATTTATTT
252. GGGTCTCATTATAGTTAATAT
253. TAACAGCATGTCAGGTAATAA
254. GCAGAGCCCTATTCCTTTAAA
255. CTCCTAAATGTTTGGAAATTT
256. TTAGCATTTCTGACCTATTTA
257. TAGCATTTCTGACCTATTTAT
258. AGCATTTCTGACCTATTTATT
259. ACAGGATATAGGGAATAATTT
260. CAGGATATAGGGAATAATTTA
261. TAGCACTGAAATGCCTATATT
262. AGCACTGAAATGCCTATATTA
263. GAGCAGAAATCAATGAAATAT
264. ACTTGTAACCAGACCAATTTA
265. CTTGTAACCAGACCAATTTAA
266. ACTCAGTGAACAAGTAAATAA
267. TAGCCCTGTATCAAGTAAATT
268. ACAAGATTCCAGAACATTTAA
269. CAAGATTCCAGAACATTTAAA
270. GAGAGTTACCCAAAGAATTTA
271. AGAGTTACCCAAAGAATTTAA
272. AGAAGAAAGGTTTGGAAATTT
273. AGCCCAATCTATAGGATTTAT
274. GCCCAATCTATAGGATTTATA
275. CCCAATCTATAGGATTTATAT
276. AGTTCATCGTTAGTGTTATAT
277. GTTCATCGTTAGTGTTATATA
278. ACCACCATGCCCAGCTAATTT
279. ATGACCCATTTGAAGTTAATT
280. TGACCCATTTGAAGTTAATTT
281. ATGCTGGCCTCACTGAATAAA
282. TGCTGGCCTCACTGAATAAAT
283. GCTGGCCTCACTGAATAAATT
284. TGTTCCCTCCTCTTCAATTAT
285. GTTCCCTCCTCTTCAATTATT
286. TTCCCTCCTCTTCAATTATTT
287. TTGGTAGGTTGTGCGTATTTA
288. ATTAGTTGGCATGCAATTATT
289. ATTCGTAGTTCTCTGAATAAT
290. TTCCTTCTGTTGGCCTTTAAT
291. TCCTTCTGTTGGCCTTTAATT
292. CCTTCTGTTGGCCTTTAATTT
293. GAAAGCATTTAGAGCTATAAA
294. CTTTCACTACCTGCCATAAAT
295. TTTCACTACCTGCCATAAATT
296. TTCACTACCTGCCATAAATTT
297. TCCTTGACCTATTGGTTATTT
298. CCTTGACCTATTGGTTATTTA
299. CTTGACCTATTGGTTATTTAA
300. TTGTGATCACAGAAGATATTT
301. TGTATAATCGCAGTCTATTAA
302. TCGCAGTCTATTAACATTTAT
303. CGCAGTCTATTAACATTTATT
304. GAGTGGTAAAGTCTCTATTAT
305. AGTGGTAAAGTCTCTATTATT
306. AGCATAAGCTATGTCATTAAA
307. CTCTTCATTTCCTTCAATATT
308. TGAGATACCTAGAACAATATA
309. GAGATACCTAGAACAATATAA
310. CTCTTTCTCTGTGAGATTATA
311. ACAACAGCCTGGAAGTATAAT
312. CAACAGCCTGGAAGTATAATT
313. ACAGCCTGGAAGTATAATTAA
314. ATTCAAACTGATGCCAATTTA
315. TTCAAACTGATGCCAATTTAA
316. AGTCAACACACCAATATTAAA
317. AGCTCCTGTTTGAAGTAAATT
318. GCTCCTGTTTGAAGTAAATTT
319. GCCTTCCAAGGTTTCTATTAA
320. TGTGGGTCTCTTTGGATTTAT
321. TATGGTTCTGTAGAGATATTT
322. TGTTCTCAATTTCCCTATATA
323. GTTCTCAATTTCCCTATATAA
324. AGGTTGGAACATTTCAAATAA
325. TTACATGGGCTGTTCTATAAA
326. TACATGGGCTGTTCTATAAAT
327. TGTTACTTAAGGTGGTTAATA
329. GTTACTTAAGGTGGTTAATAA
328. GTTGCTCAAGTCTTCTATATT
330. CAACATGCAGGTTTGTTATAT
331. ACATGCAGGTTTGTTATATAT
332. ACGTGTGCATGTGTCTTTATA
333. CTTTATAGCAGCATGATTTAT
334. TGTGTCTTTGGCTGCATAAAT
335. CTTTGTAGATTCTGGATATTA
336. GCAGAAGCTCTTTAGTTTAAT
337. TTTCCCAGCACCATTTATTAA
338. TTCCCAGCACCATTTATTAAA
339. TCCCAGCACCATTTATTAAAT
340. CCCAGCACCATTTATTAAATA
341. GTTGTAGATGTGTGGTATTAT
342. TTGTAGATGTGTGGTATTATT
343. TGTAGATGTGTGGTATTATTT
344. GTTCTGTTCCATTGGTTTATA
345. TTCTGTTCCATTGGTTTATAT
346. GGATGGCATTGAATCTATAAA
347. GATGGCATTGAATCTATAAAT
348. CCTAATTGAATACCCTTTATT
349. GGCTGTGGGTTTGTCATAAAT
350. GCTGTGGGTTTGTCATAAATA
351. TGTCCCATCAATACCTAATTT
352. GTCCCATCAATACCTAATTTA
353. TCCCATCAATACCTAATTTAT
354. CCCATCAATACCTAATTTATT
355. TTGTCTTTGGTTCTGTTTATA
356. TGTCTTTGGTTCTGTTTATAT
357. AGCATGCTTTGCTGGTATTAA
358. GCATGCTTTGCTGGTATTAAT
359. CATGCTTTGCTGGTATTAATA
360. ATGCTTTGCTGGTATTAATAT
361. TGCTTTGCTGGTATTAATATA
362. GAGAGTTAGAACCTCAATATA
363. AGAGTTAGAACCTCAATATAT
364. CCACCACGCCTGGCTTTATAA
365. CACCACGCCTGGCTTTATAAT
366. ACCACGCCTGGCTTTATAATT
367. CCACGCCTGGCTTTATAATTT
368. GTCTAGCTCCAAGTGATATAT
369. TTTGCTTCTGTCTGAAATATA
370. TTGCTTCTGTCTGAAATATAT
371. TCTTAAGTCTGTGAGTTTATA
372. TCTCTGATTTCCACCTATATT
373. CTCTGATTTCCACCTATATTA
374. GTATATACTCCAGAGAAATAA
375. CACCAGAACTTGAACATTAAT
376. ACCAGAACTTGAACATTAATT
377. CCAGAACTTGAACATTAATTT
378. GTCTGGAACTCCTGGAATTAA
379. CTTTCTGTGCCTGGCTTATTT
380. CAGGATTTACCTTCCTTTAAA
381. TCTGTTGATGGGCACTTAAAT
382. CTGTTGATGGGCACTTAAATT
383. TCATAGCGGCTGTACTAATTT
384. CATAGCGGCTGTACTAATTTA
385. TTTACCCATTTCCAGATATAT
386. GTGTAAATAAGGGTCTAATTT
387. TGGTTATGTCATCAGTAATTA
388. CCTTGCATCCTAAGGATAAAT
389. GTGAATCCAGTTTGCTAATAT
390. TGAATCCAGTTTGCTAATATA
391. GAATCCAGTTTGCTAATATAT
392. CCATGTTCATCAGGGATATTA
393. GTGTCTTTCTCTGGCTTTAAT
394. TGTCTTTCTCTGGCTTTAATA
395. GTCTTTCTCTGGCTTTAATAA
396. TTGTGATCCTTCTTCTTTATT
397. GTAGGTTTGTATCGCTATAAA
398. TAGGTTTGTATCGCTATAAAT
399. AGGTTTGTATCGCTATAAATT
400. GGTTTGTATCGCTATAAATTT
401. TCATTTGTCTCAAGGTAATTT
402. TTTGGGAGCATATTGTTTAAT
403. TTGGGAGCATATTGTTTAATT
404. TGGGAGCATATTGTTTAATTT
405. TGGATGGAATGTTTCATATAT
406. AGTATTGAGGTCCCGTATTAT
407. GTATTGAGGTCCCGTATTATA
408. CTCCCTCTTCACATCATTTAA
409. TCCCTCTTCACATCATTTAAA
410. TATGTCTTCTTGGTGAATTAA
411. ATGTCTTCTTGGTGAATTAAT
412. CTGCTCTCAATTTCCATTTAT
413. TGCTCTCAATTTCCATTTATA
414. GCTCTCAATTTCCATTTATAT
415. TCCTTCAGCACTTTGAATATA
416. TCAGCTATTACTTCCTTAAAT
417. CAGCTATTACTTCCTTAAATA
418. TCCTTAAGGACCTCCTATTAT
419. GCTTGACCTCTAAACATATAA
420. CTTGACCTCTAAACATATAAA
421. ACCAATACCTTGTGTAATAAA
422. TTGACACTGGCTCTCTTTATA
423. TGACACTGGCTCTCTTTATAA
424. CAGAAGATGTGTTTGATAATA
425. GTTTGACGTGAAGAGTTTAAA
426. CTCTGAGCTTCAGTGAATTAT
427. AGGGTTGAATGCTGGATTTAA
428. GGGTTGAATGCTGGATTTAAA
429. GGTTGAATGCTGGATTTAAAT
430. GTTGAATGCTGGATTTAAATA
431. AGTGAAAGCAAAGAGAATAAA
432. CATGATGTTCCAAACTTTAAA
433. AGGTGGCCAAGGGCCTTAATA
434. GCCCAGCTGGCTCTGTAATAA
435. CCCAGCTGGCTCTGTAATAAA
436. CCAGCTGGCTCTGTAATAAAT
437. TGGTGTCATCGGGCCATATTT
438. TAACTAGGTCAACAGAATATT
439. GCAAAGTTAATCCTCATATAA
440. GCAGCATCTGTTCTGATTAAA
441. CAGCATCTGTTCTGATTAAAT
442. AGCATCTGTTCTGATTAAATA
443. GCATCTGTTCTGATTAAATAT
444. CTGCACAACTGACCCTTTATT
445. TGCACAACTGACCCTTTATTA
446. TGATGGCTTTCCTACTATTTA
447. GATGGCTTTCCTACTATTTAA
448. AGCTCAGCCATCTGGTTTAAA
449. CTGACTGCCTTAGACTAATAT
450. TGACTGCCTTAGACTAATATA
451. CAGCATTTGACAAAGAAATAA
452. AGGATATGGATGATGTATATA
453. GTCGACCCTAAGAAGATAATA
454. AGAAACCTGAACAAGTAATAA
455. GAAACCTGAACAAGTAATAAT
456. AGTCACACAACAGACTAAATT
457. GTCACACAACAGACTAAATTT
458. AGCGAGAGTGTCCTCATTTAT
459. GCGAGAGTGTCCTCATTTATA
460. CGAGAGTGTCCTCATTTATAT
461. ATCCTCTATCTCTGTTAATAA
462. AGCAAGTGCTATTCCATATTT
463. CTGGAGTCCATGGAGAAATAT
464. CCTCCAAGGACATACTTTATA
465. CTCCAAGGACATACTTTATAA
466. TCCAAGGACATACTTTATAAT
467. CCAAGGACATACTTTATAATA
468. GTGAGGGCAATGGTGAATATA
469. TGAGGGCAATGGTGAATATAA
470. CGAAAGGCAAGCTTCTTAAAT
471. GAAAGGCAAGCTTCTTAAATA
472. GGCAAGCAAGGCAGGATTTAA
473. ACAGGAACCTGGCTCTAATTT
474. GGGACCTCCTGACATTTAATT
475. GGACCTCCTGACATTTAATTA
477. GACCTCCTGACATTTAATTAA
476. GTGGGAGCTTGTTACATATAT
478. CTAAGTCTTCTCATCTATATA
479. TTAGGTGGTCATGAGATAAAT
480. TAGGTGGTCATGAGATAAATA
481. AGGTGGTCATGAGATAAATAT
482. GGTGGTCATGAGATAAATATA
483. GTGGTCATGAGATAAATATAT
484. CACAAAGCACATGGCTAAATA
485. ACAAAGCACATGGCTAAATAA
486. CGGTTATGTAGAAGCTATTTA
487 .GGTTATGTAGAAGCTATTTAA
488. AGCCACCCTTAATTGAATAAT
489. ACTACCCTTTACTGGTAATTT
SEQ ID NO: 490
Stem loop artificial/synthetic sequence.
CTCGAG
SEQ ID NO: 491
Stem loop artificial/synthetic sequence.
TCAAGAG
SEQ ID NO: 492
Stem loop artificial/synthetic sequence.
TTCG
SEQ ID NO: 493
Stem loop artificial/synthetic sequence.
GAAGCTTG
SEQ ID NO: 494
shRNA artificial/synthetic sequence.
5′-ATATCACCTTACAGAAATTACTCGAGTAATTTCTGTAAGGTGATAT-3′
SEQ ID NO: 495
shRNA artificial/synthetic sequence.
5′-TATCACCTTACAGAAATTACTCGAGTAATTTCTGTAAGGTGATA-3′
SEQ ID NO: 496
shRNA artificial/synthetic sequence.
5′-ATCACCTTACAGAAATTACTCGAGTAATTTCTGTAAGGTGAT-3′
SEQ ID NO: 497
shRNA artificial/synthetic sequence.
5′-TCACCTTACAGAAATTACTCGAGTAATTTCTGTAAGGTGA-3′
SEQ ID NO: 498
shRNA artificial/synthetic sequence.
5′-GATATCACCTTACAGAAATTCTCGAGAATTTCTGTAAGGTGATATC-3′
SEQ ID NO: 499
shRNA artificial/synthetic sequence.
5′-GATATCACCTTACAGAAATCTCGAGATTTCTGTAAGGTGATATC-3′
SEQ ID NO: 500
shRNA artificial/synthetic sequence.
5′-GATATCACCTTACAGAACTCGAGTTCTGTAAGGTGATATC-3′
SEQ ID NO: 501
shRNA artificial/synthetic sequence.
5′-GATATCACCTTACAGACTCGAGTCTGTAAGGTGATATC-3′
SEQ ID NO: 502
shRNA artificial/synthetic sequence.
5′-TGCTCTTCTTTCTACTTTATTCTCGAGAATAAAGTAGAAAGAAGAGCA-3′
SEQ ID NO: 503
shRNA artificial/synthetic sequence.
5′-CTCAATCCAATAACCTAATTTCTCGAGAAATTAGGTTATTGGATTGAG-3′
SEQ ID NO: 504
shRNA artificial/synthetic sequence.
5′-TTAGTCACATCCCACAAATTTCTCGAGAAATTTGTGGGATGTGACTAA-3′
SEQ ID NO: 505
shRNA artificial/synthetic sequence.
5′-TCCTAGGTCATAATGATAATTCTCGAGAATTATCATTATGACCTAGGA-3′
SEQ ID NO: 506
shRNA artificial/synthetic sequence.
5′-GATATCACCTTACAGAAATTAnnnnnnnnTAATTTCTGTAAGGTGATATC-3′,
wherein nnnnnnnn can be CTCGAG (SEQ ID NO: 490), TCAAGAG (SEQ ID
NO: 491), TTCG (SEQ ID NO: 492) or GAAGCTTG (SEQ ID NO: 493).
```

What is claimed is:

1. A polynucleotide sequence comprising:
(SEQ ID NO: 2)
5′-GATATCACCTTACAGAAATTACTCGAGTAATTTCTGTAAGGTGATA
TC-3′

2. An expression vector comprising the polynucleotide sequence of claim 1.

3. The expression vector according to claim 2, further comprising a promoter.

4. The expression vector according to claim 3, wherein the promotor is a neuron specific promoter.

5. The expression vector according to claim 4, wherein neuron specific promoter is neuron-specific enolase (NSE), synapsin I (Syn), or Ca2+/CaM-activated protein kinase II alpha (CaMKIIalpha).

6. The expression vector according to claim 3, wherein the promotor is a U6 promoter or a H1 promoter.

7. The expression vector according to claim 2, wherein the expression vector is an adeno-associated viral (AAV) vector or a lentiviral vector.

8. The expression vector according to claim 7, wherein the expression vector is AAV1, AAV2, AAV3, AAVS, AAV6, AAV7, AAV8, AAV9, or AAV10.

9. A pharmaceutical composition comprising the polynucleotide sequence according to claim 1 and a pharmaceutically acceptable carrier.

10. The pharmaceutical composition according to claim 9, wherein the polynucleotide sequence is contained within an expression vector.

11. The pharmaceutical composition according to claim 10, wherein the expression vector is an AAV vector or a lentivirus vector.

12. A polynucleotide encoding a shRNA comprising a nucleotide sequence that is at least 85%, at least 90%, at least 95%, or 100% complementary to a RNA encoded by any of SEQ ID NOs: 3-489.

13. The polynucleotide of claim 12, wherein the polynucleotide is SEQ ID NO: 2.

14. The polynucleotide of claim 12, wherein the shRNA causes activation of, or an increase in, expression of paternal UBE3A.

15. The polynucleotide of claim 12, wherein the shRNA causes a reduction of expression of paternal UBE3A ATS.

16. An expression vector comprising the polynucleotide of claim 12 and a promoter.

17. The expression vector of claim 14, wherein the promoter is a neuron specific promoter.

18. The expression vector according to claim 17, wherein neuron specific promoter is neuron-specific enolase (NSE), synapsin I (Syn), or Ca2+/CaM-activated protein kinase II alpha (CaMKIIalpha).

19. The expression vector of claim 16, wherein the promoter is a U6 promoter or a H1 promoter.

20. The expression vector according to claim 16, wherein the expression vector is an adeno-associated viral (AAV) vector or a lentiviral vector.

21. The expression vector according to claim 20, wherein the expression vector is AAV1, AAV2, AAV3, AAVS, AAV6, AAV7, AAV8, AAV9, or AAV10.

22. The expression vector of claim 16, wherein the polynucleotide is a DNA polynucleotide.

23. A pharmaceutical composition comprising the polynucleotide sequence according to claim 12 and a pharmaceutically acceptable carrier.

24. The pharmaceutical composition according to claim 23, wherein the polynucleotide sequence is contained within an expression vector.

25. The pharmaceutical composition according to claim 24, wherein the expression vector is an AAV vector or a lentivirus vector.

26. A method of treating Angelman syndrome comprising administering to a patient in need thereof the polynucleotide according to claim 1.

27. The method of treating Angelman syndrome according to claim 26, wherein the polynucleotide encodes a shRNA which causes a reduction of expression of paternal UBE3A ATS.

28. The method of treating Angelman syndrome according to claim 26, wherein the polynucleotide encodes a shRNA which causes activation of, or an increase in, expression of paternal UBE3A gene.

29. A method of treating Angelman syndrome comprising administering to a patient in need thereof a polynucleotide according to claim 12.

30. The method of treating Angelman syndrome according to claim 29, wherein the polynucleotide encodes a shRNA which causes a reduction of expression of paternal UBE3A ATS.

31. The method of treating Angelman syndrome according to claim 29, wherein the polynucleotide encodes a shRNA which causes activation of, or an increase in, expression of paternal UBE3A gene.

32. A polynucleotide comprising SEQ ID NO: 2 encoding a shRNA wherein the shRNA is capable of inhibiting the silencing of paternal UBE3A.

33. A method of inhibiting the silencing of a paternal UBE3A gene by an RNA antisense transcript encoded by SEQ ID NO: 1 comprising administering to a patient in need thereof, an amount of the polynucleotide of claim 1 encoding a shRNA effective to cut the RNA antisense transcript encoded by SEQ ID NO: 1.

34. The method of claim 33, wherein the polynucleotide is contained within an expression vector.

35. The method of claim 34, wherein the expression vector is a AAV vector or a lentivirus vector.

36. The method of claim 33, wherein the polynucleotide is administered to the patient's brain.

37. The method of claim 33, wherein the polynucleotide is administered to neurons of the patient.

38. The method of claim 33, wherein the shRNA reduces or terminates transcription of a polynucleotide comprising the sequence of SEQ ID NO: 1.

39. The method of claim 33, wherein the shRNA reduces the levels of the RNA antisense transcript encoded by SEQ ID NO: 1.

40. A method of inhibiting the silencing of paternal UBE3A gene by the RNA antisense transcript encoded by SEQ ID NO: 1 comprising administering to a patient in need thereof, an amount of the polynucleotide of claim 12 encoding a shRNA effective to cut the RNA antisense transcript encoded by SEQ ID NO: 1.

41. The method of claim 40, wherein the polynucleotide is contained within an expression vector.

42. The method of claim 41, wherein the expression vector is a AAV vector or a lentivirus vector.

43. The method of claim 40, wherein the polynucleotide is administered to the patient's brain.

44. The method of claim 40, wherein the polynucleotide is administered to neurons of the patient.

45. The method of claim 40, wherein the shRNA reduces or terminates transcription of a polynucleotide comprising the sequence of SEQ ID NO: 1.

46. The method of claim 40, wherein the shRNA reduces the levels of the RNA antisense transcript encoded by SEQ ID NO: 1.

47. The polynucleotide of claim 1, for use in treating Angelman syndrome, for use in activating paternal UBE3A, or for use in inhibiting the silencing of paternal UBE3A gene by the RNA antisense transcript encoded by SEQ ID NO: 1.

48. The polynucleotide of claim 12, for use in treating Angelman syndrome, for use in activating paternal UBE3A, or for use in inhibiting the silencing of paternal UBE3A gene by the RNA antisense transcript encoded by SEQ ID NO: 1.

49. Use of the polynucleotide of claim 1, in the manufacture of a medicament for the treatment of Angelman syndrome, for activation of paternal UBE3A, or for inhibition of the silencing of paternal UBE3A gene by the RNA antisense transcript encoded by SEQ ID NO: 1.

50. Use of the polynucleotide of claim 12, in the manufacture of a medicament for the treatment of Angelman syndrome, for activation of paternal UBE3A, or for inhibition of the silencing of paternal UBE3A gene by the RNA antisense transcript encoded by SEQ ID NO: 1.

51. A shRNA encoded by a portion of SEQ ID NO: 2, wherein the portion of SEQ ID NO: 2 defines a first segment defined by the bold nucleotides which has been shortened by one, two, three or four nucleotides at either end of the first segment, and a second segment defined by the italicized nucleotides which has been shortened by one, two or three nucleotides at either end of the italicized nucleotides.

52. The shRNA of claim 51, wherein the shRNA is encoded by SEQ ID NO: 494, SEQ ID NO: 495, SEQ ID NO: 496, SEQ ID NO: 497, SEQ ID NO: 498, SEQ ID NO: 499, SEQ ID NO: 500 or SEQ ID NO: 501.

53. A polynucleotide sequence comprising:
(SEQ ID NO: 506)
5′-GATATCACCTTACAGAAATTAnnnnnnnnTAATTTCTGTAAGGTGA
TATC-3′, wherein nnnnnnnn can be
(SEQ ID NO: 490)
CTCGAG,
(SEQ ID NO: 491)
TCAAGAG,
(SEQ ID NO: 492)
TTCG
or
(SEQ ID NO: 493)
GAAGCTTG.

54. A polynucleotide sequence comprising a first portion, a second portion and a third portion, the first portion comprising any of SEQ ID NOs: 3-489, the second portion comprising any of SEQ ID Nos: 490, 491, 492, or 493, and the third portion comprising respective nucleotide sequences complementary to those in SEQ ID NOs: 3-489.


SEQUENCE LISTING (506 sequences)

SEQ ID NO: 1 - DNA, 68641 bp, Homo sapiens
  misc_feature 1..68641: note = Human UBE3A-ATS genomic sequence
  source 1..68641: mol_type = genomic DNA
tgagatgacctaaacaactgtggagaatcattgatatatttccttttttcactgttcatgttgggtgaaaataatcttgtagtgaaattcacatgttctaaatattgtttttttacatctttatctggcacattcataacatagatgtttctatacatattagtactgtaatcataccatatattattctgttaccccacttactccttaaacttttagttaattaaagagtttttataaagtcccccaatagattttttttttttgagacatagtctcactctgttgcccaggctggagtgcagtggcgtgattttggctcactgcaacctccccatcctgggttcaagcaattctcctgcctcagcctgccaagtagctgggattacaggtgcctgccaccacgcccgactaatttttgtattttcagtagtgacagggtttcaccatcttggccaggctggtcttgaactcctgacctcgtgatccacccgccttggcctctcagagtgctaggattacaggcttgagccactgcacccggccagattttaatctaattttattagaacaattcagtcatatgttttttcatgctatgtatatgagagttccattattcagatactaaacaaatgtctactgtacatttactgttctcactgatgatgcattagataaccatgcacaaaataagcctggctgtggaaacgcttatttgttgggagggtgcttgtttggatcgatgatgagaataattgtctgaggatgctgagggactcattccagatgtcaatctgaggtccagatgtgcggccctccaataggacaaataagactctcagagcctggctctatttggggatccctcagtgacaacatagtacccctgtgagcgtgccttttctatctcttcgaagagggcagtggcatcctgtcttatgagtcagtgtgcactttagtgtgcctagtgacccaagacttgctttaattgtagatagatacttacatataggaaatatttcttaagtaacaaatgaaaaactttagaagattgaattaagggtcaagcaactgtgatatgtctgaaaatctcattagtgttgtgctgaaagaaggaaatatggcatgcctctattaaataatgacagtggaaccaagtttattgctttgttatttttactgtggagtattttctaagattatttttgctttttttttctttcatgttttgctgagatagaaggcctggaatctgatcctccacttcagagaacaggggtgagtagctaagccattatcttttgaaattcatatgtcatgtgctctttgctaggtctttaggtcgttttgtacatcttttcagaagcttattggaggacattttcatgatatgtccttttcctcattgagaccctcaccatgtcacctacactattgaatccttatcatttctcttttaattttaactctcttttgcttttatggaaaaatgtagaatttaagagaatttttggcaatttcatattggatcaaaatgtattgtagtgaaatccaggtgtgccaaaatattaacagattttccccatctgtttaattattggggtttcagaatagagactccatggttcataatatctttgtggtcatactacattatatttctgcttctaatttaattattaaatattgacttgaattagtcttttcctcattgttgcaacaaggtaagttatataggaaattttcttctcttgatggcatgtctgagataatcatagatataagacacctggctggtttctagaatgcatgtaatttttatttcttgatctgtgtgttgagtacttgctgtgattgcatcatgcaaatacattgagctttacaattttagtgtatgcacttttctacatgtatattattcttcgatagaaagtaaaaaaaacttatcgaactagtcaaaatattggttaatacataaaaaaagtctcaagtagattgtgtattacatggtgcttgttgattgatgccctcataatagatcaagtgggttctctctttagcacagggctttttagcaaatcatgtcatgagtagttactcaagtatttttattttaacacatttatattttttctatgtatattcttaaattctcttatacttttttctctgttataaaaacatgctgaacaatctcaagtcttaaggattgcagtattgtccccacatattcatgtattttggtactcaattctttatactttctttgacagatcacttgaactggcacatgtctcttgttttgcagagagggaattaatgtgataccttcatgcttttctattctatgtgctacataattgaatatacaagcaaatatagttgttaagatttagtgtgattatttctacaccacatgcaaagaagtttctcatagatcttaatagaggcccacatgcattgtacagtttagaatttggggaaatattgatgaagttgggtaaagtataaagccaaaagtcagaacagtgaactccttgcttaaggatttccttggagattacttagtcaatacacaactgataaatttaagtgcttttcaccttttgagttctcgacatactaaagctaaaatgtgtttcaacttttaatcctgcttccctgattttcccttttttagtctgagatcaaagagtttcagccataaattactgccaagagtaatcacttcattttaagaaagcttaacaatatagaagaatataaaattatttatgacagatgtatttttaaccttttccccatgctttccagaggaaatatgtttaatcatctgccctatattagggaaaaactttctatgctaatacaagtatctatcaatccatttatctttctatataagatgtattgatcataaccaattaactttactgtaaatgagctttagatttgacattttggtagtaatatatgttgtacaatctcctgaggtcctataggtcttgaggtctctatgtcaaaaactatagatgtgccagtgtcctcagtgaatgtgaaggacaccacattttccttagccatttcttgttttcagaataatggttatcaacattttgctactgcaagataccatacatttataatcggaatatgccagtttttatgcactcatgcctctgtttctgtagagcattcccagaatgagtaatgcttgaaaattaggtccatgtgatttctttaatgagttatagtcaaatcatgaaatattcaggttaccattatttcagtaatgtattagaatgtcaaggtaaagttatctacattgtatatatacacacaacatagatataatttatacaacatctatatttatacaacagatatatatttatgtatatatttatacaacataaaatatattttattatttaaacataaaatatattttattatttaatatagattcttaagtgataaatatgtttaatattattaaaataggttaaaataggttatagttagtacagtgaaaattggcagcccttttacaaaacatatgccataactatagcatttatcactgacagtcataccaggatagtcttttatttccaatcacttaaatattcctaattgcaaaagaaatttgaagactaaaattcagaagttttgaaagagccattgcctgggtaaactatacaggtttcagttttatttataataattatgaggccaggcgcagtggctcacacctgtaatcccaacactttgggaggccgaggcgggtggatcacgaggtcaggagatcgagaccatcctggctaacacggtgaaaccccatctctactaaaaatacaaaaaattagccgggcgtggtggcgggcgcctgtagtcccagctactcgggaggctgaggcaggagaatggcgtgaacctggtaggcggagcttgcagtgagccgagatcgcgccactgccctccagcctgggagacagtgcgagactccgtctcaaaaaaaaaaattatgtatatatttataaattaatacttaataaattaataacttgtgataggcaatgcaaagatgacagtaaaaggacaaaattgattagattgataaagtcctgttaacatgaagaaattgaccaggataccatccacactataaagttagagaaatttatcaggacaattccctaaaatactcttctcaattttaacattgtaacaggaatttttaaaattttggtattatgtgtgtttccttccagataatttgaacagattcatatttggtatttttaaaagccatatctttgtccttagtgctggcaatgtattcttgagaatgaacaaataagagatacgtaaaagcataagagaaggtatcaggttgaagtagtcaatcagttatacagaacacaaagaattttatcttgtataatgtttatatagctttatagaagtgtgctgaaagggctataaaacatggacattattatctcattgaaaggtccaatacgtactgaaatacatgcttttattttgaaccaaccaccctataaacgttgtatggcttatttagatgagagcccaggttgtgtgtgtctgtgtgtgtgtgtgtgtgtgtgtgtgtgtgtgtgtgtgtgtgtgtacctgacagggaagcaagaacatcgagttgccaatgcactctgtctatggttagaatcatgctgaaaacatggctcccccagttctggaatgagcccacagatcaagcattccccaaagacatagcaggctcaaatccctgtgtacacaatattttatgattatcttatgtcagtactttcaaagtatacagtttgtgtgaagacaaatccaatgtcatttttcttggctagcctatatgtgtggtaaatccattatttacttacttgcttcctgaaaattacaattagattaacaaactgcagcaaagtgggcatgatgagatagagattgaagtgtaagcttatgttaatgatgcccttggtttggataaacacatctaagagaaaaatggaaaaacacacatggcagggaagccttgatagagccaaaatataggattgtatgtagtaatgcaatccatagatgagcatttggcagtaatattatttttcagatatggataaaaattgcttaggagagtaaagagagacaaagttgaaagcaggtttatagtaggtgttgttttagtgttgatccctttttgctccaataatcaaagtgataaatattgaaaattgattcatgcagcattacttactccattctaatttttatatatgtcaaaagtgccatctcccaaactgtgctatccccttcaggagaagagactctgctgaagtttataaggttgacatattgccagcttcaataatgtaaagatgaagtgtatactgaattcttaatgcaaataacaactctattggaaagtaacccagttatagaagtgctaatttgtcaggagctgccttaccaagatcatgatgagtacagttatctcaggattctgaaagattgttttccgatttcaactagtctagctgaatgttccttgatagaaagagaggacttttagaattggttcaatatgatgacctcctgaattatctcacatagcccgtttgtacatgcctttcttttctctcagaaaatggcactatcataatagctttcttacacagacttcaccttagggttttacattaagggaggggtctggtgtttcatttattttgaagtatttgttgttgattgtgtacagtgcttgagtaaaaaattgaatatagaaacatctagaatatttttttaaaggatcagtgtttataaagtgaattattagtgtcaataatgttgggaaagttttaagagaatataggaaacttgaacattacacaactacaatgggaccaaattgtggggtctcattatagttaatatttatgtatttttttccaattgatttgtgtgctttttttctgcatgtttttggcagatagaatggctataacaagtaacagcatgtcaggtaataaaaataagcagagccctattcctttaaaaatcttcactgatgggagggccataaaataagtcttaatacatttaaagaattaaattcatgtaaaccatgttaatttaattccacaatgatattgaattagaaataagaggaatatctcttgaacatctcctaaatgtttggaaatttaaattagcatttctgacctatttattggttaaaaaagatacaaagaaaggaaaattgaaaagtcttttgaactgaataaaaataaaaatatagaatctaaaactttatgggatactgacaaaacaggatatagggaataatttatagcactgaaatgcctatattagaaaagaaaaaaggttttaaatcagtaaatttgtattttaccttaagaaacttagaaaagaacaaattaacccagacttaagtaaaataaaggcactaataaagataagagcagaaatcaatgaaatataaaacaacaaaacacagagaaaaattgagaaaatttaaaaatagcctagtgagaagatattgataaacttgtaaccagaccaatttaagaaaaaaagtcaaaacacaaataccaatatttgaaaatgtaggagggcaaatcattacagattctatgaatactaaaatgataataaggaaaaattatttaaaaggggcatgtcagccaggcatggtggcttacccctgtaatcccagcactttggcaggccgaggtgggaggattgctagagctcaggcattcgagaccagcctgggcaacatgttgaaaccttgtctacacaaaaagtacaaaaattagctgggtgtggtgttgcacacttgtagtcccagtcacttgggaggctgaggcgagaggatcacttgagcccaggaggttgaggctgcagtgagccatgtttgtaccactgccctccagcctgggtgacaaagtaagaccctatgtaaaaaaaaaaaaatgtatgccaacatttttcaataacttaaatgaaatggaaaaattccttgaaagacacgaactacaaaaactcagtgaacaagtaaataacctgaatagccctgtatcaagtaaattgaatttgtagttaaaagccttccaacagagaaaacttcaggtacctatagcttcatatgaaatgaaaaaaaaaataccaatcctctacaagattccagaacatttaaaagaagggaatatttcccaacttattccatttggacagcaatacccaggtaagaaaaagagacacagaaatttaaaaagaagaatatacattattccttaggaacataaatgcaaagaaatctaatcaaaattttggcaaatgaaatgtagaaatactttatgaccaagtgagagttacccaaagaatttaaggttggttttatatgtaaagatcaaccaatataggaaaatcacttctggaaagtcagagtaagaaactccaaaaatctactcctccataaaaccaataacagccttgatagaaatagttgaaattaattttccaaaactttggaaattaaccaaaggcttacaaaattccagagaacattaattcaagaaaaatggctgaatcagtaagaacagccagctttgtggcattttaatatgaccccttcccatgcttttctccctagtgctgagatagtcttaaaaattagcaggatagcaaccactggagaagaaaggtttggaaatttcccaaaaagttccatccccatagaattatcactatttgacctctaaagcccaatctataggatttatattcatttggactgactcagagctcactcagtaaggaaatctcaatttcaaggtattggtcaaaaagaatccatggcaattgttgactatcacaactgcctgaagtcttggtaacagttgggataaacaagaagctgatcaaaaactgaaaactaaaatcttgggaatgagatatctacaggatgcttcaaaaagctttgatacattcctgtttatctagaaagctacatgcaggctgggtgcagtggctcacacctgtaatcccagcactttgggaggccgaggcgtgcggatcatgaggtcaggagtttgaggccagcctgaccaacatggtctctactaaaaacacaaaaattagccaggcgtggtggcgtgcatctgtaatcctagctactcaggaggctgagacaggagaatcgcttgaacccgggaggcggaggttgcagtgagccaagattgtgccactgcactccagcctgggcgacagagcaaggctctgtctcaaaaaaaaaaaaaaaaaaagccacatgcatgtaattgtttacctctggctttccttttatgctctgggcaagctaaggaagagttgtgaactacctaagtgctgaatgggaaccataacacacacacacacacacacacacacacacacagcaccttagtaaagggtgagaggcatgttagttagaagcattaaaggaaatctctttctagtcattatctgtgcactaacctaactgagcagagacttcagtatccacatactacagggcatataaactttacagaattagtccaggaaaatcatatctaaaaaaaaaaaaaagcagtaacaaaaataaactctgggaaaggggagaatatgatttaaagagttgccacattatacataatatgtctagtgttcaacaaaaaattacgagacatgcaaagaaatagaaaaatatggcacaaagaggataagatgaagtcagtgaaactatcctcgaggaagaccagatgttggtcttactagacacagacattgaaccagctattaaaaatacgtacacagaactaagaaaaacatgtcaaaagggttaaagaaaggtataaaaatagtgtcttaccaaatatagactaccaataaagagatagaaattataagaaaagacaacatgaaaaaatataaagcaaaaaaattagacaattgaaacaagagggcctctattcgcagatttgagcaggcagaagaaagaatcagtgaacttgaagatatgtcaactgagattatccagtctgagcaacaaaggtgggaaaaaatgaagaaaactgagcaacaaagaactgtagaacagcatctctcataccaatggatacataaactggagccctagaaggatgaaaaaaggagaaggaaagaaaacttcccaaattttaagaaaaacattaatttatatacccgagatgaccaataaaatccaattaagataatctcaaagagaccaacacctatacacatcatagtcagcgtgtcaaaagacaaacataaggagagaattcttgaagatagtaagaaaaaaattattcataacatacacaccatcctcaataagtctgacaattgacttctcactgtaaaccatgcaggctaaaaaggcaatgtacataaccaaagtgatgaaataaaaaccttcaaccaatcattctagatccaacaaaactattgttcaaaaagaagaaatgaagacattcctaaacaaaatctcagagaaatgttctctataagacttgtcctaatagaaatgctaaaggaactccttcggtctgaaatagaaaggcactggagagtaaatcaaatccacgagaagaaataaagagaaccagtataagtaactacatgtgtaagtttaaaacaaagtataaatttattttgtttgtaacatttgtcttttcctatttgatttaaaatataatctcaattataaacttgtgttgatggtattatataaagatgtaattttgggttatagcaccaaaatggcagaataggaattttctgtaggtgtttcccacataagtatcaattttgacaaccatccatgggcaagagtaccttgtgggagttcaggagttgacagtaaaacttcagcacaccagaggagtaaagaaatctaagaatagattcattggaaagggtataaacagtttcactttacctgcatcaccaacccccaaaagtggcacagctcagtaacaagagcccattatttcttccacagaggaaaaggagagtataataagtaagtgtccagtttctcaagacatacaggcccctgcccaagagatccactttattttcatctcacccagaatattgaggtgatcagcaaggtggagtggttgggagagggtaaaagcaggaaagagagatggggactcaaacagcagcccatacttggaactgccatagatcctaccagttacttcatggactccatcaggaacccacctataagccacaggggatgcatccctcccatcttgccaacaaaaccccgaatgctccaaatgcctcacccactctttggctggctcccaagtgcgctcctgtgaaaagcgagtgagtatctctgcagatggcttgcaagcacatgttgacagctggctccactctgtagaactggaaaaaagctcacatacatgagaatttcaggacactaccctaaaaaaaaaaatgagacttcagcacctggcctggctttatgcaacctagagaaggtgatatgatttggctctgtgtccccacgtaaatctcatgtcaaattgtaatccccacgtgttggacaagggacttggtgggaggtgattgaatcatgcgggtggacttccctcttgctgttcttgtgatagagttgttatgagatccagttatttgaaagtatgtagcatgtcccccttcactctcttgcactcctgctccaccttggtaagacttgcttgctacccctttgccttctgccatgattgtaagtttcctgaggcctcccagctatgcttcctgtatggcctgcagaactgaactgtgagccaattaaacctcttttcttcataaattatccagtcttaggtagttctttatagcagtgtgagaatggactaatgcagaaggcatacaacctttagaatttgcccccttgagggaacaagatgtgtgaagcaggttcatccatagaaaatgtctgagagaacctcaaaatccctaacctgactaactgatgaaagtgtttctctcctaaggccagtcagtaaagaccagagggggtgactgtttctttaaatgcaaaggcagcagcacaataattcaagaaacatgaaaaatcaagaaaacatgacaccaccagaagaacacaatcattttccaataaccaactccccaaaaatggagatttacaaattggtttataatgaattcagaacaattatgttaaggaagctcagcaaactaaaaggaacaccaatagactactctgtgaagtcaggcaaacaattcatgaacaaaactagaaattcaaaaaagagaaaaattatcttaaaagaaaacccagaagttatggagctaaagaatacaatgcatgaaatgaaggagcgtatcaacagcaaagttgatcaagcataagaaaaaaaaaatctgtgaaactgaagactggctatttgaaattattcatcagaggattaaaaaaaaaagaatgaaaagaaataaagaaagcctacaggatgtataaaacaccatcaagagaactaatataaggattattggagtcataaaggagaagagagaaaagggtagaaaacttatttaagaaataatggctgaaaactctccaaatctaggaaaagatatgagcatccaggtatatgaagctcaaagatccccgtacaggatacattccaaaaagacttcaccaaaacacatgataatcaaactgtcaaaagcaaaatcaagacgatgaataaaccaccaatcactaagagggagacaggattcttatgttgtcattattatttacatataatttcagtaaatgttattggaaaatttataatgttttaaaaaaagaaatttgaaagcaccgaaagaaaagagactcatcacatacagggaacccttttaaggcattcaagagatttctcagtagaaaccttacaaaataggagagagtgggatgaactatacaagtgctgcaaggaaaaaaatgccaaccaacgctttacctggcaaatctgttcctcagaaatgaaggagagagaagaactttcctagacaaacaaaagctgaggcagttcatcaccactagacctgccttacaagacatactaaggggagttcttcaagctgaaatgatatggcaatagttagtaatatgaaatgataaacctcactggtaaaggaaagtacatagtcaaatttagaacactttgatactataatgatggtgtataaataattttactgtgctatgaaggttaaaagacaaaagtattaaaaaaaacccatagctgcaatagcttgtcaatgcatactacagtataaaaagatgtaaattagaacattaaaaacatagcatgcaagggtagggaagtaaaagtgtagttttcatatgtaatcaaatttaatttgttatcagcttaaaataaattgttatacctatgttttatgtaagtgtcatggtaactataaaggaaaaacctctagtagatacacaaaagaaaaagagaaaggaatcaaaacataacactacagaaaattatcaaattacaaaggaagacagcaagggaggaacaaagtaaaaagagcaagaaaaaatttaacataatgaaaacagtaagtccttacgtgtcaataattactttaaatgtaaatggattaaattatccaaacaaaaaaacagactggacaaatggattttagaaacaacaacaacaacaaacaccgcacacacacacacacacacacaaaccacccagccccaactatgtgctgcctacaagagatttacttccactttaaggacacatacaggctgaaattaaaagaacagaaaaagatattgcatgcagatagaaaccagaagagaggagaggcatctatacttacagcatacagaaaagattttaagttaaaaactatatcaaaaggctaagaaggtcaaaatggtgaagcagttaattgttcaagaagacataaaaattgtaaatatttatacacccaatattgaagcacctaaatatataaggcaaatattaatacatataaaaggagaaatatacagcaatacagtaatagtagtgaacttcagtgcctccctttcaaaaatggataatccagacataaaatcaataaggaaacatttaacttaaacttcactttagaccaaatggatctaacagacattatactgaacatttcatccaacagtggtagaattcacattcttctcaagcacacatggaacattctccaggatagattatatgttagctcacaaaataatattacaaaatttaataaagctgaaatatcaattattttgcaccacaatagtataacactagaaatcaataacaagatggaaactggaaatttacaaatatatagcattaacatattcctgaacaaccaatgggtcaaagaaaaaaatcaaaataattttgtgacagcaaagtagaaacacaacataccaaaacatacaggacacagcaaaagcagttctatgaggtaagcttatattgataaacacatttaaaaaaagattttaaataaacaacattacacctcaaggaactacaaggaagaaaaaaaaacaagccccatgttatcaaagggaaggaactaacaaagatcagacagaaataaatgaaacatagactagaaaaacaatagagactattaataaaacttagagttagttttttaaaaaataaaatcaacaaacctttagctagactaaaaaaagagaagactcaaataaaataaaaaatgaaagaggagacattacaactgataccacagacatacaaattaagagaaaactatatgccaacatattagttaacttgcaatgggtaaatccctagaaacatacaacctacaaaaactgaatcatgaagaaatggaagatctgaacagatcaataatgaataagggaattgaatcaatattcaaaaatctcacaaaaagaaaagctcaggatcagatggcttcactggtgaagactgccaaccatttaaaaaaattaataccactctttattaagctcttccaaaaaaattgaagaggagaaaacactttcaaattcattataagaggccagttttaccttgatatcaaagatttaaaaagaacactttgagaaaggaaaattacaggccaaaacccttgataaatatagatgcaaaaatgctcagcaaaatactagcaaacctaattcagcaacacattataatggcatacatcatgaccaagtgagattcatgcctcggatgcaggatagttcaatataatcaaatcaacaaatgttacactactttaacagaatgaaggataaaaatcatatgatcatctcgatggttgaactagtttacagtcccaccaacagtgtaaaaatgttcctatttctccacatcctctgcagcacctgttgtttcctaactttttacagatcaccattctaactggtgtgagatggtatcttattgtggttttgatttgcatttctctgatggccagtgatggtgagcatttttcaagtgtctgttggctgcataaatgtcttcttttgagacgtgtctgttcatatccttcacctactttttgatggggttgtttgtttttttcttgtaaatttgtttgagttctttgtagattctggatattagccctttgtcagatgagtagattgcaaaaattttctcccattctgtaggttgtctgttcactctgatggtagtttcttttgctgtgcagaagctctttagtttaattagaccccatttgtcaattttgtcttttgttgccattgcttttggtgttttagacatgaagacagtgtggtgattcctcaaggatctagaactagaaataccatttgacccagccatcctgttactgggtatatacccagaggattataaatcacgctgctataagccataaaaaatgatgagttcatgtcctttgtagggacatggatgaagctggaaaccatcattctcagcaaactatcacaaggacaaaaaaccaaacaccgcatgttctcactcataggtgggaattgaacaatgagaacacatggacccaggaaggggaacatcacacactggggatggttgtggggtggggggaggggagagggatagcattaggagatatacctaatgctaaatgacgagttaatgggtgcagcacaccaacacggcacatgtgcacatatgtaacaaacctgcacgttgtgcacatgtaccctaaaacttaaagtataattaaaaaaaataatgctgctataaagacacgtgcacacgtatgttcattgcggcactattcacaatagcaaagacttggaaccaacccaaatgtccatcaatgatagactggattaagaaaatgtggcacatatacaccatggaatactatgcagccataaaaaagatgagttcatgtcctttgtagggacatggatgaagctggaaaccatcattctcagcaaactatcacaaggacaaaaaaccaaacactgcatgttctcactcataggtgggaattgaacaatgagaacacttggacacaggaaggggaacatcacacactggggcctgtcgtggggtcagggtagggggagggatagcattaggagatatacctaatgtaaatgacgagttaatgggtgcagcacaccaacacagcacatgtgtgcacatgtaccctagaacttaaagtataataaaaaataaatcatatcatcatctcagtagatttagaaaagcatttaacaatattcaacatcctttcagaactaaaaactctcaataaatcaggtatagaaagaatgtgcctcaacactataaaagccacatatgacaaacctggaggtaatatactcaatggtgaaaagtaaaaagctttgactctaagatcagaaccaaaacaaggatgtccattctcaccacttatatttaacatagtagttgaaattctagctagagcaattaggcaagaaaaaaggcacccaagttggaaagaatgaagttaaattgtctctgtagatgacatgatcttatatatagaaaacactaaagactccaccaaaatgctgttttaattagagcttaaaaaaataattcactaaagttgcaggatacaaaatcagtatacaaaaatcagttgcatttctaaacaccaaaaacaagttatccaaaaaattaagaaaacaatcctatttgtgatatcatcaaaaaataaaatactaaagaataccaaagaaactgaaaataaatggaaataaatggaaagatagcccatgttcatggattagatgaattaatactgttaaaatgttcatactacccaaagcaacctacagattaagtgcaattcctactaaaattccaatgacatttttcacagaaatagaaaacacactcctaaagtttgcatggaaccgcaaaagactcaaatagatgaagcaattttgagcaagaacagtaaagctggagacatcacactacctaacttcaaaattttattaatcaaaacagcatgacataaaaacagacagaaaagaccaatggaacagaatagagagcccagaaataaactcacgtttatagagtcaactaatattcaacgaaggtgccaagagtacacaatggggaaagtatagtctctacaataaacagcactgggaagacaatatccaaatgcaaaagaatgaaattacacccttatcttataccatacacacaaatcaaatcaaaattgataaagacttaaatataagacctgaaaccatacaatctttaggcaaaaactcattgacattggtcttggcaatgatttttttgatatgacaccagaagcacaggcaacaaaagcaaacctaaacaagtgggaccctaacaaactaaaaagtttctgcacagcaaaggaaacaatcaatcatcagaattaaaaggcaatttatgaaatgggagaaaatgtttgcaaaccacatatctgataaagggttaatatccaaaaatatataaggaatgcataaaattcaatagaaataaacaaacaaataatccaattttaaaatgggtgaagaacctgaatagacattttttcaaagaagacttacagataggcaacaggcatatgaaaaaatgcttgacatcactaataatcagggaaatgcaaatcaaagctccagtgaaataccacctccaactattaggatggctattatcaaaaactcaaaagaaaacaagctgggggaatgtagagaaaagggaaaagaagatccttatacactgttggtgtgaatttaaactggaatagcccttatggaaaacagcatggaggttcctcaaaaaattaaaaatagaactactatatgatccagcaatttcactattgagtatatatccaaatgaattaaaatcactgtcttgaagaggtatttgcacactcatatttatttcagcattattcacaatagccaagacatggaatcaacctaagtgttcatcagtagatgattagataaagagaacgtggtatatagacacagtggaatctatttagtgttcaaaaagaaggaaatccaacttttaaaatcctttaaaaagttaaactcataaaaacagagaggagaatggcggtttccaggaactggagggtgggagaatggggagatgttggtcaaaaggtacaaactttcagttataaaatgaataagttctagagatctagtgtacaacagcattactatagttaataataatattttttatacttgaaatttgctaagagtaaatatcaatattctcaatacacacaaaacagaactatctgaaggcactgatatgttaattatcttcattatagtaatcatttcacaatgtataatgaatatcaaaacaatagtgtacatcttaaatatatacagttttgatttgttaatcatacatcaatgaagctagaaaaaatgttgtaatttttaaaacaatagtaatataaattaggggtgaaggaattgacctatgttggagaaaagtttttgtaaactatttaaattaattggtatccattcaagctagattatttttaattgttaatttaattgtaatactaaggcaaccactaaaaaagcctttaaaaaaatatagcacttgaggctgggcatggtggccctcaattatatatataatatatataaaatatatattatatatatagtagatgaacaacaatgggatttaaatggtacactagaaaatatctgtttaacaaaaagaaagcaatagtggagaaatataagaacaaaaccatgtaagatttatagaaaatgaatagcaaattggttgacctaaaccctatcttataattatattaaagataaatgaaataaatactacatcaaaaggcagagattatcagaatagagaagaaaaatccaaaccataattcaaccttatgttatctgtatttagaatatttagagtcaagacacaaatagatagagttccatttgggcgtgaaaatatttaccatgcaaaatgtaattaaaatagagctagagcagcaatactaatatctgacaaaatatactttaacaaaaattgttgctaaagacaaaaaagaacattttataataagacacaacaattataaacatatacaccaaacaatagagcccaaaatatacaaagcaaaaactgctagaattgaagaaagatagaaaaatcaatgattacagttggaggtgtcaataccaaactttcagtaatacacagaacaacaagtcaaaagcaaaaaggaaataaaaaactacacattatcatacaacaccttaatacgatatccaacaatagcagaatacacattattctgaagtgcacatggaatattcttcaggatgacacatattaggctgtaaaacatatcttaataatttaaaagaattgaaataatacagcacatattctctgactgcaaaatattaaaccaattacagaagaaaatgtgggaaattcacaatgatatggagatttaaaaatacttcaaagtaaccaatgaatcaaagaacaaatcacaagagaaattagaaaatattttagatgaataaaactgaagaaacaacataccaaaattttgtggttgtagctaaagcagtgcttcaagggaaatgtataacagtatatccctaaattttaaaaaaatctcaatccaataacctaatttttcaccttaagaaatgagagtgaaagagcagacttaacccaaagtaaacagaaggaagaaataataaagattagcatggagataagtagtgaaaataaaaacaatagagaaaattaataaacttgaaagttggttcttctgatatatcagtaaaattgacgtattattagtttgaccgagaaagaacaaaagagagaagattcaagttactagacataaatgaaagtatagatatcaccttacagaaattaaaagaattctaacagaatattgtgaaaaagtgaatgtcaaaaaattaaattatatgagatacacaaatccctctaaaggcacaaactaccacagctggtgtaaaaacatgaatacaccatttacagttaaaaagactgaatagataaaaattttaagaagaaattgagtaagtaatttaattttcaaactataatcccaagaccagatgttttcattggtgaattctaccaaactttaaaaggattaatatctatttttcatacactctttccagaaaatagaaaaggagggaacactctataactcactgtatgaggtccgtattacccttatatcaagaccaaacatcataagaaaagaagactaaagacttatggtttctgctctgatatgtttggaagtcatcactactattgtcacaaggaaaaatctgaacaaactaaagtcaacgatttcttaaactaaccatagaattgaggtaacgggcgaaaactggagatgtaggcaaatacaaaaaaaatcacagtttatcaggagcagaaactgctgaaaccagcaactcgtatgaacatgttaagtggtaattgacaaatttctggagattgaatgtggactggattgagagttaggaactcctaagtgcccagtttttgatgaccccacacacttttgtaaactagacttccaagagccccagcaagtttcttacagtgaagactgcagaaaaatcccctgatgcttcagataggaggaagggaaaagcaactattttgaaataagcccaggggacaagtagttatttctaaacactctcagagcattttctttcacacggcagggggctccctgcaagggaagctactttgcctgagccttgtctgatgtaggagaaaaggaattggatggctcaagctccatctagcctttctgatttatataagggaagcaaaaaataggttaagaaactcttctgaaagtcacaactcagatttattttatatttatttatttatttatattttttgagacggagtctcgctctgttgcccaggctggagtgcagtggcgcgatctcggctcactgcaagctccacctcccgggttcacgccattcttctgcctcagcctcctgagtagctgggactacaggcgcccagctaattttttgtatttttagtagagacggggtttcaccacgttagccaggatggtctcgatctcctgacctcgtgatccacctgcctctgcctcccaaagtgctgggattacaggcgtgagccaccgcacctggcctcacaactcagatttaaccataagattatagaacacttctcctcccaaaacagagatatagatcaatggaacagaacagagccctcagaaataacgccgcatatctacaactatctgatctttgacaaacccgagaaaaacaagcaatggggaaaggattccctatttaataaatggtgctgggaaaactggctagccatatgtaaaaagctgaaactggatcccttccttacaccttacacaaaaattaattcaagatggattaaagacttaaacgttaggcctaaaaccataaaaaccctagaagaaaacctaggcattaccattcaggacataggcatgggcaaggacttcatgtctaaaacaccaaaagcaatggcaacaaaagacaaaattgacaaacgggatctcattaaactaaagagcttctgcacagcaaaagaaactaccatcagagtgaacaggcaacctacaaaattttcacaacctactcatctgacaaagggctaatatccagaatctacaatgaactcagacaaatttacaagaaaaaaacaaacaacctcatcaaaaagtgggcaaaggatataagcagacacttctcaaaagaagacatttatgcagccaacagacacatgaaaaaatgctcatcatcactggccgtcagagaaatgcaaatcaaaaccacaatgagataccatctcacaccagttagaatggcgatcattaaaaagtcaggaaacaacaggtgctggagaggatgtggagaaataggaacacttttacactgttggtgggactgtaaactagttcaaccattgtggaagtcagtgtggcgattcctcagggatctagaactagaaataccatttgacccagccatcccattgctatatatatatatatatacccaaaggactataaatcatgctgctataaagacatatgcacaggtatgtttattgcagcactattcacaatagcaaagacttggaaccaacccaaatgtccaacaatgatagactggattaagaaaatgtggcacatatacaccatggaatactatgcagccataaaaaagatgagttcatgtcttttgtagggacatggatgaaattggaaatcatcattctcagtaaactatcgcaaggacaaaaaaccaaacactgcatgttctcactcatagatgggaattgaacaatgagaacacatggacacaggaaggggaacatcacactctggggactgttgtggggtcgggggaggggggagggatagcattaggagatatacctaatggtaaatgacgagttaatgggtgtagcacaccagcatggcacatgtatacatatgtaacaaacctgcacattgtgcacatgtaccctaaaacttaaagtataataataataaaaaaaggctacctaaaaaaaaaaaaaagaacacttctcctcccaacaccatatcaccacatcaaccaggactccagtgtaatagcagtgaattctaactgaaagaggtgaaagacactgattgtatttaagaaagatcttctaaggaaatccaaaaatagtaggggagatcaaaacaaagatactagaggaaattgaatatgtgacacctatagctacaaaaaaattaaacataacatagccctaaccatataaacataaaacctcacacaaagacctattatctgagattctgttgcctgatacattgcgttttatttcaataaaaaaattagagggtatgttaaaaagcaggaaaagttagtctaaagagacaaattgagcctcagaagtaggctcagatatggcagagatttggcaattatacctagagtttaatataaatgattaatataataagtgttctaacagaaaaaggcaacatgcaagaacggatgggtaatgtgatcagcaagaaggaaactctaagaaagaagtcaaaaggaaatgctaggaataaaaacctacaagaaataaagaatgcctgtgatgggttcctcagtagactggacaaggtcaaagaatcagtggatttgaaaatatgtcaacaaaaactgccccactgaaatacaaaagaaaaatagaattttaaaaacgtaacacaatctccaaaacagtgggacaattacaaaagatgtaatgtgcctaatgcaaatgacagtaggagtataaagggagaaaggaatagaaaatctgaagtaataatggcagagagttttccaaaattaatgctaaaccacagatacagcaagcccagagaacaacaaggaggaaatttagtaaagcgtctgcaaccaagtatgtcatattcagactgacaaaaccaaaggtgaagagaaaatattgaaagaagacaaagaggaaaataaatatcaagaaaatacatacgaaatacatcatacatacataagaaatacatcagaccatacaagcaagaagagaatggagtgaaatgtttaaaatgttgaaaaaaaaactatcaatttgcaattctgtatccagtgagattatctttcaaaagtgaagagggaaatggcagagaagtcatctccaagacctatggtttcccttcacagaaacactgaaaaatatgaacaaaagtggtcagaattaactttctaagaattctataaaatggtaaaatgtttacaccagtaaagcaaatgctgaattgagaaggcaacttaaaaaggtgaagaaaacttcgtattatttttatgtgtccttgccccacgtccttccctaccttagtcttgaagatggcagcccacatttctactgtggggctctggtttctgtttcctggttcaagagggagaataacagaccttacttttagtcattattatttccttctttctgatttccttgggtttattttgctcttctttctactttattgaaatgagaactaagattatgatttgagacatttttctaatgtaagcatttagtgctataaatttccatctcaacactgctttagtcacatcccacaaatttttatatgttgtaatttcactttcatttagttctatttttaaattttttctttttatacttcctctgactcacagattacttagaattgtgttgttcagttttcaaggatattgtagattttcctgtttctctgttgtctaatagttctgttccattttgtacagatagctcacgctgtatgatttcaattttttaaaaaattgtgctttgttttatggcccagatatggtctgtgctgtgaatattccatgttattataaagtatgcctgttatattattatatatatataatatatataattataaagcatgcccgttttgtattgttagcagagtattctagaaatgtcaatgagatcttgttggttgatggtgcttttcagttctatatctttgctaattttttttttttttgcttagtagctatatgagattctgagagaggaaattgaagtctccaaccataattgtggatttgtctatttctcctatcagttctatcagtttgtgcatcacatatttgaggctctgttgtttggtgcatacacaagtggaatcattgtgccctcttggtggcttattttatgattatatagtgcctatctttgtggtatttttatttgctcttaaatctactttgtgttatattcatatacccattcttttttaaaaaaaattgtttgcgtgatacatcttttccattcttttaatctcagcctatctgtgccattgaatttgaagtgagttttcatatagagaacatattattgaatcatcattttaaaaattccttttgccaatcttttttatactgaggtaaaattgacataaaatttatcattttaaagtgtacaatacagtggcatttggtaatacacatgttatgcaacgttaactctacctggctcctaaatgttttcatcatccccaaaaggaaacttcatactcattaagcagttaattcccattccttctctcggccactggcatccgcaaacctacttttctgtctctatgaatttacctattatggatattttgtataaattgaattatacaataagtgacctttatgtttggcttcgttcgcttcgcatactatttttcgatattcaaccatgttgtagtatgtatcagttttatttgaataactcaattctttttgttgtatagctaaaagttgattcctaggtcataatgataattctatgtttagtttattgaatagctgccaaagtttttccacagtggctctgtcattttaaaatcccactagcaatggatgagagttccaatatctccacatccttaccaatattgttattttatatttttataattataattttcctagtgaatacgcaatggtatctcattgtgtttttggtttgcctttccctaatgactaatgatgttgagcatcttacaatgtacttgttaactatttgtgttctttagagaaatgtctattcaagtgccttgtccattttaaaaatcgagttgtcttgttgacttatgagttctttaatacagtaaacgcttatcagatatgatttataagtattttaacccattctgaaggtcaccttttcacttttgtggtagaccattatgcacaaaggttttaattttgataaatccaatttatccgcttttgttgttgtttttgttgttcgtgcttttgcaaaacctagtgtcatgaggttttctcattatctttggagaattttatagtttgggtctatacatgtagattattgatctaatttccattaatttgtgtgtatgctacgaggtaggggtccaaattcaatttttgcattgaattgaaaattcatattttcagtttcaaatttcaactgcatattcagttgttgcagcacaatttgttgaagagatcattctttaccacagggaatgatctgggacccttgtcaaaaatcaattgatcatagatgtatgggattatttcagactttagatcttgttccatgaatatgcctatttttatgccagcactgcagtattttcattactgtagctttatcataaattttgaaatcaggaagtatgtatcctccaagtgtgattttcatttgctagagtgttttgactatttggggtctttgcaattccatatgaatttcagaattggcttttcatttaaaaaaatggtagttgagattttcataggaattatattgaatctacagatcactttgggtagtattgccatcttaacaatattgtattccaatccataaacacggatgtattgccattcatatctttttttcttttctttcggcaacatttagtatatgacacttgtaactccttggttaaatttatacctaagcattttatcctttctgatgctgtttaaatggaattattttattaatttctcttttagatggcttgttgcaggtgtatagaaatagaactgattattgtgcttttattgaattatctgaaactttgctgcatttattaactctagtaggttttttttttctttaaagttgtctatatatcttgctctgtgaatagataattttacttcttgatctccaatatggatgcctttctttctttttcttacctaattgctgtagctagaattttcagtataatgtatgatagaagttgttcacaactatccttgtcttctttctgatcctaggggtatagctttcagtctttcaccattaagcacaatgttagctgtggggttttcttagatgccatttaatatattaaggaagtgctctgctatttctaattggttgagtatttttattataaaatggtgttagattttatgactttatgtactgcataattgagattatcatgtggtcttttcattcgattaatgtggtatattttatgattttcatatgttgatccacctttatattcttggggcaaatcccactgtgtacacgggttttttagggtctcctaattttcttcattctttttcttttctctccctgagactgaataacgttaactgacctatcttcaagtttactattttttcttctgctgttcaaatctgcttgaacctatagagtgaatttttcattttacttattgaacttttcagctccaaaatttctctttggctactttgtataatatctatctctctatagatattctctatttggaaagacatttttctcctggttttctttacttatttgtattttttaaagtctttaagcatatttgagacagttgatttaagtatttgtgtactaagtccattgcctaagcttttgcatagagattttatattaatttcttttttttctgtgaacaggtcttttttttttttttttttttttttttttttttagaaaactggacattttgagtagtataaacgtggctattttagaaataattttcctccctccttaggttttgtttccacttgttgcatgttgctgttgtttgctcattagtgacttttctaaagtattttgaaaagtctgtgttcttgatcatgtgggtccattgaattctgtattctgttaatttcatagtcagctagtgttctgaaagttcccttaagtgcatagagccaataaaagaaaaagaaaagaaacacagaaaaagaaaagaggaaaaaaggagggaaagagatttaaaaaaataatgtcggttgggcatggtggctcatgcctgtaatcccagcacttttgggaggccgaggcaggcaaatcacttaaggttgaccatcctggccaacacggagaaaccttgtctctactgaaaatataaaattagccgggcatggtggcacatgcctgtaatcccagctactcgggaggctgagaggtaggagaatcacttgaactcgggaggtggaggttgcagtgaactgagatcacaccactgcactccagcctgggcaacaagaatgaaactccatctcaaaaaaaaataataataataatgcctttgccgatttgctctgtgtttgggccctcattcaatgcctagtcaggccatttacaactctctcttaactttcactacctgcttgtgtactgactgaaggccacctataggtgaaagcttaacatcatctcagatctttttggaccatgaatcctaccctgggtatgcacatgatcttcttaatttcccagtagatgcaagagttttagtgttttaaaagtccttattccctcatctatcttcttttctgacctttttcagtctgcttattgttcatctgaactgacatcctttgccccaagcggctgtggcaaaaacttttacctttaaatgctttcaccacaagccacttgggaagctgccccagacctgggactgctctgaccctgatgaaacaaagacaagaccttgtacagccaggcagccataagacaagtccatacccaaaccacagttctttcagaataaggtctatattggattctctggccctagtaaccagcatgagtctgggcttgccatcttcatggccacttgtcttagtttgctagggctgccataacaaaatatgagagactgagtggcttaaacaagagaaatttattttttcacaattctgaagaccagaagtccattactctgaaatctctctccttggcttgcagatactgccttcttgccgtgtcctcacacagccttttctctgtgtacacatccctggtgttttttaagcctgtccaaattttctgttcttctgaggacaccaatcagattggattagcgaccacccatatgatcattttaccttaagtacctcttcaaaggtatcagagtagtatatttgctacagttgatgaacctgcatacaacatcatcacttgaggtctgcagtttacattagagttcatcgttagtgttatatattctagggtttgttttgttttgagacagaacagagtcttgctgtgttacccaggctggagcgcagtggtacaatctcattgctacctctgcttcccagattcaagcaattctcatacctcagcctcctaagtagctggaactacaggcgcacaccaccatgcccagctaattttcgtatttttagtggagatggggtttcaccatgttggccaggcaggtcttgaactccttgcctcaagtgatctgcccgcctcagcctcccccagtgttgggataacaggcatgagtcaccatgcctggccttattctatgggttttgaaatgtataatgacatgtatccatcgttgtagtattagatagaatagcttcattaccctaaaagtcttctttgcactgggttgagttttaaaagctctttgattattttatgacagttccttattagatatatcttttgcaagtatttttatcagtctgtggttatcttgtcttttacagagcagtaatttttaattttaataaaatccaatttgtcaattacttatctcatatgactctggtgttacatctaaaatgttaccaccatactcaaggtcacctaggttttctctaggaattttatggttttgcattttacatttggtgtatgacccatttgaagttaatttttgtgaggttgtaaggtctgtgcctagattcattttttttttttttggcatgttacttcagtattcataagaaacattgttctgtaatcttctttcttatagtatcttagtcttgctttagtttttgggcaatgctggcctcactgaataaattcaaagtgttccctcctcttcaattatttggaaaagtttgagaaagactattgttaactgttttctaaatttttggtggaatttaccagtgaaccaactggtcctaggcttttctccagtaggtggttttgattatgctttcaatctctttacaagttacacatctatacagacttttataattcagtcttggtaggttgtgcgtatttaggaatctgaccacttcatctaagttatccaattagttggcatgcaattattcgtagttctctgaataatcatttttatttccacaaaattggtaatatcccagtttccattttttatttcattgaatcttcttttttcttagctaatctagctaaatgtttgccaattttgttgatcttttggaagaaccaacttttgatttattaattttctctactctttttctgttctttatattatttatttccacactaatctttactattttcttccttctgttggcctttaattttttttttttaatttttaaggtgtaaatttaggttgagaaattttttaaatgaaagcatttagagctataaattttccttctggtgttcctttcactacctgccataaattttgatatgttgagtttttgtttgtcttggagtattttctaatttgtcttctaatttcttccttgacctattggttatttaaatgtatttaatttttgcatattgtggatttcccagttttccttctgttattgatttctagttttattccattgtgatcacagaagatattttgtataatcgcagtctattaacatttattaagtcttgtggcctaacagaggatctatgttggagaatgttccaagtgcaattgagaatactattctggtgctattaggtgaagtattctctatatgtctgttaagtccaattcatctatagtgttgacgtttcctgttccttactgattttctgacttattattctatccattattaaaagtggagtggtaaagtctctattattgtagaactctctgtttttcaagtctatcaatatctgtttcatatattttggagctctgtttgctgcatatgtgtttacaattgttatatcttcttggcaaattgaccagtttcatcaacataaaatataattcttattgtcttctaacagtttatttttctttttacataaagcctattttatctgatcttagattccctcacactccaccccagcacgcttttggttacatttacataatatatcttttccatcctttcattttcaacctgtttgtgtctttagatctaaagtgaatgtcttacagacagcataagctatgtcattaaaaaaatccattctgcttatctctgccttttgactggggagtttaatccatttgcatttaaagtaatcactgatcattaaatactttcagtattttgttgttttatgtatgtcttataactcttttgctcttcatttccttcaatattgcctttgtgtttagcttatctttttgtgtcacactttgatccccttctcatttcttttttatattttctttgtggttaccaggaggacaatgtatcaacttttaaagttattacaattttatttttttaaatctctcccattcggggattttaggaaggttaaataataatgtaaatgagatacctagaacaatataagcattcaggaattattaactcaattccaatccttcctccacctccacctctttctctgtgagattatagaaaagatgacaaaaaggatgttttctgagccctttaattgttgagaatgatctttgagaaaaagaaaaaaaatgaaagcactaggaatgtacaacagcctggaagtataattaagtgtaaattaaatagataaaagttataagcagaggaaagtatagtagaactcagtatttaaaagagaatcaatgtgaaaattatataaatttatgtaaaataaaactaccagacaaatctgatatccttaggatttttctttctttcatgtgatttctaattgctacatatgacactaaaccattgatctgagctgtaagagaaactggaaattgttctgttatcttttgtaagatttctagaacattttgccctcagacttaaatgccaacgtatttctcacttattgtttactgcttttggatttacatatgatttgattctttcttatctcttatccttacaatgtaattcaaactgatgccaatttaagttcaattgcgtacaaaaactctactcctatgcagctccgccccatctaatttacattattgatgtcgcaaattccatctttgtacatagtttacatattaacatggatttatacatttttatgtatttggtttttaaatcctgtagaaaataaaaagtcaacacaccaatattaaaataatactggtttttatatttgtccatgtgcttacctttatcagtgttctttacatctttatacgggtttgagttactgtcttgtgtcctttagttccaacctaaagaactccctttagcatttttatagggcaggtctagtggtaatgaactctctgagtttttatttagggatgtcttaatttctagctcctgtttgaagtaaatttttctggatatacaattctctgttgattgatttttgttcattttcccttcagctcttttaaatacattatcccactttcttctgccttccaaggtttctattaacaaaattcagcttataatcttattaaagatctcatgtacatgagtggcttctctcttgctgttttcaagattctgtgactttggtttctgatagtttaaatataatgtgtcttattgtgggtctctttggatttatcctagagtttcttggtcttcttacgttggtatatccatgtatttcaagacatttgagtagttttcagccattatttcttcaaacaatctctcctctttggggacttccattttacctatattggttcttttgatggtgtggcaccagtcccctagactttgttcacttttttccagtcttttatttctgcttctcagactcaacagcttcaagtgttctgtattcaagtctgctgactctttcttcttccagctcaaatctgctgttggatccccccttgtaaaatttttaattccattttagtgtttttcaagttaagtatttttattaggttcctttttataatttctttttgttgatattctcattttattacacataatttgtctgatttccattagtttttttctttgttttcctttagctctttgaaaatatttaagacattttaaaagtctttatccaagttcaatttctatggttctgtagagatattttctgccagtttatgttcttcttttccatgggccatgttttcctgtttctttgtatactttctaatttttggttgaaaactgagcatttgaaaatagagccaactttcccagtttctgcagagagtctttatgccacagtattcgttcactgattaactgggtatatctaagcttagggagcagctgagtcaaaagtttaaggtcttctcaggtcttttctgagtatacatgtttcctatgcctgtgtgaaatgttctcaatttccctatataaacagctacttcttcttttttttttttttttgagatggagtctcgctctgtcacccaggctggagtgcagtcatctcagctcactgcatcctccacctcccaggttcaaacaattctcctatacaggtgtgtgccaccacgtctggccaatttttgtatttttagtagagacagggtttcgccgtgttggccaggctggtctcaatctcctgacctcaggaggattacaggcttgagccacagtgtccagcctaaacagctacttttgaatgctttaatttcctgaatagtctcaacccagtttttccttgaggtcttaggtggtccattgtatgtctccacccatagttgcttgccccaggcatctgtgggtctgtggtaccactgcagctttcaccacctgtagctgccacctttccctatctgagatccaggttaggtgagagagatcattccttcacgcagtcccatgacaggttggaacatttcaaataaggtctgttctgctcctctggttgaagggagaaaattgggaaccggtttcccaccttctacaaaccaagatctcatgttgccacgggagtggcagggcaagtgcaagtgaaaatgccatacaattttctaccattttgaacgcgggtttttcttcaatggtcatttgcttggttgctgtaggcctttcactgttttccagagctcccataagattactttagccagttttttgttctttcctgatgcttccctggcagagtaagggttggaacttccaccattttgctgattcataactctgtagtcagttttaaatatattgatacttgagtttgttttatggcccagaatatggtcttggtaaatgtttcacatatactcagaaagaatgtgtattctgatgttgttacatgggctgttctataaatgttacttaaggtggttaataatgttgctcaagtcttctatattcttgctgattttcttttatttatttattttattttttttattatacttttaagttctagggtacatgtgcacaacatgcaggtttgttatatatgtatacatgtgccatgttggtgtgctgcatccattaactcatcatttacattaggtatatctcctaatgctgtccctccctgctccccccaccccatgacaggccccagtgtgtgatgttcccctttcctgtgtccaagcgttctcattgttcaattcccacctatgagtgagaacttgcggtgtttgtttttttgtccttgtgatagtttgctgagaatgatggtttccagcttcatccatgtccctacaaaggacgtgaactcatccttttttatgactgcatagtattccatggtgtatatgtgctacattttcttaatccagtctatcattgatggacatttgggttggttccaagtctttgctattgtgaacagtgctgcaatgaacatacgtgtgcatgtgtctttatagcagcatgatttataatcctttgagtatatactcagtaatgggatgggtgggtcaaatggtatttctagttctagatcttgaggaatcaccacactgtcttccacaatggttgaactagtttacagtcccaccaacagtgtaaaagtgttcctatttcttcacatcctctccagcacctgttgtttcctgactttttaatgatcgccattctaactggtgtgagatgggatctcattgtggttttgatctgcatttttctgatggccagtgatgatgagcattttttcatgtgtctttggctgcataaatgtcttcttttgagaagtgtctgttcatatcctttgcccactttttgatggggttgttttgttcttgtatatttgtttgagttctttgtagattctggatattagccctttgtcagatgaggagattgcaaaaattttctcccattctgtaggttggctgttcactctgatggtagtttcttttgctgtgcagaagctctttagtttaatgagaccccatttgtcaattttggcttttgttgccattgcttttggtgttttagacatgaagtcctcgcccatgcctatgtcctgaatggtattgcctaggttttcttctagggttttttatggttttaggtctaacatttaagtctttaatccatcttgaattaatttttgtataaggtgtaaggaagggatccagtttcagctttttacatatggctagccagttttcccagcaccatttattaaatagggaatcctttccccatttcttgtttttgtcaggtttgtcaaagatcagatggttgtagatgtgtggtattatttctgagggctctgttctgttccattggtttatatctgttttggtaccagtaccatgctgttttggttactgtagcctcgtagtatagtttgaagtcaggtagtatgatgcctccagatttgtccttttggcttaggattgtcttggcaatacaggctcttttttggttccatatgaattttaaagtagttttttccaattctgtgaaggaagtcattggtaacttaatggggatggcattgaatctataaattaccttgggcagtatggccattttcacgatactgattcttcctatccatgagcacggaatgttcttccatttgtttgtgtcctcttctatttcgttgagcagtggtttgtatttctgcttgaagaggtccttcacgtcccttgtaagttggattcctaggtattttgttctctttgacgcaactgtgaatgggagttcactcatgatttggctctctgttagtctgttactggtgtataagaatgcttgtgatttttgcacattgattttgtatcctgagactttgctgaagttgcttatcagcttaaggagattttgggctgagacgatggggttttctaaatatacaatcatgtcatctgcaaacagggacaatttgacttcctcttttcctaattgaataccctttatttctttctcctgcctgattgccctggccagaacttccaacactatgttgaataggagcggtgagagagggcattcctgtcttgtgccagttttcaaagggaatgcttccagtttttgcccattcagtatgacattggctgtgggtttgtcataaatagctcttattattttgagatatgtcccatcaatacctaatttattgagagtttttagcatgaagggctgctgaattttgtcgaaggccttttctgcatctattgagataaacatatggtttttgtctttggttctgtttatatgatggattatgtttattgatttgtgtatgttgaaccagccttgcaacccagggatgaagcccacttgatcatggtggataagctttttgatgtgctgctggattcagtttgccagtattttactgaggatttttgcatcaatgttcatcagggaaattggtctaaaattctctttttttgttgtgtctctgccaggctttggtatcaggatgatgctggcctcataaaatgagttagggaggattctctctttttcctatttactgtacatttattccaccagtgactaaaacaggtgtatcaataaaatctgttccctcaggttttgcttcaggtattttgagggtctgttatcaggtgcataaacaagattgttatgtcctattcttaaattaatctccttataattatgaagttaattttttttttcttgagatgcagttttgctctgtcgcccaggctggagtgcagtggcacaatctcggctcagtgcaacctctgcctcctgggttcaagcatttctttgcctcagcctcccgagtagctggggttacaggtacctgccaccacgcccggctaatttttttgtatttttagtagagatggggtttcaccatcttggccaagctggtcttgaactcctgaactcttgatccacccaccttggcctcccaaggtgctgggattacaggtgtgagccactgcgcctggacctggcccgaagtaaacttctttacccttgctaatgatctttgctctgaagcatgctttgctggtattaatatagtcattccttctttctttgattcatgtttgcagggtatatctgtttccattcttttacttttaacctattgtctttatatttaaagtgcatttcttgtaagtataattggtttcttaaaatccaattatctgccttttaaatgtcatttttatatgatttgcataaatatgattattattacagctaaattgaaatctgtcatcttgctatttggtttctatttatcccatttttttcccctctttttttgctttccttgagattgaacattgtattagttttctagggttgctgtaagaaagtgacataaagtggatggcttaaaacaacagaaatttattgtttcagtttggaggctagtcatctgaaaccaaggtgtcatcagggccgtattctctctgaaacctgtagggaagaattctttcctgcctgttctagcttctagcattttccagcaattcttggcattcctttgcttgtagatgtatccctccaatctctgcctctatcataacatagccatcttctccctttatctgtctattcttctcatcttataagaacattaattactgaattggggcccagcatagattaggcctaatctcatcttgaataggttacatctgccaaagattcttcttccaaataagatcacttttacagcttttacaggtactgagagttagaacctcaatatatcctttggtggggaccgacctcttacccataaaaagtattttatatgattccattttatctcctttttaggttattaactacaatttttttttcttttttttgagatggagttttgctcttgtagcccaggctggagcgcgattttggctcactgaaacctctgcctcccgggttcaagtgattgtcctgcctcagcctcctgagtagctgggattacaggcatgtgccaccgtgcctggccaatttttgtatttttagtagaaacagggtttcaccatgttggctagggtgggtctcaaattcctgaccttaggtgatccacccacctcggcctcccaaagtgctgggattacaggcgagagccaccacgcctggctttataatttttttttaattcagtggatgttttagggtttatagtatacatctttatcacagtctagctccaagtgatatatccctttatgtatagtacatgacccttacagtagtgcatttccatttttcctctctggcatttaggctattgcacacacatacactcaattcatccctttgtgtagatccgtatttccagctgctatcttttgcttctgtctgaaatatatgtatgatttcttttatgactattttgagaaatttggttatgtgcatttgtctatttttcttatgtacttttagctcatcaatcttaagtctgtgagtttatagtttttaaaaacaaatttgaaattatttggctattatttcctcaaatatttttttctgccgcccctgctttccctttccttagggatctctgatttccacctatattactctgattgaagttgttccacgtctcttttgaaaatctctgaaaaatcttttatcatttggataatctgtatttgtacatcttcaagtacattaatattttcttttgcaatgtttaatctgctgttaatcccatgtagtggattcttcatctcaggtattgctgttttaatctctagaaattccattgaagtcttacttgatgaaaaaggcagataaaaacaaatgttggcaaagatatggagaaatcagaatctgcacacactgatgatgggaatgtaaaatggtcaaggcaatttggaaagcagtctggcagtttctcaaaaggctaaacgtagttcccatatgacagcaatcattcatctaggtatatactccagagaaataaaaacatatccacaccagaacttgaacattaattttcatagcaacattattcctaggggtttaaaattttttacattatttttatttaaaatagagacagggtctcactacgttgcataggctggtctggaactcctggaattaagcattcctcctgcctttgtcctgttttctccactggaaaagaataaaactttgtacacttggactaacaactcccgattccctcctttaccaacatgccccacagcctctagtaacttactctctactttcatgaattcaacttttttaagattccacatataagtgagatcatacaatatttgtctttctgtgcctggcttatttctcttagcataatgtcctccagattcatacatgttgtcttaaatgacaggatttaccttcctttaaaggctgactagtacttcattgtgtatatgtatcacattttctttatccacttatctgttgatgggcacttaaattgtttcaatgtcttggctactgtaagtaatgcttcaataaacatgggaatgaagatatcccttcaacatattgatttctgttcttttggataatactcagaagtgagattactggatcatatggttgttctatttttttcagaaacctccatactgtttttcatagcggctgtactaatttacattcccactaacaatgcatgagttcacttttctggacatcctccccaacacttgttatctttcatctttttcataaaagccattatataataggtgtgaagtgacatctcactgtggttttgatttgcattactctaataattagtgtgagcattttttttttttcatgtaccgaatgtcttttgagaaaggtctcttcattcctttgcccattttaaaatcaggtggttttcttgctcttgagttgtttgagttccttatgtattttagatatttacccatttccagatatatcatttatattttttcctattctttgagttccctcttcactgtgttgtttccattgctgtgcaggtcttttattttgatgccaccccatttgtctatttttgccatgcttttgcagtcatatccaaaaaaatcattcccaagaccaatgctgtggagatttccccctatgttttcttcagtaggtgtacagttttaggtcttatatgttaagttttaaatctatttttttatatggtgtaaataagggtctaatttaattcttttgcatgtggatatccagttttcccaacaccatttattgaagaccctgtccttttatacttttcagtatgcagatcttttacctccttaaatttacacctcagtatttaatatttgttgctattatgagattttcataatttccttttcagatagctcattaatagtagatggaaacactactgatttctgtaagatgattttgtattacggaactttactgagtttgtgtatcagttctaccaggttttagttttgttctggtggagacattacagttttttgtatatggttatgtcatcagtaattacagatgatttaacctattcctttcctattaggatgccttttttttctttctcttgtccaactgctctggttaggacttctagtactatgtcaaaaagtgatgagggtcgtacatggcctccatacctaatctgttgagagtttttaccatgaaaccaggttgaattttgtcaaatgctttttctgcatccattgagatgatcatatgatttgatttacaccctccattttgttatgtggtatatcacactttttgatgtgcatatgttgaaccacccttgcatcctaaggataaatcccacttcatcatggtgaatcattctttgtattcgtgaatccagtttgctaatatattgttgaggatttttgcatccatgttcatcagggatattactttgtaagtttctgtccttaaagtgtctttctctggctttaataacagtgtaacactacccttgtaaaatgaatttagaagtattccctctgcttcattgttttggaaaagtttgagaatttttattagttctttaaatgtctggtaaaattcagtagtgaagctgcctaatcctgggctttcctttggtgggatactttttattactggctcaatctcttttcttgttattggcttattcagatttgtttcttcatgattcactctttgtaggttgtatatgtctaggaatttattcatttctttaggtcatccaatttgatggtgcataacttcatagtagtttcttataatcctttgtattttggtgatatcagtagtaaatgtctcctctttcatttctgatcttatttgagtactctttttttctcctagtctaggtaagaatttgttgattttatctttcaaaaaaaaaaaaaaccaactcttagcaactcttagttttgttcatttttttccagtctttatttcaactgtgatctttgttacttacttctttatgctaactttcgggcttagtctgttcttttcctagttcctttaggtgaaaagtgagattgtgatccttcttctttattggcgtaggtttgtatcgctataaatttccattaggactgattttgctgcatcacataagttttgtttccattttcatttgtctcaaggtaattttttatttactttttgacttcttctgtgaactattagttgtttgggagcatattgtttaatttccacatattgctgtattttccaccagaattgattcttgttcttgatttctagtttcacgccattgtaatcagaaaagggatttgatatgatttctgtccacttaaacttaagattagttttgtggactaacatatatcctggagaatgttccatgggcatttgagaacaaaatgtattttgctgcttctggatggaatgtttcatatatgcctgttaagtccgtttggtctaaagtgtaattgaaatccattgtttctttattgattttctgtctaggtgatcaatctgcccatggtgaaaagtagagtattgaggtcccgtattatagtattgcagcctatctccctcttcacatcatttaaaaattgctttatgtatttaggtgggtcaatgttgggtgcatatacttttacaattgttatgtcttcttggtgaattaatccctttatcattatataacaaacttctttctttttatagtattgacttaaagtctattttgtctgatagaagtatagctacccctgctctcaatttccatttatatagaatatctttttccatcccttcactttcagtctatgtgtatgtttagtagaaaagtgaatctcttgccgggcgcagtggctcactcctgtaatcccagcactttgggaggccaaggcggcgggatcaccagaggttgggagttagagaccagcctgaccaacatggagaaaacctgtctctattaaaaatacaaaattagccaggcgtggttgtgggcccttgtaatcccagctactcgagaggctgaggcaggagaattgcttaaacccgggaggtggaggttgcggtgagctgagatcatgccattgcactccagccttgacaatagcaaaactccgtctcaaaaaaagaaaaagaaaaagaaaaaaaagtgagtctcttataggcagtatgtggttgtgacttaaaaaaaaaaaaaaatccattctgtcattctatgtctttttgttggagaatctaatccgtttacattcaaggtaactattggtaagaaactgctagtgtcattttgtaatttgttttctgattgttttgtaggtcccttgtttcttttttccctattgctaccttcctttgtggtttggtggttttctgtggtggtgtgctttgaatcctttcttttatagtatatgcgattaccattgtatttgctgtaaggctaacttaaaacattttatccttaggctactttaagcgataaaaacttgcctttagttgcatacaaaaactctacgctttcacaacccccccgcctttcgtgattttgatgtcaaagtttacacttttttaaatttgtatctcttattgtagctacagttacaattacttcttgtagctacaacttattgtagctacagttgtttttaatagtttcatcttttaaacctcctaataggaataacattgctttacctgccacctttacaatactagagaattctgactatgaattacttataccatttagttttttacttttatgtttctcattaactagcagtcttttatttaagcctaaagaactcccttttagtaattccagtagagcaggcctagtagtgacaaactcccttaggcattgtttatctggaaaagtgtttatttctccctttgccaagtaaagtattcttaattaacagctttgtttccttcagcactttgaatataccatcccactctctactggcctgtaaggcttttgctgataaagccactgaatctgtattggggctaccttgaatgtgatgtttcttatctttgctgctttcagtattctttgtctttgatttttgataacttggttgtgatgtgtcttggtgaactctttaggttgaatctgatcggtgacctcagcttcctgttcctgaattttgtcatcttttctcagatttgggaatttttcagctattacttccttaaatatgctttctaggcctttttctttcttttctccttaaggacctcctattatgcaaaagttagctagcttgatgtgtcctgtaattcttataggcattcttttttgtttttgtttctcattggataatttcaaatgtcttacctttgagctcattgattcttctgcttgatcaagtctgctgttgaagcgttctacttagattttcagttcagttactgtattctttatctttagaatttctattgtttttgtttatatttgttaaacttctcattctgttcagatgttgttttccaaatttcatttttctatccatattttcttgtactttgttgaacttaagaggattagtctaaattatttgtcatttcacaggtctccatttcttctgagtctgttactggagctttcatatccaatgtacaccagaaactttgctaattccacagtaaatgttagaaatgggtttttattgataaaatcagtggcttgctaattagtatgttagaagctggcttgcattggtgaagttgagggtattcgatatacgtatcccggctaaaatacttctaaagaacctctccagctttgcaagaatagagtataggaatctgaggaggcctttaacatggccctcagaaaggacacacagggcttgacctctaaacatataaacatatacagaaacgaggtcagaacatgttttgactgatgccagtagtaacacagcagagcctattgctggtcactgttaccaataccttgtgtaataaacctttcatttaaattgagaggttgtaagccccgaaaaagcaaagggcttacagtacctcttgcctgaacactcctgtgtgtgctttctaagaaatgcttttaaaataatgcctgtggcacaaattgatttgacactggctctctttataagctgctatcaaggttgtggggaatagctaactcttacccactttccctgacaaataggaacccgtagttacctaccaaaagtatgtaaaagctatatccgacccaaaggccagttaggtcaaaacaccttgaaaggagctcagtttaaggaaatcagtggtgtcacagtgccccactagcgtgtaaaagttttcatagtttgactatcggatgtgcttagtttcacagctgagtcttaccttgtgatacttttagagcaaaagtcaaatcaaagatgatttaaaaaaacatttcaaagattcataaagaataaatgcctcctcaggaagtcttctggagttctgcccacttcccttaggtggctctttactgtgcccatctcatagcctgtctccttccacttggtgtattgcagagtaaagctcactgtttacaggggtggtagaaagtgtggtcctttcccagactcctttttcccttcctcttctcatttttcagaagatgtgtttgataataaacgaaacaaaatgactaacacattgagctgagctacataagcagatgtcagtttgacgtgaagagtttaaaagatctatgcattatctggggactcctcccccagacctgaaggatcaggtgctgccttctatgccacctgtgcagacagcaaaagaggaaaaccatacccacgttcagtatgaacaaaggggacatttgaactctgtgtggacccctcattggaagggtgtttcttctcctgctgcatccacaaagagcactccttagccttgccttttgtcagttctctctccaataaggcttgagcagagacaacccagtgcagttcagagagactgaagtctggtgttccaggtctgagtcctagctctagctctctgtgtaactttgggatgtcccaaagtaacttttcacaacttgataggtgttaacttgaattttggatacaggtgactcttagcccatcccctctctgtgcttcagatatgtcatcacttgggccatatgacctctggacacctttcctactttccacaatttcagagcagcagagcagactggagctcctgctgcctctgagcttcagtgaattatcactcgttggagggaagcttcaagcattttgttatctttcaagagcaaacacagtgtctgtcagcaagaatatgtagcagatgctagtgaacagcagtgattagggttgaatgctggatttaaatatggagcttaggctgtgaaggaagcctgaagaacctagagccccatgaagctgccctctgtgagatctgacaccatttcccactgatgagccatgggtctaatgggtagcactgattccacatacagtatgtgagtgcaatacagtgaaagcaaagagaataaaatgatggctaacatgatgttccaaactttaaacaggagaaaaacacacaattccattatgtataagaacccacacagagatcaggagaataacctcattggagaatgaatgacctgtgtggggaatttagggtagagttgagattgaaaaaatgggccgaagtcaggtggccaagggccttaataccttgtatacaagatgtgtagtcaaggaagaccatgccttacttatgcatcaattcccttgggcctacaaagagctgcctagcctgggactgttgtagagaaaagctacagtgttccaatgacacagggactcctccatgtatgtatgagtgcccagctggctctgtaataaatcttatttttatttattaacttttcttgcgcattggcttgatgcatcagttggaagtcagaggccaaacgaagtgaacactgagccaaaggaagttctcagcctttagggaggcagaattcactttaaacacaataaacaaatgaactcacattatacaggagagagtcagaagatcccagtggctggtgtcatcgggccatatttgcccgaagtgcctattccttataggaacccactcccagggttgatgggctacatccttaggaggctttatgcctatgttctcctgaccactggctcctccagggctggccttttttagtctctctgtagaggttcctgtagctggttggatataggctttcacagaagggtcagtgccttgggtctggttacaggactgttaatcttgctttgttaagagtaatgttatttccccatttccaaattctccagggagatggaatgtcaaagatagtatgactgtagcacctaaatcctgggttccagaagcagagaagagaatacactgaagctgtagaaaggcctattagtccatgtcagagattaactggatgcgaggaccatttctgggatggtgtatacagaactggagaactggatagggagtcagaaacaaagagctgaagatgatgcctttgaagtttccaacgtggataactaggtcaacagaatattactcaagaaagtattaacataggattgagatgcagtcagtagtaatggaattgaaatttcaaagtatatacctcatggatctcagggggtgttgagctgatcacctgggctaacactcctatgaccctggggaaaatcaaatgacctggtactgtagccatggtaggggtgtcatcaccttaatccaactgggacagtgctgtttgatattcatctggaacttggtgcagacccacattttgctgggtttcaccacaaccaaggcttttttgattcttttctcttttaacatcagtacatcactgcaaagttaatcctcatataataggagatgaaactaattgcttataaaaacaagatttttacaacactaaaattgttcaagcatatgggcatatttatagttgcaggcagtgtttcagatgcagactgttcttggctgcagtggttgtttacaggcagcatctgttctgattaaatatttgatgattatccccgaatgttttaaagcatagtactgggctctgctgactgtacaacaaactggcatttttgacctatagggcactgggctaggagatacagttctgagggaagtgaaagatagttaacaactgcacaactgaccctttattagttgcaataaagcaatccaaccacccaacccactgtgatggctttcctactatttaaggttggtggtgtcaaagagacaccctcctgtacagtgtgcagtgaatcaacatcatttccacaaaacctccttcctgcacaaaggaattatcatactttgttacgaagtaaaattttcctgtatcagtcacaggagttcaccagttaagatactgttagttgaagacttctggggtgacttaatgaaatagctcagccatctggtttaaaaactggattcttctatccctccacacagctgtccatgcacctgcatcatctcaaggctggtctccctggtggtagaaaggctgacagtaaaaactggagccacatgattccttgcttaggcagtgtttcttcatactctcacatgagagcaggcatgttctttccctaggctcacagtaaacactctgtcaaatctcacaggcccaaagtgcttttggttatccccattccaagccaatctgtggcatggaagatagcattaccctgactgccttagactaatatacctactccacttctggggctgggaataattttgaggtcaaccatccaaactgcatgacagccattccatggaagaggtatggcctaaatctttggggcaacctgaattcatgaaaactctcttagatttatgtaactatttttagaattcacttctgtatcatttaattttactaataaaaataccacctttaccctaaatgtcagccaagcgtaaggctccgttgggacagaaggaactatcaaagctttgtgtttttatacattagcagcatttgacaaagaaataactctgaaggaaggagaataaccaggcagagtctagatgcatggaaaagaagtctttgagaaggcttcgcaggctgaaggaaagggcaggactacttcaggaatacaacgtttaagtaaaagaggttggggtctgttgatcttgaggagagatgaggatggactggaaaataggagtgagatatagtaggagaggaaaggatatggatgatgtatatactgtatgggtaaccatcagtcgaccctaagaagataatagttgttaaatggttagctatttaatgaaagaaacctgaacaagtaataatcttgagttgcaaagtggctggagtcacacaacagactaaatttctggtagaacaaatccagcagcttatgtgaaggttaccatgtctgaagctggatagaaaaggtctaacttccaaccaaagtcacagttcttgagctcggtacacagagacagactgctaacagctgatgtgtcctcagcgagagtgtcctcatttatatcctcgttctcttctgcctcctttttttgttttaaacttttgggaagtctcatcattcaatacagttctaatatatcacaataacagaggcggcccaatttctacaaattgactaattctatccctgaaaagttcatacaataaaactatacaaagcatcattttcaaccatcctatagaaaaatttccctattaaattttaactctaaatcctctatctctgttaataaccttactaaaaacttccccatcactgcctgccagggagatcaaaagaaaccaaatttaagaaacctccaacacctgtacctgactgaaaagcaaacatacagacctttcagtcctgcccctactattcagctcctatcacagagacatggtggatgtccccttgggaagcggatagctcttaggtggaagctaggcctgcaataagcaggccaggaaacatgtctcccaggccccatactcttggcagaagcacttggccaccagacagcatgtcctgtcaacctacagagttcttaaaaacaaacactgggacccagaatagtaccctgtggtcatagtgcccacagttcactaagcaccctcacaggtctttgacagaacactgactgccaggtcacctggtgggcagagaaatggaagattcccaggcccaactagcatctcagggaagaaccacaagcagagcaactttcagagctggtcggccagcgttggcacccagggaagcaagtgctattccatatttggagagaacataaaactcaaggaaacagagaagtcctactcaataccgttctcaactgaaaacaagagaacttgcaaaaaagaaacccagttttctggagtccatggagaaatatgaagccaatgctcggctaacagagcagaaagccttttataaataatgccagtcaaagctccaaaggtcagagctgatgcatgccggattgttctgacaatttcttaggtttctaggcaacaaggagctggtcaaacagctcacctccaaggacatactttataataccaccctggtagacgagagcgaggcagcagtgaaggcaatagtcaagaacaggagagggaggtaaagaacaagatgctcttcagacagtctggacaaagacagtccctacccagctctcaggagttgcctccttaagaaggtggaggcccaggcagctcccagggtgaccctaaagacagactccaaggaaaagggtgtgagggcaatggtgaatataaggaggtcccctttcagtaggcagagcaagtcagttcaggtcttatttttagagtctctgaacagtgaagagaagctttctgtggacagcattccaccaccatgggaggggaaaggtgccatgagagacttctccagtggggcatacaagcattgtgcagtgatccccaagatccagccactggcaggaaatcgaaaggcaagcttcttaaatacatagtatatggagacagaagtgtggagcactcccaaaatgaaaggccaagacccaggaacaacctccacaacctggagtatatacaaatgaacccagccctgctgactcagatccacaccatcctaaagcaggggtttctcatgaatgaaagggctaagtatttggtggagagaatgagaagtctcccactgcccacacattttttcttcagagatttcttttccaagagccccttgaataaaggaagggagggagcactgaatgccccagaaatcagaatgcatggtgcgggaagacgacaggaaatagttctcaaagagatcagaaaataaattggaagcaatttcattaccacaaagaacagacattttttctaaggcacccctcccctttccacccaattgtcattcagcaactactgaatacttaccaatgaaaaacgttacccctgacctcaaggctgctcctgagctctggtagaaaatgctttccttgtctataaaacatggcaagcaaggcaggatttaacagtaagggcacagtagcactgcaagccttcaaatggaaacctggagacaagcagtgagaacaggaagcaaaagcagggcaggtgaagctaggaccagggcatctggaactttccacacaggttggatctccatgccagacaacagttttcaaggaaaaatatctaagaggaacatgactttgggaaactttttggcagtactgcttactgtatactagagagtaaaagaatttggggaacattcaccaatttgcttcttcaggggcttgggtagggaacgtgaacaggaacctggctctaatttctgaacttttttatcagtaaaaacaatccaacaaacgaaagctagtcagtgagagaactgggagggtctgccctccttccctgagtcaagccttctggggggacctcctgacatttaattaagcaaagacaacgcccactgaaggaagctgacctgaaagtgacacgctactgtgaaatgagcatgaagtgggagcttgttacatatatgaaatggccagcgatcctgagcaaagcgcttcagagcttgagacctaagtcttctcatctatatactgagggctggacaagatgatctgtcaagccatttttatccctaatccaccaaaatccaatgctttagtttattgtcacaaaagcaggtatcgaatggctatcctgcagtgcctccaatcaacattcagactttttccctgaggcaatataagataacagttaacatgtttttatcaattaggtggtcatgagataaatatatatgggaagtggtagtttttcacttaaatgcatataataatggtacagctctctttgaatagtatttgtttatttcttaaatatttaagttcctaaagacggtaagaataacccaaggaagtgaaatcaatgtcacaaagcacatggctaaataactgcaggtttgcagtgccatgtgtgagatcagatgacagaagggagaactacctttaggcagaggcttctcatgtcccctggagtggccatgtgctgttctacatgactacttccacttcggttatgtagaagctatttaaagcacacagatgtttgtgatgagaaaaaagccacccttaattgaataatggaaattataagcatgatttgagggtgggggtggaggtgggagtagagatgggtagaaaggagtgcaatggaaacaaaggagccttcataaaattcaagtcacttcttaggataacgtgattgatttactcaccaccttcttaggaacataaagcaaacaagtgggttttccttttactgcttttctgaaatgagctacactcaagaaagcagcacgggggttgtgctgtccctgcacagtggcaggagagtatgaggagcaggtgaatgccacaacagctccatccaagatcatttttcacatgcaggaaccattcttatactaccctttactggtaatttctgtagaaatctggaagtctggttgacaccctcctgtacagtgtgcagtgaatcaacatcatttcccttggactttgcaatcacggtggcattcatacattcattcaacaagtatgtatgtacaggacagtggagtaaagaaaacagatagttcttactctcacgaggcttaaaatttcaggagggaactaggcagtcatgaagtaaacataaaaacacagattgtaataggcactagagaataatgaagactttggggaacacaatttaaattggaaaaatatctcagttccgttaacacatgttgaaggggaatagcattgttttgctggatttggggcctggatgcaagcatgttgggtatgcattgtagggtaatgcatttccttccatttgggcccaagtgtatatttaccacccagttgtgatgagctgggatcctcctgctcaatctcagcttgaagcacttggaggttatctgcctgctgtgggtgattattttggagcaaggtacttcatttgcctcaagaaacagatttgataccactactgtgcccttttggaacagagaagtaggcaagaccccagtgtgaggcagagtgatgggatctttagggacataattgatgatgtaactgatgatgattttggagtttatacatttccaaagtttcaaaatattttcacttggttgatttatctttatggtgatgacactacgtagatatatgcccttcttaaaagttacagtaagaggctgggagcggtggctcacgcctataatcccagcactttggaaggccgaggtgggcagatcatgaggtcaggagattgagaccattctggctaacacggtgaaactccgtctctactaaaaatacaaaaaaattagccgggcgtggtggcgggcgcctgtagtcccagctactcaggaagctgaggcaggagaatggcgtgaacccgggaggcggagcttgcagtgagctgagattccgccactgcactccagccagggcaatgagcgagactccgtctcaaaaaaaaaaaaaaaaaaaaaaaaaaagttacagtgagagttgacattgagaaaagggaggcccagccagggttatgcaaagacacagtagggaggaggatgggaagtttctgagacaccccagtaactgcatgggtccacaaagcacatgtacagtctgctctatgcactgcaggtacagccacgaataagacaaagtctctgccctcatggagcttggtgtctgtctactggagcagacaaaaacaaacctgctttttctcatttgcctctactgtgggttccgtgtataactctcaaatgctagcatttccatgcattccatccttgtccttctctcctctctgccttttccaggagaattttcctagccacaggttcagctatggtctatgtgctggagtcatacatttttatcttctgtgtacgtttttctctagacccatatttttaattgccttaggacagctctccctggatatccctcagacacaactaaaacatcattcaattgtactattaataattttcccttcaaaatccactcctcttctctatagacctaacagcaacactgtccgtccagctctcaaaacctgaaatgtgggagttgtctttgactcttatctttcccatgcatgagcactgaatcaatcagagtccctagcatgtcttggactgtggcctcccttctatcttcagattatctgcctttttagaattatggcaataacttaactgcaaagaccacatgtgcacttgtgctctttcagtttgtaataaatataaaatgaaaagtgattatgtctttgcttaacgtctgcaaaataaaagtccaaagtccttggtatggtctataaggccctcattatctgacctgcctgccttcccaatctcatcttaatcccttacagcctgacactcagacatactagactttccacctaactcactcacatacacctatcctgtatgtcaagattcggcttgaccttcatcacctacctatctgcagtatttttggatctgaattctctgcccacattgtactttcacatacttcttttacccttagtggtttgtacttatgcctggtctaactagattgtctccctgtaacagacttcttgattcaacaaagcagctctgaatcagcctgaaacctatggcacactgcaaaatggcaaacattcaataggtatttgcccagtaaatgttaatgaaaggaaaaaaattcaaacttcagttggaataggattagagacaagttaaaaaaaagtttcccatagaaatcttcctcatcgtaaatatcatcatcctaaatgtcccgagtctttccatgggtctaatttaccaaatcatgaagactctctttctcactgtgtatgtgtagtgggagaggcagagacagaacggtttttttttttttttgcaagtctgctctctggattcgtttttctggggatgaaatctcaggctatgtagcttttctggtccctttttggtaatcaacaaatcatcagtttctctaggtattaaaaagcctacacttttgaaacaccaagaggccaaactctctcttaattgaaaacataattctgcctcttactgaggtctttgggtgggaagcttaaagacagcagtgttggggcaattggtttttcttttcctccctccaccttctttcccattcaagagctgttgctgccctttctggaggggagggaattagaaaaaaagatcctgccttacccagtcactgttaattattactttgagccagggtggggaatgactttctttcctggaatacaccctgctggaaaccacagctgagattctctaagctggcagcttccaacctctcctccctcaacagcttcaacattatatggcactcagctgcagggtgcctggctccaggccggcagtccatttgtgcagggtagttttcaagatggctcatagcccatctctttcagtgaccagcgtggcctatgggaaacatacatcttgactccattattggtaggagtactctttagttaactgccacaggccagttcagacacgtcttaccctgagagccttttcagagtcaggtaccatccctgtgtcccccagcatctgaagatcacaggtgaaggtctccaagtagttcacttaaaaaatacctcagtaggtttaagaacagggaaagctcccaattcattctgtcaggagtatgactctcatccccaaactggacagatatataatatgaaaactacagaccaatattccttatgaatatagatgcaaacattctaaacaaaatactagcaaaccaaatccagcagcatagaacaaggtttatgtagcatggacaactgagattatcccaggaatgcaaagttaattcaatatacaaaagtccattaatacaacatattaataaaggacaaaacaacatgaaaatcttgatacagaagaagcatttaacaaaacccacagcccctcctttttttttttttgattaaaaaacactcagtaaactaggtttaataaaagaatttcctcaacttgaagcaaacccacagctagctaacatcatactcaatggtgaaagactgaatgctttccccttaaaatcaggaacaagaaaaagatgtctgctcttgtcacttctatccaatattgtactggaggtgctagccagcacactaaggcaagaaaatgaaataataggaatctagactggaaaggaagaagtaaaagtatttctgttcacagatgcatgatctcatatgcaaaaaatactaagtgacaaaaaaagaattttagaaaagctacaatatacaaaatcaatatacaaaaattaattttatttctaacaccagtgatgaatacaaaaataaaaattagaaaataatcatataattaaagtggcatcagtaaaatacttagaaaaaaattaaagacgtcaagacttgcacactgaaatctctaaaacatcactgaaataaagacctaagcaaatgcaaagacatcccacagtcatggaacagaaaacctaacaccattaaaatagcagttatttctcaaatttatctaccaattcaactaaatccctatcaagatcccagctgttttgcaggaattgacacaatgatcatataaaaattcatatgcaatgcaagggacccaaaacagacaaaatgattttggggaaaaaaaaaaaaaaaatggaggacttgcacatcccaaatctaaacttactaccaagctacagccatcaagacagtgcggtgctggtataacgacagacatatagggcaatggagtaagactgagaatccagaaagtcttatatttatggtcaactgttctttgacaagggtaccaggaccattcatggggaaataatagtcttttcaacaactggtgcttgggcagatggatatacagataccaatgcacttatgcaaaaaatggatgaaataggaaacttcactacattctactgcatgctcggtcattttcaatcatttaggtggcaacactgacaagataacagaaagatggaggtaataacatgtgaaaggcaaaatggtttgtttttttttttaaaaatgacagtctctatcatgaatttacttacactccaggcaaaggttattagaagaaaaaaagatgtaagaaaattccttaactgaaatgtggaaagagtatcaagaggagaccctaagacactctgtaagaatcccagtgactcctcactgttcaactaagaaatgtaccccattatgctgtgctaccacggaagcattggaggcactttgggggttgatgaagtcttcatggatgaggtactttaaatatctggtcatgaagagtatttgagaaatatgacaagtgaagatgctgggtaggaatgcagcgaggaaagtgtgtaacacaaggccaaattggaaaggtcagtgagggggcaatctgtggaggcactgaatgcagaggatattaaaattagcaagatagtgttctagcacaatgaaaaggattggaagagggagatgagagtcagggagtgaaattaggcagctgctaagaatgtccgggtgagtcagaggatcaggacctgtaagggcagtataaagaaggaaggaatgatgggagaggtttagggggaaagaagtgacaacctgtgacaattgaggaatgagagattttgatgattggggtgaaggttacatttcttaaaaagaaacaaagaatggtcggtcgatagagatgcaagatgaagaattttgtttttaggactactgactatgaggtaacaaagaaatcccagagacagaggtctgagcaaaagatatgggattggggaagaatctttgggagtgactgaggttgactagagggaactgggcaagaacaggtaaggactttaagcagaattggaggagtatccatctaaaatctgggtaaactgggatggaaaaacaagtagccagagaagcaacagcccaagctagggtgttgtcaaggttatagatgttactgattttggcaatgaggagattataaggatccttgaagactgtactttcggtgaaactaccatgcattagtagcctttcacagtgatatctaccccacacctgcatcaaaatcttgcagtgcctattaataaatgccaacccactaacggggaggggagaaaagacttgggactctgtacttgtaaaaccctctaccctccccaggcaattctttacacactaacactgttgaggtaaaaggagacaagtgagggagcaaatggaaggtgtgtttttggattatacagtggctcatggaagggtgggaggggtacagatggcccttagacactggcggaaagtcagagaaaaaaaattacagtaagcaggtaaagggatcaagactacacagggactagtcttgggacgacatttcttcctctgacagtttgtatggaattcttgagaaaaattcctcgaaggggcctgaaatctcagaatggtcatgtttttaatgggaatagggagtgatgctgccccattacaaccatctgttctaacagaatgtctgtaccgaggagggatgagtaacatcggcaagttctgttcgaagcctttttcaagtttctttttgatatgtatctatctatctatcatctccctatacaagcaagcatccccaaaagtagttgtctcaggaaaccagggttaggatgaccagctcatttgctggagctgccaaggtccagaaaagtttggctgcaggctgttttcatgttttatgtatgtttgaaatgttctataataataaaaggttaaaaaagtttacatttatttggaaaaccagttactttagtttatggttcctttttttccctccagagcttcctggagattgaggtctaattcaaagaaaaccaaaatatataatagagtacctgggcaaaaaaagtacttttataacataacatttggggtagaggaagtatccactgtagtcaaaatgtctatgttttgctcttccttattgttcagggacattccattaaatagtaatgaaaaggcagcaaaagtaagaggagtgacaacatgcccggcataattaagcaagctagagcagctattctgtgcaaccgacgattttttttctctaaaattttaagggtaggttcattctgactctgttaaaagtctacttgatgtgaacaactctatatctgataacctatttcaattaccactttaaaacttgtcatatggatacgttattacaattgtagaactttaataaataccataataataaaacttgagaactgaagagcacacatttcttcacgaatttattatataaaacgccctcagagtatttaatttctcctcactttaattacacattaagaagcacagtggatgagaagcctttaagatgactacagttgcacgaaggtccctttcatcaaggtagcgtatgtaccctaacagtgttctaaaggctggcccagaaaaaccccatgttaccttatcacaatatggaaagcattgtcttctttttccactaaattaaattatggtgaaaagtgccacagttttatttagcattatggtacataacaaacagttctgtctcaattatgaaaaaaattaattaaaataatcctgaaagacatcctttttctccccccaatgatttgaaagctgcatttttcctgccaatttcaaacaaacaaatcatcaggttgatctacagtaatcagttaaaacaatcagtcaatcaatcaatcaatcaccaaggcacaagctcagcacattagctatagcttgtagcaaaaggatatatcaatgtctcaccttagttaaaaatacataatccttttattttataatgcaataaaagaaattaacaacatcacatacacagaagactaggaaaggggaaactacttacttctggaaatcagtaatgtaaacctacttgtacttttccatagtacatgaaagtaacgtttaacatgttttgaattaattaattaaatttaatctgtggggctatacaatgtaattcttaggagtaatagtttcattcatttccaggtcagcttactgtatgattaagtaacacaaggcacagtagccatctttttcattatgttgcaacactgatcacgtgcctcgataaaatggctgattcaacaagatgatggcaacacgaaggggagactttggattgtctatttaaaatctaggtaataagtaagtaattaataaaaactctatcttaagtgcactttcacatgctttttgtttataataaacaaacaacaaacttcctaactttgttgcaataggcttgactaccatttcatttggccaaatgcactttccccagtaaacttaaaacaacaacgagaacaacaagaacaaaaatccctgtcctttcatatactaagaaagaggattggctactgaaacagttcattgcaagacacatgaagacgacatactgtggcatgagttgtttttgtttttaatttgttgtgctgttactaaagttctgagggctgcagttaaaacattccaatttctcccttccttccatctttctttattgattgattctcaagattttgcacagaaaactctttgggggctagaacagcagtaattgcatcacactgttttcaagacttcaagtttcaaaagcaaatcattaaaaaaaatacagttcctgatttgagttagatacagggacaaaaaagtagcacatacttgaaggttacgtggtctacaaatggtggcaatattttccttgggagagtagttctgttggtatatattttttaaatactcaaaaggctcaacctcaagcagtaataaacacaagcaaaagtgatttaacccttaaaataaatattcagaaaaacctctctgtacatacaagtgaaagaatatgtaacactttcacgcaaaaaaataattataataataataaaggatttgttcatatatgtagctgaaatctgctgttccagcccacatgtccccaataaagaagggaggcacagacataggtgactactgtggttgactatcttacagcctttttgtactgggacactatcaccaccaaaaatttatccctcgttatatttttaaaattttttaaattttttctttttttttccttccttttttttgttttattttgttttgttttgttttacagcatgccaaatcctttggcatacgtgatggccttcaacaatctctctttaagtttttctttgcttgagtattccggaagtaaaagcacattaaagcaagtatgagatgtaggtaacctaaatagagaaaaggggaaaaaaacaggaaaactgtaagtcatgggaaatacacttagaattaaatgctcctatttttagattgtatatagttgagacggtctgcaatgcaaactatacattaatgcaaatcataaactttttgttgtgtaactaccaagttgcctttatcctataaattactcaaagctagtgacgatgataagatactgtatccattgagtttttactacataacagataccattttaggtactgaattcttacagttcatttaactaaatctttccacaacaaaaccacagagaggacatcagtaaatgcactttagaggttaggtcactgagaagtcaagtaacttcctctaaggtggagaaaatactcaaacctgttttacaagactgcaaagtgtgtgctcttaaatgcttattagaaacactgctggcaatatgactaagaaaatgatttgataacaggattctagcacaatcaaatgataatcttccgagcctcaatgtaaccattctaaatagatgatcatgttatatggctttcaattaacaagctgggaatcaaaaaagtaaatgaatcacactaatttgattccaaaccaatgtgagccccataataatttttaactagggcaatttcttaaaagtttcctcacacaatgacagcaaagtattttctcaattgtctaatatgatttggggatttgtatataaaatcacgaatgtgctcagaaactataaagacagttcatatgtatgtgacgaggaatgcaaggttttcggtaggtatacagtcacaagttaataattacctacctttctgtgtctgggccatttttggctataatcatctttaattttcctagtcctcccacaggtgctctgtctgtgcccgttgtaaactgcaagaagagtcttttctgttcatctgtaaatgaatgaacgatttcccagaactccctaatgagaaaaaatacaatactggtttcagtttggcattcattatgactggtactaacataagcttatgatttgcattaaaactatattaagagacaacttggaagttaattatcaggatagtatcacttctggtgatttaaaaatttccaagcaaatttatcctgaacagctctgaatacgtaaaaatttagattagattacaatatagtaaaatattagtactacaatagtaaaaaattgagaaaacccaagtgtgtagtaacaggaagtgactatttaaactatggtataatcacattatgcagtcagtaatgagcaaaagacaaaatcctatgaattgaaaaagactgaaatgaatatttggaaaaattaactccaggtgcctttgggtatatattttatttatcctttctcctttattctcctggtctactcatcctttaactcaactttgggaggaaaaagtgatacagattaaggacaaaagaaaaaaagaccccctgccccaaccaactggccccaaatgcaaacaactacatacctaatagcagtaatacaaaagttcaatttttatatgaactagaatactttaaacaagtaatatgtgctgtgtatggaggaggagaattattctgacatttctgccacctgcagttattttaagagaatgatttatcctgtggcattcctaaaatctatgtaataaaagctatgttttaatgacacttatgttagtttgagttctaaaaaacgaaatacaagctcataaagtgcaatcttgaagtttatttgaataatcaggcatctataaaacatatatacactagttcatagctaaataaatttttttttttttgagacagagtcttgctctgtcgctcaggctggagtgcagtggcgcgatctcggctcattgcaagctccgcctcctgggttcgcgccattgtcctgcctcagcctcccaagtggctgggactataggtgcctgccaccacgcctggctaattttctgtacgtttaagtagaggcaggatttcaccatgttagccaggatgggtctcgatctcctgacctcgtgatccgcccaccttggcctcccaaagtgctgggattgcaggcatgagccaccgcgcctggcctatagctaaataatgttaagattaaaaaattaaaaaaaaaattaaaagtatttttagttgcttatataatataaaatgcattttaatagttatttagaaagttctttccagagccaggtgtggtggctcatgcctgtaatcccagcactttgggaggctgaggccagtggatcacctgaggtcaggggttcaagatcagcctgaccaacgtggtgaaaccctgtctctactaaaatacaaaaattagccgggcgtggtggcagatgcctgtaatctcagctacttgggaggccaaggcagaagaattgcttgaacatgggaggcggaggctgcaatgagctgagatcacaccattgcactctagcctgggaaacattttgagccatctcaaaaaaaaaaaaaaatttcatctcaaaaaaaaaaaaaaaaaaaattccagaatgttagcagagaacatcacacatcccaaaccagtaattacaattttctaccttttgatattctaatatcccaacatactatttttattaccaaaatgctggcatttttggtgctgcaacaccatttatctgaaataacttaaaagttttattagaattcttggctttctccaatctcttgccacttcccttccctgcccctttacaaatccaccagtcagcaagagaaaacaaaaaagaaacacacaacaacagaaaactggacatgaaacttgcaggagcatctctcaggtaaggaggagaggaatcggaccccagttcatgatactccttttggggaggctgacaagagaaaacaagtctgctgtagcacagaaccctccaacatagcagaaaagctgccctgcagtgggtggtaataaagcctcctcacactgcaggagggctctgaagcttaactgaaccagctacacctaaggaggtagaccaaagagcaccaccgaagaacagagacagaccctgcaggaagtggcagatcagcagtggtcacaatgaccagtctggctatattattacggagctgtttctttggacttttaaacgaaacctttaaaaattttatacaatgaaaacagggaacaaaatgcatctctattcctataagtgttatgtgtgttacattaacattttgaattaaacaagaatgcatatatttagaaagctgaagaaaacacgaagaagcttccaggtttccacataaaagtggtggctgtatcgatcacatcctactcacatctctaaaaatctacccggatcaaaaaggggaaaataaaaaaaaaacctatgacttaggtctagagtaaaactaggagacagaacaataaactccaaataccaaagaagtggggaaatgagcagggtccagcaggagctacatcgaagagtggcttgtgcggaatcatactgattagggatcacccaaggccagaccccaccccctccaccacctgcccctcaacagagcactactgagtgtaggttagagcactggcaacagggtggttaaaggaaggactacagaaaagtacttgggtccagcaatggcaggctcaggaaggcaccatgcatgaaaggaaggggggtaccttcaaaagcagaagtgtcccttaagcggctgtaaaggggtggaagcagctaatgaaaaagagtcttcattaagctacatggagctgaaaaatcagaaagtggtgattctgcccttactaaagcaacagaagagggatcccttcaaccaagacccctaaagccacacaattcctccctgctccaccatgaggtctagtaataacaggtccagaaaaaagtaacatctgttatagaaacataaacaagaaaaacagaattcggaagtctaaataaagttattatgggaagagtctggcgaatgagaagcaaaactttccggtagacaaaagtagaccagaaaaattcagtcataaacaatgggaaactagtaacaccacattccaacacaaaaggagaatgcagcagcagacaagacagaccaccagtgatgagaaatacaaattcagaaagaaatgagcacatcttaaataaaaataagaatactaaaaaatactgaagaaatatgcaaggtaactttggaaataaaggctaattttattataaggtgaccaaagaagaagcagtatgactaaaatttctactgaggacagactctagaaaattaaaaaacaaagaaaatgaataaaataaacataactaaagaaatgcatatcaaagcataataacgaaaaagattaatagctaactacataaacataaaacctttcatttggcaaaaaaatcctataagcaaggttaaaagacaaatgacaaactgggaaaaaatatttgcgaatttaccagcgttaaaggactaatctccttaacatataaagtttctaaaagtgaagaaaacgaccagctgagcagcaaaatgagcaaaagactatataaaactataaacagctctacaagaaaaaatgtccattatcattcataattgaggtaagctcaaaaattgtaactatagtaaaataccatttctcaattggcaaataagtaaacatccacatttaacaacacattctgtttggaaagttttaagaaaagagatactgttgtaaactgctggtgggaatgcaaaatgttatttttctgtgaaggaggatttggcagtatctagcaaaattacacatgcatctaccatttgatgcaacaatcccacttctaacaatttatcttaatgatactttacatatgtacggaataatgcatgaacattaagaggattcactgtgacattagtactaacagcaaaaggttaaaaataacctcgatgcccataaatattgataagctatgctgcatccacacaaaggagcagtattcagtcatacaaaacaaaatggaaaacacctctgcaataatgtgggatgattctcaggatacactaggaagattattttcctagttctgtcctctgataaggcctacaagcagtaacgttcaaaagcaaaggccatactttgcatctaaaatctgacttctaaatgccattctccaacaatttattggaaaaataacttattccaggcctgagaatgaatgttcgagattagttagaaatctcaaaatctaatagggtcattttaaaagcacacgatagctaagcaatttgaatatcatttagagtaatgactgtgaaacgtattaaatacaaaaacattcattagttcgtaatattcaaaaaggcagcaaaaaccaaccaaaaaacaaaattaaattgtcactattaaaattattacattaactccttactctaaaaaccttaaatctattttatcatgccttttctgtagaactgtttttcaaggtaatcaaatggcacccaatgatgagaaaaaagaatgccaggtatatatgtaggacaagcagatggaaattttttttccccaaacagccattttgcaatccccaatgagataacagattaaggtaatgatactgataaatacgaaaatcaggtgaagggcaggtagcaggttctggaaagatgatgatggcaacgacatagttattattattattattttttgttttattccctcccaacctcccctacaaaaacagcaaactgaatgagaaaaccaaagacccagagacattatctacaacaaaaccatgaaccattgcatataattggacagaaacaaagctctgacaactacaagactggttagtaaggaagcagatgcaaggtaactgactgggtttctgacagccctgagaacactgccaacccactggaaagcacaggccaatctgaaaacagggcttaaagttttaaaaagttctacaggatctaatttgcagatgattacaaagggatcatgatgtagtaagctctgggcccttaaaacacccgaataccaaacccccaccagaaacaaaccttgtaccgaggaaaaacttctgggaataacttccgaatggaccaggtcagtaataaaaaccaaaaaaaagttcaaatatatgtgtgggatagaggagtaaagaaggcagtttcagaaagcacaagggcatatttttgaccattttacaaaaacacagacttctcctcgtccctaaaaagcgaaaaaagttatcctggcccatctctccctcgtccaagtatgagaaactcatttcactcacacacacacacacacacacacaaataaataaataacagaacagagtgaaacagtgtaattattagagaaaaagtatgtgcatatacatgagaatggtgtccctacagaaaatcaaagtacatgaaacaatatgcaaataaaacatgaaaactgtaaacaaatttcaaactgagctaaaaagaatttaaaaaataacaaatcattagaaatgggaaatttctgaatgagggtaactgaaaaaaaggaagacatgacacaactaaggagtaattagaaatgcaaggaaaaaaatcccatcaaatacaaagaaactaaaactaaactagaagaaacataaaggtgaataaaacagaacaataataccagaacctgaaggcagaagaaaaatcttaaaatcaaaaagaaacaaacccatgcttgagactctccttgctggtccagagccacagtgctgctgtgtactggcaggaggaattctgctataattggtcctggcagtgttcacctttcctgcaagtgttccacagcccagggacacaatgtggtcaggagcactgccaggaacaccagcaagggggagcctgccacaacaggcacaaggctcaggaagcactctccagcccacgaaaatctagtgggggtcctcttccctcacccaaacacactctgcgcagct

SEQ ID NO: 2 - DNA, 48 bp, synthetic construct
  misc_feature 1..48: note = shRNA artificial/synthetic sequence
  source 1..48: mol_type = other DNA
gatatcaccttacagaaattactcgagtaatttctgtaaggtgatatc

SEQ ID NO: 3 - DNA, 21 bp, synthetic construct
  misc_feature 1..21: note = UBE3A-ATS artificial/synthetic target sequence
  source 1..21: mol_type = other DNA
gatatcaccttacagaaatta

SEQ ID NO: 4 - DNA, 21 bp, synthetic construct
  misc_feature 1..21: note = UBE3A-ATS artificial/synthetic sequence
  source 1..21: mol_type = other DNA
ctgaagtgcacatggaatatt

SEQ ID NO: 5 - DNA, 21 bp, synthetic construct
  misc_feature 1..21: note = UBE3A-ATS artificial/synthetic sequence
  source 1..21: mol_type = other DNA
tcttcaggatgacacatatta

SEQ ID NO: 6 - DNA, 21 bp, synthetic construct
  misc_feature 1..21: note = UBE3A-ATS artificial/synthetic sequence
  source 1..21: mol_type = other DNA
cacaatgatatggagatttaa

SEQ ID NO: 7 - DNA, 21 bp, synthetic construct
  misc_feature 1..21: note = UBE3A-ATS artificial/synthetic sequence
  source 1..21: mol_type = other DNA
ctcaatccaataacctaattt

SEQ ID NO: 8 - DNA, 21 bp, synthetic construct
  misc_feature 1..21: note = UBE3A-ATS artificial/synthetic sequence
  source 1..21: mol_type = other DNA
gtaaacagaaggaagaaataa

SEQ ID NO: 9 - DNA, 21 bp, synthetic construct
  misc_feature 1..21: note = UBE3A-ATS artificial/synthetic sequence
  source 1..21: mol_type = other DNA
aggtccgtattacccttatat

SEQ ID NO: 10 - DNA, 21 bp, synthetic construct
  misc_feature 1..21: note = UBE3A-ATS artificial/synthetic sequence
  source 1..21: mol_type = other DNA
catctagcctttctgatttat

SEQ ID NO: 11 - DNA, 21 bp, synthetic construct
  misc_feature 1..21: note = UBE3A-ATS artificial/synthetic sequence
  source 1..21: mol_type = other DNA
atctagcctttctgatttata

SEQ ID NO: 12 - DNA, 21 bp, synthetic construct
  misc_feature 1..21: note = UBE3A-ATS artificial/synthetic sequence
  source 1..21: mol_type = other DNA
tctagcctttctgatttatat

SEQ ID NO: 13 - DNA, 21 bp, synthetic construct
  misc_feature 1..21: note = UBE3A-ATS artificial/synthetic sequence
  source 1..21: mol_type = other DNA
ctagcctttctgatttatata

SEQ ID NO: 14 - DNA, 21 bp, synthetic construct
  misc_feature 1..21: note = UBE3A-ATS artificial/synthetic sequence
  source 1..21: mol_type = other DNA
agtcacaactcagatttattt

SEQ ID NO: 15 - DNA, 21 bp, synthetic construct
  misc_feature 1..21: note = UBE3A-ATS artificial/synthetic sequence
  source 1..21: mol_type = other DNA
ggcctcacaactcagatttaa

SEQ ID NO: 16 - DNA, 21 bp, synthetic construct
  misc_feature 1..21: note = UBE3A-ATS artificial/synthetic sequence
  source 1..21: mol_type = other DNA
gaacagagccctcagaaataa

SEQ ID NO: 17 - DNA, 21 bp, synthetic construct
  misc_feature 1..21: note = UBE3A-ATS artificial/synthetic sequence
  source 1..21: mol_type = other DNA
ggaaaggattccctatttaat

SEQ ID NO: 18 - DNA, 21 bp, synthetic construct
  misc_feature 1..21: note = UBE3A-ATS artificial/synthetic sequence
  source 1..21: mol_type = other DNA
gaaaggattccctatttaata

SEQ ID NO: 19 - DNA, 21 bp, synthetic construct
  misc_feature 1..21: note = UBE3A-ATS artificial/synthetic sequence
  source 1..21: mol_type = other DNA
gacaaacgggatctcattaaa

SEQ ID NO: 20 - DNA, 21 bp, synthetic construct
  misc_feature 1..21: note = UBE3A-ATS artificial/synthetic sequence
  source 1..21: mol_type = other DNA
catctgacaaagggctaatat

SEQ ID NO: 21 - DNA, 21 bp, synthetic construct
  misc_feature 1..21: note = UBE3A-ATS artificial/synthetic sequence
  source 1..21: mol_type = other DNA
acaatgaactcagacaaattt

SEQ ID NO: 22 - DNA, 21 bp, synthetic construct
  misc_feature 1..21: note = UBE3A-ATS artificial/synthetic sequence
  source 1..21: mol_type = other DNA
caatgaactcagacaaattta

SEQ ID NO: 23 - DNA, 21 bp, synthetic construct
  misc_feature 1..21: note = UBE3A-ATS artificial/synthetic sequence
  source 1..21: mol_type = other DNA
gttagaatggcgatcattaaa

SEQ ID NO: 24 - DNA, 21 bp, synthetic construct
  misc_feature 1..21: note = UBE3A-ATS artificial/synthetic sequence
  source 1..21: mol_type = other DNA
cagccatcccattgctatata

SEQ ID NO: 25 - DNA, 21 bp, synthetic construct
  misc_feature 1..21: note = UBE3A-ATS artificial/synthetic sequence
  source 1..21: mol_type = other DNA
agccatcccattgctatatat

SEQ ID NO: 26 - DNA, 21 bp, synthetic construct
  misc_feature 1..21: note = UBE3A-ATS artificial/synthetic sequence
  source 1..21: mol_type = other DNA
gccatcccattgctatatata

SEQ ID NO: 27 - DNA, 21 bp, synthetic construct
  misc_feature 1..21: note = UBE3A-ATS artificial/synthetic sequence
  source 1..21: mol_type = other DNA
ccatcccattgctatatatat

SEQ ID NO: 28 - DNA, 21 bp, synthetic construct
  misc_feature 1..21: note = UBE3A-ATS artificial/synthetic sequence
  source 1..21: mol_type = other DNA
catcccattgctatatatata

SEQ ID NO: 29 - DNA, 21 bp, synthetic construct
  misc_feature 1..21: note = UBE3A-ATS artificial/synthetic sequence
  source 1..21: mol_type = other DNA
atatacccaaaggactataaa

SEQ ID NO: 30 - DNA, 21 bp, synthetic construct
  misc_feature 1..21: note = UBE3A-ATS artificial/synthetic sequence
  source 1..21: mol_type = other DNA
tatacccaaaggactataaat

SEQ ID NO: 31 - DNA, 21 bp, synthetic construct
  misc_feature 1..21: note = UBE3A-ATS artificial/synthetic sequence
  source 1..21: mol_type = other DNA
atatgcacaggtatgtttatt

SEQ ID NO: 32 - DNA, 21 bp, synthetic construct
  misc_feature 1..21: note = UBE3A-ATS artificial/synthetic sequence
  source 1..21: mol_type = other DNA
gaaagacactgattgtattta

SEQ ID NO: 33 - DNA, 21 bp, synthetic construct
  misc_feature 1..21: note = UBE3A-ATS artificial/synthetic sequence
  source 1..21: mol_type = other DNA
acatagccctaaccatataaa

SEQ ID NO: 34 - DNA, 21 bp, synthetic construct
  misc_feature 1..21: note = UBE3A-ATS artificial/synthetic sequence
  source 1..21: mol_type = other DNA
cctcacacaaagacctattat

SEQ ID NO: 35 - DNA, 21 bp, synthetic construct
  misc_feature 1..21: note = UBE3A-ATS artificial/synthetic sequence
  source 1..21: mol_type = other DNA
tggcagagatttggcaattat

SEQ ID NO: 36 - DNA, 21 bp, synthetic construct
  misc_feature 1..21: note = UBE3A-ATS artificial/synthetic sequence
  source 1..21: mol_type = other DNA
ggcagagatttggcaattata

SEQ ID NO: 37 - DNA, 21 bp, synthetic construct
  misc_feature 1..21: note = UBE3A-ATS artificial/synthetic sequence
  source 1..21: mol_type = other DNA
agaacaacaaggaggaaattt

SEQ ID NO: 38 - DNA, 21 bp, synthetic construct
  misc_feature 1..21: note = UBE3A-ATS artificial/synthetic sequence
  source 1..21: mol_type = other DNA
gaacaacaaggaggaaattta

SEQ ID NO: 39 - DNA, 21 bp, synthetic construct
  misc_feature 1..21: note = UBE3A-ATS artificial/synthetic sequence
  source 1..21: mol_type = other DNA
gaatggagtgaaatgtttaaa

SEQ ID NO: 40 - DNA, 21 bp, synthetic construct
  misc_feature 1..21: note = UBE3A-ATS artificial/synthetic sequence
  source 1..21: mol_type = other DNA
tctgatttccttgggtttatt

SEQ ID NO: 41 - DNA, 21 bp, synthetic construct
  misc_feature 1..21: note = UBE3A-ATS artificial/synthetic sequence
  source 1..21: mol_type = other DNA
ctgatttccttgggtttattt

SEQ ID NO: 42 - DNA, 21 bp, synthetic construct
  misc_feature 1..21: note = UBE3A-ATS artificial/synthetic sequence
  source 1..21: mol_type = other DNA
tgctcttctttctactttatt

SEQ ID NO: 43 - DNA, 21 bp, synthetic construct
  misc_feature 1..21: note = UBE3A-ATS artificial/synthetic sequence
  source 1..21: mol_type = other DNA
gtaagcatttagtgctataaa

SEQ ID NO: 44 - DNA, 21 bp, synthetic construct
  misc_feature 1..21: note = UBE3A-ATS artificial/synthetic sequence
  source 1..21: mol_type = other DNA
ttagtcacatcccacaaattt

SEQ ID NO: 45 - DNA, 21 bp, synthetic construct
  misc_feature 1..21: note = UBE3A-ATS artificial/synthetic sequence
  source 1..21: mol_type = other DNA
atggtctgtgctgtgaatatt

SEQ ID NO: 46 - DNA, 21 bp, synthetic construct
  misc_feature 1..21: note = UBE3A-ATS artificial/synthetic sequence
  source 1..21: mol_type = other DNA
ttgaagtctccaaccataatt

SEQ ID NO: 47 - DNA, 21 bp, synthetic construct
  misc_feature 1..21: note = UBE3A-ATS artificial/synthetic sequence
  source 1..21: mol_type = other DNA
cagtttgtgcatcacatattt

SEQ ID NO: 48 - DNA, 21 bp, synthetic construct
  misc_feature 1..21: note = UBE3A-ATS artificial/synthetic sequence
  source 1..21: mol_type = other DNA
tgccctcttggtggcttattt

SEQ ID NO: 49 - DNA, 21 bp, synthetic construct
  misc_feature 1..21: note = UBE3A-ATS artificial/synthetic sequence
  source 1..21: mol_type = other DNA
tcctaggtcataatgataatt

SEQ ID NO: 50 - DNA, 21 bp, synthetic construct
  misc_feature 1..21: note = UBE3A-ATS artificial/synthetic sequence
  source 1..21: mol_type = other DNA
ctccacatccttaccaatatt

SEQ ID NO: 51 - DNA, 21 bp, synthetic construct
  misc_feature 1..21: note = UBE3A-ATS artificial/synthetic sequence
  source 1..21: mol_type = other DNA
cgcttatcagatatgatttat

SEQ ID NO: 52 - DNA, 21 bp, synthetic construct
  misc_feature 1..21: note = UBE3A-ATS artificial/synthetic sequence
  source 1..21: mol_type = other DNA
ggtctatacatgtagattatt

SEQ ID NO: 53 - DNA, 21 bp, synthetic construct
  misc_feature 1..21: note = UBE3A-ATS artificial/synthetic sequence
  source 1..21: mol_type = other DNA
tcatagatgtatgggattatt

SEQ ID NO: 54 - DNA, 21 bp, synthetic construct
  misc_feature 1..21: note = UBE3A-ATS artificial/synthetic sequence
  source 1..21: mol_type = other DNA
catagatgtatgggattattt

SEQ ID NO: 55 - DNA, 21 bp, synthetic construct
  misc_feature 1..21: note = UBE3A-ATS artificial/synthetic sequence
  source 1..21: mol_type = other DNA
cttgtaactccttggttaaat

SEQ ID NO: 56 - DNA, 21 bp, synthetic construct
  misc_feature 1..21: note = UBE3A-ATS artificial/synthetic sequence
  source 1..21: mol_type = other DNA
ttgtaactccttggttaaatt

SEQ ID NO: 57 - DNA, 21 bp, synthetic construct
  misc_feature 1..21: note = UBE3A-ATS artificial/synthetic sequence
  source 1..21: mol_type = other DNA
tgtaactccttggttaaattt

SEQ ID NO: 58 - DNA, 21 bp, synthetic construct
  misc_feature 1..21: note = UBE3A-ATS artificial/synthetic sequence
  source 1..21: mol_type = other DNA
gtaactccttggttaaattta

SEQ ID NO: 59 - DNA, 21 bp, synthetic construct
  misc_feature 1..21: note = UBE3A-ATS artificial/synthetic sequence
  source 1..21: mol_type = other DNA
tcctttctgatgctgtttaaa

SEQ ID NO: 60 - DNA, 21 bp, synthetic construct
  misc_feature 1..21: note = UBE3A-ATS artificial/synthetic sequence
  source 1..21: mol_type = other DNA
cctttctgatgctgtttaaat

SEQ ID NO: 61 - DNA, 21 bp, synthetic construct
  misc_feature 1..21: note = UBE3A-ATS artificial/synthetic sequence
  source 1..21: mol_type = other DNA
ctgaaactttgctgcatttat

SEQ ID NO: 62 - DNA, 21 bp, synthetic construct
  misc_feature 1..21: note = UBE3A-ATS artificial/synthetic sequence
  source 1..21: mol_type = other DNA
tgaaactttgctgcatttatt

SEQ ID NO: 63 - DNA, 21 bp, synthetic construct
  misc_feature 1..21: note = UBE3A-ATS artificial/synthetic sequence
  source 1..21: mol_type = other DNA
gaaactttgctgcatttatta

SEQ ID NO: 64 - DNA, 21 bp, synthetic construct
  misc_feature 1..21: note = UBE3A-ATS artificial/synthetic sequence
  source 1..21: mol_type = other DNA
ttgctctgtgaatagataatt

SEQ ID NO: 65 - DNA, 21 bp, synthetic construct
  misc_feature 1..21: note = UBE3A-ATS artificial/synthetic sequence
  source 1..21: mol_type = other DNA
tgctctgtgaatagataattt

SEQ ID NO: 66 - DNA, 21 bp, synthetic construct
  misc_feature 1..21: note = UBE3A-ATS artificial/synthetic sequence
  source 1..21: mol_type = other DNA
atatgttgatccacctttata

SEQ ID NO: 67 - DNA, 21 bp, synthetic construct
  misc_feature 1..21: note = UBE3A-ATS artificial/synthetic sequence
  source 1..21: mol_type = other DNA
tatgttgatccacctttatat

SEQ ID NO: 68 - DNA, 21 bp, synthetic construct
  misc_feature 1..21: note = UBE3A-ATS artificial/synthetic sequence
  source 1..21: mol_type = other DNA
atgttgatccacctttatatt

SEQ ID NO: 69 - DNA, 21 bp, synthetic construct
  misc_feature 1..21: note = UBE3A-ATS artificial/synthetic sequence
  source 1..21: mol_type = other DNA
tctttggctactttgtataat

SEQ ID NO: 70 - DNA, 21 bp, synthetic construct
  misc_feature 1..21: note = UBE3A-ATS artificial/synthetic sequence
  source 1..21: mol_type = other DNA
ctttggctactttgtataata

SEQ ID NO: 71 - DNA, 21 bp, synthetic construct
  misc_feature 1..21: note = UBE3A-ATS artificial/synthetic sequence
  source 1..21: mol_type = other DNA
taagtgcatagagccaataaa

SEQ ID NO: 72 - DNA, 21 bp, synthetic construct
  misc_feature 1..21: note = UBE3A-ATS artificial/synthetic sequence
  source 1..21: mol_type = other DNA
atgcacatgatcttcttaatt

SEQ ID NO: 73 - DNA, 21 bp, synthetic construct
  misc_feature 1..21: note = UBE3A-ATS artificial/synthetic sequence
  source 1..21: mol_type = other DNA
tgcacatgatcttcttaattt

SEQ ID NO: 74 - DNA, 21 bp, synthetic construct
  misc_feature 1..21: note = UBE3A-ATS artificial/synthetic sequence
  source 1..21: mol_type = other DNA
tggcttaaacaagagaaattt

SEQ ID NO: 75 - DNA, 21 bp, synthetic construct
  misc_feature 1..21: note = UBE3A-ATS artificial/synthetic sequence
  source 1..21: mol_type = other DNA
ggcttaaacaagagaaattta

SEQ ID NO: 76 - DNA, 21 bp, synthetic construct
  misc_feature 1..21: note = UBE3A-ATS artificial/synthetic sequence
  source 1..21: mol_type = other DNA
aggtatcagagtagtatattt

SEQ ID NO: 77 - DNA, 21 bp, synthetic construct
  misc_feature 1..21: note = UBE3A-ATS artificial/synthetic sequence
  source 1..21: mol_type = other DNA
ttaaagagttgccacattata

SEQ ID NO: 78 - DNA, 21 bp, synthetic construct
  misc_feature 1..21: note = UBE3A-ATS artificial/synthetic sequence
  source 1..21: mol_type = other DNA
gttgccacattatacataata

SEQ ID NO: 79 - DNA, 21 bp, synthetic construct
  misc_feature 1..21: note = UBE3A-ATS artificial/synthetic sequence
  source 1..21: mol_type = other DNA
agacattgaaccagctattaa

SEQ ID NO: 80 - DNA, 21 bp, synthetic construct
  misc_feature 1..21: note = UBE3A-ATS artificial/synthetic sequence
  source 1..21: mol_type = other DNA
gacattgaaccagctattaaa

SEQ ID NO: 81 - DNA, 21 bp, synthetic construct
  misc_feature 1..21: note = UBE3A-ATS artificial/synthetic sequence
  source 1..21: mol_type = other DNA
agggttaaagaaaggtataaa

SEQ ID NO: 82 - DNA, 21 bp, synthetic construct
  misc_feature 1..21: note = UBE3A-ATS artificial/synthetic sequence
  source 1..21: mol_type = other DNA
atacccgagatgaccaataaa

SEQ ID NO: 83 - DNA, 21 bp, synthetic construct
  misc_feature 1..21: note = UBE3A-ATS artificial/synthetic sequence
  source 1..21: mol_type = other DNA
caaatccacgagaagaaataa

SEQ ID NO: 84 - DNA, 21 bp, synthetic construct
  misc_feature 1..21: note = UBE3A-ATS artificial/synthetic sequence
  source 1..21: mol_type = other DNA
acttgtgttgatggtattata

SEQ ID NO: 85 - DNA, 21 bp, synthetic construct
  misc_feature 1..21: note = UBE3A-ATS artificial/synthetic sequence
  source 1..21: mol_type = other DNA
cttgtgttgatggtattatat

SEQ ID NO: 86 - DNA, 21 bp, synthetic construct
  misc_feature 1..21: note = UBE3A-ATS artificial/synthetic sequence
  source 1..21: mol_type = other DNA
attcattggaaagggtataaa

SEQ ID NO: 87 - DNA, 21 bp, synthetic construct
  misc_feature 1..21: note = UBE3A-ATS artificial/synthetic sequence
  source 1..21: mol_type = other DNA
cagtaacaagagcccattatt

SEQ ID NO: 88 - DNA, 21 bp, synthetic construct
  misc_feature 1..21: note = UBE3A-ATS artificial/synthetic sequence
  source 1..21: mol_type = other DNA
agtaacaagagcccattattt

SEQ ID NO: 89 - DNA, 21 bp, synthetic construct
  misc_feature 1..21: note = UBE3A-ATS artificial/synthetic sequence
  source 1..21: mol_type = other DNA
gcccaagagatccactttatt

SEQ ID NO: 90 - DNA, 21 bp, synthetic construct
  misc_feature 1..21: note = UBE3A-ATS artificial/synthetic sequence
  source 1..21: mol_type = other DNA
cccaagagatccactttattt

SEQ ID NO: 91 - DNA, 21 bp, synthetic construct
  misc_feature 1..21: note = UBE3A-ATS artificial/synthetic sequence
  source 1..21: mol_type = other DNA
tttcatctcacccagaatatt

SEQ ID NO: 92 - DNA, 21 bp, synthetic construct
  misc_feature 1..21: note = UBE3A-ATS artificial/synthetic sequence
  source 1..21: mol_type = other DNA
tgttatgagatccagttattt

SEQ ID NO: 93 - DNA, 21 bp, synthetic construct
  misc_feature 1..21: note = UBE3A-ATS artificial/synthetic sequence
  source 1..21: mol_type = other DNA
actgaactgtgagccaattaa

SEQ ID NO: 94 - DNA, 21 bp, synthetic construct
  misc_feature 1..21: note = UBE3A-ATS artificial/synthetic sequence
  source 1..21: mol_type = other DNA
ctgaactgtgagccaattaaa

SEQ ID NO: 95 - DNA, 21 bp, synthetic construct
  misc_feature 1..21: note = UBE3A-ATS artificial/synthetic sequence
  source 1..21: mol_type = other DNA
agtcttaggtagttctttata

SEQ ID NO: 96 - DNA, 21 bp, synthetic construct
  misc_feature 1..21: note = UBE3A-ATS artificial/synthetic sequence
  source 1..21: mol_type = other DNA
caaaggcagcagcacaataat

SEQ ID NO: 97 - DNA, 21 bp, synthetic construct
  misc_feature 1..21: note = UBE3A-ATS artificial/synthetic sequence
  source 1..21: mol_type = other DNA
agactggctatttgaaattat

SEQ ID NO: 98 - DNA, 21 bp, synthetic construct
  misc_feature 1..21: note = UBE3A-ATS artificial/synthetic sequence
  source 1..21: mol_type = other DNA
gactggctatttgaaattatt

SEQ ID NO: 99 - DNA, 21 bp, synthetic construct
  misc_feature 1..21: note = UBE3A-ATS artificial/synthetic sequence
  source 1..21: mol_type = other DNA
caccatcaagagaactaatat

SEQ ID NO: 100 - DNA, 21 bp, synthetic construct
  misc_feature 1..21: note = UBE3A-ATS artificial/synthetic sequence
  source 1..21: mol_type = other DNA
accatcaagagaactaatata

SEQ ID NO: 101 - DNA, 21 bp, synthetic construct
  misc_feature 1..21: note = UBE3A-ATS artificial/synthetic sequence
  source 1..21: mol_type = other DNA
ccatcaagagaactaatataa

SEQ ID NO: 102 - DNA, 21 bp, synthetic construct
  misc_feature 1..21: note = UBE3A-ATS artificial/synthetic sequence
  source 1..21: mol_type = other DNA
ggaaagtacatagtcaaattt

SEQ ID NO: 103 - DNA, 21 bp, synthetic construct
  misc_feature 1..21: note = UBE3A-ATS artificial/synthetic sequence
  source 1..21: mol_type = other DNA
caatgcatactacagtataaa

SEQ ID NO: 104 - DNA, 21 bp, synthetic construct
  misc_feature 1..21: note = UBE3A-ATS artificial/synthetic sequence
  source 1..21: mol_type = other DNA
agtccttacgtgtcaataatt

SEQ ID NO: 105 - DNA, 21 bp, synthetic construct
  misc_feature 1..21: note = UBE3A-ATS artificial/synthetic sequence
  source 1..21: mol_type = other DNA
gtccttacgtgtcaataatta

SEQ ID NO: 106 - DNA, 21 bp, synthetic construct
  misc_feature 1..21: note = UBE3A-ATS artificial/synthetic sequence
  source 1..21: mol_type = other DNA
gacacatacaggctgaaatta

SEQ ID NO: 107 - DNA, 21 bp, synthetic construct
  misc_feature 1..21: note = UBE3A-ATS artificial/synthetic sequence
  source 1..21: mol_type = other DNA
acacatacaggctgaaattaa

SEQ ID NO: 108 - DNA, 21 bp, synthetic construct
  misc_feature 1..21: note = UBE3A-ATS artificial/synthetic sequence
  source 1..21: mol_type = other DNA
cacatacaggctgaaattaaa

SEQ ID NO: 109 - DNA, 21 bp, synthetic construct
  misc_feature 1..21: note = UBE3A-ATS artificial/synthetic sequence
  source 1..21: mol_type = other DNA
caatattgaagcacctaaata

SEQ ID NO: 110 - DNA, 21 bp, synthetic construct
  misc_feature 1..21: note = UBE3A-ATS artificial/synthetic sequence
  source 1..21: mol_type = other DNA
atggatctaacagacattata

SEQ ID NO: 111 - DNA, 21 bp, synthetic construct
  misc_feature 1..21: note = UBE3A-ATS artificial/synthetic sequence
  source 1..21: mol_type = other DNA
cattctccaggatagattata

SEQ ID NO: 112 - DNA, 21 bp, synthetic construct
  misc_feature 1..21: note = UBE3A-ATS artificial/synthetic sequence
  source 1..21: mol_type = other DNA
attctccaggatagattatat

SEQ ID NO: 113 - DNA, 21 bp, synthetic construct
  misc_feature 1..21: note = UBE3A-ATS artificial/synthetic sequence
  source 1..21: mol_type = other DNA
caagatggaaactggaaattt

SEQ ID NO: 114 - DNA, 21 bp, synthetic construct
  misc_feature 1..21: note = UBE3A-ATS artificial/synthetic sequence
  source 1..21: mol_type = other DNA
ttctatgaggtaagcttatat

SEQ ID NO: 115 - DNA, 21 bp, synthetic construct
  misc_feature 1..21: note = UBE3A-ATS artificial/synthetic sequence
  source 1..21: mol_type = other DNA
tctatgaggtaagcttatatt

SEQ ID NO: 116 - DNA, 21 bp, synthetic construct
  misc_feature 1..21: note = UBE3A-ATS artificial/synthetic sequence
  source 1..21: mol_type = other DNA
acaaagatcagacagaaataa

SEQ ID NO: 117 - DNA, 21 bp, synthetic construct
  misc_feature 1..21: note = UBE3A-ATS artificial/synthetic sequence
  source 1..21: mol_type = other DNA
caaagatcagacagaaataaa

SEQ ID NO: 118 - DNA, 21 bp, synthetic construct
  misc_feature 1..21: note = UBE3A-ATS artificial/synthetic sequence
  source 1..21: mol_type = other DNA
ataccacagacatacaaatta

SEQ ID NO: 119 - DNA, 21 bp, synthetic construct
  misc_feature 1..21: note = UBE3A-ATS artificial/synthetic sequence
  source 1..21: mol_type = other DNA
tgaagactgccaaccatttaa

SEQ ID NO: 120 - DNA, 21 bp, synthetic construct
  misc_feature 1..21: note = UBE3A-ATS artificial/synthetic sequence
  source 1..21: mol_type = other DNA
gaagactgccaaccatttaaa

SEQ ID NO: 121 - DNA, 21 bp, synthetic construct
  misc_feature 1..21: note = UBE3A-ATS artificial/synthetic sequence
  source 1..21: mol_type = other DNA
ctaattcagcaacacattata

SEQ ID NO: 122 - DNA, 21 bp, synthetic construct
  misc_feature 1..21: note = UBE3A-ATS artificial/synthetic sequence
  source 1..21: mol_type = other DNA
gatgcaggatagttcaatata

SEQ ID NO: 123 - DNA, 21 bp, synthetic construct
  misc_feature 1..21: note = UBE3A-ATS artificial/synthetic sequence
  source 1..21: mol_type = other DNA
atgcaggatagttcaatataa

SEQ ID NO: 124 - DNA, 21 bp, synthetic construct
  misc_feature 1..21: note = UBE3A-ATS artificial/synthetic sequence
  source 1..21: mol_type = other DNA
tgcaggatagttcaatataat

SEQ ID NO: 125 - DNA, 21 bp, synthetic construct
  misc_feature 1..21: note = UBE3A-ATS artificial/synthetic sequence
  source 1..21: mol_type = other DNA
gtgtctgttggctgcataaat

SEQ ID NO: 126 - DNA, 21 bp, synthetic construct
  misc_feature 1..21: note = UBE3A-ATS artificial/synthetic sequence
  source 1..21: mol_type = other DNA
ctttgtagattctggatatta

SEQ ID NO: 127 - DNA, 21 bp, synthetic construct
  misc_feature 1..21: note = UBE3A-ATS artificial/synthetic sequence
  source 1..21: mol_type = other DNA
gcagaagctctttagtttaat

SEQ ID NO: 128 - DNA, 21 bp, synthetic construct
  misc_feature 1..21: note = UBE3A-ATS artificial/synthetic sequence
  source 1..21: mol_type = other DNA
cagaagctctttagtttaatt

SEQ ID NO: 129 - DNA, 21 bp, synthetic construct
  misc_feature 1..21: note = UBE3A-ATS artificial/synthetic sequence
  source 1..21: mol_type = other DNA
catcctgttactgggtatata

SEQ ID NO: 130 - DNA, 21 bp, synthetic construct
  misc_feature 1..21: note = UBE3A-ATS artificial/synthetic sequence
  source 1..21: mol_type = other DNA
gtatatacccagaggattata

SEQ ID NO: 131 - DNA, 21 bp, synthetic construct
  misc_feature 1..21: note = UBE3A-ATS artificial/synthetic sequence
  source 1..21: mol_type = other DNA
tatatacccagaggattataa

SEQ ID NO: 132 - DNA, 21 bp, synthetic construct
  misc_feature 1..21: note = UBE3A-ATS artificial/synthetic sequence
  source 1..21: mol_type = other DNA
atatacccagaggattataaa

SEQ ID NO: 133 - DNA, 21 bp, synthetic construct
  misc_feature 1..21: note = UBE3A-ATS artificial/synthetic sequence
  source 1..21: mol_type = other DNA
tatacccagaggattataaat

SEQ ID NO: 134 - DNA, 21 bp, synthetic construct
  misc_feature 1..21: note = UBE3A-ATS artificial/synthetic sequence
  source 1..21: mol_type = other DNA
ccctagaacttaaagtataat

SEQ ID NO: 135 - DNA, 21 bp, synthetic construct
  misc_feature 1..21: note = UBE3A-ATS artificial/synthetic sequence
  source 1..21: mol_type = other DNA
tgacaaacctggaggtaatat

SEQ ID NO: 136 - DNA, 21 bp, synthetic construct
  misc_feature 1..21: note = UBE3A-ATS artificial/synthetic sequence
  source 1..21: mol_type = other DNA
gacaaacctggaggtaatata

SEQ ID NO: 137 - DNA, 21 bp, synthetic construct
  misc_feature 1..21: note = UBE3A-ATS artificial/synthetic sequence
  source 1..21: mol_type = other DNA
gtccattctcaccacttatat

SEQ ID NO: 138 - DNA, 21 bp, synthetic construct
  misc_feature 1..21: note = UBE3A-ATS artificial/synthetic sequence
  source 1..21: mol_type = other DNA
tccattctcaccacttatatt

SEQ ID NO: 139 - DNA, 21 bp, synthetic construct
  misc_feature 1..21: note = UBE3A-ATS artificial/synthetic sequence
  source 1..21: mol_type = other DNA
ccattctcaccacttatattt

SEQ ID NO: 140 - DNA, 21 bp, synthetic construct
  misc_feature 1..21: note = UBE3A-ATS artificial/synthetic sequence
  source 1..21: mol_type = other DNA
cattctcaccacttatattta

SEQ ID NO: 141 - DNA, 21 bp, synthetic construct
  misc_feature 1..21: note = UBE3A-ATS artificial/synthetic sequence
  source 1..21: mol_type = other DNA
gtagatgacatgatcttatat

SEQ ID NO: 142 - DNA, 21 bp, synthetic construct
  misc_feature 1..21: note = UBE3A-ATS artificial/synthetic sequence
  source 1..21: mol_type = other DNA
gaatagagagcccagaaataa

SEQ ID NO: 143 - DNA, 21 bp, synthetic construct
  misc_feature 1..21: note = UBE3A-ATS artificial/synthetic sequence
  source 1..21: mol_type = other DNA
atgcttgacatcactaataat

SEQ ID NO: 144 - DNA, 21 bp, synthetic construct
  misc_feature 1..21: note = UBE3A-ATS artificial/synthetic sequence
  source 1..21: mol_type = other DNA
tacactgttggtgtgaattta

SEQ ID NO: 145 - DNA, 21 bp, synthetic construct
  misc_feature 1..21: note = UBE3A-ATS artificial/synthetic sequence
  source 1..21: mol_type = other DNA
acactgttggtgtgaatttaa

SEQ ID NO: 146 - DNA, 21 bp, synthetic construct
  misc_feature 1..21: note = UBE3A-ATS artificial/synthetic sequence
  source 1..21: mol_type = other DNA
cactgttggtgtgaatttaaa

SEQ ID NO: 147 - DNA, 21 bp, synthetic construct
  misc_feature 1..21: note = UBE3A-ATS artificial/synthetic sequence
  source 1..21: mol_type = other DNA
ggtatttgcacactcatattt

SEQ ID NO: 148 - DNA, 21 bp, synthetic construct
  misc_feature 1..21: note = UBE3A-ATS artificial/synthetic sequence
  source 1..21: mol_type = other DNA
gtatttgcacactcatattta

SEQ ID NO: 149 - DNA, 21 bp, synthetic construct
  misc_feature 1..21: note = UBE3A-ATS artificial/synthetic sequence
  source 1..21: mol_type = other DNA
ataaagagaacgtggtatata

SEQ ID NO: 150 - DNA, 21 bp, synthetic construct
  misc_feature 1..21: note = UBE3A-ATS artificial/synthetic sequence
  source 1..21: mol_type = other DNA
agacacagtggaatctattta

SEQ ID NO: 151 - DNA, 21 bp, synthetic construct
  misc_feature 1..21: note = UBE3A-ATS artificial/synthetic sequence
  source 1..21: mol_type = other DNA
ggtacaaactttcagttataa

SEQ ID NO: 152 - DNA, 21 bp, synthetic construct
  misc_feature 1..21: note = UBE3A-ATS artificial/synthetic sequence
  source 1..21: mol_type = other DNA
gaaggcactgatatgttaatt

SEQ ID NO: 153 - DNA, 21 bp, synthetic construct
  misc_feature 1..21: note = UBE3A-ATS artificial/synthetic sequence
  source 1..21: mol_type = other DNA
aggcactgatatgttaattat

SEQ ID NO: 154 - DNA, 21 bp, synthetic construct
  misc_feature 1..21: note = UBE3A-ATS artificial/synthetic sequence
  source 1..21: mol_type = other DNA
atccattcaagctagattatt

SEQ ID NO: 155 - DNA, 21 bp, synthetic construct
  misc_feature 1..21: note = UBE3A-ATS artificial/synthetic sequence
  source 1..21: mol_type = other DNA
tccattcaagctagattattt

SEQ ID NO: 156 - DNA, 21 bp, synthetic construct
  misc_feature 1..21: note = UBE3A-ATS artificial/synthetic sequence
  source 1..21: mol_type = other DNA
ggcatggtggccctcaattat

SEQ ID NO: 157 - DNA, 21 bp, synthetic construct
  misc_feature 1..21: note = UBE3A-ATS artificial/synthetic sequence
  source 1..21: mol_type = other DNA
gcatggtggccctcaattata

SEQ ID NO: 158 - DNA, 21 bp, synthetic construct
  misc_feature 1..21: note = UBE3A-ATS artificial/synthetic sequence
  source 1..21: mol_type = other DNA
catggtggccctcaattatat

SEQ ID NO: 159 - DNA, 21 bp, synthetic construct
  misc_feature 1..21: note = UBE3A-ATS artificial/synthetic sequence
  source 1..21: mol_type = other DNA
atggtggccctcaattatata

SEQ ID NO: 160 - DNA, 21 bp, synthetic construct
  misc_feature 1..21: note = UBE3A-ATS artificial/synthetic sequence
  source 1..21: mol_type = other DNA
tggtggccctcaattatatat

SEQ ID NO: 161 - DNA, 21 bp, synthetic construct
  misc_feature 1..21: note = UBE3A-ATS artificial/synthetic sequence
  source 1..21: mol_type = other DNA
ggtggccctcaattatatata

SEQ ID NO: 162 - DNA, 21 bp, synthetic construct
  misc_feature 1..21: note = UBE3A-ATS artificial/synthetic sequence
  source 1..21: mol_type = other DNA
gtggccctcaattatatatat

SEQ ID NO: 163 - DNA, 21 bp, synthetic construct
  misc_feature 1..21: note = UBE3A-ATS artificial/synthetic sequence
  source 1..21: mol_type = other DNA
tggccctcaattatatatata

SEQ ID NO: 164 - DNA, 21 bp, synthetic construct
  misc_feature 1..21: note = UBE3A-ATS artificial/synthetic sequence
  source 1..21: mol_type = other DNA
ggccctcaattatatatataa

SEQ ID NO: 165 - DNA, 21 bp, synthetic construct
  misc_feature 1..21: note = UBE3A-ATS artificial/synthetic sequence
  source 1..21: mol_type = other DNA
atgaacaacaatgggatttaa

SEQ ID NO: 166 - DNA, 21 bp, synthetic construct
  misc_feature 1..21: note = UBE3A-ATS artificial/synthetic sequence
  source 1..21: mol_type = other DNA
tgaacaacaatgggatttaaa

SEQ ID NO: 167 - DNA, 21 bp, synthetic construct
  misc_feature 1..21: note = UBE3A-ATS artificial/synthetic sequence
  source 1..21: mol_type = other DNA
gaacaacaatgggatttaaat

SEQ ID NO: 168 - DNA, 21 bp, synthetic construct
  misc_feature 1..21: note = UBE3A-ATS artificial/synthetic sequence
  source 1..21: mol_type = other DNA
agcaatagtggagaaatataa

SEQ ID NO: 169 - DNA, 21 bp, synthetic construct
  misc_feature 1..21: note = UBE3A-ATS artificial/synthetic sequence
  source 1..21: mol_type = other DNA
gacctaaaccctatcttataa

SEQ ID NO: 170 - DNA, 21 bp, synthetic construct
  misc_feature 1..21: note = UBE3A-ATS artificial/synthetic sequence
  source 1..21: mol_type = other DNA
acctaaaccctatcttataat

SEQ ID NO: 171 - DNA, 21 bp, synthetic construct
  misc_feature 1..21: note = UBE3A-ATS artificial/synthetic sequence
  source 1..21: mol_type = other DNA
cctaaaccctatcttataatt

SEQ ID NO: 172 - DNA, 21 bp, synthetic construct
  misc_feature 1..21: note = UBE3A-ATS artificial/synthetic sequence
  source 1..21: mol_type = other DNA
ctagagcagcaatactaatat

SEQ ID NO: 173 - DNA, 21 bp, synthetic construct
  misc_feature 1..21: note = UBE3A-ATS artificial/synthetic sequence
  source 1..21: mol_type = other DNA
gtgtagctatgactgatattt

SEQ ID NO: 174 - DNA, 21 bp, synthetic construct
  misc_feature 1..21: note = UBE3A-ATS artificial/synthetic sequence
  source 1..21: mol_type = other DNA
tgtagctatgactgatattta

SEQ ID NO: 175 - DNA, 21 bp, synthetic construct
  misc_feature 1..21: note = UBE3A-ATS artificial/synthetic sequence
  source 1..21: mol_type = other DNA
gtagctatgactgatatttat

SEQ ID NO: 176 - DNA, 21 bp, synthetic construct
  misc_feature 1..21: note = UBE3A-ATS artificial/synthetic sequence
  source 1..21: mol_type = other DNA
tagtctttgcttgcctattta

SEQ ID NO: 177 - DNA, 21 bp, synthetic construct
  misc_feature 1..21: note = UBE3A-ATS artificial/synthetic sequence
  source 1..21: mol_type = other DNA
agtctttgcttgcctatttat

SEQ ID NO: 178 - DNA, 21 bp, synthetic construct
  misc_feature 1..21: note = UBE3A-ATS artificial/synthetic sequence
  source 1..21: mol_type = other DNA
gtctttgcttgcctatttatt

SEQ ID NO: 179 - DNA, 21 bp, synthetic construct
  misc_feature 1..21: note = UBE3A-ATS artificial/synthetic sequence
  source 1..21: mol_type = other DNA
ctgtgtttagttgtcttattt

SEQ ID NO: 180 - DNA, 21 bp, synthetic construct
  misc_feature 1..21: note = UBE3A-ATS artificial/synthetic sequence
  source 1..21: mol_type = other DNA
ctaccctggatagggaatata

SEQ ID NO: 181 - DNA, 21 bp, synthetic construct
  misc_feature 1..21: note = UBE3A-ATS artificial/synthetic sequence
  source 1..21: mol_type = other DNA
ttctattgcaacaggataaat

SEQ ID NO: 182 - DNA, 21 bp, synthetic construct
  misc_feature 1..21: note = UBE3A-ATS artificial/synthetic sequence
  source 1..21: mol_type = other DNA
tctattgcaacaggataaata

SEQ ID NO: 183 - DNA, 21 bp, synthetic construct
  misc_feature 1..21: note = UBE3A-ATS artificial/synthetic sequence
  source 1..21: mol_type = other DNA
ctattgcaacaggataaataa

SEQ ID NO: 184 - DNA, 21 bp, synthetic construct
  misc_feature 1..21: note = UBE3A-ATS artificial/synthetic sequence
  source 1..21: mol_type = other DNA
actggtgaactcctcaattat

SEQ ID NO: 185 - DNA, 21 bp, synthetic construct
  misc_feature 1..21: note = UBE3A-ATS artificial/synthetic sequence
  source 1..21: mol_type = other DNA
tcaaggaggaaacagattata

SEQ ID NO: 186 - DNA, 21 bp, synthetic construct
  misc_feature 1..21: note = UBE3A-ATS artificial/synthetic sequence
  source 1..21: mol_type = other DNA
caaggaggaaacagattataa

SEQ ID NO: 187 - DNA, 21 bp, synthetic construct
  misc_feature 1..21: note = UBE3A-ATS artificial/synthetic sequence
  source 1..21: mol_type = other DNA
aggaggaaacagattataaat

SEQ ID NO: 188 - DNA, 21 bp, synthetic construct
  misc_feature 1..21: note = UBE3A-ATS artificial/synthetic sequence
  source 1..21: mol_type = other DNA
tgcctgtatgataagaaatta

SEQ ID NO: 189 - DNA, 21 bp, synthetic construct
  misc_feature 1..21: note = UBE3A-ATS artificial/synthetic sequence
  source 1..21: mol_type = other DNA
gcctgtatgataagaaattat

SEQ ID NO: 190 - DNA, 21 bp, synthetic construct
  misc_feature 1..21: note = UBE3A-ATS artificial/synthetic sequence
  source 1..21: mol_type = other DNA
gaaccaatcttgtgctttatt

SEQ ID NO: 191 - DNA, 21 bp, synthetic construct
  misc_feature 1..21: note = UBE3A-ATS artificial/synthetic sequence
  source 1..21: mol_type = other DNA
gcattctctgatctgatttat

SEQ ID NO: 192 - DNA, 21 bp, synthetic construct
  misc_feature 1..21: note = UBE3A-ATS artificial/synthetic sequence
  source 1..21: mol_type = other DNA
cattctctgatctgatttatt

SEQ ID NO: 193 - DNA, 21 bp, synthetic construct
  misc_feature 1..21: note = UBE3A-ATS artificial/synthetic sequence
  source 1..21: mol_type = other DNA
agaggcttaaaggagattatt

SEQ ID NO: 194 - DNA, 21 bp, synthetic construct
  misc_feature 1..21: note = UBE3A-ATS artificial/synthetic sequence
  source 1..21: mol_type = other DNA
ttgttcaaggattccttatta

SEQ ID NO: 195 - DNA, 21 bp, synthetic construct
  misc_feature 1..21: note = UBE3A-ATS artificial/synthetic sequence
  source 1..21: mol_type = other DNA
tgttcaaggattccttattat

SEQ ID NO: 196 - DNA, 21 bp, synthetic construct
  misc_feature 1..21: note = UBE3A-ATS artificial/synthetic sequence
  source 1..21: mol_type = other DNA
gttcaaggattccttattatt

SEQ ID NO: 197 - DNA, 21 bp, synthetic construct
  misc_feature 1..21: note = UBE3A-ATS artificial/synthetic sequence
  source 1..21: mol_type = other DNA
tgtggagaatcattgatatat

SEQ ID NO: 198 - DNA, 21 bp, synthetic construct
  misc_feature 1..21: note = UBE3A-ATS artificial/synthetic sequence
  source 1..21: mol_type = other DNA
gtggagaatcattgatatatt

SEQ ID NO: 199 - DNA, 21 bp, synthetic construct
  misc_feature 1..21: note = UBE3A-ATS artificial/synthetic sequence
  source 1..21: mol_type = other DNA
gtatatgagagttccattatt

SEQ ID NO: 200 - DNA, 21 bp, synthetic construct
  misc_feature 1..21: note = UBE3A-ATS artificial/synthetic sequence
  source 1..21: mol_type = other DNA
tggctgtggaaacgcttattt

SEQ ID NO: 201 - DNA, 21 bp, synthetic construct
  misc_feature 1..21: note = UBE3A-ATS artificial/synthetic sequence
  source 1..21: mol_type = other DNA
tggatcgatgatgagaataat

SEQ ID NO: 202 - DNA, 21 bp, synthetic construct
  misc_feature 1..21: note = UBE3A-ATS artificial/synthetic sequence
  source 1..21: mol_type = other DNA
ggatcgatgatgagaataatt

SEQ ID NO: 203 - DNA, 21 bp, synthetic construct
  misc_feature 1..21: note = UBE3A-ATS artificial/synthetic sequence
  source 1..21: mol_type = other DNA
gccctccaataggacaaataa

SEQ ID NO: 204 - DNA, 21 bp, synthetic construct
  misc_feature 1..21: note = UBE3A-ATS artificial/synthetic sequence
  source 1..21: mol_type = other DNA
tgacccaagacttgctttaat

SEQ ID NO: 205 - DNA, 21 bp, synthetic construct
  misc_feature 1..21: note = UBE3A-ATS artificial/synthetic sequence
  source 1..21: mol_type = other DNA
gacccaagacttgctttaatt

SEQ ID NO: 206 - DNA, 21 bp, synthetic construct
  misc_feature 1..21: note = UBE3A-ATS artificial/synthetic sequence
  source 1..21: mol_type = other DNA
tgtgctgaaagaaggaaatat

SEQ ID NO: 207 - DNA, 21 bp, synthetic construct
  misc_feature 1..21: note = UBE3A-ATS artificial/synthetic sequence
  source 1..21: mol_type = other DNA
atatggcatgcctctattaaa

SEQ ID NO: 208 - DNA, 21 bp, synthetic construct
  misc_feature 1..21: note = UBE3A-ATS artificial/synthetic sequence
  source 1..21: mol_type = other DNA
tatggcatgcctctattaaat

SEQ ID NO: 209 - DNA, 21 bp, synthetic construct
  misc_feature 1..21: note = UBE3A-ATS artificial/synthetic sequence
  source 1..21: mol_type = other DNA
atggcatgcctctattaaata

SEQ ID NO: 210 - DNA, 21 bp, synthetic construct
  misc_feature 1..21: note = UBE3A-ATS artificial/synthetic sequence
  source 1..21: mol_type = other DNA
tggcatgcctctattaaataa

SEQ ID NO: 211 - DNA, 21 bp, synthetic construct
  misc_feature 1..21: note = UBE3A-ATS artificial/synthetic sequence
  source 1..21: mol_type = other DNA
ggcatgcctctattaaataat

SEQ ID NO: 212 - DNA, 21 bp, synthetic construct
  misc_feature 1..21: note = UBE3A-ATS artificial/synthetic sequence
  source 1..21: mol_type = other DNA
gacagtggaaccaagtttatt

SEQ ID NO: 213 - DNA, 21 bp, synthetic construct
  misc_feature 1..21: note = UBE3A-ATS artificial/synthetic sequence
  source 1..21: mol_type = other DNA
gagactccatggttcataata

SEQ ID NO: 214 - DNA, 21 bp, synthetic construct
  misc_feature 1..21: note = UBE3A-ATS artificial/synthetic sequence
  source 1..21: mol_type = other DNA
agactccatggttcataatat

SEQ ID NO: 215 - DNA, 21 bp, synthetic construct
  misc_feature 1..21: note = UBE3A-ATS artificial/synthetic sequence
  source 1..21: mol_type = other DNA
ttgtggtcatactacattata

SEQ ID NO: 216 - DNA, 21 bp, synthetic construct
  misc_feature 1..21: note = UBE3A-ATS artificial/synthetic sequence
  source 1..21: mol_type = other DNA
tgtggtcatactacattatat

SEQ ID NO: 217 - DNA, 21 bp, synthetic construct
  misc_feature 1..21: note = UBE3A-ATS artificial/synthetic sequence
  source 1..21: mol_type = other DNA
gtggtcatactacattatatt

SEQ ID NO: 218 - DNA, 21 bp, synthetic construct
  misc_feature 1..21: note = UBE3A-ATS artificial/synthetic sequence
  source 1..21: mol_type = other DNA
ttgcaacaaggtaagttatat

SEQ ID NO: 219 - DNA, 21 bp, synthetic construct
  misc_feature 1..21: note = UBE3A-ATS artificial/synthetic sequence
  source 1..21: mol_type = other DNA
tgcaacaaggtaagttatata

SEQ ID NO: 220 - DNA, 21 bp, synthetic construct
  misc_feature 1..21: note = UBE3A-ATS artificial/synthetic sequence
  source 1..21: mol_type = other DNA
ttgattgatgccctcataata

SEQ ID NO: 221 - DNA, 21 bp, synthetic construct
  misc_feature 1..21: note = UBE3A-ATS artificial/synthetic sequence
  source 1..21: mol_type = other DNA
tgaagttgggtaaagtataaa

SEQ ID NO: 222 - DNA, 21 bp, synthetic construct
  misc_feature 1..21: note = UBE3A-ATS artificial/synthetic sequence
  source 1..21: mol_type = other DNA
gtcaatacacaactgataaat

SEQ ID NO: 223 - DNA, 21 bp, synthetic construct
  misc_feature 1..21: note = UBE3A-ATS artificial/synthetic sequence
  source 1..21: mol_type = other DNA
caaagagtttcagccataaat

SEQ ID NO: 224 - DNA, 21 bp, synthetic construct
  misc_feature 1..21: note = UBE3A-ATS artificial/synthetic sequence
  source 1..21: mol_type = other DNA
catgctttccagaggaaatat

SEQ ID NO: 225 - DNA, 21 bp, synthetic construct
  misc_feature 1..21: note = UBE3A-ATS artificial/synthetic sequence
  source 1..21: mol_type = other DNA
tccagaggaaatatgtttaat

SEQ ID NO: 226 - DNA, 21 bp, synthetic construct
  misc_feature 1..21: note = UBE3A-ATS artificial/synthetic sequence
  source 1..21: mol_type = other DNA
tttaatcatctgccctatatt

SEQ ID NO: 227 - DNA, 21 bp, synthetic construct
  misc_feature 1..21: note = UBE3A-ATS artificial/synthetic sequence
  source 1..21: mol_type = other DNA
ttaatcatctgccctatatta

SEQ ID NO: 228 - DNA, 21 bp, synthetic construct
  misc_feature 1..21: note = UBE3A-ATS artificial/synthetic sequence
  source 1..21: mol_type = other DNA
tgcaagataccatacatttat

SEQ ID NO: 229 - DNA, 21 bp, synthetic construct
  misc_feature 1..21: note = UBE3A-ATS artificial/synthetic sequence
  source 1..21: mol_type = other DNA
gcaagataccatacatttata

SEQ ID NO: 230 - DNA, 21 bp, synthetic construct
  misc_feature 1..21: note = UBE3A-ATS artificial/synthetic sequence
  source 1..21: mol_type = other DNA
ggtccatgtgatttctttaat

SEQ ID NO: 231 - DNA, 21 bp, synthetic construct
  misc_feature 1..21: note = UBE3A-ATS artificial/synthetic sequence
  source 1..21: mol_type = other DNA
tgccataactatagcatttat

SEQ ID NO: 232 - DNA, 21 bp, synthetic construct
  misc_feature 1..21: note = UBE3A-ATS artificial/synthetic sequence
  source 1..21: mol_type = other DNA
ggataccatccacactataaa

SEQ ID NO: 233 - DNA, 21 bp, synthetic construct
  misc_feature 1..21: note = UBE3A-ATS artificial/synthetic sequence
  source 1..21: mol_type = other DNA
tgtgtttccttccagataatt

SEQ ID NO: 234 - DNA, 21 bp, synthetic construct
  misc_feature 1..21: note = UBE3A-ATS artificial/synthetic sequence
  source 1..21: mol_type = other DNA
gtgtttccttccagataattt

SEQ ID NO: 235 - DNA, 21 bp, synthetic construct
  misc_feature 1..21: note = UBE3A-ATS artificial/synthetic sequence
  source 1..21: mol_type = other DNA
gtgtgctgaaagggctataaa

SEQ ID NO: 236 - DNA, 21 bp, synthetic construct
  misc_feature 1..21: note = UBE3A-ATS artificial/synthetic sequence
  source 1..21: mol_type = other DNA
ttgaaccaaccaccctataaa

SEQ ID NO: 237 - DNA, 21 bp, synthetic construct
  misc_feature 1..21: note = UBE3A-ATS artificial/synthetic sequence
  source 1..21: mol_type = other DNA
taaacgttgtatggcttattt

SEQ ID NO: 238 - DNA, 21 bp, synthetic construct
  misc_feature 1..21: note = UBE3A-ATS artificial/synthetic sequence
  source 1..21: mol_type = other DNA
atccctgtgtacacaatattt

SEQ ID NO: 239 - DNA, 21 bp, synthetic construct
  misc_feature 1..21: note = UBE3A-ATS artificial/synthetic sequence
  source 1..21: mol_type = other DNA
atgtgtggtaaatccattatt

SEQ ID NO: 240 - DNA, 21 bp, synthetic construct
  misc_feature 1..21: note = UBE3A-ATS artificial/synthetic sequence
  source 1..21: mol_type = other DNA
tgtgtggtaaatccattattt

SEQ ID NO: 241 - DNA, 21 bp, synthetic construct
  misc_feature 1..21: note = UBE3A-ATS artificial/synthetic sequence
  source 1..21: mol_type = other DNA
atgagcatttggcagtaatat

SEQ ID NO: 242 - DNA, 21 bp, synthetic construct
  misc_feature 1..21: note = UBE3A-ATS artificial/synthetic sequence
  source 1..21: mol_type = other DNA
tgagcatttggcagtaatatt

SEQ ID NO: 243 - DNA, 21 bp, synthetic construct
  misc_feature 1..21: note = UBE3A-ATS artificial/synthetic sequence
  source 1..21: mol_type = other DNA
gagcatttggcagtaatatta

SEQ ID NO: 244 - DNA, 21 bp, synthetic construct
  misc_feature 1..21: note = UBE3A-ATS artificial/synthetic sequence
  source 1..21: mol_type = other DNA
agcatttggcagtaatattat

SEQ ID NO: 245 - DNA, 21 bp, synthetic construct
  misc_feature 1..21: note = UBE3A-ATS artificial/synthetic sequence
  source 1..21: mol_type = other DNA
gcatttggcagtaatattatt

SEQ ID NO: 246 - DNA, 21 bp, synthetic construct
  misc_feature 1..21: note = UBE3A-ATS artificial/synthetic sequence
  source 1..21: mol_type = other DNA
gagactctgctgaagtttata

SEQ ID NO: 247 - DNA, 21 bp, synthetic construct
  misc_feature 1..21: note = UBE3A-ATS artificial/synthetic sequence
  source 1..21: mol_type = other DNA
agactctgctgaagtttataa

SEQ ID NO: 248 - DNA, 21 bp, synthetic construct
  misc_feature 1..21: note = UBE3A-ATS artificial/synthetic sequence
  source 1..21: mol_type = other DNA
cagttatagaagtgctaattt

SEQ ID NO: 249 - DNA, 21 bp, synthetic construct
  misc_feature 1..21: note = UBE3A-ATS artificial/synthetic sequence
  source 1..21: mol_type = other DNA
tatgatgacctcctgaattat

SEQ ID NO: 250 - DNA, 21 bp, synthetic construct
  misc_feature 1..21: note = UBE3A-ATS artificial/synthetic sequence
  source 1..21: mol_type = other DNA
gggtctggtgtttcatttatt

SEQ ID NO: 251 - DNA, 21 bp, synthetic construct
  misc_feature 1..21: note = UBE3A-ATS artificial/synthetic sequence
  source 1..21: mol_type = other DNA
ggtctggtgtttcatttattt

SEQ ID NO: 252 - DNA, 21 bp, synthetic construct
  misc_feature 1..21: note = UBE3A-ATS artificial/synthetic sequence
  source 1..21: mol_type = other DNA
gggtctcattatagttaatat

SEQ ID NO: 253 - DNA, 21 bp, synthetic construct
  misc_feature 1..21: note = UBE3A-ATS artificial/synthetic sequence
  source 1..21: mol_type = other DNA
taacagcatgtcaggtaataa

SEQ ID NO: 254 - DNA, 21 bp, synthetic construct
  misc_feature 1..21: note = UBE3A-ATS artificial/synthetic sequence
  source 1..21: mol_type = other DNA
gcagagccctattcctttaaa

SEQ ID NO: 255 - DNA, 21 bp, synthetic construct
  misc_feature 1..21: note = UBE3A-ATS artificial/synthetic sequence
  source 1..21: mol_type = other DNA
ctcctaaatgtttggaaattt

SEQ ID NO: 256 - DNA, 21 bp, synthetic construct
  misc_feature 1..21: note = UBE3A-ATS artificial/synthetic sequence
  source 1..21: mol_type = other DNA
ttagcatttctgacctattta

SEQ ID NO: 257 - DNA, 21 bp, synthetic construct
  misc_feature 1..21: note = UBE3A-ATS artificial/synthetic sequence
  source 1..21: mol_type = other DNA
tagcatttctgacctatttat

SEQ ID NO: 258 - DNA, 21 bp, synthetic construct
  misc_feature 1..21: note = UBE3A-ATS artificial/synthetic sequence
  source 1..21: mol_type = other DNA
agcatttctgacctatttatt

SEQ ID NO: 259 - DNA, 21 bp, synthetic construct
  misc_feature 1..21: note = UBE3A-ATS artificial/synthetic sequence
  source 1..21: mol_type = other DNA
acaggatatagggaataattt

SEQ ID NO: 260 - DNA, 21 bp, synthetic construct
  misc_feature 1..21: note = UBE3A-ATS artificial/synthetic sequence
  source 1..21: mol_type = other DNA
caggatatagggaataattta

SEQ ID NO: 261 - DNA, 21 bp, synthetic construct
  misc_feature 1..21: note = UBE3A-ATS artificial/synthetic sequence
  source 1..21: mol_type = other DNA
tagcactgaaatgcctatatt

SEQ ID NO: 262 - DNA, 21 bp, synthetic construct
  misc_feature 1..21: note = UBE3A-ATS artificial/synthetic sequence
  source 1..21: mol_type = other DNA
agcactgaaatgcctatatta

SEQ ID NO: 263 - DNA, 21 bp, synthetic construct
  misc_feature 1..21: note = UBE3A-ATS artificial/synthetic sequence
  source 1..21: mol_type = other DNA
gagcagaaatcaatgaaatat

SEQ ID NO: 264 - DNA, 21 bp, synthetic construct
  misc_feature 1..21: note = UBE3A-ATS artificial/synthetic sequence
  source 1..21: mol_type = other DNA
acttgtaaccagaccaattta

SEQ ID NO: 265 - DNA, 21 bp, synthetic construct
  misc_feature 1..21: note = UBE3A-ATS artificial/synthetic sequence
  source 1..21: mol_type = other DNA
cttgtaaccagaccaatttaa

SEQ ID NO: 266 - DNA, 21 bp, synthetic construct
  misc_feature 1..21: note = UBE3A-ATS artificial/synthetic sequence
  source 1..21: mol_type = other DNA
actcagtgaacaagtaaataa

SEQ ID NO: 267 - DNA, 21 bp, synthetic construct
  misc_feature 1..21: note = UBE3A-ATS artificial/synthetic sequence
  source 1..21: mol_type = other DNA
tagccctgtatcaagtaaatt

SEQ ID NO: 268 - DNA, 21 bp, synthetic construct
  misc_feature 1..21: note = UBE3A-ATS artificial/synthetic sequence
  source 1..21: mol_type = other DNA
acaagattccagaacatttaa

SEQ ID NO: 269 - DNA, 21 bp, synthetic construct
  misc_feature 1..21: note = UBE3A-ATS artificial/synthetic sequence
  source 1..21: mol_type = other DNA
caagattccagaacatttaaa

SEQ ID NO: 270 - DNA, 21 bp, synthetic construct
  misc_feature 1..21: note = UBE3A-ATS artificial/synthetic sequence
  source 1..21: mol_type = other DNA
gagagttacccaaagaattta

SEQ ID NO: 271 - DNA, 21 bp, synthetic construct
  misc_feature 1..21: note = UBE3A-ATS artificial/synthetic sequence
  source 1..21: mol_type = other DNA
agagttacccaaagaatttaa

SEQ ID NO: 272 - DNA, 21 bp, synthetic construct
  misc_feature 1..21: note = UBE3A-ATS artificial/synthetic sequence
  source 1..21: mol_type = other DNA
agaagaaaggtttggaaattt

SEQ ID NO: 273 - DNA, 21 bp, synthetic construct
  misc_feature 1..21: note = UBE3A-ATS artificial/synthetic sequence
  source 1..21: mol_type = other DNA
agcccaatctataggatttat

SEQ ID NO: 274 - DNA, 21 bp, synthetic construct
  misc_feature 1..21: note = UBE3A-ATS artificial/synthetic sequence
  source 1..21: mol_type = other DNA
gcccaatctataggatttata

SEQ ID NO: 275 - DNA, 21 bp, synthetic construct
  misc_feature 1..21: note = UBE3A-ATS artificial/synthetic sequence
  source 1..21: mol_type = other DNA
cccaatctataggatttatat

SEQ ID NO: 276 - DNA, 21 bp, synthetic construct
  misc_feature 1..21: note = UBE3A-ATS artificial/synthetic sequence
  source 1..21: mol_type = other DNA
agttcatcgttagtgttatat

SEQ ID NO: 277 - DNA, 21 bp, synthetic construct
  misc_feature 1..21: note = UBE3A-ATS artificial/synthetic sequence
  source 1..21: mol_type = other DNA
gttcatcgttagtgttatata

SEQ ID NO: 278 - DNA, 21 bp, synthetic construct
  misc_feature 1..21: note = UBE3A-ATS artificial/synthetic sequence
  source 1..21: mol_type = other DNA
accaccatgcccagctaattt

SEQ ID NO: 279 - DNA, 21 bp, synthetic construct
  misc_feature 1..21: note = UBE3A-ATS artificial/synthetic sequence
  source 1..21: mol_type = other DNA
atgacccatttgaagttaatt

SEQ ID NO: 280 - DNA, 21 bp, synthetic construct
  misc_feature 1..21: note = UBE3A-ATS artificial/synthetic sequence
  source 1..21: mol_type = other DNA
tgacccatttgaagttaattt

SEQ ID NO: 281 - DNA, 21 bp, synthetic construct
  misc_feature 1..21: note = UBE3A-ATS artificial/synthetic sequence
  source 1..21: mol_type = other DNA
atgctggcctcactgaataaa

SEQ ID NO: 282 - DNA, 21 bp, synthetic construct
  misc_feature 1..21: note = UBE3A-ATS artificial/synthetic sequence
  source 1..21: mol_type = other DNA
tgctggcctcactgaataaat

SEQ ID NO: 283 - DNA, 21 bp, synthetic construct
  misc_feature 1..21: note = UBE3A-ATS artificial/synthetic sequence
  source 1..21: mol_type = other DNA
gctggcctcactgaataaatt

SEQ ID NO: 284 - DNA, 21 bp, synthetic construct
  misc_feature 1..21: note = UBE3A-ATS artificial/synthetic sequence
  source 1..21: mol_type = other DNA
tgttccctcctcttcaattat

SEQ ID NO: 285 - DNA, 21 bp, synthetic construct
  misc_feature 1..21: note = UBE3A-ATS artificial/synthetic sequence
  source 1..21: mol_type = other DNA
gttccctcctcttcaattatt

SEQ ID NO: 286 - DNA, 21 bp, synthetic construct
  misc_feature 1..21: note = UBE3A-ATS artificial/synthetic sequence
  source 1..21: mol_type = other DNA
ttccctcctcttcaattattt

SEQ ID NO: 287 - DNA, 21 bp, synthetic construct
  misc_feature 1..21: note = UBE3A-ATS artificial/synthetic sequence
  source 1..21: mol_type = other DNA
ttggtaggttgtgcgtattta

SEQ ID NO: 288 - DNA, 21 bp, synthetic construct
  misc_feature 1..21: note = UBE3A-ATS artificial/synthetic sequence
  source 1..21: mol_type = other DNA
attagttggcatgcaattatt

SEQ ID NO: 289 - DNA, 21 bp, synthetic construct
  misc_feature 1..21: note = UBE3A-ATS artificial/synthetic sequence
  source 1..21: mol_type = other DNA
attcgtagttctctgaataat

SEQ ID NO: 290 - DNA, 21 bp, synthetic construct
  misc_feature 1..21: note = UBE3A-ATS artificial/synthetic sequence
  source 1..21: mol_type = other DNA
ttccttctgttggcctttaat

SEQ ID NO: 291 - DNA, 21 bp, synthetic construct
  misc_feature 1..21: note = UBE3A-ATS artificial/synthetic sequence
  source 1..21: mol_type = other DNA
tccttctgttggcctttaatt

SEQ ID NO: 292 - DNA, 21 bp, synthetic construct
  misc_feature 1..21: note = UBE3A-ATS artificial/synthetic sequence
  source 1..21: mol_type = other DNA
ccttctgttggcctttaattt

SEQ ID NO: 293 - DNA, 21 bp, synthetic construct
  misc_feature 1..21: note = UBE3A-ATS artificial/synthetic sequence
  source 1..21: mol_type = other DNA
gaaagcatttagagctataaa

SEQ ID NO: 294 - DNA, 21 bp, synthetic construct
  misc_feature 1..21: note = UBE3A-ATS artificial/synthetic sequence
  source 1..21: mol_type = other DNA
ctttcactacctgccataaat

SEQ ID NO: 295 - DNA, 21 bp, synthetic construct
  misc_feature 1..21: note = UBE3A-ATS artificial/synthetic sequence
  source 1..21: mol_type = other DNA
tttcactacctgccataaatt

SEQ ID NO: 296 - DNA, 21 bp, synthetic construct
  misc_feature 1..21: note = UBE3A-ATS artificial/synthetic sequence
  source 1..21: mol_type = other DNA
ttcactacctgccataaattt

SEQ ID NO: 297 - DNA, 21 bp, synthetic construct
  misc_feature 1..21: note = UBE3A-ATS artificial/synthetic sequence
  source 1..21: mol_type = other DNA
tccttgacctattggttattt

SEQ ID NO: 298 - DNA, 21 bp, synthetic construct
  misc_feature 1..21: note = UBE3A-ATS artificial/synthetic sequence
  source 1..21: mol_type = other DNA
ccttgacctattggttattta

SEQ ID NO: 299 - DNA, 21 bp, synthetic construct
  misc_feature 1..21: note = UBE3A-ATS artificial/synthetic sequence
  source 1..21: mol_type = other DNA
cttgacctattggttatttaa

SEQ ID NO: 300 - DNA, 21 bp, synthetic construct
  misc_feature 1..21: note = UBE3A-ATS artificial/synthetic sequence
  source 1..21: mol_type = other DNA
ttgtgatcacagaagatattt

SEQ ID NO: 301 - DNA, 21 bp, synthetic construct
  misc_feature 1..21: note = UBE3A-ATS artificial/synthetic sequence
  source 1..21: mol_type = other DNA
tgtataatcgcagtctattaa

SEQ ID NO: 302 - DNA, 21 bp, synthetic construct
  misc_feature 1..21: note = UBE3A-ATS artificial/synthetic sequence
  source 1..21: mol_type = other DNA
tcgcagtctattaacatttat

SEQ ID NO: 303 - DNA, 21 bp, synthetic construct
  misc_feature 1..21: note = UBE3A-ATS artificial/synthetic sequence
  source 1..21: mol_type = other DNA
cgcagtctattaacatttatt

SEQ ID NO: 304 - DNA, 21 bp, synthetic construct
  misc_feature 1..21: note = UBE3A-ATS artificial/synthetic sequence
  source 1..21: mol_type = other DNA
gagtggtaaagtctctattat

SEQ ID NO: 305 - DNA, 21 bp, synthetic construct
  misc_feature 1..21: note = UBE3A-ATS artificial/synthetic sequence
  source 1..21: mol_type = other DNA
agtggtaaagtctctattatt

SEQ ID NO: 306 - DNA, 21 bp, synthetic construct
  misc_feature 1..21: note = UBE3A-ATS artificial/synthetic sequence
  source 1..21: mol_type = other DNA
agcataagctatgtcattaaa

SEQ ID NO: 307 - DNA, 21 bp, synthetic construct
  misc_feature 1..21: note = UBE3A-ATS artificial/synthetic sequence
  source 1..21: mol_type = other DNA
ctcttcatttccttcaatatt

SEQ ID NO: 308 - DNA, 21 bp, synthetic construct
  misc_feature 1..21: note = UBE3A-ATS artificial/synthetic sequence
  source 1..21: mol_type = other DNA
tgagatacctagaacaatata

SEQ ID NO: 309 - DNA, 21 bp, synthetic construct
  misc_feature 1..21: note = UBE3A-ATS artificial/synthetic sequence
  source 1..21: mol_type = other DNA
gagatacctagaacaatataa

SEQ ID NO: 310 - DNA, 21 bp, synthetic construct
  misc_feature 1..21: note = UBE3A-ATS artificial/synthetic sequence
  source 1..21: mol_type = other DNA
ctctttctctgtgagattata

SEQ ID NO: 311 - DNA, 21 bp, synthetic construct
  misc_feature 1..21: note = UBE3A-ATS artificial/synthetic sequence
  source 1..21: mol_type = other DNA
acaacagcctggaagtataat

SEQ ID NO: 312 - DNA, 21 bp, synthetic construct
  misc_feature 1..21: note = UBE3A-ATS artificial/synthetic sequence
  source 1..21: mol_type = other DNA
caacagcctggaagtataatt

SEQ ID NO: 313 - DNA, 21 bp, synthetic construct
  misc_feature 1..21: note = UBE3A-ATS artificial/synthetic sequence
  source 1..21: mol_type = other DNA
acagcctggaagtataattaa

SEQ ID NO: 314 - DNA, 21 bp, synthetic construct
  misc_feature 1..21: note = UBE3A-ATS artificial/synthetic sequence
  source 1..21: mol_type = other DNA
attcaaactgatgccaattta

SEQ ID NO: 315 - DNA, 21 bp, synthetic construct
  misc_feature 1..21: note = UBE3A-ATS artificial/synthetic sequence
  source 1..21: mol_type = other DNA
ttcaaactgatgccaatttaa

SEQ ID NO: 316 - DNA, 21 bp, synthetic construct
  misc_feature 1..21: note = UBE3A-ATS artificial/synthetic sequence
  source 1..21: mol_type = other DNA
agtcaacacaccaatattaaa

SEQ ID NO: 317 - DNA, 21 bp, synthetic construct
  misc_feature 1..21: note = UBE3A-ATS artificial/synthetic sequence
  source 1..21: mol_type = other DNA
agctcctgtttgaagtaaatt

SEQ ID NO: 318 - DNA, 21 bp, synthetic construct
  misc_feature 1..21: note = UBE3A-ATS artificial/synthetic sequence
  source 1..21: mol_type = other DNA
gctcctgtttgaagtaaattt

SEQ ID NO: 319 - DNA, 21 bp, synthetic construct
  misc_feature 1..21: note = UBE3A-ATS artificial/synthetic sequence
  source 1..21: mol_type = other DNA
gccttccaaggtttctattaa

SEQ ID NO: 320 - DNA, 21 bp, synthetic construct
  misc_feature 1..21: note = UBE3A-ATS artificial/synthetic sequence
  source 1..21: mol_type = other DNA
tgtgggtctctttggatttat

SEQ ID NO: 321 - DNA, 21 bp, synthetic construct
  misc_feature 1..21: note = UBE3A-ATS artificial/synthetic sequence
  source 1..21: mol_type = other DNA
tatggttctgtagagatattt

SEQ ID NO: 322 - DNA, 21 bp, synthetic construct
  misc_feature 1..21: note = UBE3A-ATS artificial/synthetic sequence
  source 1..21: mol_type = other DNA
tgttctcaatttccctatata

SEQ ID NO: 323 - DNA, 21 bp, synthetic construct
  misc_feature 1..21: note = UBE3A-ATS artificial/synthetic sequence
  source 1..21: mol_type = other DNA
gttctcaatttccctatataa

SEQ ID NO: 324 - DNA, 21 bp, synthetic construct
  misc_feature 1..21: note = UBE3A-ATS artificial/synthetic sequence
  source 1..21: mol_type = other DNA
aggttggaacatttcaaataa

SEQ ID NO: 325 - DNA, 21 bp, synthetic construct
  misc_feature 1..21: note = UBE3A-ATS artificial/synthetic sequence
  source 1..21: mol_type = other DNA
ttacatgggctgttctataaa

SEQ ID NO: 326 - DNA, 21 bp, synthetic construct
  misc_feature 1..21: note = UBE3A-ATS artificial/synthetic sequence
  source 1..21: mol_type = other DNA
tacatgggctgttctataaat

SEQ ID NO: 327 - DNA, 21 bp, synthetic construct
  misc_feature 1..21: note = UBE3A-ATS artificial/synthetic sequence
  source 1..21: mol_type = other DNA
tgttacttaaggtggttaata

SEQ ID NO: 328 - DNA, 21 bp, synthetic construct
  misc_feature 1..21: note = UBE3A-ATS artificial/synthetic sequence
  source 1..21: mol_type = other DNA
gttacttaaggtggttaataa

SEQ ID NO: 329 - DNA, 21 bp, synthetic construct
  misc_feature 1..21: note = UBE3A-ATS artificial/synthetic sequence
  source 1..21: mol_type = other DNA
gttgctcaagtcttctatatt

SEQ ID NO: 330 - DNA, 21 bp, synthetic construct
  misc_feature 1..21: note = UBE3A-ATS artificial/synthetic sequence
  source 1..21: mol_type = other DNA
caacatgcaggtttgttatat

SEQ ID NO: 331 - DNA, 21 bp, synthetic construct
  misc_feature 1..21: note = UBE3A-ATS artificial/synthetic sequence
  source 1..21: mol_type = other DNA
acatgcaggtttgttatatat

SEQ ID NO: 332 - DNA, 21 bp, synthetic construct
  misc_feature 1..21: note = UBE3A-ATS artificial/synthetic sequence
  source 1..21: mol_type = other DNA
acgtgtgcatgtgtctttata

SEQ ID NO: 333 - DNA, 21 bp, synthetic construct
  misc_feature 1..21: note = UBE3A-ATS artificial/synthetic sequence
  source 1..21: mol_type = other DNA
ctttatagcagcatgatttat

SEQ ID NO: 334 - DNA, 21 bp, synthetic construct
  misc_feature 1..21: note = UBE3A-ATS artificial/synthetic sequence
  source 1..21: mol_type = other DNA
tgtgtctttggctgcataaat

SEQ ID NO: 335 - DNA, 21 bp, synthetic construct
  misc_feature 1..21: note = UBE3A-ATS artificial/synthetic sequence
  source 1..21: mol_type = other DNA
ctttgtagattctggatatta

SEQ ID NO: 336 - DNA, 21 bp, synthetic construct
  misc_feature 1..21: note = UBE3A-ATS artificial/synthetic sequence
  source 1..21: mol_type = other DNA
gcagaagctctttagtttaat

SEQ ID NO: 337 - DNA, 21 bp, synthetic construct
  misc_feature 1..21: note = UBE3A-ATS artificial/synthetic sequence
  source 1..21: mol_type = other DNA
tttcccagcaccatttattaa

SEQ ID NO: 338 - DNA, 21 bp, synthetic construct
  misc_feature 1..21: note = UBE3A-ATS artificial/synthetic sequence
  source 1..21: mol_type = other DNA
ttcccagcaccatttattaaa

SEQ ID NO: 339 - DNA, 21 bp, synthetic construct
  misc_feature 1..21: note = UBE3A-ATS artificial/synthetic sequence
  source 1..21: mol_type = other DNA
tcccagcaccatttattaaat

SEQ ID NO: 340 - DNA, 21 bp, synthetic construct
  misc_feature 1..21: note = UBE3A-ATS artificial/synthetic sequence
  source 1..21: mol_type = other DNA
cccagcaccatttattaaata

SEQ ID NO: 341 - DNA, 21 bp, synthetic construct
  misc_feature 1..21: note = UBE3A-ATS artificial/synthetic sequence
  source 1..21: mol_type = other DNA
gttgtagatgtgtggtattat

SEQ ID NO: 342 - DNA, 21 bp, synthetic construct
  misc_feature 1..21: note = UBE3A-ATS artificial/synthetic sequence
  source 1..21: mol_type = other DNA
ttgtagatgtgtggtattatt

SEQ ID NO: 343 - DNA, 21 bp, synthetic construct
  misc_feature 1..21: note = UBE3A-ATS artificial/synthetic sequence
  source 1..21: mol_type = other DNA
tgtagatgtgtggtattattt

SEQ ID NO: 344 - DNA, 21 bp, synthetic construct
  misc_feature 1..21: note = UBE3A-ATS artificial/synthetic sequence
  source 1..21: mol_type = other DNA
gttctgttccattggtttata

SEQ ID NO: 345 - DNA, 21 bp, synthetic construct
  misc_feature 1..21: note = UBE3A-ATS artificial/synthetic sequence
  source 1..21: mol_type = other DNA
ttctgttccattggtttatat

SEQ ID NO: 346 - DNA, 21 bp, synthetic construct
  misc_feature 1..21: note = UBE3A-ATS artificial/synthetic sequence
  source 1..21: mol_type = other DNA
ggatggcattgaatctataaa

SEQ ID NO: 347 - DNA, 21 bp, synthetic construct
  misc_feature 1..21: note = UBE3A-ATS artificial/synthetic sequence
  source 1..21: mol_type = other DNA
gatggcattgaatctataaat

SEQ ID NO: 348 - DNA, 21 bp, synthetic construct
  misc_feature 1..21: note = UBE3A-ATS artificial/synthetic sequence
  source 1..21: mol_type = other DNA
cctaattgaataccctttatt

SEQ ID NO: 349 - DNA, 21 bp, synthetic construct
  misc_feature 1..21: note = UBE3A-ATS artificial/synthetic sequence
  source 1..21: mol_type = other DNA
ggctgtgggtttgtcataaat

SEQ ID NO: 350 - DNA, 21 bp, synthetic construct
  misc_feature 1..21: note = UBE3A-ATS artificial/synthetic sequence
  source 1..21: mol_type = other DNA
gctgtgggtttgtcataaata

SEQ ID NO: 351 - DNA, 21 bp, synthetic construct
  misc_feature 1..21: note = UBE3A-ATS artificial/synthetic sequence
  source 1..21: mol_type = other DNA
tgtcccatcaatacctaattt

SEQ ID NO: 352 - DNA, 21 bp, synthetic construct
  misc_feature 1..21: note = UBE3A-ATS artificial/synthetic sequence
  source 1..21: mol_type = other DNA
gtcccatcaatacctaattta

SEQ ID NO: 353 - DNA, 21 bp, synthetic construct
  misc_feature 1..21: note = UBE3A-ATS artificial/synthetic sequence
  source 1..21: mol_type = other DNA
tcccatcaatacctaatttat

SEQ ID NO: 354 - DNA, 21 bp, synthetic construct
  misc_feature 1..21: note = UBE3A-ATS artificial/synthetic sequence
  source 1..21: mol_type = other DNA
cccatcaatacctaatttatt

SEQ ID NO: 355 - DNA, 21 bp, synthetic construct
  misc_feature 1..21: note = UBE3A-ATS artificial/synthetic sequence
  source 1..21: mol_type = other DNA
ttgtctttggttctgtttata

SEQ ID NO: 356 - DNA, 21 bp, synthetic construct
  misc_feature 1..21: note = UBE3A-ATS artificial/synthetic sequence
  source 1..21: mol_type = other DNA
tgtctttggttctgtttatat

SEQ ID NO: 357 - DNA, 21 bp, synthetic construct
  misc_feature 1..21: note = UBE3A-ATS artificial/synthetic sequence
  source 1..21: mol_type = other DNA
agcatgctttgctggtattaa

SEQ ID NO: 358 - DNA, 21 bp, synthetic construct
  misc_feature 1..21: note = UBE3A-ATS artificial/synthetic sequence
  source 1..21: mol_type = other DNA
gcatgctttgctggtattaat

SEQ ID NO: 359 - DNA, 21 bp, synthetic construct
  misc_feature 1..21: note = UBE3A-ATS artificial/synthetic sequence
  source 1..21: mol_type = other DNA
catgctttgctggtattaata

SEQ ID NO: 360 - DNA, 21 bp, synthetic construct
  misc_feature 1..21: note = UBE3A-ATS artificial/synthetic sequence
  source 1..21: mol_type = other DNA
atgctttgctggtattaatat

SEQ ID NO: 361 - DNA, 21 bp, synthetic construct
  misc_feature 1..21: note = UBE3A-ATS artificial/synthetic sequence
  source 1..21: mol_type = other DNA
tgctttgctggtattaatata

SEQ ID NO: 362 - DNA, 21 bp, synthetic construct
  misc_feature 1..21: note = UBE3A-ATS artificial/synthetic sequence
  source 1..21: mol_type = other DNA
gagagttagaacctcaatata

SEQ ID NO: 363 - DNA, 21 bp, synthetic construct
  misc_feature 1..21: note = UBE3A-ATS artificial/synthetic sequence
  source 1..21: mol_type = other DNA
agagttagaacctcaatatat

SEQ ID NO: 364 - DNA, 21 bp, synthetic construct
  misc_feature 1..21: note = UBE3A-ATS artificial/synthetic sequence
  source 1..21: mol_type = other DNA
ccaccacgcctggctttataa

SEQ ID NO: 365 - DNA, 21 bp, synthetic construct
  misc_feature 1..21: note = UBE3A-ATS artificial/synthetic sequence
  source 1..21: mol_type = other DNA
caccacgcctggctttataat

SEQ ID NO: 366 - DNA, 21 bp, synthetic construct
  misc_feature 1..21: note = UBE3A-ATS artificial/synthetic sequence
  source 1..21: mol_type = other DNA
accacgcctggctttataatt

SEQ ID NO: 367 - DNA, 21 bp, synthetic construct
  misc_feature 1..21: note = UBE3A-ATS artificial/synthetic sequence
  source 1..21: mol_type = other DNA
ccacgcctggctttataattt

SEQ ID NO: 368 - DNA, 21 bp, synthetic construct
  misc_feature 1..21: note = UBE3A-ATS artificial/synthetic sequence
  source 1..21: mol_type = other DNA
gtctagctccaagtgatatat

SEQ ID NO: 369 - DNA, 21 bp, synthetic construct
  misc_feature 1..21: note = UBE3A-ATS artificial/synthetic sequence
  source 1..21: mol_type = other DNA
tttgcttctgtctgaaatata

SEQ ID NO: 370 - DNA, 21 bp, synthetic construct
  misc_feature 1..21: note = UBE3A-ATS artificial/synthetic sequence
  source 1..21: mol_type = other DNA
ttgcttctgtctgaaatatat

SEQ ID NO: 371 - DNA, 21 bp, synthetic construct
  misc_feature 1..21: note = UBE3A-ATS artificial/synthetic sequence
  source 1..21: mol_type = other DNA
tcttaagtctgtgagtttata

SEQ ID NO: 372 - DNA, 21 bp, synthetic construct
  misc_feature 1..21: note = UBE3A-ATS artificial/synthetic sequence
  source 1..21: mol_type = other DNA
tctctgatttccacctatatt

SEQ ID NO: 373 - DNA, 21 bp, synthetic construct
  misc_feature 1..21: note = UBE3A-ATS artificial/synthetic sequence
  source 1..21: mol_type = other DNA
ctctgatttccacctatatta

SEQ ID NO: 374 - DNA, 21 bp, synthetic construct
  misc_feature 1..21: note = UBE3A-ATS artificial/synthetic sequence
  source 1..21: mol_type = other DNA
gtatatactccagagaaataa

SEQ ID NO: 375 - DNA, 21 bp, synthetic construct
  misc_feature 1..21: note = UBE3A-ATS artificial/synthetic sequence
  source 1..21: mol_type = other DNA
caccagaacttgaacattaat

SEQ ID NO: 376 - DNA, 21 bp, synthetic construct
  misc_feature 1..21: note = UBE3A-ATS artificial/synthetic sequence
  source 1..21: mol_type = other DNA
accagaacttgaacattaatt

SEQ ID NO: 377 - DNA, 21 bp, synthetic construct
  misc_feature 1..21: note = UBE3A-ATS artificial/synthetic sequence
  source 1..21: mol_type = other DNA
ccagaacttgaacattaattt

SEQ ID NO: 378 - DNA, 21 bp, synthetic construct
  misc_feature 1..21: note = UBE3A-ATS artificial/synthetic sequence
  source 1..21: mol_type = other DNA
gtctggaactcctggaattaa

SEQ ID NO: 379 - DNA, 21 bp, synthetic construct
  misc_feature 1..21: note = UBE3A-ATS artificial/synthetic sequence
  source 1..21: mol_type = other DNA
ctttctgtgcctggcttattt

SEQ ID NO: 380 - DNA, 21 bp, synthetic construct
  misc_feature 1..21: note = UBE3A-ATS artificial/synthetic sequence
  source 1..21: mol_type = other DNA
caggatttaccttcctttaaa

SEQ ID NO: 381 - DNA, 21 bp, synthetic construct
  misc_feature 1..21: note = UBE3A-ATS artificial/synthetic sequence
  source 1..21: mol_type = other DNA
tctgttgatgggcacttaaat

SEQ ID NO: 382 - DNA, 21 bp, synthetic construct
  misc_feature 1..21: note = UBE3A-ATS artificial/synthetic sequence
  source 1..21: mol_type = other DNA
ctgttgatgggcacttaaatt

SEQ ID NO: 383 - DNA, 21 bp, synthetic construct
  misc_feature 1..21: note = UBE3A-ATS artificial/synthetic sequence
  source 1..21: mol_type = other DNA
tcatagcggctgtactaattt

SEQ ID NO: 384 - DNA, 21 bp, synthetic construct
  misc_feature 1..21: note = UBE3A-ATS artificial/synthetic sequence
  source 1..21: mol_type = other DNA
catagcggctgtactaattta

SEQ ID NO: 385 - DNA, 21 bp, synthetic construct
  misc_feature 1..21: note = UBE3A-ATS artificial/synthetic sequence
  source 1..21: mol_type = other DNA
tttacccatttccagatatat

SEQ ID NO: 386 - DNA, 21 bp, synthetic construct
  misc_feature 1..21: note = UBE3A-ATS artificial/synthetic sequence
  source 1..21: mol_type = other DNA
gtgtaaataagggtctaattt

SEQ ID NO: 387 - DNA, 21 bp, synthetic construct
  misc_feature 1..21: note = UBE3A-ATS artificial/synthetic sequence
  source 1..21: mol_type = other DNA
tggttatgtcatcagtaatta

SEQ ID NO: 388 - DNA, 21 bp, synthetic construct
  misc_feature 1..21: note = UBE3A-ATS artificial/synthetic sequence
  source 1..21: mol_type = other DNA
ccttgcatcctaaggataaat

SEQ ID NO: 389 - DNA, 21 bp, synthetic construct
  misc_feature 1..21: note = UBE3A-ATS artificial/synthetic sequence
  source 1..21: mol_type = other DNA
gtgaatccagtttgctaatat

SEQ ID NO: 390 - DNA, 21 bp, synthetic construct
  misc_feature 1..21: note = UBE3A-ATS artificial/synthetic sequence
  source 1..21: mol_type = other DNA
tgaatccagtttgctaatata

SEQ ID NO: 391 - DNA, 21 bp, synthetic construct
  misc_feature 1..21: note = UBE3A-ATS artificial/synthetic sequence
  source 1..21: mol_type = other DNA
gaatccagtttgctaatatat

SEQ ID NO: 392 - DNA, 21 bp, synthetic construct
  misc_feature 1..21: note = UBE3A-ATS artificial/synthetic sequence
  source 1..21: mol_type = other DNA
ccatgttcatcagggatatta

SEQ ID NO: 393 - DNA, 21 bp, synthetic construct
  misc_feature 1..21: note = UBE3A-ATS artificial/synthetic sequence
  source 1..21: mol_type = other DNA
gtgtctttctctggctttaat

SEQ ID NO: 394 - DNA, 21 bp, synthetic construct
  misc_feature 1..21: note = UBE3A-ATS artificial/synthetic sequence
  source 1..21: mol_type = other DNA
tgtctttctctggctttaata

SEQ ID NO: 395 - DNA, 21 bp, synthetic construct
  misc_feature 1..21: note = UBE3A-ATS artificial/synthetic sequence
  source 1..21: mol_type = other DNA
gtctttctctggctttaataa

SEQ ID NO: 396 - DNA, 21 bp, synthetic construct
  misc_feature 1..21: note = UBE3A-ATS artificial/synthetic sequence
  source 1..21: mol_type = other DNA
ttgtgatccttcttctttatt

SEQ ID NO: 397 - DNA, 21 bp, synthetic construct
  misc_feature 1..21: note = UBE3A-ATS artificial/synthetic sequence
  source 1..21: mol_type = other DNA
gtaggtttgtatcgctataaa

SEQ ID NO: 398 - DNA, 21 bp, synthetic construct
  misc_feature 1..21: note = UBE3A-ATS artificial/synthetic sequence
  source 1..21: mol_type = other DNA
taggtttgtatcgctataaat

SEQ ID NO: 399 - DNA, 21 bp, synthetic construct
  misc_feature 1..21: note = UBE3A-ATS artificial/synthetic sequence
  source 1..21: mol_type = other DNA
aggtttgtatcgctataaatt

SEQ ID NO: 400 - DNA, 21 bp, synthetic construct
  misc_feature 1..21: note = UBE3A-ATS artificial/synthetic sequence
  source 1..21: mol_type = other DNA
ggtttgtatcgctataaattt

SEQ ID NO: 401 - DNA, 21 bp, synthetic construct
  misc_feature 1..21: note = UBE3A-ATS artificial/synthetic sequence
  source 1..21: mol_type = other DNA
tcatttgtctcaaggtaattt

SEQ ID NO: 402 - DNA, 21 bp, synthetic construct
  misc_feature 1..21: note = UBE3A-ATS artificial/synthetic sequence
  source 1..21: mol_type = other DNA
tttgggagcatattgtttaat

SEQ ID NO: 403 - DNA, 21 bp, synthetic construct
  misc_feature 1..21: note = UBE3A-ATS artificial/synthetic sequence
  source 1..21: mol_type = other DNA
ttgggagcatattgtttaatt

SEQ ID NO: 404 - DNA, 21 bp, synthetic construct
  misc_feature 1..21: note = UBE3A-ATS artificial/synthetic sequence
  source 1..21: mol_type = other DNA
tgggagcatattgtttaattt

SEQ ID NO: 405 - DNA, 21 bp, synthetic construct
  misc_feature 1..21: note = UBE3A-ATS artificial/synthetic sequence
  source 1..21: mol_type = other DNA
tggatggaatgtttcatatat

SEQ ID NO: 406 - DNA, 21 bp, synthetic construct
  misc_feature 1..21: note = UBE3A-ATS artificial/synthetic sequence
  source 1..21: mol_type = other DNA
agtattgaggtcccgtattat

SEQ ID NO: 407 - DNA, 21 bp, synthetic construct
  misc_feature 1..21: note = UBE3A-ATS artificial/synthetic sequence
  source 1..21: mol_type = other DNA
gtattgaggtcccgtattata

SEQ ID NO: 408 - DNA, 21 bp, synthetic construct
  misc_feature 1..21: note = UBE3A-ATS artificial/synthetic sequence
  source 1..21: mol_type = other DNA
ctccctcttcacatcatttaa

SEQ ID NO: 409 - DNA, 21 bp, synthetic construct
  misc_feature 1..21: note = UBE3A-ATS artificial/synthetic sequence
  source 1..21: mol_type = other DNA
tccctcttcacatcatttaaa

SEQ ID NO: 410 - DNA, 21 bp, synthetic construct
  misc_feature 1..21: note = UBE3A-ATS artificial/synthetic sequence
  source 1..21: mol_type = other DNA
tatgtcttcttggtgaattaa

SEQ ID NO: 411 - DNA, 21 bp, synthetic construct
  misc_feature 1..21: note = UBE3A-ATS artificial/synthetic sequence
  source 1..21: mol_type = other DNA
atgtcttcttggtgaattaat

SEQ ID NO: 412 - DNA, 21 bp, synthetic construct
  misc_feature 1..21: note = UBE3A-ATS artificial/synthetic sequence
  source 1..21: mol_type = other DNA
ctgctctcaatttccatttat

SEQ ID NO: 413 - DNA, 21 bp, synthetic construct
  misc_feature 1..21: note = UBE3A-ATS artificial/synthetic sequence
  source 1..21: mol_type = other DNA
tgctctcaatttccatttata

SEQ ID NO: 414 - DNA, 21 bp, synthetic construct
  misc_feature 1..21: note = UBE3A-ATS artificial/synthetic sequence
  source 1..21: mol_type = other DNA
gctctcaatttccatttatat

SEQ ID NO: 415 - DNA, 21 bp, synthetic construct
  misc_feature 1..21: note = UBE3A-ATS artificial/synthetic sequence
  source 1..21: mol_type = other DNA
tccttcagcactttgaatata

SEQ ID NO: 416 - DNA, 21 bp, synthetic construct
  misc_feature 1..21: note = UBE3A-ATS artificial/synthetic sequence
  source 1..21: mol_type = other DNA
tcagctattacttccttaaat

SEQ ID NO: 417 - DNA, 21 bp, synthetic construct
  misc_feature 1..21: note = UBE3A-ATS artificial/synthetic sequence
  source 1..21: mol_type = other DNA
cagctattacttccttaaata

SEQ ID NO: 418 - DNA, 21 bp, synthetic construct
  misc_feature 1..21: note = UBE3A-ATS artificial/synthetic sequence
  source 1..21: mol_type = other DNA
tccttaaggacctcctattat

SEQ ID NO: 419 - DNA, 21 bp, synthetic construct
  misc_feature 1..21: note = UBE3A-ATS artificial/synthetic sequence
  source 1..21: mol_type = other DNA
gcttgacctctaaacatataa

SEQ ID NO: 420 - DNA, 21 bp, synthetic construct
  misc_feature 1..21: note = UBE3A-ATS artificial/synthetic sequence
  source 1..21: mol_type = other DNA
cttgacctctaaacatataaa

SEQ ID NO: 421 - DNA, 21 bp, synthetic construct
  misc_feature 1..21: note = UBE3A-ATS artificial/synthetic sequence
  source 1..21: mol_type = other DNA
accaataccttgtgtaataaa

SEQ ID NO: 422 - DNA, 21 bp, synthetic construct
  misc_feature 1..21: note = UBE3A-ATS artificial/synthetic sequence
  source 1..21: mol_type = other DNA
ttgacactggctctctttata

SEQ ID NO: 423 - DNA, 21 bp, synthetic construct
  misc_feature 1..21: note = UBE3A-ATS artificial/synthetic sequence
  source 1..21: mol_type = other DNA
tgacactggctctctttataa

SEQ ID NO: 424 - DNA, 21 bp, synthetic construct
  misc_feature 1..21: note = UBE3A-ATS artificial/synthetic sequence
  source 1..21: mol_type = other DNA
cagaagatgtgtttgataata

SEQ ID NO: 425 - DNA, 21 bp, synthetic construct
  misc_feature 1..21: note = UBE3A-ATS artificial/synthetic sequence
  source 1..21: mol_type = other DNA
gtttgacgtgaagagtttaaa

SEQ ID NO: 426 - DNA, 21 bp, synthetic construct
  misc_feature 1..21: note = UBE3A-ATS artificial/synthetic sequence
  source 1..21: mol_type = other DNA
ctctgagcttcagtgaattat

SEQ ID NO: 427 - DNA, 21 bp, synthetic construct
  misc_feature 1..21: note = UBE3A-ATS artificial/synthetic sequence
  source 1..21: mol_type = other DNA
agggttgaatgctggatttaa

SEQ ID NO: 428 - DNA, 21 bp, synthetic construct
  misc_feature 1..21: note = UBE3A-ATS artificial/synthetic sequence
  source 1..21: mol_type = other DNA
gggttgaatgctggatttaaa

SEQ ID NO: 429 - DNA, 21 bp, synthetic construct
  misc_feature 1..21: note = UBE3A-ATS artificial/synthetic sequence
  source 1..21: mol_type = other DNA
ggttgaatgctggatttaaat

SEQ ID NO: 430 - DNA, 21 bp, synthetic construct
  misc_feature 1..21: note = UBE3A-ATS artificial/synthetic sequence
  source 1..21: mol_type = other DNA
gttgaatgctggatttaaata

SEQ ID NO: 431 - DNA, 21 bp, synthetic construct
  misc_feature 1..21: note = UBE3A-ATS artificial/synthetic sequence
  source 1..21: mol_type = other DNA
agtgaaagcaaagagaataaa

SEQ ID NO: 432 - DNA, 21 bp, synthetic construct
  misc_feature 1..21: note = UBE3A-ATS artificial/synthetic sequence
  source 1..21: mol_type = other DNA
catgatgttccaaactttaaa

SEQ ID NO: 433 - DNA, 21 bp, synthetic construct
  misc_feature 1..21: note = UBE3A-ATS artificial/synthetic sequence
  source 1..21: mol_type = other DNA
aggtggccaagggccttaata

SEQ ID NO: 434 - DNA, 21 bp, synthetic construct
  misc_feature 1..21: note = UBE3A-ATS artificial/synthetic sequence
  source 1..21: mol_type = other DNA
gcccagctggctctgtaataa

SEQ ID NO: 435 - DNA, 21 bp, synthetic construct
  misc_feature 1..21: note = UBE3A-ATS artificial/synthetic sequence
  source 1..21: mol_type = other DNA
cccagctggctctgtaataaa

SEQ ID NO: 436 - DNA, 21 bp, synthetic construct
  misc_feature 1..21: note = UBE3A-ATS artificial/synthetic sequence
  source 1..21: mol_type = other DNA
ccagctggctctgtaataaat

SEQ ID NO: 437 - DNA, 21 bp, synthetic construct
  misc_feature 1..21: note = UBE3A-ATS artificial/synthetic sequence
  source 1..21: mol_type = other DNA
tggtgtcatcgggccatattt

SEQ ID NO: 438 - DNA, 21 bp, synthetic construct
  misc_feature 1..21: note = UBE3A-ATS artificial/synthetic sequence
  source 1..21: mol_type = other DNA
taactaggtcaacagaatatt

SEQ ID NO: 439 - DNA, 21 bp, synthetic construct
  misc_feature 1..21: note = UBE3A-ATS artificial/synthetic sequence
  source 1..21: mol_type = other DNA
gcaaagttaatcctcatataa

SEQ ID NO: 440 - DNA, 21 bp, synthetic construct
  misc_feature 1..21: note = UBE3A-ATS artificial/synthetic sequence
  source 1..21: mol_type = other DNA
gcagcatctgttctgattaaa

SEQ ID NO: 441 - DNA, 21 bp, synthetic construct
  misc_feature 1..21: note = UBE3A-ATS artificial/synthetic sequence
  source 1..21: mol_type = other DNA
cagcatctgttctgattaaat

SEQ ID NO: 442 - DNA, 21 bp, synthetic construct
  misc_feature 1..21: note = UBE3A-ATS artificial/synthetic sequence
  source 1..21: mol_type = other DNA
agcatctgttctgattaaata

SEQ ID NO: 443 - DNA, 21 bp, synthetic construct
  misc_feature 1..21: note = UBE3A-ATS artificial/synthetic sequence
  source 1..21: mol_type = other DNA
gcatctgttctgattaaatat

SEQ ID NO: 444 - DNA, 21 bp, synthetic construct
  misc_feature 1..21: note = UBE3A-ATS artificial/synthetic sequence
  source 1..21: mol_type = other DNA
ctgcacaactgaccctttatt

SEQ ID NO: 445 - DNA, 21 bp, synthetic construct
  misc_feature 1..21: note = UBE3A-ATS artificial/synthetic sequence
  source 1..21: mol_type = other DNA
tgcacaactgaccctttatta

SEQ ID NO: 446 - DNA, 21 bp, synthetic construct
  misc_feature 1..21: note = UBE3A-ATS artificial/synthetic sequence
  source 1..21: mol_type = other DNA
tgatggctttcctactattta

SEQ ID NO: 447 - DNA, 21 bp, synthetic construct
  misc_feature 1..21: note = UBE3A-ATS artificial/synthetic sequence
  source 1..21: mol_type = other DNA
gatggctttcctactatttaa

SEQ ID NO: 448 - DNA, 21 bp, synthetic construct
  misc_feature 1..21: note = UBE3A-ATS artificial/synthetic sequence
  source 1..21: mol_type = other DNA
agctcagccatctggtttaaa

SEQ ID NO: 449 - DNA, 21 bp, synthetic construct
  misc_feature 1..21: note = UBE3A-ATS artificial/synthetic sequence
  source 1..21: mol_type = other DNA
ctgactgccttagactaatat

SEQ ID NO: 450 - DNA, 21 bp, synthetic construct
  misc_feature 1..21: note = UBE3A-ATS artificial/synthetic sequence
  source 1..21: mol_type = other DNA
tgactgccttagactaatata

SEQ ID NO: 451 - DNA, 21 bp, synthetic construct
  misc_feature 1..21: note = UBE3A-ATS artificial/synthetic sequence
  source 1..21: mol_type = other DNA
cagcatttgacaaagaaataa

SEQ ID NO: 452 - DNA, 21 bp, synthetic construct
  misc_feature 1..21: note = UBE3A-ATS artificial/synthetic sequence
  source 1..21: mol_type = other DNA
aggatatggatgatgtatata

SEQ ID NO: 453 - DNA, 21 bp, synthetic construct
  misc_feature 1..21: note = UBE3A-ATS artificial/synthetic sequence
  source 1..21: mol_type = other DNA
gtcgaccctaagaagataata

SEQ ID NO: 454 - DNA, 21 bp, synthetic construct
  misc_feature 1..21: note = UBE3A-ATS artificial/synthetic sequence
  source 1..21: mol_type = other DNA
agaaacctgaacaagtaataa

SEQ ID NO: 455 - DNA, 21 bp, synthetic construct
  misc_feature 1..21: note = UBE3A-ATS artificial/synthetic sequence
  source 1..21: mol_type = other DNA
gaaacctgaacaagtaataat

SEQ ID NO: 456 - DNA, 21 bp, synthetic construct
  misc_feature 1..21: note = UBE3A-ATS artificial/synthetic sequence
  source 1..21: mol_type = other DNA
agtcacacaacagactaaatt

SEQ ID NO: 457 - DNA, 21 bp, synthetic construct
  misc_feature 1..21: note = UBE3A-ATS artificial/synthetic sequence
  source 1..21: mol_type = other DNA
gtcacacaacagactaaattt

SEQ ID NO: 458 - DNA, 21 bp, synthetic construct
  misc_feature 1..21: note = UBE3A-ATS artificial/synthetic sequence
  source 1..21: mol_type = other DNA
agcgagagtgtcctcatttat

SEQ ID NO: 459 - DNA, 21 bp, synthetic construct
  misc_feature 1..21: note = UBE3A-ATS artificial/synthetic sequence
  source 1..21: mol_type = other DNA
gcgagagtgtcctcatttata

SEQ ID NO: 460 - DNA, 21 bp, synthetic construct
  misc_feature 1..21: note = UBE3A-ATS artificial/synthetic sequence
  source 1..21: mol_type = other DNA
cgagagtgtcctcatttatat

SEQ ID NO: 461 - DNA, 21 bp, synthetic construct
  misc_feature 1..21: note = UBE3A-ATS artificial/synthetic sequence
  source 1..21: mol_type = other DNA
atcctctatctctgttaataa

SEQ ID NO: 462 - DNA, 21 bp, synthetic construct
  misc_feature 1..21: note = UBE3A-ATS artificial/synthetic sequence
  source 1..21: mol_type = other DNA
agcaagtgctattccatattt

SEQ ID NO: 463 - DNA, 21 bp, synthetic construct
  misc_feature 1..21: note = UBE3A-ATS artificial/synthetic sequence
  source 1..21: mol_type = other DNA
ctggagtccatggagaaatat

SEQ ID NO: 464 - DNA, 21 bp, synthetic construct
  misc_feature 1..21: note = UBE3A-ATS artificial/synthetic sequence
  source 1..21: mol_type = other DNA
cctccaaggacatactttata

SEQ ID NO: 465 - DNA, 21 bp, synthetic construct
  misc_feature 1..21: note = UBE3A-ATS artificial/synthetic sequence
  source 1..21: mol_type = other DNA
ctccaaggacatactttataa

SEQ ID NO: 466 - DNA, 21 bp, synthetic construct
  misc_feature 1..21: note = UBE3A-ATS artificial/synthetic sequence
  source 1..21: mol_type = other DNA
tccaaggacatactttataat

SEQ ID NO: 467 - DNA, 21 bp, synthetic construct
  misc_feature 1..21: note = UBE3A-ATS artificial/synthetic sequence
  source 1..21: mol_type = other DNA
ccaaggacatactttataata

SEQ ID NO: 468 - DNA, 21 bp, synthetic construct
  misc_feature 1..21: note = UBE3A-ATS artificial/synthetic sequence
  source 1..21: mol_type = other DNA
gtgagggcaatggtgaatata

SEQ ID NO: 469 - DNA, 21 bp, synthetic construct
  misc_feature 1..21: note = UBE3A-ATS artificial/synthetic sequence
  source 1..21: mol_type = other DNA
tgagggcaatggtgaatataa

SEQ ID NO: 470 - DNA, 21 bp, synthetic construct
  misc_feature 1..21: note = UBE3A-ATS artificial/synthetic sequence
  source 1..21: mol_type = other DNA
cgaaaggcaagcttcttaaat

SEQ ID NO: 471 - DNA, 21 bp, synthetic construct
  misc_feature 1..21: note = UBE3A-ATS artificial/synthetic sequence
  source 1..21: mol_type = other DNA
gaaaggcaagcttcttaaata

SEQ ID NO: 472 - DNA, 21 bp, synthetic construct
  misc_feature 1..21: note = UBE3A-ATS artificial/synthetic sequence
  source 1..21: mol_type = other DNA
ggcaagcaaggcaggatttaa

SEQ ID NO: 473 - DNA, 21 bp, synthetic construct
  misc_feature 1..21: note = UBE3A-ATS artificial/synthetic sequence
  source 1..21: mol_type = other DNA
acaggaacctggctctaattt

SEQ ID NO: 474 - DNA, 21 bp, synthetic construct
  misc_feature 1..21: note = UBE3A-ATS artificial/synthetic sequence
  source 1..21: mol_type = other DNA
gggacctcctgacatttaatt

SEQ ID NO: 475 - DNA, 21 bp, synthetic construct
  misc_feature 1..21: note = UBE3A-ATS artificial/synthetic sequence
  source 1..21: mol_type = other DNA
ggacctcctgacatttaatta

SEQ ID NO: 476 - DNA, 21 bp, synthetic construct
  misc_feature 1..21: note = UBE3A-ATS artificial/synthetic sequence
  source 1..21: mol_type = other DNA
gacctcctgacatttaattaa

SEQ ID NO: 477 - DNA, 21 bp, synthetic construct
  misc_feature 1..21: note = UBE3A-ATS artificial/synthetic sequence
  source 1..21: mol_type = other DNA
gtgggagcttgttacatatat

SEQ ID NO: 478 - DNA, 21 bp, synthetic construct
  misc_feature 1..21: note = UBE3A-ATS artificial/synthetic sequence
  source 1..21: mol_type = other DNA
ctaagtcttctcatctatata

SEQ ID NO: 479 - DNA, 21 bp, synthetic construct
  misc_feature 1..21: note = UBE3A-ATS artificial/synthetic sequence
  source 1..21: mol_type = other DNA
ttaggtggtcatgagataaat

SEQ ID NO: 480 - DNA, 21 bp, synthetic construct
  misc_feature 1..21: note = UBE3A-ATS artificial/synthetic sequence
  source 1..21: mol_type = other DNA
taggtggtcatgagataaata

SEQ ID NO: 481 - DNA, 21 bp, synthetic construct
  misc_feature 1..21: note = UBE3A-ATS artificial/synthetic sequence
  source 1..21: mol_type = other DNA
aggtggtcatgagataaatat

SEQ ID NO: 482 - DNA, 21 bp, synthetic construct
  misc_feature 1..21: note = UBE3A-ATS artificial/synthetic sequence
  source 1..21: mol_type = other DNA
ggtggtcatgagataaatata

SEQ ID NO: 483 - DNA, 21 bp, synthetic construct
  misc_feature 1..21: note = UBE3A-ATS artificial/synthetic sequence
  source 1..21: mol_type = other DNA
gtggtcatgagataaatatat

SEQ ID NO: 484 - DNA, 21 bp, synthetic construct
  misc_feature 1..21: note = UBE3A-ATS artificial/synthetic sequence
  source 1..21: mol_type = other DNA
cacaaagcacatggctaaata

SEQ ID NO: 485 - DNA, 21 bp, synthetic construct
  misc_feature 1..21: note = UBE3A-ATS artificial/synthetic sequence
  source 1..21: mol_type = other DNA
acaaagcacatggctaaataa

SEQ ID NO: 486 - DNA, 21 bp, synthetic construct
  misc_feature 1..21: note = UBE3A-ATS artificial/synthetic sequence
  source 1..21: mol_type = other DNA
cggttatgtagaagctattta

SEQ ID NO: 487 - DNA, 21 bp, synthetic construct
  misc_feature 1..21: note = UBE3A-ATS artificial/synthetic sequence
  source 1..21: mol_type = other DNA
ggttatgtagaagctatttaa

SEQ ID NO: 488 - DNA, 21 bp, synthetic construct
  misc_feature 1..21: note = UBE3A-ATS artificial/synthetic sequence
  source 1..21: mol_type = other DNA
agccacccttaattgaataat

SEQ ID NO: 489 - DNA, 21 bp, synthetic construct
  misc_feature 1..21: note = UBE3A-ATS artificial/synthetic sequence
  source 1..21: mol_type = other DNA
actaccctttactggtaattt

SEQ ID NO: 490-493 - skipped (4 entries)

SEQ ID NO: 494 - DNA, 46 bp, synthetic construct
  misc_feature 1..46: note = shRNA artificial/synthetic sequence
  source 1..46: mol_type = other DNA
atatcaccttacagaaattactcgagtaatttctgtaaggtgatat

SEQ ID NO: 495 - DNA, 44 bp, synthetic construct
  misc_feature 1..44: note = shRNA artificial/synthetic sequence
  source 1..44: mol_type = other DNA
tatcaccttacagaaattactcgagtaatttctgtaaggtgata

SEQ ID NO: 496 - DNA, 42 bp, synthetic construct
  misc_feature 1..42: note = shRNA artificial/synthetic sequence
  source 1..42: mol_type = other DNA
atcaccttacagaaattactcgagtaatttctgtaaggtgat

SEQ ID NO: 497 - DNA, 40 bp, synthetic construct
  misc_feature 1..40: note = shRNA artificial/synthetic sequence
  source 1..40: mol_type = other DNA
tcaccttacagaaattactcgagtaatttctgtaaggtga

SEQ ID NO: 498 - DNA, 46 bp, synthetic construct
  misc_feature 1..46: note = shRNA artificial/synthetic sequence
  source 1..46: mol_type = other DNA
gatatcaccttacagaaattctcgagaatttctgtaaggtgatatc

SEQ ID NO: 499 - DNA, 44 bp, synthetic construct
  misc_feature 1..44: note = shRNA artificial/synthetic sequence
  source 1..44: mol_type = other DNA
gatatcaccttacagaaatctcgagatttctgtaaggtgatatc

SEQ ID NO: 500 - DNA, 40 bp, synthetic construct
  misc_feature 1..40: note = shRNA artificial/synthetic sequence
  source 1..40: mol_type = other DNA
gatatcaccttacagaactcgagttctgtaaggtgatatc

SEQ ID NO: 501 - DNA, 38 bp, synthetic construct
  misc_feature 1..38: note = shRNA artificial/synthetic sequence
  source 1..38: mol_type = other DNA
gatatcaccttacagactcgagtctgtaaggtgatatc

SEQ ID NO: 502 - DNA, 48 bp, synthetic construct
  misc_feature 1..48: note = shRNA artificial/synthetic sequence
  source 1..48: mol_type = other DNA
tgctcttctttctactttattctcgagaataaagtagaaagaagagca

SEQ ID NO: 503 - DNA, 48 bp, synthetic construct
  misc_feature 1..48: note = shRNA artificial/synthetic sequence
  source 1..48: mol_type = other DNA
ctcaatccaataacctaatttctcgagaaattaggttattggattgag

SEQ ID NO: 504 - DNA, 48 bp, synthetic construct
  misc_feature 1..48: note = shRNA artificial/synthetic sequence
  source 1..48: mol_type = other DNA
ttagtcacatcccacaaatttctcgagaaatttgtgggatgtgactaa

SEQ ID NO: 505 - DNA, 48 bp, synthetic construct
  misc_feature 1..48: note = shRNA artificial/synthetic sequence
  source 1..48: mol_type = other DNA
tcctaggtcataatgataattctcgagaattatcattatgacctagga

SEQ ID NO: 506 - DNA, 50 bp, synthetic construct
  misc_feature 1..50: note = shRNA artificial/synthetic sequence
  variation 22..29: note = some n may or may not be present; "nnnnnnnn" can be CTCGAG (SEQ ID NO: 490), TCAAGAG (SEQ ID NO: 491), TTCG (SEQ ID NO: 492) or GAAGCTTG (SEQ ID NO: 493)
  source 1..50: mol_type = other DNA
gatatcaccttacagaaattannnnnnnntaatttctgtaaggtgatatc